SEQUENCE LISTING (1095 sequences)

SEQ ID NO: 1 - protein, 29 aa, Artificial Sequence
  Description of Artificial Sequence Synthetic peptide
MLSTKKTKTHDHYPSGRMRDPGWHDWRAS

SEQ ID NO: 2 - protein, 29 aa, Artificial Sequence
  Description of Artificial Sequence Synthetic peptide
MLSTAKGKTHDHWGGGRMRPPPWHDWRFS

SEQ ID NO: 3 - protein, 29 aa, Artificial Sequence
  Description of Artificial Sequence Synthetic peptide
MSSGAKGKWHDHYPSGRMRPPPWHDWRFS

SEQ ID NO: 4 - protein, 29 aa, Artificial Sequence
  Description of Artificial Sequence Synthetic peptide
WPSGAKGKWHDHYPGGRMRPPPWHDWRFS

SEQ ID NO: 5 - protein, 29 aa, Artificial Sequence
  Description of Artificial Sequence Synthetic peptide
WGSGAKGKTHDHWGSGRWRDPGWHDWRGS

SEQ ID NO: 6 - protein, 29 aa, Artificial Sequence
  Description of Artificial Sequence Synthetic peptide
FGSGAKGKTHDHWGGGRHRPPPWHDWRGS

SEQ ID NO: 7 - protein, 29 aa, Artificial Sequence
  Description of Artificial Sequence Synthetic peptide
FGSGAKGKTHDHYPSGRMRPPPWHDWRVR

SEQ ID NO: 8 - protein, 29 aa, Artificial Sequence
  Description of Artificial Sequence Synthetic peptide
MPSAAKGKWHDHYPGGRMRDPKWHDWRFS

SEQ ID NO: 9 - protein, 29 aa, Artificial Sequence
  Description of Artificial Sequence Synthetic peptide
VTSTAKGKTHDHNWSGRYRPPPWHDWRAR

SEQ ID NO: 10 - protein, 29 aa, Artificial Sequence
  Description of Artificial Sequence Synthetic peptide
FVSGAKGKWHDHWPSGRWRDPPWHDWRAS

SEQ ID NO: 11 - protein, 29 aa, Artificial Sequence
  Description of Artificial Sequence Synthetic peptide
MASAAKGKWHDHWGSGRHRPPPWHDWRVS

SEQ ID NO: 12 - protein, 29 aa, Artificial Sequence
  Description of Artificial Sequence Synthetic peptide
MPSAAKGKWHDHYPSGRMRPPPWHDWRAR

SEQ ID NO: 13 - protein, 29 aa, Artificial Sequence
  Description of Artificial Sequence Synthetic peptide
MPSGAKGKTHDHYGGGRMRPPPWHDWRFS

SEQ ID NO: 14 - protein, 29 aa, Artificial Sequence
  Description of Artificial Sequence Synthetic peptide
FSSAAKGKWHDHWPGGRYRDPPWHDWRVS

SEQ ID NO: 15 - protein, 29 aa, Artificial Sequence
  Description of Artificial Sequence Synthetic peptide
FLSGAKGKTHDHYPSGRYRPPPWHDWRAR

SEQ ID NO: 16 - protein, 29 aa, Artificial Sequence
  Description of Artificial Sequence Synthetic peptide
MPSAAKTKWHDHYPGGRMRPPPWHDWRGR

SEQ ID NO: 17 - protein, 29 aa, Artificial Sequence
  Description of Artificial Sequence Synthetic peptide
WSSAAKGKTHDHWGGGRYRPPPWHDWRAS

SEQ ID NO: 18 - protein, 29 aa, Artificial Sequence
  Description of Artificial Sequence Synthetic peptide
PVSAAKGKWHDHNWSGRMRPPPWHDWRAR

SEQ ID NO: 19 - protein, 29 aa, Artificial Sequence
  Description of Artificial Sequence Synthetic peptide
WSSGAKTKWHDHYGSGRHRDPKWHDWRGS

SEQ ID NO: 20 - protein, 29 aa, Artificial Sequence
  Description of Artificial Sequence Synthetic peptide
WASGAKGKWHDHYGGGRYRDPGWHDWRGS

SEQ ID NO: 21 - protein, 29 aa, Artificial Sequence
  Description of Artificial Sequence Synthetic peptide
WGSAAKGKTHDHWPSGRYRDPKWHDWRVS

SEQ ID NO: 22 - protein, 29 aa, Artificial Sequence
  Description of Artificial Sequence Synthetic peptide
MLSGAKGKTHDHYWGGRMRPPPWHDWRGS

SEQ ID NO: 23 - protein, 29 aa, Artificial Sequence
  Description of Artificial Sequence Synthetic peptide
FGSAAKGKTHDHWPSGRHRDPGWHDWRFS

SEQ ID NO: 24 - protein, 29 aa, Artificial Sequence
  Description of Artificial Sequence Synthetic peptide
WVSAAKGKWHDHYGSGRMRDPPWHDWWGS

SEQ ID NO: 25 - protein, 29 aa, Artificial Sequence
  Description of Artificial Sequence Synthetic peptide
FVSAAKGKTHDHYPSGRYRPPPWHDWRAR

SEQ ID NO: 26 - protein, 29 aa, Artificial Sequence
  Description of Artificial Sequence Synthetic peptide
WPSAAKGKTHDHNPSGRMRPPPWHDWRFS

SEQ ID NO: 27 - protein, 29 aa, Artificial Sequence
  Description of Artificial Sequence Synthetic peptide
MASAAKTKWHDHYPSGRMRPPPWHDWRAR

SEQ ID NO: 28 - protein, 29 aa, Artificial Sequence
  Description of Artificial Sequence Synthetic peptide
MPSAAKTKWHDHYPSGRMRPPPWHDWRGR

SEQ ID NO: 29 - protein, 29 aa, Artificial Sequence
  Description of Artificial Sequence Synthetic peptide
MVSTAKGKTHDHYPSGRYRPPPWHDWRFS

SEQ ID NO: 30 - protein, 29 aa, Artificial Sequence
  Description of Artificial Sequence Synthetic peptide
WVSAAKGKTHDHWGSGRHRDPPWHDWRAR

SEQ ID NO: 31 - protein, 29 aa, Artificial Sequence
  Description of Artificial Sequence Synthetic peptide
WGSAAKGKTHDHWPSGRHRDPPWHDWRVR

SEQ ID NO: 32 - protein, 29 aa, Artificial Sequence
  Description of Artificial Sequence Synthetic peptide
WGSAAKGKTHDHYGSGRYRDPPWHDWRAR

SEQ ID NO: 33 - protein, 29 aa, Artificial Sequence
  Description of Artificial Sequence Synthetic peptide
FASAAKGKTHDHYWSGRYRPPPWHDWRAS

SEQ ID NO: 34 - protein, 29 aa, Artificial Sequence
  Description of Artificial Sequence Synthetic peptide
FGSAAKGKTHDHYWSGRMRPPPWHDWRGS

SEQ ID NO: 35 - protein, 29 aa, Artificial Sequence
  Description of Artificial Sequence Synthetic peptide
WSSTAKGKTHDHWPSGRYRPPPWHDWRAR

SEQ ID NO: 36 - protein, 29 aa, Artificial Sequence
  Description of Artificial Sequence Synthetic peptide
FLSTKKTKTHDHYPSGRMRDPGWHDWRAS

SEQ ID NO: 37 - protein, 29 aa, Artificial Sequence
  Description of Artificial Sequence Synthetic peptide
KLSTKKTKTHDHYPSGRMRDPGWHDWRAS

SEQ ID NO: 38 - protein, 29 aa, Artificial Sequence
  Description of Artificial Sequence Synthetic peptide
PLSTKKTKTHDHYPSGRMRDPGWHDWRAS

SEQ ID NO: 39 - protein, 29 aa, Artificial Sequence
  Description of Artificial Sequence Synthetic peptide
RLSTKKTKTHDHYPSGRMRDPGWHDWRAS

SEQ ID NO: 40 - protein, 29 aa, Artificial Sequence
  Description of Artificial Sequence Synthetic peptide
MASTKKTKTHDHYPSGRMRDPGWHDWRAS

SEQ ID NO: 41 - protein, 29 aa, Artificial Sequence
  Description of Artificial Sequence Synthetic peptide
MGSTKKTKTHDHYPSGRMRDPGWHDWRAS

SEQ ID NO: 42 - protein, 29 aa, Artificial Sequence
  Description of Artificial Sequence Synthetic peptide
MKSTKKTKTHDHYPSGRMRDPGWHDWRAS

SEQ ID NO: 43 - protein, 29 aa, Artificial Sequence
  Description of Artificial Sequence Synthetic peptide
MPSTKKTKTHDHYPSGRMRDPGWHDWRAS

SEQ ID NO: 44 - protein, 29 aa, Artificial Sequence
  Description of Artificial Sequence Synthetic peptide
MQSTKKTKTHDHYPSGRMRDPGWHDWRAS

SEQ ID NO: 45 - protein, 29 aa, Artificial Sequence
  Description of Artificial Sequence Synthetic peptide
MRSTKKTKTHDHYPSGRMRDPGWHDWRAS

SEQ ID NO: 46 - protein, 29 aa, Artificial Sequence
  Description of Artificial Sequence Synthetic peptide
MSSTKKTKTHDHYPSGRMRDPGWHDWRAS

SEQ ID NO: 47 - protein, 29 aa, Artificial Sequence
  Description of Artificial Sequence Synthetic peptide
MTSTKKTKTHDHYPSGRMRDPGWHDWRAS

SEQ ID NO: 48 - protein, 29 aa, Artificial Sequence
  Description of Artificial Sequence Synthetic peptide
MWSTKKTKTHDHYPSGRMRDPGWHDWRAS

SEQ ID NO: 49 - protein, 29 aa, Artificial Sequence
  Description of Artificial Sequence Synthetic peptide
MYSTKKTKTHDHYPSGRMRDPGWHDWRAS

SEQ ID NO: 50 - protein, 29 aa, Artificial Sequence
  Description of Artificial Sequence Synthetic peptide
MLQTKKTKTHDHYPSGRMRDPGWHDWRAS

SEQ ID NO: 51 - protein, 29 aa, Artificial Sequence
  Description of Artificial Sequence Synthetic peptide
MLRTKKTKTHDHYPSGRMRDPGWHDWRAS

SEQ ID NO: 52 - protein, 29 aa, Artificial Sequence
  Description of Artificial Sequence Synthetic peptide
MLSRKKTKTHDHYPSGRMRDPGWHDWRAS

SEQ ID NO: 53 - protein, 29 aa, Artificial Sequence
  Description of Artificial Sequence Synthetic peptide
MLSTRKTKTHDHYPSGRMRDPGWHDWRAS

SEQ ID NO: 54 - protein, 29 aa, Artificial Sequence
  Description of Artificial Sequence Synthetic peptide
MLSTKRTKTHDHYPSGRMRDPGWHDWRAS

SEQ ID NO: 55 - protein, 29 aa, Artificial Sequence
  Description of Artificial Sequence Synthetic peptide
MLSTKTTKTHDHYPSGRMRDPGWHDWRAS

SEQ ID NO: 56 - protein, 29 aa, Artificial Sequence
  Description of Artificial Sequence Synthetic peptide
MLSTKKKKTHDHYPSGRMRDPGWHDWRAS

SEQ ID NO: 57 - protein, 29 aa, Artificial Sequence
  Description of Artificial Sequence Synthetic peptide
MLSTKKRKTHDHYPSGRMRDPGWHDWRAS

SEQ ID NO: 58 - protein, 29 aa, Artificial Sequence
  Description of Artificial Sequence Synthetic peptide
MLSTKKTKRHDHYPSGRMRDPGWHDWRAS

SEQ ID NO: 59 - protein, 29 aa, Artificial Sequence
  Description of Artificial Sequence Synthetic peptide
MLSTKKTKTKDHYPSGRMRDPGWHDWRAS

SEQ ID NO: 60 - protein, 29 aa, Artificial Sequence
  Description of Artificial Sequence Synthetic peptide
MLSTKKTKTRDHYPSGRMRDPGWHDWRAS

SEQ ID NO: 61 - protein, 29 aa, Artificial Sequence
  Description of Artificial Sequence Synthetic peptide
MLSTKKTKTHFHYPSGRMRDPGWHDWRAS

SEQ ID NO: 62 - protein, 29 aa, Artificial Sequence
  Description of Artificial Sequence Synthetic peptide
MLSTKKTKTHGHYPSGRMRDPGWHDWRAS

SEQ ID NO: 63 - protein, 29 aa, Artificial Sequence
  Description of Artificial Sequence Synthetic peptide
MLSTKKTKTHHHYPSGRMRDPGWHDWRAS

SEQ ID NO: 64 - protein, 29 aa, Artificial Sequence
  Description of Artificial Sequence Synthetic peptide
MLSTKKTKTHIHYPSGRMRDPGWHDWRAS

SEQ ID NO: 65 - protein, 29 aa, Artificial Sequence
  Description of Artificial Sequence Synthetic peptide
MLSTKKTKTHKHYPSGRMRDPGWHDWRAS

SEQ ID NO: 66 - protein, 29 aa, Artificial Sequence
  Description of Artificial Sequence Synthetic peptide
MLSTKKTKTHPHYPSGRMRDPGWHDWRAS

SEQ ID NO: 67 - protein, 29 aa, Artificial Sequence
  Description of Artificial Sequence Synthetic peptide
MLSTKKTKTHRHYPSGRMRDPGWHDWRAS

SEQ ID NO: 68 - protein, 29 aa, Artificial Sequence
  Description of Artificial Sequence Synthetic peptide
MLSTKKTKTHTHYPSGRMRDPGWHDWRAS

SEQ ID NO: 69 - protein, 29 aa, Artificial Sequence
  Description of Artificial Sequence Synthetic peptide
MLSTKKTKTHVHYPSGRMRDPGWHDWRAS

SEQ ID NO: 70 - protein, 29 aa, Artificial Sequence
  Description of Artificial Sequence Synthetic peptide
MLSTKKTKTHWHYPSGRMRDPGWHDWRAS

SEQ ID NO: 71 - protein, 29 aa, Artificial Sequence
  Description of Artificial Sequence Synthetic peptide
MLSTKKTKTHYHYPSGRMRDPGWHDWRAS

SEQ ID NO: 72 - protein, 29 aa, Artificial Sequence
  Description of Artificial Sequence Synthetic peptide
MLSTKKTKTHDKYPSGRMRDPGWHDWRAS

SEQ ID NO: 73 - protein, 29 aa, Artificial Sequence
  Description of Artificial Sequence Synthetic peptide
MLSTKKTKTHDRYPSGRMRDPGWHDWRAS

SEQ ID NO: 74 - protein, 29 aa, Artificial Sequence
  Description of Artificial Sequence Synthetic peptide
MLSTKKTKTHDHGPSGRMRDPGWHDWRAS

SEQ ID NO: 75 - protein, 29 aa, Artificial Sequence
  Description of Artificial Sequence Synthetic peptide
MLSTKKTKTHDHKPSGRMRDPGWHDWRAS

SEQ ID NO: 76 - protein, 29 aa, Artificial Sequence
  Description of Artificial Sequence Synthetic peptide
MLSTKKTKTHDHRPSGRMRDPGWHDWRAS

SEQ ID NO: 77 - protein, 29 aa, Artificial Sequence
  Description of Artificial Sequence Synthetic peptide
MLSTKKTKTHDHWPSGRMRDPGWHDWRAS

SEQ ID NO: 78 - protein, 29 aa, Artificial Sequence
  Description of Artificial Sequence Synthetic peptide
MLSTKKTKTHDHYPRGRMRDPGWHDWRAS

SEQ ID NO: 79 - protein, 29 aa, Artificial Sequence
  Description of Artificial Sequence Synthetic peptide
MLSTKKTKTHDHYPSGRGRDPGWHDWRAS

SEQ ID NO: 80 - protein, 29 aa, Artificial Sequence
  Description of Artificial Sequence Synthetic peptide
MLSTKKTKTHDHYPSGRHRDPGWHDWRAS

SEQ ID NO: 81 - protein, 29 aa, Artificial Sequence
  Description of Artificial Sequence Synthetic peptide
MLSTKKTKTHDHYPSGRKRDPGWHDWRAS

SEQ ID NO: 82 - protein, 29 aa, Artificial Sequence
  Description of Artificial Sequence Synthetic peptide
MLSTKKTKTHDHYPSGRRRDPGWHDWRAS

SEQ ID NO: 83 - protein, 29 aa, Artificial Sequence
  Description of Artificial Sequence Synthetic peptide
MLSTKKTKTHDHYPSGRWRDPGWHDWRAS

SEQ ID NO: 84 - protein, 29 aa, Artificial Sequence
  Description of Artificial Sequence Synthetic peptide
MLSTKKTKTHDHYPSGRMRPPGWHDWRAS

SEQ ID NO: 85 - protein, 29 aa, Artificial Sequence
  Description of Artificial Sequence Synthetic peptide
MLSTKKTKTHDHYPSGRMRRPGWHDWRAS

SEQ ID NO: 86 - protein, 29 aa, Artificial Sequence
  Description of Artificial Sequence Synthetic peptide
MLSTKKTKTHDHYPSGRMRDPPWHDWRAS

SEQ ID NO: 87 - protein, 29 aa, Artificial Sequence
  Description of Artificial Sequence Synthetic peptide
MLSTKKTKTHDHYPSGRMRDPGWHPWRAS

SEQ ID NO: 88 - protein, 29 aa, Artificial Sequence
  Description of Artificial Sequence Synthetic peptide
MLSTKKTKTHDHYPSGRMRDPGWHRWRAS

SEQ ID NO: 89 - protein, 29 aa, Artificial Sequence
  Description of Artificial Sequence Synthetic peptide
MLSTKKTKTHDHYPSGRMRDPGWHDWWAS

SEQ ID NO: 90 - protein, 29 aa, Artificial Sequence
  Description of Artificial Sequence Synthetic peptide
MLSTKKTKTHDHYPSGRMRDPGWHDWRFS

SEQ ID NO: 91 - protein, 29 aa, Artificial Sequence
  Description of Artificial Sequence Synthetic peptide
MLSTKKTKTHDHYPSGRMRDPGWHDWRGS

SEQ ID NO: 92 - protein, 29 aa, Artificial Sequence
  Description of Artificial Sequence Synthetic peptide
MLSTKKTKTHDHYPSGRMRDPGWHDWRVS

SEQ ID NO: 93 - protein, 29 aa, Artificial Sequence
  Description of Artificial Sequence Synthetic peptide
MLSTKKTKTHDHYPSGRMRDPGWHDWRYS

SEQ ID NO: 94 - protein, 29 aa, Artificial Sequence
  Description of Artificial Sequence Synthetic peptide
SLSTKKTKTHDHYPSGRMRDPGWHDWRAS

SEQ ID NO: 95 - protein, 29 aa, Artificial Sequence
  Description of Artificial Sequence Synthetic peptide
VLSTKKTKTHDHYPSGRMRDPGWHDWRAS

SEQ ID NO: 96 - protein, 29 aa, Artificial Sequence
  Description of Artificial Sequence Synthetic peptide
WLSTKKTKTHDHYPSGRMRDPGWHDWRAS

SEQ ID NO: 97 - protein, 29 aa, Artificial Sequence
  Description of Artificial Sequence Synthetic peptide
MFSTKKTKTHDHYPSGRMRDPGWHDWRAS

SEQ ID NO: 98 - protein, 29 aa, Artificial Sequence
  Description of Artificial Sequence Synthetic peptide
MNSTKKTKTHDHYPSGRMRDPGWHDWRAS

SEQ ID NO: 99 - protein, 29 aa, Artificial Sequence
  Description of Artificial Sequence Synthetic peptide
MVSTKKTKTHDHYPSGRMRDPGWHDWRAS

SEQ ID NO: 100 - protein, 29 aa, Artificial Sequence
  Description of Artificial Sequence Synthetic peptide
MLSAKKTKTHDHYPSGRMRDPGWHDWRAS

SEQ ID NO: 101 - protein, 29 aa, Artificial Sequence
  Description of Artificial Sequence Synthetic peptide
MLSGKKTKTHDHYPSGRMRDPGWHDWRAS

SEQ ID NO: 102 - protein, 29 aa, Artificial Sequence
  Description of Artificial Sequence Synthetic peptide
MLSTAKTKTHDHYPSGRMRDPGWHDWRAS

SEQ ID NO: 103 - protein, 29 aa, Artificial Sequence
  Description of Artificial Sequence Synthetic peptide
MLSTKKGKTHDHYPSGRMRDPGWHDWRAS

SEQ ID NO: 104 - protein, 29 aa, Artificial Sequence
  Description of Artificial Sequence Synthetic peptide
MLSTKKTKWHDHYPSGRMRDPGWHDWRAS

SEQ ID NO: 105 - protein, 29 aa, Artificial Sequence
  Description of Artificial Sequence Synthetic peptide
MLSTKKTKTHDHNPSGRMRDPGWHDWRAS

SEQ ID NO: 106 - protein, 29 aa, Artificial Sequence
  Description of Artificial Sequence Synthetic peptide
MLSTKKTKTHDHYGSGRMRDPGWHDWRAS

SEQ ID NO: 107 - protein, 29 aa, Artificial Sequence
  Description of Artificial Sequence Synthetic peptide
MLSTKKTKTHDHYWSGRMRDPGWHDWRAS

SEQ ID NO: 108 - protein, 29 aa, Artificial Sequence
  Description of Artificial Sequence Synthetic peptide
MLSTKKTKTHDHYPGGRMRDPGWHDWRAS

SEQ ID NO: 109 - protein, 29 aa, Artificial Sequence
  Description of Artificial Sequence Synthetic peptide
MLSTKKTKTHDHYPSGRYRDPGWHDWRAS

SEQ ID NO: 110 - protein, 29 aa, Artificial Sequence
  Description of Artificial Sequence Synthetic peptide
MLSTKKTKTHDHYPSGRMRDFGWHDWRAS

SEQ ID NO: 111 - protein, 29 aa, Artificial Sequence
  Description of Artificial Sequence Synthetic peptide
MLSTKKTKTHDHYPSGRMRDPKWHDWRAS

SEQ ID NO: 112 - protein, 29 aa, Artificial Sequence
  Description of Artificial Sequence Synthetic peptide
MLSTKKTKTHDHYPSGRMRDPWWHDWRAS

SEQ ID NO: 113 - protein, 29 aa, Artificial Sequence
  Description of Artificial Sequence Synthetic peptide
MLSTKKTKTHDHYPSGRMRDPGWHDWRWS

SEQ ID NO: 114 - protein, 29 aa, Artificial Sequence
  Description of Artificial Sequence Synthetic peptide
MLSTKKTKTHDHYPSGRMRDPGWHDWRAR

SEQ ID NO: 115 - protein, 29 aa, Artificial Sequence
  Description of Artificial Sequence Synthetic peptide
  MOD_RES (1)..(2): Any naturally occurring amino acid
  MOD_RES (4)..(4): Any naturally occurring amino acid
  MOD_RES (7)..(7): Any naturally occurring amino acid
  MOD_RES (9)..(9): Any naturally occurring amino acid
  MOD_RES (13)..(15): Any naturally occurring amino acid
  MOD_RES (18)..(18): Any naturally occurring amino acid
  MOD_RES (20)..(20): Any naturally occurring amino acid
  MOD_RES (22)..(22): Any naturally occurring amino acid
  MOD_RES (27)..(29): Any naturally occurring amino acid
XXSXAKXKXHDHXXXGRXRXPXWHDWXXX

SEQ ID NO: 116 - protein, 29 aa, Artificial Sequence
  Description of Artificial Sequence Synthetic peptide
  MOD_RES (1)..(7): Any naturally occurring amino acid
  MOD_RES (9)..(15): Any naturally occurring amino acid
  MOD_RES (18)..(18): Any naturally occurring amino acid
  MOD_RES (20)..(22): Any naturally occurring amino acid
  MOD_RES (25)..(25): Any naturally occurring amino acid
  MOD_RES (27)..(29): Any naturally occurring amino acid
XXXXXXXKXXXXXXXGRXRXXXWHXWXXX

SEQ ID NO: 117 - protein, 37 aa, Artificial Sequence
  Description of Artificial Sequence Synthetic polypeptide
MKVRPSVKPMCEKCKIIRRKGRVMVICENPKHKQRQG

SEQ ID NO: 118 - protein, 37 aa, Artificial Sequence
  Description of Artificial Sequence Synthetic polypeptide
MKVRPSVKPICEKCKIIRRKGRVMVICENPKHKQKQG

SEQ ID NO: 119 - protein, 37 aa, Artificial Sequence
  Description of Artificial Sequence Synthetic polypeptide
MKVRPSVKPMCEKCKIIKRKGKVMVICENPKHKQRQG

SEQ ID NO: 120 - protein, 37 aa, Artificial Sequence
  Description of Artificial Sequence Synthetic polypeptide
MKVRPSVKPMCEKCKIIKRKGKVMVICENPKHKQKQG

SEQ ID NO: 121 - protein, 37 aa, Artificial Sequence
  Description of Artificial Sequence Synthetic polypeptide
MKVRPSVKPICEKCKIIRRKGRVMVICQNPKHKQKQG

SEQ ID NO: 122 - protein, 37 aa, Artificial Sequence
  Description of Artificial Sequence Synthetic polypeptide
MKVRPSVKPICEKCKIIKRKGRVMVICENPKHKQKQG

SEQ ID NO: 123 - protein, 37 aa, Artificial Sequence
  Description of Artificial Sequence Synthetic polypeptide
MKVRPSVKPMCEKCKVIKRKGKVMVICENPKHKQRQG

SEQ ID NO: 124 - protein, 37 aa, Artificial Sequence
  Description of Artificial Sequence Synthetic polypeptide
MKVRPSVKPMCEKCKVIKRKGRVMVICENPKHKQKQG

SEQ ID NO: 125 - protein, 37 aa, Artificial Sequence
  Description of Artificial Sequence Synthetic polypeptide
MKVRPSVKPICEKCKVIRRKGRVMVICENPKHKQKQG

SEQ ID NO: 126 - protein, 37 aa, Artificial Sequence
  Description of Artificial Sequence Synthetic polypeptide
MKVKPSVKPICEKCKIIKRKGRVMVICENPKHKQKQG

SEQ ID NO: 127 - protein, 37 aa, Artificial Sequence
  Description of Artificial Sequence Synthetic polypeptide
MKVRPSVKPMCEKCKVIKRKGKVMVICENPKHKQKQG

SEQ ID NO: 128 - protein, 37 aa, Artificial Sequence
  Description of Artificial Sequence Synthetic polypeptide
MKVRPSVKKICEKCKIIKRKGRVMVICENPKHKQKQG

SEQ ID NO: 129 - protein, 37 aa, Artificial Sequence
  Description of Artificial Sequence Synthetic polypeptide
MKIRPSVKPMCEKCRIIKRKGRVMVICENPKHKQKQG

SEQ ID NO: 130 - protein, 37 aa, Artificial Sequence
  Description of Artificial Sequence Synthetic polypeptide
MKVRPSVKPICEKCKVIRRKGKVMVICENPKHKQKQG

SEQ ID NO: 131 - protein, 37 aa, Artificial Sequence
  Description of Artificial Sequence Synthetic polypeptide
MKVRPSVKPICEKCKIIKRKGKVMVICENPKHKQKQG

SEQ ID NO: 132 - protein, 37 aa, Artificial Sequence
  Description of Artificial Sequence Synthetic polypeptide
MKVRPSVKPICEKCKIIRRKGKVMVICQNPKHKQKQG

SEQ ID NO: 133 - protein, 37 aa, Artificial Sequence
  Description of Artificial Sequence Synthetic polypeptide
MKVRPSVKPICEKCKVIKRKGRVMVICENPKHKQKQG

SEQ ID NO: 134 - protein, 37 aa, Artificial Sequence
  Description of Artificial Sequence Synthetic polypeptide
MKVKPSVKTICEKCKIIRRKGRVMVICENPKHKQKQG

SEQ ID NO: 135 - protein, 37 aa, Artificial Sequence
  Description of Artificial Sequence Synthetic polypeptide
MKVRPSVKPICEKCKVIKRKGKVMVICENPKHKQRQG

SEQ ID NO: 136 - protein, 38 aa, Artificial Sequence
  Description of Artificial Sequence Synthetic polypeptide
MKVRPSVKPICDKCRIIKRKGRVMVICENPKHKQRQGN

SEQ ID NO: 137 - protein, 37 aa, Artificial Sequence
  Description of Artificial Sequence Synthetic polypeptide
MKVKPSVKPICEKCKVIRRKGRVMVICQNPKHKQRQG

SEQ ID NO: 138 - protein, 37 aa, Artificial Sequence
  Description of Artificial Sequence Synthetic polypeptide
MKVKPSVKTICEKCKIIKRKGRVMVICENPKHKQKQG

SEQ ID NO: 139 - protein, 37 aa, Artificial Sequence
  Description of Artificial Sequence Synthetic polypeptide
MKVQPSVKKICEKCKIIKRKGRVMVICENPKHKQKQG

SEQ ID NO: 140 - protein, 37 aa, Artificial Sequence
  Description of Artificial Sequence Synthetic polypeptide
MKVRSSVKPICEKCKIIRRKGSIRVICENPKHKQRQG

SEQ ID NO: 141 - protein, 37 aa, Artificial Sequence
  Description of Artificial Sequence Synthetic polypeptide
MKVKPSVKPICEKCKVIKRKGRVMVICQNPKHKQRQG

SEQ ID NO: 142 - protein, 37 aa, Artificial Sequence
  Description of Artificial Sequence Synthetic polypeptide
MKVRSSVKPICEKCKIIKRKGRIRVICENPKHKQRQG

SEQ ID NO: 143 - protein, 37 aa, Artificial Sequence
  Description of Artificial Sequence Synthetic polypeptide
MKVRPSVKPICEKCKVIKRKGKVMVICENPKHKQKQG

SEQ ID NO: 144 - protein, 37 aa, Artificial Sequence
  Description of Artificial Sequence Synthetic polypeptide
MKVKPSVKKICEKCKIIKRKGRVMVICENPKHKQKQG

SEQ ID NO: 145 - protein, 37 aa, Artificial Sequence
  Description of Artificial Sequence Synthetic polypeptide
MKVRPSVKPICEKCKVIKRKGKVMVICQNPKHKQRQG

SEQ ID NO: 146 - protein, 37 aa, Artificial Sequence
  Description of Artificial Sequence Synthetic polypeptide
MKVKPSVKTICEKCKIIRRKGRVMIICENPKHKQKQG

SEQ ID NO: 147 - protein, 37 aa, Artificial Sequence
  Description of Artificial Sequence Synthetic polypeptide
MKVRPSVKPICEKCKVIKRKGHVMVICENPKHKQKQG

SEQ ID NO: 148 - protein, 37 aa, Artificial Sequence
  Description of Artificial Sequence Synthetic polypeptide
MKVRPSVKPICDKCRVIKRKGRVMVICENPKHKQRQG

SEQ ID NO: 149 - protein, 39 aa, Artificial Sequence
  Description of Artificial Sequence Synthetic polypeptide
MKVRSSVKPICEKCKIIRRKGSIRVICENPKHKQRQDRH

SEQ ID NO: 150 - protein, 38 aa, Artificial Sequence
  Description of Artificial Sequence Synthetic polypeptide
MKVRPSVKPICEYCKVIRRNGRVMVICPTNPKHKQRQG

SEQ ID NO: 151 - protein, 37 aa, Artificial Sequence
  Description of Artificial Sequence Synthetic polypeptide
MKVRSSVKPICEKCKIIKRKGSIRVICENPKHKQRQG

SEQ ID NO: 152 - protein, 37 aa, Artificial Sequence
  Description of Artificial Sequence Synthetic polypeptide
MKVKPSVKPICEKCKVIKRKGRVMIICANPKHKQRQG

SEQ ID NO: 153 - protein, 37 aa, Artificial Sequence
  Description of Artificial Sequence Synthetic polypeptide
MKVKPSVKTICEKCKIIKRKGRVMIICENPKHKQKQG

SEQ ID NO: 154 - protein, 37 aa, Artificial Sequence
  Description of Artificial Sequence Synthetic polypeptide
MKTRSSVKPMCDKCKVIKRKGRVAVICENPKHKQRQG

SEQ ID NO: 155 - protein, 37 aa, Artificial Sequence
  Description of Artificial Sequence Synthetic polypeptide
MKVRPSVKPMCDKCKVIKRKGKVMVICQEPKHKQRQG

SEQ ID NO: 156 - protein, 36 aa, Artificial Sequence
  Description of Artificial Sequence Synthetic polypeptide
KVRSSVKPICEKCKVIKRKGIVRVICENPKHKQRQG

SEQ ID NO: 157 - protein, 37 aa, Artificial Sequence
  Description of Artificial Sequence Synthetic polypeptide
MKVRASVKPICDKCKVIKRKGIVRVICENPKHKQRQG

SEQ ID NO: 158 - protein, 37 aa, Artificial Sequence
  Description of Artificial Sequence Synthetic polypeptide
MKVRPSVKKMCDKCKIIRRHGKILVICENPRHKQRQG

SEQ ID NO: 159 - protein, 37 aa, Artificial Sequence
  Description of Artificial Sequence Synthetic polypeptide
MKVRPSVKKMCDKCKVIKRKGKILVICENPKHKQRQG

SEQ ID NO: 160 - protein, 37 aa, Artificial Sequence
  Description of Artificial Sequence Synthetic polypeptide
MKVRSSVKPICEKCKVIKRKGSVRIICENPKHKQRQG

SEQ ID NO: 161 - protein, 41 aa, Artificial Sequence
  Description of Artificial Sequence Synthetic polypeptide
  MOD_RES (1)..(1): Any naturally occurring amino acid
  MOD_RES (3)..(5): Any naturally occurring amino acid
  MOD_RES (9)..(10): Any naturally occurring amino acid
  MOD_RES (12)..(13): Any naturally occurring amino acid
  MOD_RES (15)..(17): Any naturally occurring amino acid
  MOD_RES (19)..(19): Any naturally occurring amino acid
  MOD_RES (21)..(21): Any naturally occurring amino acid
  MOD_RES (23)..(26): Any naturally occurring amino acid
  MOD_RES (29)..(29): P or absent
  MOD_RES (30)..(31): Any naturally occurring amino acid
  MOD_RES (33)..(33): Any naturally occurring amino acid
  MOD_RES (37)..(37): Any naturally occurring amino acid
  MOD_RES (39)..(41): This region may encompass any of the following “G”, “GN”, or “DRH”
  See specification as filed for detailed description of substitutions and preferred embodiments
XKXXXSVKXXCXXCXXXIXRXGXXXXICXXXPXHKQXQXXX

SEQ ID NO: 162 - protein, 37 aa, Artificial Sequence; identical to SEQ ID NO: 117
  Description of Artificial Sequence Synthetic polypeptide

SEQ ID NO: 163 - protein, 39 aa, Artificial Sequence
  Description of Artificial Sequence Synthetic polypeptide
FLSTKKTKTHDHYPSGRMRDPGWHDWRASGGGGSGGGGS

SEQ ID NO: 164 - protein, 39 aa, Artificial Sequence
  Description of Artificial Sequence Synthetic polypeptide
KLSTKKTKTHDHYPSGRMRDPGWHDWRASGGGGSGGGGS

SEQ ID NO: 165 - protein, 39 aa, Artificial Sequence
  Description of Artificial Sequence Synthetic polypeptide
PLSTKKTKTHDHYPSGRMRDPGWHDWRASGGGGSGGGGS

SEQ ID NO: 166 - protein, 39 aa, Artificial Sequence
  Description of Artificial Sequence Synthetic polypeptide
RLSTKKTKTHDHYPSGRMRDPGWHDWRASGGGGSGGGGS

SEQ ID NO: 167 - protein, 39 aa, Artificial Sequence
  Description of Artificial Sequence Synthetic polypeptide
MASTKKTKTHDHYPSGRMRDPGWHDWRASGGGGSGGGGS

SEQ ID NO: 168 - protein, 39 aa, Artificial Sequence
  Description of Artificial Sequence Synthetic polypeptide
MGSTKKTKTHDHYPSGRMRDPGWHDWRASGGGGSGGGGS

SEQ ID NO: 169 - protein, 39 aa, Artificial Sequence
  Description of Artificial Sequence Synthetic polypeptide
MKSTKKTKTHDHYPSGRMRDPGWHDWRASGGGGSGGGGS

SEQ ID NO: 170 - protein, 39 aa, Artificial Sequence
  Description of Artificial Sequence Synthetic polypeptide
MPSTKKTKTHDHYPSGRMRDPGWHDWRASGGGGSGGGGS

SEQ ID NO: 171 - protein, 39 aa, Artificial Sequence
  Description of Artificial Sequence Synthetic polypeptide
MQSTKKTKTHDHYPSGRMRDPGWHDWRASGGGGSGGGGS

SEQ ID NO: 172 - protein, 39 aa, Artificial Sequence
  Description of Artificial Sequence Synthetic polypeptide
MRSTKKTKTHDHYPSGRMRDPGWHDWRASGGGGSGGGGS

SEQ ID NO: 173 - protein, 39 aa, Artificial Sequence
  Description of Artificial Sequence Synthetic polypeptide
MSSTKKTKTHDHYPSGRMRDPGWHDWRASGGGGSGGGGS

SEQ ID NO: 174 - protein, 39 aa, Artificial Sequence
  Description of Artificial Sequence Synthetic polypeptide
MTSTKKTKTHDHYPSGRMRDPGWHDWRASGGGGSGGGGS

SEQ ID NO: 175 - protein, 39 aa, Artificial Sequence
  Description of Artificial Sequence Synthetic polypeptide
MWSTKKTKTHDHYPSGRMRDPGWHDWRASGGGGSGGGGS

SEQ ID NO: 176 - protein, 39 aa, Artificial Sequence
  Description of Artificial Sequence Synthetic polypeptide
MYSTKKTKTHDHYPSGRMRDPGWHDWRASGGGGSGGGGS

SEQ ID NO: 177 - protein, 39 aa, Artificial Sequence
  Description of Artificial Sequence Synthetic polypeptide
MLQTKKTKTHDHYPSGRMRDPGWHDWRASGGGGSGGGGS

SEQ ID NO: 178 - protein, 39 aa, Artificial Sequence
  Description of Artificial Sequence Synthetic polypeptide
MLRTKKTKTHDHYPSGRMRDPGWHDWRASGGGGSGGGGS

SEQ ID NO: 179 - protein, 39 aa, Artificial Sequence
  Description of Artificial Sequence Synthetic polypeptide
MLSRKKTKTHDHYPSGRMRDPGWHDWRASGGGGSGGGGS

SEQ ID NO: 180 - protein, 39 aa, Artificial Sequence
  Description of Artificial Sequence Synthetic polypeptide
MLSTRKTKTHDHYPSGRMRDPGWHDWRASGGGGSGGGGS

SEQ ID NO: 181 - protein, 39 aa, Artificial Sequence
  Description of Artificial Sequence Synthetic polypeptide
MLSTKRTKTHDHYPSGRMRDPGWHDWRASGGGGSGGGGS

SEQ ID NO: 182 - protein, 39 aa, Artificial Sequence
  Description of Artificial Sequence Synthetic polypeptide
MLSTKTTKTHDHYPSGRMRDPGWHDWRASGGGGSGGGGS

SEQ ID NO: 183 - protein, 39 aa, Artificial Sequence
  Description of Artificial Sequence Synthetic polypeptide
MLSTKKKKTHDHYPSGRMRDPGWHDWRASGGGGSGGGGS

SEQ ID NO: 184 - protein, 39 aa, Artificial Sequence
  Description of Artificial Sequence Synthetic polypeptide
MLSTKKRKTHDHYPSGRMRDPGWHDWRASGGGGSGGGGS

SEQ ID NO: 185 - protein, 39 aa, Artificial Sequence
  Description of Artificial Sequence Synthetic polypeptide
MLSTKKTKRHDHYPSGRMRDPGWHDWRASGGGGSGGGGS

SEQ ID NO: 186 - protein, 39 aa, Artificial Sequence
  Description of Artificial Sequence Synthetic polypeptide
MLSTKKTKTKDHYPSGRMRDPGWHDWRASGGGGSGGGGS

SEQ ID NO: 187 - protein, 39 aa, Artificial Sequence
  Description of Artificial Sequence Synthetic polypeptide
MLSTKKTKTRDHYPSGRMRDPGWHDWRASGGGGSGGGGS

SEQ ID NO: 188 - protein, 39 aa, Artificial Sequence
  Description of Artificial Sequence Synthetic polypeptide
MLSTKKTKTHFHYPSGRMRDPGWHDWRASGGGGSGGGGS

SEQ ID NO: 189 - protein, 39 aa, Artificial Sequence
  Description of Artificial Sequence Synthetic polypeptide
MLSTKKTKTHGHYPSGRMRDPGWHDWRASGGGGSGGGGS

SEQ ID NO: 190 - protein, 39 aa, Artificial Sequence
  Description of Artificial Sequence Synthetic polypeptide
MLSTKKTKTHHHYPSGRMRDPGWHDWRASGGGGSGGGGS

SEQ ID NO: 191 - protein, 39 aa, Artificial Sequence
  Description of Artificial Sequence Synthetic polypeptide
MLSTKKTKTHIHYPSGRMRDPGWHDWRASGGGGSGGGGS

SEQ ID NO: 192 - protein, 39 aa, Artificial Sequence
  Description of Artificial Sequence Synthetic polypeptide
MLSTKKTKTHKHYPSGRMRDPGWHDWRASGGGGSGGGGS

SEQ ID NO: 193 - protein, 39 aa, Artificial Sequence
  Description of Artificial Sequence Synthetic polypeptide
MLSTKKTKTHPHYPSGRMRDPGWHDWRASGGGGSGGGGS

SEQ ID NO: 194 - protein, 39 aa, Artificial Sequence
  Description of Artificial Sequence Synthetic polypeptide
MLSTKKTKTHRHYPSGRMRDPGWHDWRASGGGGSGGGGS

SEQ ID NO: 195 - protein, 39 aa, Artificial Sequence
  Description of Artificial Sequence Synthetic polypeptide
MLSTKKTKTHTHYPSGRMRDPGWHDWRASGGGGSGGGGS

SEQ ID NO: 196 - protein, 39 aa, Artificial Sequence
  Description of Artificial Sequence Synthetic polypeptide
MLSTKKTKTHVHYPSGRMRDPGWHDWRASGGGGSGGGGS

SEQ ID NO: 197 - protein, 39 aa, Artificial Sequence
  Description of Artificial Sequence Synthetic polypeptide
MLSTKKTKTHWHYPSGRMRDPGWHDWRASGGGGSGGGGS

SEQ ID NO: 198 - protein, 39 aa, Artificial Sequence
  Description of Artificial Sequence Synthetic polypeptide
MLSTKKTKTHYHYPSGRMRDPGWHDWRASGGGGSGGGGS

SEQ ID NO: 199 - protein, 39 aa, Artificial Sequence
  Description of Artificial Sequence Synthetic polypeptide
MLSTKKTKTHDKYPSGRMRDPGWHDWRASGGGGSGGGGS

SEQ ID NO: 200 - protein, 39 aa, Artificial Sequence
  Description of Artificial Sequence Synthetic polypeptide
MLSTKKTKTHDRYPSGRMRDPGWHDWRASGGGGSGGGGS

SEQ ID NO: 201 - protein, 39 aa, Artificial Sequence
  Description of Artificial Sequence Synthetic polypeptide
MLSTKKTKTHDHGPSGRMRDPGWHDWRASGGGGSGGGGS

SEQ ID NO: 202 - protein, 39 aa, Artificial Sequence
  Description of Artificial Sequence Synthetic polypeptide
MLSTKKTKTHDHKPSGRMRDPGWHDWRASGGGGSGGGGS

SEQ ID NO: 203 - protein, 39 aa, Artificial Sequence
  Description of Artificial Sequence Synthetic polypeptide
MLSTKKTKTHDHRPSGRMRDPGWHDWRASGGGGSGGGGS

SEQ ID NO: 204 - protein, 39 aa, Artificial Sequence
  Description of Artificial Sequence Synthetic polypeptide
MLSTKKTKTHDHWPSGRMRDPGWHDWRASGGGGSGGGGS

SEQ ID NO: 205 - protein, 39 aa, Artificial Sequence
  Description of Artificial Sequence Synthetic polypeptide
MLSTKKTKTHDHYPRGRMRDPGWHDWRASGGGGSGGGGS

SEQ ID NO: 206 - protein, 39 aa, Artificial Sequence
  Description of Artificial Sequence Synthetic polypeptide
MLSTKKTKTHDHYPSGRGRDPGWHDWRASGGGGSGGGGS

SEQ ID NO: 207 - protein, 39 aa, Artificial Sequence
  Description of Artificial Sequence Synthetic polypeptide
MLSTKKTKTHDHYPSGRHRDPGWHDWRASGGGGSGGGGS

SEQ ID NO: 208 - protein, 39 aa, Artificial Sequence
  Description of Artificial Sequence Synthetic polypeptide
MLSTKKTKTHDHYPSGRKRDPGWHDWRASGGGGSGGGGS

SEQ ID NO: 209 - protein, 39 aa, Artificial Sequence
  Description of Artificial Sequence Synthetic polypeptide
MLSTKKTKTHDHYPSGRRRDPGWHDWRASGGGGSGGGGS

SEQ ID NO: 210 - protein, 39 aa, Artificial Sequence
  Description of Artificial Sequence Synthetic polypeptide
MLSTKKTKTHDHYPSGRWRDPGWHDWRASGGGGSGGGGS

SEQ ID NO: 211 - protein, 39 aa, Artificial Sequence
  Description of Artificial Sequence Synthetic polypeptide
MLSTKKTKTHDHYPSGRMRPPGWHDWRASGGGGSGGGGS

SEQ ID NO: 212 - protein, 39 aa, Artificial Sequence
  Description of Artificial Sequence Synthetic polypeptide
MLSTKKTKTHDHYPSGRMRRPGWHDWRASGGGGSGGGGS

SEQ ID NO: 213 - protein, 39 aa, Artificial Sequence
  Description of Artificial Sequence Synthetic polypeptide
MLSTKKTKTHDHYPSGRMRDPPWHDWRASGGGGSGGGGS

SEQ ID NO: 214 - protein, 39 aa, Artificial Sequence
  Description of Artificial Sequence Synthetic polypeptide
MLSTKKTKTHDHYPSGRMRDPGWHPWRASGGGGSGGGGS

SEQ ID NO: 215 - protein, 39 aa, Artificial Sequence
  Description of Artificial Sequence Synthetic polypeptide
MLSTKKTKTHDHYPSGRMRDPGWHRWRASGGGGSGGGGS

SEQ ID NO: 216 - protein, 39 aa, Artificial Sequence
  Description of Artificial Sequence Synthetic polypeptide
MLSTKKTKTHDHYPSGRMRDPGWHDWWASGGGGSGGGGS

SEQ ID NO: 217 - protein, 39 aa, Artificial Sequence
  Description of Artificial Sequence Synthetic polypeptide
MLSTKKTKTHDHYPSGRMRDPGWHDWRFSGGGGSGGGGS

SEQ ID NO: 218 - protein, 39 aa, Artificial Sequence
  Description of Artificial Sequence Synthetic polypeptide
MLSTKKTKTHDHYPSGRMRDPGWHDWRGSGGGGSGGGGS

SEQ ID NO: 219 - protein, 39 aa, Artificial Sequence
  Description of Artificial Sequence Synthetic polypeptide
MLSTKKTKTHDHYPSGRMRDPGWHDWRVSGGGGSGGGGS

SEQ ID NO: 220 - protein, 39 aa, Artificial Sequence
  Description of Artificial Sequence Synthetic polypeptide
MLSTKKTKTHDHYPSGRMRDPGWHDWRYSGGGGSGGGGS

SEQ ID NO: 221 - protein, 39 aa, Artificial Sequence
  Description of Artificial Sequence Synthetic polypeptide
SLSTKKTKTHDHYPSGRMRDPGWHDWRASGGGGSGGGGS

SEQ ID NO: 222 - protein, 39 aa, Artificial Sequence
  Description of Artificial Sequence Synthetic polypeptide
VLSTKKTKTHDHYPSGRMRDPGWHDWRASGGGGSGGGGS

SEQ ID NO: 223 - protein, 39 aa, Artificial Sequence
  Description of Artificial Sequence Synthetic polypeptide
WLSTKKTKTHDHYPSGRMRDPGWHDWRASGGGGSGGGGS

SEQ ID NO: 224 - protein, 39 aa, Artificial Sequence
  Description of Artificial Sequence Synthetic polypeptide
MFSTKKTKTHDHYPSGRMRDPGWHDWRASGGGGSGGGGS

SEQ ID NO: 225 - protein, 39 aa, Artificial Sequence
  Description of Artificial Sequence Synthetic polypeptide
MNSTKKTKTHDHYPSGRMRDPGWHDWRASGGGGSGGGGS

SEQ ID NO: 226 - protein, 39 aa, Artificial Sequence
  Description of Artificial Sequence Synthetic polypeptide
MVSTKKTKTHDHYPSGRMRDPGWHDWRASGGGGSGGGGS

SEQ ID NO: 227 - protein, 39 aa, Artificial Sequence
  Description of Artificial Sequence Synthetic polypeptide
MLSAKKTKTHDHYPSGRMRDPGWHDWRASGGGGSGGGGS

SEQ ID NO: 228 - protein, 39 aa, Artificial Sequence
  Description of Artificial Sequence Synthetic polypeptide
MLSGKKTKTHDHYPSGRMRDPGWHDWRASGGGGSGGGGS

SEQ ID NO: 229 - protein, 39 aa, Artificial Sequence
  Description of Artificial Sequence Synthetic polypeptide
MLSTAKTKTHDHYPSGRMRDPGWHDWRASGGGGSGGGGS

SEQ ID NO: 230 - protein, 39 aa, Artificial Sequence
  Description of Artificial Sequence Synthetic polypeptide
MLSTKKGKTHDHYPSGRMRDPGWHDWRASGGGGSGGGGS

SEQ ID NO: 231 - protein, 39 aa, Artificial Sequence
  Description of Artificial Sequence Synthetic polypeptide
MLSTKKTKWHDHYPSGRMRDPGWHDWRASGGGGSGGGGS

SEQ ID NO: 232 - protein, 39 aa, Artificial Sequence
  Description of Artificial Sequence Synthetic polypeptide
MLSTKKTKTHDHNPSGRMRDPGWHDWRASGGGGSGGGGS

SEQ ID NO: 233 - protein, 39 aa, Artificial Sequence
  Description of Artificial Sequence Synthetic polypeptide
MLSTKKTKTHDHYGSGRMRDPGWHDWRASGGGGSGGGGS

SEQ ID NO: 234 - protein, 39 aa, Artificial Sequence
  Description of Artificial Sequence Synthetic polypeptide
MLSTKKTKTHDHYWSGRMRDPGWHDWRASGGGGSGGGGS

SEQ ID NO: 235 - protein, 39 aa, Artificial Sequence
  Description of Artificial Sequence Synthetic polypeptide
MLSTKKTKTHDHYPGGRMRDPGWHDWRASGGGGSGGGGS

SEQ ID NO: 236 - protein, 39 aa, Artificial Sequence
  Description of Artificial Sequence Synthetic polypeptide
MLSTKKTKTHDHYPSGRYRDPGWHDWRASGGGGSGGGGS

SEQ ID NO: 237 - protein, 39 aa, Artificial Sequence
  Description of Artificial Sequence Synthetic polypeptide
MLSTKKTKTHDHYPSGRMRDFGWHDWRASGGGGSGGGGS

SEQ ID NO: 238 - protein, 39 aa, Artificial Sequence
  Description of Artificial Sequence Synthetic polypeptide
MLSTKKTKTHDHYPSGRMRDPKWHDWRASGGGGSGGGGS

SEQ ID NO: 239 - protein, 39 aa, Artificial Sequence
  Description of Artificial Sequence Synthetic polypeptide
MLSTKKTKTHDHYPSGRMRDPWWHDWRASGGGGSGGGGS

SEQ ID NO: 240 - protein, 39 aa, Artificial Sequence
  Description of Artificial Sequence Synthetic polypeptide
MLSTKKTKTHDHYPSGRMRDPGWHDWRWSGGGGSGGGGS

SEQ ID NO: 241 - protein, 39 aa, Artificial Sequence
  Description of Artificial Sequence Synthetic polypeptide
MLSTKKTKTHDHYPSGRMRDPGWHDWRARGGGGSGGGGS

SEQ ID NO: 242 - protein, 29 aa, Artificial Sequence
  Description of Artificial Sequence Synthetic peptide
MSSAAKGKTHDHNWSGRYRPPPWHDWRVR

SEQ ID NO: 243 - protein, 29 aa, Artificial Sequence
  Description of Artificial Sequence Synthetic peptide
MSSGAKGKWHDHYGSGRHRPPPWHDWRVR

SEQ ID NO: 244 - protein, 29 aa, Artificial Sequence
  Description of Artificial Sequence Synthetic peptide
FASAAKGKTHDHWGSGRYRPPPWHDWRGS

SEQ ID NO: 245 - protein, 29 aa, Artificial Sequence
  Description of Artificial Sequence Synthetic peptide
MASGAKGKTHDHNWSGRMRPPPWHDWRGR

SEQ ID NO: 246 - protein, 29 aa, Artificial Sequence
  Description of Artificial Sequence Synthetic peptide
MPSAAKGKTHDHYWSGRMRPPPWHDWRVS

SEQ ID NO: 247 - protein, 29 aa, Artificial Sequence
  Description of Artificial Sequence Synthetic peptide
MGSAAKTKWHDHYGGGRMRPPPWHDWRAR

SEQ ID NO: 248 - protein, 29 aa, Artificial Sequence
  Description of Artificial Sequence Synthetic peptide
FVSTAKGKTHDHNWSGRMRPPPWHDWRGR

SEQ ID NO: 249 - protein, 29 aa, Artificial Sequence
  Description of Artificial Sequence Synthetic peptide
MLSTAKGKTHDHYGGGRMRPPPWHDWRFS

SEQ ID NO: 250 - protein, 29 aa, Artificial Sequence
  Description of Artificial Sequence Synthetic peptide
WSSAAKGKTHDHNWSGRHRPPPWHDWRFS

SEQ ID NO: 251 - protein, 29 aa, Artificial Sequence
  Description of Artificial Sequence Synthetic peptide
VVSTAKGKTHDHYPGGRMRPPPWHDWRAR

SEQ ID NO: 252 - protein, 29 aa, Artificial Sequence
  Description of Artificial Sequence Synthetic peptide
MASAAKGKTHDHYPGGRMRPPPWHDWRAS

SEQ ID NO: 253 - protein, 29 aa, Artificial Sequence
  Description of Artificial Sequence Synthetic peptide
WSSAAKGKTHDHYPSGRHRPPPWHDWRVS

SEQ ID NO: 254 - protein, 29 aa, Artificial Sequence
  Description of Artificial Sequence Synthetic peptide
FASAAKGKTHDHYPGGRMRPPPWHDWRFS

SEQ ID NO: 255 - protein, 29 aa, Artificial Sequence
  Description of Artificial Sequence Synthetic peptide
FLSAAKGKTHDHNWGGRMRPPPWHDWRFS

SEQ ID NO: 256 - protein, 29 aa, Artificial Sequence
  Description of Artificial Sequence Synthetic peptide
WGSAAKGKTHDHWPSGRMRDPPWHDWRVS

SEQ ID NO: 257 - protein, 29 aa, Artificial Sequence
  Description of Artificial Sequence Synthetic peptide
VLSTAKGKTHDHYPGGRMRPPPWHDWRAR

SEQ ID NO: 258 - protein, 29 aa, Artificial Sequence
  Description of Artificial Sequence Synthetic peptide
MVSAAKTKTHDHNGGGRMRPPPWHDWRFS

SEQ ID NO: 259 - protein, 29 aa, Artificial Sequence
  Description of Artificial Sequence Synthetic peptide
VVSTAKGKTHDHYWSGRYRPPPWHDWRAR

SEQ ID NO: 260 - protein, 29 aa, Artificial Sequence
  Description of Artificial Sequence Synthetic peptide
PLSAAKGKWHDHYWSGRMRDPPWHDWRVS

SEQ ID NO: 261 - protein, 29 aa, Artificial Sequence
  Description of Artificial Sequence Synthetic peptide
SPSGAKGKTHDHYPSGRMRPPPWHDWRFS

SEQ ID NO: 262 - protein, 29 aa, Artificial Sequence
  Description of Artificial Sequence Synthetic peptide
MGSAAKGKWHDHYGSGRMRPPPWHDWRVR

SEQ ID NO: 263 - protein, 29 aa, Artificial Sequence
  Description of Artificial Sequence Synthetic peptide
MASAAKGKTHDHYPSGRMRPPPWHDWRFS

SEQ ID NO: 264 - protein, 29 aa, Artificial Sequence
  Description of Artificial Sequence Synthetic peptide
PSSAAKGKTHDHYPGGRYRPPPWHDWRVS

SEQ ID NO: 265 - protein, 29 aa, Artificial Sequence
  Description of Artificial Sequence Synthetic peptide
PLSAAKTKWHDHYGGGRMRPPPWHDWRFS

SEQ ID NO: 266 - protein, 29 aa, Artificial Sequence
  Description of Artificial Sequence Synthetic peptide
MVSTAKGKTHDHYGGGRYRPPPWHDWRAR

SEQ ID NO: 267 - protein, 29 aa, Artificial Sequence
  Description of Artificial Sequence Synthetic peptide
FQSTAKGKTHDHWPSGRMRPPPWHDWRAS

SEQ ID NO: 268 - protein, 29 aa, Artificial Sequence
  Description of Artificial Sequence Synthetic peptide
MGSGAKGKTHDHWPSGRMRPPPWHDWRVS

SEQ ID NO: 269 - protein, 29 aa, Artificial Sequence
  Description of Artificial Sequence Synthetic peptide
MLSAAKGKTHDHYPSGRYRPPPWHDWRAR

SEQ ID NO: 270 - protein, 29 aa, Artificial Sequence
  Description of Artificial Sequence Synthetic peptide
MPSAAKTKWHDHNWSGRMRPPPWHDWRAR

SEQ ID NO: 271 - protein, 29 aa, Artificial Sequence
  Description of Artificial Sequence Synthetic peptide
WASGAKGKTHDHWPGGRHRDPPWHDWRVS

SEQ ID NO: 272 - protein, 29 aa, Artificial Sequence
  Description of Artificial Sequence Synthetic peptide
WTSAAKTKWHDHYGGGRMRPPPWHDWRAR

SEQ ID NO: 273 - protein, 29 aa, Artificial Sequence
  Description of Artificial Sequence Synthetic peptide
MWSGAKGKTHDHNWSGRMRPPPWHDWRGR

SEQ ID NO: 274 - protein, 29 aa, Artificial Sequence
  Description of Artificial Sequence Synthetic peptide
WSSAAKGKTHDHYGSGRMRDFGWHDWRFS

SEQ ID NO: 275 - protein, 29 aa, Artificial Sequence
  Description of Artificial Sequence Synthetic peptide
WGSAAKGKWHDHYPSGRMRPPPWHDWRVR

SEQ ID NO: 276 - protein, 29 aa, Artificial Sequence
  Description of Artificial Sequence Synthetic peptide
MGSGAKTKTHDHYPGGRYRPPPWHDWRVS

SEQ ID NO: 277 - protein, 29 aa, Artificial Sequence
  Description of Artificial Sequence Synthetic peptide
MPSTAKGKWHDHNPSGRMRPPPWHDWRGR

SEQ ID NO: 278 - protein, 29 aa, Artificial Sequence
  Description of Artificial Sequence Synthetic peptide
WASGAKGKTHDHYPSGRMRPPPWHDWRWS

SEQ ID NO: 279 - protein, 29 aa, Artificial Sequence
  Description of Artificial Sequence Synthetic peptide
MGSAAKGKWHDHNGSGRMRPPPWHDWRAR

SEQ ID NO: 280 - protein, 29 aa, Artificial Sequence
  Description of Artificial Sequence Synthetic peptide
WTSGAKGKWHDHYGGGRYRDPGWHDWRFS

SEQ ID NO: 281 - protein, 29 aa, Artificial Sequence
  Description of Artificial Sequence Synthetic peptide
FASGAKGKTHDHNWGGRYRDPGWHDWRAS

SEQ ID NO: 282 - protein, 29 aa, Artificial Sequence
  Description of Artificial Sequence Synthetic peptide
PLSAAKTKTHDHNWSGRYRPPPWHDWRAR

SEQ ID NO: 283 - protein, 29 aa, Artificial Sequence
  Description of Artificial Sequence Synthetic peptide
VSSAAKGKTHDHYPSGRMRPPPWHDWRGR

SEQ ID NO: 284 - protein, 29 aa, Artificial Sequence
  Description of Artificial Sequence Synthetic peptide
MGSGAKTKWHDHWPSGRMRPPPWHDWRFS

SEQ ID NO: 285 - protein, 29 aa, Artificial Sequence
  Description of Artificial Sequence Synthetic peptide
MGSGAKGKTHDHNWGGRHRDPGWHDWWVS

SEQ ID NO: 286 - protein, 29 aa, Artificial Sequence
  Description of Artificial Sequence Synthetic peptide
WSSGAKTKWHDHNGSGRYRPPPWHDWRGS

SEQ ID NO: 287 - protein, 29 aa, Artificial Sequence
  Description of Artificial Sequence Synthetic peptide
MASGAKGKTHDHYPGGRMRPPPWHDWRAR

SEQ ID NO: 288 - protein, 29 aa, Artificial Sequence
  Description of Artificial Sequence Synthetic peptide
MASAAKGKWHDHNPSGRHRDPGWHDWWGS

SEQ ID NO: 289 - protein, 29 aa, Artificial Sequence
  Description of Artificial Sequence Synthetic peptide
WTSAAKGKWHDHNGSGRHRDPPWHDWRAR

SEQ ID NO: 290 - protein, 29 aa, Artificial Sequence
  Description of Artificial Sequence Synthetic peptide
WASGAKGKWHDHYGSGRYRPPPWHDWRGS

SEQ ID NO: 291 - protein, 29 aa, Artificial Sequence
  Description of Artificial Sequence Synthetic peptide
VPSAAKGKWHDHNPGGRHRPPPWHDWRAS

SEQ ID NO: 292 - protein, 29 aa, Artificial Sequence
  Description of Artificial Sequence Synthetic peptide
MLSTAKGKTHDHYWGGRMRPPPWHDWRFS

SEQ ID NO: 293 - protein, 29 aa, Artificial Sequence
  Description of Artificial Sequence Synthetic peptide
MLSGAKTKTHDHNPSGRMRDPPWHDWRVR

SEQ ID NO: 294 - protein, 29 aa, Artificial Sequence
  Description of Artificial Sequence Synthetic peptide
WQSAAKTKTHDHWPSGRYRPPPWHDWRAS

SEQ ID NO: 295 - protein, 29 aa, Artificial Sequence
  Description of Artificial Sequence Synthetic peptide
WPSAAKGKTHDHYPGGRYRPPPWHDWRVR

SEQ ID NO: 296 - protein, 29 aa, Artificial Sequence
  Description of Artificial Sequence Synthetic peptide
VVSGAKGKTHDHNWGGRYRPPPWHDWRGS

SEQ ID NO: 297 - protein, 29 aa, Artificial Sequence
  Description of Artificial Sequence Synthetic peptide
VSSGAKGKTHDHYPSGRMRPPPWHDWRFS

SEQ ID NO: 298 - protein, 29 aa, Artificial Sequence
  Description of Artificial Sequence Synthetic peptide
FASAAKGKTHDHYPSGRYRPPPWHDWRAS

SEQ ID NO: 299 - protein, 29 aa, Artificial Sequence
  Description of Artificial Sequence Synthetic peptide
VGSGAKGKTHDHNGSGRYRPPPWHDWRFS

SEQ ID NO: 300 - protein, 29 aa, Artificial Sequence
  Description of Artificial Sequence Synthetic peptide
SVSAAKGKTHDHYPSGRWRDPPWHDWRAS

SEQ ID NO: 301 - protein, 29 aa, Artificial Sequence
  Description of Artificial Sequence Synthetic peptide
MSSGAKGKWHDHNWGGRMRDPGWHDWWGS

SEQ ID NO: 302 - protein, 29 aa, Artificial Sequence
  Description of Artificial Sequence Synthetic peptide
WGSAAKGKTHDHNWSGRYRPPPWHDWRVS

SEQ ID NO: 303 - protein, 29 aa, Artificial Sequence
  Description of Artificial Sequence Synthetic peptide
MSSGAKTKTHDHYPSGRMRPPPWHDWRAR

SEQ ID NO: 304 - protein, 29 aa, Artificial Sequence
  Description of Artificial Sequence Synthetic peptide
WSSAAKGKTHDHNPSGRHRPPPWHDWRVS

SEQ ID NO: 305 - protein, 29 aa, Artificial Sequence
  Description of Artificial Sequence Synthetic peptide
WSSTAKTKWHDHNGGGRMRPPPWHDWRAR

SEQ ID NO: 306 - protein, 29 aa, Artificial Sequence
  Description of Artificial Sequence Synthetic peptide
FPSGAKTKWHDHNPSGRMRDPPWHDWRFS

SEQ ID NO: 307 - protein, 29 aa, Artificial Sequence
  Description of Artificial Sequence Synthetic peptide
WLSTAKGKTHDHNWSGRYRPPPWHDWRVR

SEQ ID NO: 308 - protein, 29 aa, Artificial Sequence
  Description of Artificial Sequence Synthetic peptide
VTSAAKGKWHDHWPSGRYRDPPWHDWRVS

SEQ ID NO: 309 - protein, 29 aa, Artificial Sequence
  Description of Artificial Sequence Synthetic peptide
WTSGAKGKWHDHNGGGRMRDPGWHDWRFS

SEQ ID NO: 310 - protein, 29 aa, Artificial Sequence
  Description of Artificial Sequence Synthetic peptide
VTSAAKGKTHDHYPSGRYRPPPWHDWRGR

SEQ ID NO: 311 - protein, 29 aa, Artificial Sequence
  Description of Artificial Sequence Synthetic peptide
MPSAAKGKTHDHNGGGRMRPPPWHDWRFS

SEQ ID NO: 312 - protein, 29 aa, Artificial Sequence
  Description of Artificial Sequence Synthetic peptide
VTSAAKGKTHDHYPSGRMRPPPWHDWRFS

SEQ ID NO: 313 - protein, 29 aa, Artificial Sequence
  Description of Artificial Sequence Synthetic peptide
PASGAKGKTHDHYPSGRWRDSGWHDWRFS

SEQ ID NO: 314 - protein, 29 aa, Artificial Sequence
  Description of Artificial Sequence Synthetic peptide
MASTAKGKTHDHYGGGRMRPPPWHDWRVR

SEQ ID NO: 315 - protein, 29 aa, Artificial Sequence
  Description of Artificial Sequence Synthetic peptide
WLSAAKAKTHDHWGSGRHRPPPWHDWRGS

SEQ ID NO: 316 - protein, 29 aa, Artificial Sequence
  Description of Artificial Sequence Synthetic peptide
FASGAKGKTHDHYPSGRHRPPPWHDWRAS

SEQ ID NO: 317 - protein, 29 aa, Artificial Sequence
  Description of Artificial Sequence Synthetic peptide
MPSGAKGKWHDHYGGGRMRDPGWHDWRGS

SEQ ID NO: 318 - protein, 29 aa, Artificial Sequence
  Description of Artificial Sequence Synthetic peptide
WPSTAKGKTHDHYGGGRMRPPPWHDWRAR

SEQ ID NO: 319 - protein, 29 aa, Artificial Sequence
  Description of Artificial Sequence Synthetic peptide
MGSAAKGKWHDHYPGGRHRDPPWHDWRAS

SEQ ID NO: 320 - protein, 29 aa, Artificial Sequence
  Description of Artificial Sequence Synthetic peptide
MASTAKGKTHDHWPGGRMRPPPWHDWRGS

SEQ ID NO: 321 - protein, 29 aa, Artificial Sequence
  Description of Artificial Sequence Synthetic peptide
PVSAAKGKWHDHYPSGRWRDPPWHDWRAS

SEQ ID NO: 322 - protein, 29 aa, Artificial Sequence
  Description of Artificial Sequence Synthetic peptide
VSSAAKGKTHDHNPSGRMRPPPWHDWRFS

SEQ ID NO: 323 - protein, 29 aa, Artificial Sequence
  Description of Artificial Sequence Synthetic peptide
WASAAKGKTHDHYGGGRMRPPPWHDWRAR

SEQ ID NO: 324 - protein, 29 aa, Artificial Sequence
  Description of Artificial Sequence Synthetic peptide
SPSGAKTKWHDHNPGGRMRPPPWHDWRAR

SEQ ID NO: 325 - protein, 29 aa, Artificial Sequence
  Description of Artificial Sequence Synthetic peptide
SSSGAKGKWHDHNPSGRWRDPGWHDWRVR

SEQ ID NO: 326 - protein, 29 aa, Artificial Sequence
  Description of Artificial Sequence Synthetic peptide
MPSAAKGKWHDHYGSGRYRPPPWHDWRVR

SEQ ID NO: 327 - protein, 29 aa, Artificial Sequence
  Description of Artificial Sequence Synthetic peptide
PASAAKGKWHDHNPSGRMRDPPWHDWWGS

SEQ ID NO: 328 - protein, 29 aa, Artificial Sequence
  Description of Artificial Sequence Synthetic peptide
PASGAKTKWHDHNPSGRHRDPPWHDWRVS

SEQ ID NO: 329 - protein, 29 aa, Artificial Sequence
  Description of Artificial Sequence Synthetic peptide
PVSAAKGKTHDHWPGGRMRDPPWHDWRAS

SEQ ID NO: 330 - protein, 29 aa, Artificial Sequence
  Description of Artificial Sequence Synthetic peptide
WASGAKGKTHDHNPSGRWRDPGWHDWRGS

SEQ ID NO: 331 - protein, 29 aa, Artificial Sequence
  Description of Artificial Sequence Synthetic peptide
VGSAAKTKTHDHYGGGRMRPPPWHDWRFS

SEQ ID NO: 332 - protein, 29 aa, Artificial Sequence
  Description of Artificial Sequence Synthetic peptide
WSSAAKTKWHDHNPGGRYRPPPWHDWRFR

SEQ ID NO: 333 - protein, 29 aa, Artificial Sequence
  Description of Artificial Sequence Synthetic peptide
MPSAAKGKTHDHYPSGRMRPPPWHDWRGR

SEQ ID NO: 334 - protein, 29 aa, Artificial Sequence
  Description of Artificial Sequence Synthetic peptide
WGSGAKGKTHDHNGGGRMRPPPWHDWRAS

SEQ ID NO: 335 - protein, 29 aa, Artificial Sequence
  Description of Artificial Sequence Synthetic peptide
WSSGAKTKWHDHNPGGRMRDPPWHDWRGS

SEQ ID NO: 336 - protein, 29 aa, Artificial Sequence
  Description of Artificial Sequence Synthetic peptide
WVSAAKGKTHDHNGGGRWRDPGWHDWRFS

SEQ ID NO: 337 - protein, 29 aa, Artificial Sequence
  Description of Artificial Sequence Synthetic peptide
MGSAAKTKWHDHYPGGRMRPPPWHDWRFS

SEQ ID NO: 338 - protein, 29 aa, Artificial Sequence
  Description of Artificial Sequence Synthetic peptide
WSSAAKGKTHDHYGGGRMRPPPWHDWRAR

SEQ ID NO: 339 - protein, 29 aa, Artificial Sequence
  Description of Artificial Sequence Synthetic peptide
WPSGAKTKTHDHNWGGRMRDPPWHDWRGS

SEQ ID NO: 340 - protein, 29 aa, Artificial Sequence
  Description of Artificial Sequence Synthetic peptide
MVSTAKGKTHDHYGSGRYRPPPWHDWRGS

SEQ ID NO: 341 - protein, 29 aa, Artificial Sequence
  Description of Artificial Sequence Synthetic peptide
SASAAKGKTHDHWGGGRHRDPPWHDWRFS

SEQ ID NO: 342 - protein, 29 aa, Artificial Sequence
  Description of Artificial Sequence Synthetic peptide
MASGAKGKWHDHYGGGRMRDPGWHDWRFS

SEQ ID NO: 343 - protein, 29 aa, Artificial Sequence
  Description of Artificial Sequence Synthetic peptide
PSSAAKGKTHDHYPGGRYRDPGWHDWRGR

SEQ ID NO: 344 - protein, 29 aa, Artificial Sequence
  Description of Artificial Sequence Synthetic peptide
SSSGAKGKWHDHYGGGRHRDPPWHDWRGS

SEQ ID NO: 345 - protein, 29 aa, Artificial Sequence
  Description of Artificial Sequence Synthetic peptide
WLSAAKGKTHDHWGSGRWRDPPWHDWRFS

SEQ ID NO: 346 - protein, 29 aa, Artificial Sequence
  Description of Artificial Sequence Synthetic peptide
VHSAAKTKTHDHWGSGRMRDPPWHDWRGS

SEQ ID NO: 347 - protein, 29 aa, Artificial Sequence
  Description of Artificial Sequence Synthetic peptide
VTSAAKTKWHDHNGSGRYRDPGWHDWRGS

SEQ ID NO: 348 - protein, 29 aa, Artificial Sequence
  Description of Artificial Sequence Synthetic peptide
VPSAAKGKWHDHNGSGRMRDPGWHDWRFS

SEQ ID NO: 349 - protein, 29 aa, Artificial Sequence
  Description of Artificial Sequence Synthetic peptide
MGSAAKGKTHDHYPSGRMRPPPWHDWRGS

SEQ ID NO: 350 - protein, 29 aa, Artificial Sequence
  Description of Artificial Sequence Synthetic peptide
VSSGAKTKTHDHNPSGRMRDFKWHDWRGS

SEQ ID NO: 351 - protein, 29 aa, Artificial Sequence
  Description of Artificial Sequence Synthetic peptide
MLSAAKTKTHDHWGSGRMRDPPWHDWRVS

SEQ ID NO: 352 - protein, 29 aa, Artificial Sequence
  Description of Artificial Sequence Synthetic peptide
WQSAAKTKTHDHWPGGRMRPPPWHDWRAR

SEQ ID NO: 353 - protein, 29 aa, Artificial Sequence
  Description of Artificial Sequence Synthetic peptide
PASAAKGKTHDHWPSGRHRDPPWHDWRGS

SEQ ID NO: 354 - protein, 29 aa, Artificial Sequence
  Description of Artificial Sequence Synthetic peptide
WGSGAKTKWHDHNWSGRMRPPPWHDWRFS

SEQ ID NO: 355 - protein, 29 aa, Artificial Sequence
  Description of Artificial Sequence Synthetic peptide
FSSAAKTKWHDHYGSGRYRPPPWHDWRFS

SEQ ID NO: 356 - protein, 29 aa, Artificial Sequence
  Description of Artificial Sequence Synthetic peptide
PTSAAKGKTHDHYPGGRYRDPPWHDWRAS

SEQ ID NO: 357 - protein, 29 aa, Artificial Sequence
  Description of Artificial Sequence Synthetic peptide
WTSAAKGKTHDHYPSGRYRPPPWHDWRVR

SEQ ID NO: 358 - protein, 42 aa, Artificial Sequence
  Description of Artificial Sequence Synthetic polypeptide
AAAAAAAATHDHYPCGRMRDPGWHDWRACLTHQGIEEDEWPV

SEQ ID NO: 359 - protein, 42 aa, Artificial Sequence
  Description of Artificial Sequence Synthetic polypeptide
MLSTKKTKAAAAAACGRMRDPGWHDWRACLTHQGIEEDEWPV

SEQ ID NO: 360 - protein, 42 aa, Artificial Sequence
  Description of Artificial Sequence Synthetic polypeptide
MLSTKKTKTHDHYPCAAAAAAGWHDWRACLTHQGIEEDEWPV

SEQ ID NO: 361 - protein, 42 aa, Artificial Sequence
  Description of Artificial Sequence Synthetic polypeptide
MLSTKKTKTHDHYPCGRMRDPAAAAAAACLTHQGIEEDEWPV

SEQ ID NO: 362 - protein, 42 aa, Artificial Sequence
  Description of Artificial Sequence Synthetic polypeptide
MLSTKKTKTHDHYPCGRMRDPGWHDWRACAAAAAAAEDEWPV

SEQ ID NO: 363 - protein, 42 aa, Artificial Sequence
  Description of Artificial Sequence Synthetic polypeptide
MLSTKKTKTHDHYPCGRMRDPGWHDWRACLTHQGIEAAAAAA

SEQ ID NO: 364 - protein, 42 aa, Artificial Sequence
  Description of Artificial Sequence Synthetic polypeptide
MLSTKAAAAAAHYPCGRMRDPGWHDWRACLTHQGIEEDEWPV

SEQ ID NO: 365 - protein, 42 aa, Artificial Sequence
  Description of Artificial Sequence Synthetic polypeptide
MLSTKKTKTHDAAACAAARDPGWHDWRACLTHQGIEEDEWPV

SEQ ID NO: 366 - protein, 42 aa, Artificial Sequence
  Description of Artificial Sequence Synthetic polypeptide
MLSTKKTKTHDHYPCGRMAAAAAAAWRACLTHQGIEEDEWPV

SEQ ID NO: 367 - protein, 42 aa, Artificial Sequence
  Description of Artificial Sequence Synthetic polypeptide
MLSTKKTKTHDHYPCGRMRDPGWHDAAACAAAAGIEEDEWPV

SEQ ID NO: 368 - protein, 42 aa, Artificial Sequence
  Description of Artificial Sequence Synthetic polypeptide
MLSTKKTKTHDHYPCGRMRDPGWHDWRACLTHQAAAAAAWPV

SEQ ID NO: 369 - protein, 42 aa, Artificial Sequence
  Description of Artificial Sequence Synthetic polypeptide
MLSTKKTKTHDHYPAGRMRDPGWHDWRAALTHQGIEEDEWPV

SEQ ID NO: 370 - protein, 14 aa, Artificial Sequence
  Description of Artificial Sequence Synthetic peptide
MLSTKKTKTHDHYP

SEQ ID NO: 371 - protein, 13 aa, Artificial Sequence
  Description of Artificial Sequence Synthetic peptide
GRMRDPGWHDWRA

SEQ ID NO: 372 - protein, 13 aa, Artificial Sequence
  Description of Artificial Sequence Synthetic peptide
LTHQGIEEDEWPV

SEQ ID NO: 373 - protein, 39 aa, Artificial Sequence
  Description of Artificial Sequence Synthetic polypeptide
DLSTKKTKTHDHYPSGRMRDPGWHDWRASGGGGSGGGGS

SEQ ID NO: 374 - protein, 39 aa, Artificial Sequence
  Description of Artificial Sequence Synthetic polypeptide
ELSTKKTKTHDHYPSGRMRDPGWHDWRASGGGGSGGGGS

SEQ ID NO: 375 - protein, 39 aa, Artificial Sequence
  Description of Artificial Sequence Synthetic polypeptide
LLSTKKTKTHDHYPSGRMRDPGWHDWRASGGGGSGGGGS

SEQ ID NO: 376 - protein, 39 aa, Artificial Sequence
  Description of Artificial Sequence Synthetic polypeptide
NLSTKKTKTHDHYPSGRMRDPGWHDWRASGGGGSGGGGS

SEQ ID NO: 377 - protein, 39 aa, Artificial Sequence
  Description of Artificial Sequence Synthetic polypeptide
TLSTKKTKTHDHYPSGRMRDPGWHDWRASGGGGSGGGGS

SEQ ID NO: 378 - protein, 39 aa, Artificial Sequence
  Description of Artificial Sequence Synthetic polypeptide
MESTKKTKTHDHYPSGRMRDPGWHDWRASGGGGSGGGGS

SEQ ID NO: 379 - protein, 39 aa, Artificial Sequence
  Description of Artificial Sequence Synthetic polypeptide
MDSTKKTKTHDHYPSGRMRDPGWHDWRASGGGGSGGGGS

SEQ ID NO: 380 - protein, 39 aa, Artificial Sequence
  Description of Artificial Sequence Synthetic polypeptide
MLYTKKTKTHDHYPSGRMRDPGWHDWRASGGGGSGGGGS

SEQ ID NO: 381 - protein, 39 aa, Artificial Sequence
  Description of Artificial Sequence Synthetic polypeptide
MLWTKKTKTHDHYPSGRMRDPGWHDWRASGGGGSGGGGS

SEQ ID NO: 382 - protein, 39 aa, Artificial Sequence
  Description of Artificial Sequence Synthetic polypeptide
MLTTKKTKTHDHYPSGRMRDPGWHDWRASGGGGSGGGGS

SEQ ID NO: 383 - protein, 39 aa, Artificial Sequence
  Description of Artificial Sequence Synthetic polypeptide
MLETKKTKTHDHYPSGRMRDPGWHDWRASGGGGSGGGGS

SEQ ID NO: 384 - protein, 39 aa, Artificial Sequence
  Description of Artificial Sequence Synthetic polypeptide
MLKTKKTKTHDHYPSGRMRDPGWHDWRASGGGGSGGGGS

SEQ ID NO: 385 - protein, 39 aa, Artificial Sequence
  Description of Artificial Sequence Synthetic polypeptide
MLHTKKTKTHDHYPSGRMRDPGWHDWRASGGGGSGGGGS

SEQ ID NO: 386 - protein, 39 aa, Artificial Sequence
  Description of Artificial Sequence Synthetic polypeptide
MLLTKKTKTHDHYPSGRMRDPGWHDWRASGGGGSGGGGS

SEQ ID NO: 387 - protein, 39 aa, Artificial Sequence
  Description of Artificial Sequence Synthetic polypeptide
MLNTKKTKTHDHYPSGRMRDPGWHDWRASGGGGSGGGGS

SEQ ID NO: 388 - protein, 39 aa, Artificial Sequence
  Description of Artificial Sequence Synthetic polypeptide
MLATKKTKTHDHYPSGRMRDPGWHDWRASGGGGSGGGGS

SEQ ID NO: 389 - protein, 39 aa, Artificial Sequence
  Description of Artificial Sequence Synthetic polypeptide
MLDTKKTKTHDHYPSGRMRDPGWHDWRASGGGGSGGGGS

SEQ ID NO: 390 - protein, 39 aa, Artificial Sequence
  Description of Artificial Sequence Synthetic polypeptide
MLSYKKTKTHDHYPSGRMRDPGWHDWRASGGGGSGGGGS

SEQ ID NO: 391 - protein, 39 aa, Artificial Sequence
  Description of Artificial Sequence Synthetic polypeptide
MLSNKKTKTHDHYPSGRMRDPGWHDWRASGGGGSGGGGS

SEQ ID NO: 392 - protein, 39 aa, Artificial Sequence
  Description of Artificial Sequence Synthetic polypeptide
MLSHKKTKTHDHYPSGRMRDPGWHDWRASGGGGSGGGGS

SEQ ID NO: 393 - protein, 39 aa, Artificial Sequence
  Description of Artificial Sequence Synthetic polypeptide
MLSPKKTKTHDHYPSGRMRDPGWHDWRASGGGGSGGGGS

SEQ ID NO: 394 - protein, 39 aa, Artificial Sequence
  Description of Artificial Sequence Synthetic polypeptide
MLSSKKTKTHDHYPSGRMRDPGWHDWRASGGGGSGGGGS

SEQ ID NO: 395 - protein, 39 aa, Artificial Sequence
  Description of Artificial Sequence Synthetic polypeptide
MLSQKKTKTHDHYPSGRMRDPGWHDWRASGGGGSGGGGS

SEQ ID NO: 396 - protein, 39 aa, Artificial Sequence
  Description of Artificial Sequence Synthetic polypeptide
MLSVKKTKTHDHYPSGRMRDPGWHDWRASGGGGSGGGGS

SEQ ID NO: 397 - protein, 39 aa, Artificial Sequence
  Description of Artificial Sequence Synthetic polypeptide
MLSFKKTKTHDHYPSGRMRDPGWHDWRASGGGGSGGGGS

SEQ ID NO: 398 - protein, 39 aa, Artificial Sequence
  Description of Artificial Sequence Synthetic polypeptide
MLSLKKTKTHDHYPSGRMRDPGWHDWRASGGGGSGGGGS

SEQ ID NO: 399 - protein, 39 aa, Artificial Sequence
  Description of Artificial Sequence Synthetic polypeptide
MLSIKKTKTHDHYPSGRMRDPGWHDWRASGGGGSGGGGS

SEQ ID NO: 400 - protein, 39 aa, Artificial Sequence
  Description of Artificial Sequence Synthetic polypeptide
MLSDKKTKTHDHYPSGRMRDPGWHDWRASGGGGSGGGGS

SEQ ID NO: 401 - protein, 39 aa, Artificial Sequence
  Description of Artificial Sequence Synthetic polypeptide
MLSEKKTKTHDHYPSGRMRDPGWHDWRASGGGGSGGGGS

SEQ ID NO: 402 - protein, 39 aa, Artificial Sequence
  Description of Artificial Sequence Synthetic polypeptide
MLSTVKTKTHDHYPSGRMRDPGWHDWRASGGGGSGGGGS

SEQ ID NO: 403 - protein, 39 aa, Artificial Sequence
  Description of Artificial Sequence Synthetic polypeptide
MLSTTKTKTHDHYPSGRMRDPGWHDWRASGGGGSGGGGS

SEQ ID NO: 404 - protein, 39 aa, Artificial Sequence
  Description of Artificial Sequence Synthetic polypeptide
MLSTLKTKTHDHYPSGRMRDPGWHDWRASGGGGSGGGGS

SEQ ID NO: 405 - protein, 39 aa, Artificial Sequence
  Description of Artificial Sequence Synthetic polypeptide
MLSTSKTKTHDHYPSGRMRDPGWHDWRASGGGGSGGGGS

SEQ ID NO: 406 - protein, 39 aa, Artificial Sequence
  Description of Artificial Sequence Synthetic polypeptide
MLSTHKTKTHDHYPSGRMRDPGWHDWRASGGGGSGGGGS

SEQ ID NO: 407 - protein, 39 aa, Artificial Sequence
  Description of Artificial Sequence Synthetic polypeptide
MLSTFKTKTHDHYPSGRMRDPGWHDWRASGGGGSGGGGS

SEQ ID NO: 408 - protein, 39 aa, Artificial Sequence
  Description of Artificial Sequence Synthetic polypeptide
MLSTQKTKTHDHYPSGRMRDPGWHDWRASGGGGSGGGGS

SEQ ID NO: 409 - protein, 39 aa, Artificial Sequence
  Description of Artificial Sequence Synthetic polypeptide
MLSTNKTKTHDHYPSGRMRDPGWHDWRASGGGGSGGGGS

SEQ ID NO: 410 - protein, 39 aa, Artificial Sequence
  Description of Artificial Sequence Synthetic polypeptide
MLSTPKTKTHDHYPSGRMRDPGWHDWRASGGGGSGGGGS

SEQ ID NO: 411 - protein, 39 aa, Artificial Sequence
  Description of Artificial Sequence Synthetic polypeptide
MLSTYKTKTHDHYPSGRMRDPGWHDWRASGGGGSGGGGS

SEQ ID NO: 412 - protein, 39 aa, Artificial Sequence
  Description of Artificial Sequence Synthetic polypeptide
MLSTGKTKTHDHYPSGRMRDPGWHDWRASGGGGSGGGGS

SEQ ID NO: 413 - protein, 39 aa, Artificial Sequence
  Description of Artificial Sequence Synthetic polypeptide
MLSTIKTKTHDHYPSGRMRDPGWHDWRASGGGGSGGGGS

SEQ ID NO: 414 - protein, 39 aa, Artificial Sequence
  Description of Artificial Sequence Synthetic polypeptide
MLSTWKTKTHDHYPSGRMRDPGWHDWRASGGGGSGGGGS

SEQ ID NO: 415 - protein, 39 aa, Artificial Sequence
  Description of Artificial Sequence Synthetic polypeptide
MLSTEKTKTHDHYPSGRMRDPGWHDWRASGGGGSGGGGS

SEQ ID NO: 416 - protein, 39 aa, Artificial Sequence
  Description of Artificial Sequence Synthetic polypeptide
MLSTDKTKTHDHYPSGRMRDPGWHDWRASGGGGSGGGGS

SEQ ID NO: 417 - protein, 39 aa, Artificial Sequence
  Description of Artificial Sequence Synthetic polypeptide
MLSTKITKTHDHYPSGRMRDPGWHDWRASGGGGSGGGGS

SEQ ID NO: 418 - protein, 39 aa, Artificial Sequence
  Description of Artificial Sequence Synthetic polypeptide
MLSTKLTKTHDHYPSGRMRDPGWHDWRASGGGGSGGGGS

SEQ ID NO: 419 - protein, 39 aa, Artificial Sequence
  Description of Artificial Sequence Synthetic polypeptide
MLSTKWTKTHDHYPSGRMRDPGWHDWRASGGGGSGGGGS

SEQ ID NO: 420 - protein, 39 aa, Artificial Sequence
  Description of Artificial Sequence Synthetic polypeptide
MLSTKYTKTHDHYPSGRMRDPGWHDWRASGGGGSGGGGS

SEQ ID NO: 421 - protein, 39 aa, Artificial Sequence
  Description of Artificial Sequence Synthetic polypeptide
MLSTKFTKTHDHYPSGRMRDPGWHDWRASGGGGSGGGGS

SEQ ID NO: 422 - protein, 39 aa, Artificial Sequence
  Description of Artificial Sequence Synthetic polypeptide
MLSTKVTKTHDHYPSGRMRDPGWHDWRASGGGGSGGGGS

SEQ ID NO: 423 - protein, 39 aa, Artificial Sequence
  Description of Artificial Sequence Synthetic polypeptide
MLSTKSTKTHDHYPSGRMRDPGWHDWRASGGGGSGGGGS

SEQ ID NO: 424 - protein, 39 aa, Artificial Sequence
  Description of Artificial Sequence Synthetic polypeptide
MLSTKNTKTHDHYPSGRMRDPGWHDWRASGGGGSGGGGS

SEQ ID NO: 425 - protein, 39 aa, Artificial Sequence
  Description of Artificial Sequence Synthetic polypeptide
MLSTKGTKTHDHYPSGRMRDPGWHDWRASGGGGSGGGGS

SEQ ID NO: 426 - protein, 39 aa, Artificial Sequence
  Description of Artificial Sequence Synthetic polypeptide
MLSTKHTKTHDHYPSGRMRDPGWHDWRASGGGGSGGGGS

SEQ ID NO: 427 - protein, 39 aa, Artificial Sequence
  Description of Artificial Sequence Synthetic polypeptide
MLSTKQTKTHDHYPSGRMRDPGWHDWRASGGGGSGGGGS

SEQ ID NO: 428 - protein, 39 aa, Artificial Sequence
  Description of Artificial Sequence Synthetic polypeptide
MLSTKATKTHDHYPSGRMRDPGWHDWRASGGGGSGGGGS

SEQ ID NO: 429 - protein, 39 aa, Artificial Sequence
  Description of Artificial Sequence Synthetic polypeptide
MLSTKPTKTHDHYPSGRMRDPGWHDWRASGGGGSGGGGS

SEQ ID NO: 430 - protein, 39 aa, Artificial Sequence
  Description of Artificial Sequence Synthetic polypeptide
MLSTKETKTHDHYPSGRMRDPGWHDWRASGGGGSGGGGS

SEQ ID NO: 431 - protein, 39 aa, Artificial Sequence
  Description of Artificial Sequence Synthetic polypeptide
MLSTKDTKTHDHYPSGRMRDPGWHDWRASGGGGSGGGGS

SEQ ID NO: 432 - protein, 39 aa, Artificial Sequence
  Description of Artificial Sequence Synthetic polypeptide
MLSTKKSKTHDHYPSGRMRDPGWHDWRASGGGGSGGGGS

SEQ ID NO: 433 - protein, 39 aa, Artificial Sequence
  Description of Artificial Sequence Synthetic polypeptide
MLSTKKLKTHDHYPSGRMRDPGWHDWRASGGGGSGGGGS

SEQ ID NO: 434 - protein, 39 aa, Artificial Sequence
  Description of Artificial Sequence Synthetic polypeptide
MLSTKKWKTHDHYPSGRMRDPGWHDWRASGGGGSGGGGS

SEQ ID NO: 435 - protein, 39 aa, Artificial Sequence
  Description of Artificial Sequence Synthetic polypeptide
MLSTKKFKTHDHYPSGRMRDPGWHDWRASGGGGSGGGGS

SEQ ID NO: 436 - protein, 39 aa, Artificial Sequence
  Description of Artificial Sequence Synthetic polypeptide
MLSTKKAKTHDHYPSGRMRDPGWHDWRASGGGGSGGGGS

SEQ ID NO: 437 - protein, 39 aa, Artificial Sequence
  Description of Artificial Sequence Synthetic polypeptide
MLSTKKIKTHDHYPSGRMRDPGWHDWRASGGGGSGGGGS

SEQ ID NO: 438 - protein, 39 aa, Artificial Sequence
  Description of Artificial Sequence Synthetic polypeptide
MLSTKKHKTHDHYPSGRMRDPGWHDWRASGGGGSGGGGS

SEQ ID NO: 439 - protein, 39 aa, Artificial Sequence
  Description of Artificial Sequence Synthetic polypeptide
MLSTKKPKTHDHYPSGRMRDPGWHDWRASGGGGSGGGGS

SEQ ID NO: 440 - protein, 39 aa, Artificial Sequence
  Description of Artificial Sequence Synthetic polypeptide
MLSTKKVKTHDHYPSGRMRDPGWHDWRASGGGGSGGGGS

SEQ ID NO: 441 - protein, 39 aa, Artificial Sequence
  Description of Artificial Sequence Synthetic polypeptide
MLSTKKYKTHDHYPSGRMRDPGWHDWRASGGGGSGGGGS

SEQ ID NO: 442 - protein, 39 aa, Artificial Sequence
  Description of Artificial Sequence Synthetic polypeptide
MLSTKKEKTHDHYPSGRMRDPGWHDWRASGGGGSGGGGS

SEQ ID NO: 443 - protein, 39 aa, Artificial Sequence
  Description of Artificial Sequence Synthetic polypeptide
MLSTKKDKTHDHYPSGRMRDPGWHDWRASGGGGSGGGGS

SEQ ID NO: 444 - protein, 39 aa, Artificial Sequence
  Description of Artificial Sequence Synthetic polypeptide
MLSTKKTHTHDHYPSGRMRDPGWHDWRASGGGGSGGGGS

SEQ ID NO: 445 - protein, 39 aa, Artificial Sequence
  Description of Artificial Sequence Synthetic polypeptide
MLSTKKTPTHDHYPSGRMRDPGWHDWRASGGGGSGGGGS

SEQ ID NO: 446 - protein, 39 aa, Artificial Sequence
  Description of Artificial Sequence Synthetic polypeptide
MLSTKKTFTHDHYPSGRMRDPGWHDWRASGGGGSGGGGS

SEQ ID NO: 447 - protein, 39 aa, Artificial Sequence
  Description of Artificial Sequence Synthetic polypeptide
MLSTKKTGTHDHYPSGRMRDPGWHDWRASGGGGSGGGGS

SEQ ID NO: 448 - protein, 39 aa, Artificial Sequence
  Description of Artificial Sequence Synthetic polypeptide
MLSTKKTATHDHYPSGRMRDPGWHDWRASGGGGSGGGGS

SEQ ID NO: 449 - protein, 39 aa, Artificial Sequence
  Description of Artificial Sequence Synthetic polypeptide
MLSTKKTLTHDHYPSGRMRDPGWHDWRASGGGGSGGGGS

SEQ ID NO: 450 - protein, 39 aa, Artificial Sequence
  Description of Artificial Sequence Synthetic polypeptide
MLSTKKTQTHDHYPSGRMRDPGWHDWRASGGGGSGGGGS

SEQ ID NO: 451 - protein, 39 aa, Artificial Sequence
  Description of Artificial Sequence Synthetic polypeptide
MLSTKKTYTHDHYPSGRMRDPGWHDWRASGGGGSGGGGS

SEQ ID NO: 452 - protein, 39 aa, Artificial Sequence
  Description of Artificial Sequence Synthetic polypeptide
MLSTKKTVTHDHYPSGRMRDPGWHDWRASGGGGSGGGGS

SEQ ID NO: 453 - protein, 39 aa, Artificial Sequence
  Description of Artificial Sequence Synthetic polypeptide
MLSTKKTSTHDHYPSGRMRDPGWHDWRASGGGGSGGGGS

SEQ ID NO: 454 - protein, 39 aa, Artificial Sequence
  Description of Artificial Sequence Synthetic polypeptide
MLSTKKTTTHDHYPSGRMRDPGWHDWRASGGGGSGGGGS

SEQ ID NO: 455 - protein, 39 aa, Artificial Sequence
  Description of Artificial Sequence Synthetic polypeptide
MLSTKKTDTHDHYPSGRMRDPGWHDWRASGGGGSGGGGS

SEQ ID NO: 456 - protein, 39 aa, Artificial Sequence
  Description of Artificial Sequence Synthetic polypeptide
MLSTKKTITHDHYPSGRMRDPGWHDWRASGGGGSGGGGS

SEQ ID NO: 457 - protein, 39 aa, Artificial Sequence
  Description of Artificial Sequence Synthetic polypeptide
MLSTKKTNTHDHYPSGRMRDPGWHDWRASGGGGSGGGGS

SEQ ID NO: 458 - protein, 39 aa, Artificial Sequence
  Description of Artificial Sequence Synthetic polypeptide
MLSTKKTETHDHYPSGRMRDPGWHDWRASGGGGSGGGGS

SEQ ID NO: 459 - protein, 39 aa, Artificial Sequence
  Description of Artificial Sequence Synthetic polypeptide
MLSTKKTKLHDHYPSGRMRDPGWHDWRASGGGGSGGGGS

SEQ ID NO: 460 - protein, 39 aa, Artificial Sequence
  Description of Artificial Sequence Synthetic polypeptide
MLSTKKTKYHDHYPSGRMRDPGWHDWRASGGGGSGGGGS

SEQ ID NO: 461 - protein, 39 aa, Artificial Sequence
  Description of Artificial Sequence Synthetic polypeptide
MLSTKKTKPHDHYPSGRMRDPGWHDWRASGGGGSGGGGS

SEQ ID NO: 462 - protein, 39 aa, Artificial Sequence
  Description of Artificial Sequence Synthetic polypeptide
MLSTKKTKKHDHYPSGRMRDPGWHDWRASGGGGSGGGGS

SEQ ID NO: 463 - protein, 39 aa, Artificial Sequence
  Description of Artificial Sequence Synthetic polypeptide
MLSTKKTKNHDHYPSGRMRDPGWHDWRASGGGGSGGGGS

SEQ ID NO: 464 - protein, 39 aa, Artificial Sequence
  Description of Artificial Sequence Synthetic polypeptide
MLSTKKTKFHDHYPSGRMRDPGWHDWRASGGGGSGGGGS

SEQ ID NO: 465 - protein, 39 aa, Artificial Sequence
  Description of Artificial Sequence Synthetic polypeptide
MLSTKKTKSHDHYPSGRMRDPGWHDWRASGGGGSGGGGS

SEQ ID NO: 466 - protein, 39 aa, Artificial Sequence
  Description of Artificial Sequence Synthetic polypeptide
MLSTKKTKHHDHYPSGRMRDPGWHDWRASGGGGSGGGGS

SEQ ID NO: 467 - protein, 39 aa, Artificial Sequence
  Description of Artificial Sequence Synthetic polypeptide
MLSTKKTKQHDHYPSGRMRDPGWHDWRASGGGGSGGGGS

SEQ ID NO: 468 - protein, 39 aa, Artificial Sequence
  Description of Artificial Sequence Synthetic polypeptide
MLSTKKTKAHDHYPSGRMRDPGWHDWRASGGGGSGGGGS

SEQ ID NO: 469 - protein, 39 aa, Artificial Sequence
  Description of Artificial Sequence Synthetic polypeptide
MLSTKKTKVHDHYPSGRMRDPGWHDWRASGGGGSGGGGS

SEQ ID NO: 470 - protein, 39 aa, Artificial Sequence
  Description of Artificial Sequence Synthetic polypeptide
MLSTKKTKEHDHYPSGRMRDPGWHDWRASGGGGSGGGGS

SEQ ID NO: 471 - protein, 39 aa, Artificial Sequence
  Description of Artificial Sequence Synthetic polypeptide
MLSTKKTKIHDHYPSGRMRDPGWHDWRASGGGGSGGGGS

SEQ ID NO: 472 - protein, 39 aa, Artificial Sequence
  Description of Artificial Sequence Synthetic polypeptide
MLSTKKTKDHDHYPSGRMRDPGWHDWRASGGGGSGGGGS

SEQ ID NO: 473 - protein, 39 aa, Artificial Sequence
  Description of Artificial Sequence Synthetic polypeptide
MLSTKKTKTWDHYPSGRMRDPGWHDWRASGGGGSGGGGS

SEQ ID NO: 474 - protein, 39 aa, Artificial Sequence
  Description of Artificial Sequence Synthetic polypeptide
MLSTKKTKTFDHYPSGRMRDPGWHDWRASGGGGSGGGGS

SEQ ID NO: 475 - protein, 39 aa, Artificial Sequence
  Description of Artificial Sequence Synthetic polypeptide
MLSTKKTKTPDHYPSGRMRDPGWHDWRASGGGGSGGGGS

SEQ ID NO: 476 - protein, 39 aa, Artificial Sequence
  Description of Artificial Sequence Synthetic polypeptide
MLSTKKTKTQDHYPSGRMRDPGWHDWRASGGGGSGGGGS

SEQ ID NO: 477 - protein, 39 aa, Artificial Sequence
  Description of Artificial Sequence Synthetic polypeptide
MLSTKKTKTLDHYPSGRMRDPGWHDWRASGGGGSGGGGS

SEQ ID NO: 478 - protein, 39 aa, Artificial Sequence
  Description of Artificial Sequence Synthetic polypeptide
MLSTKKTKTTDHYPSGRMRDPGWHDWRASGGGGSGGGGS

SEQ ID NO: 479 - protein, 39 aa, Artificial Sequence
  Description of Artificial Sequence Synthetic polypeptide
MLSTKKTKTYDHYPSGRMRDPGWHDWRASGGGGSGGGGS

SEQ ID NO: 480 - protein, 39 aa, Artificial Sequence
  Description of Artificial Sequence Synthetic polypeptide
MLSTKKTKTDDHYPSGRMRDPGWHDWRASGGGGSGGGGS

SEQ ID NO: 481 - protein, 39 aa, Artificial Sequence
  Description of Artificial Sequence Synthetic polypeptide
MLSTKKTKTEDHYPSGRMRDPGWHDWRASGGGGSGGGGS

SEQ ID NO: 482 - protein, 39 aa, Artificial Sequence
  Description of Artificial Sequence Synthetic polypeptide
MLSTKKTKTVDHYPSGRMRDPGWHDWRASGGGGSGGGGS

SEQ ID NO: 483 - protein, 39 aa, Artificial Sequence
  Description of Artificial Sequence Synthetic polypeptide
MLSTKKTKTADHYPSGRMRDPGWHDWRASGGGGSGGGGS

SEQ ID NO: 484 - protein, 39 aa, Artificial Sequence
  Description of Artificial Sequence Synthetic polypeptide
MLSTKKTKTIDHYPSGRMRDPGWHDWRASGGGGSGGGGS

SEQ ID NO: 485 - protein, 39 aa, Artificial Sequence
  Description of Artificial Sequence Synthetic polypeptide
MLSTKKTKTHEHYPSGRMRDPGWHDWRASGGGGSGGGGS

SEQ ID NO: 486 - protein, 39 aa, Artificial Sequence
  Description of Artificial Sequence Synthetic polypeptide
MLSTKKTKTHDNYPSGRMRDPGWHDWRASGGGGSGGGGS

SEQ ID NO: 487 - protein, 39 aa, Artificial Sequence
  Description of Artificial Sequence Synthetic polypeptide
MLSTKKTKTHDWYPSGRMRDPGWHDWRASGGGGSGGGGS

SEQ ID NO: 488 - protein, 39 aa, Artificial Sequence
  Description of Artificial Sequence Synthetic polypeptide
MLSTKKTKTHDLYPSGRMRDPGWHDWRASGGGGSGGGGS

SEQ ID NO: 489 - protein, 39 aa, Artificial Sequence
  Description of Artificial Sequence Synthetic polypeptide
MLSTKKTKTHDFYPSGRMRDPGWHDWRASGGGGSGGGGS

SEQ ID NO: 490 - protein, 39 aa, Artificial Sequence
  Description of Artificial Sequence Synthetic polypeptide
MLSTKKTKTHDTYPSGRMRDPGWHDWRASGGGGSGGGGS

SEQ ID NO: 491 - protein, 39 aa, Artificial Sequence
  Description of Artificial Sequence Synthetic polypeptide
MLSTKKTKTHDQYPSGRMRDPGWHDWRASGGGGSGGGGS

SEQ ID NO: 492 - protein, 39 aa, Artificial Sequence
  Description of Artificial Sequence Synthetic polypeptide
MLSTKKTKTHDGYPSGRMRDPGWHDWRASGGGGSGGGGS

SEQ ID NO: 493 - protein, 39 aa, Artificial Sequence
  Description of Artificial Sequence Synthetic polypeptide
MLSTKKTKTHDAYPSGRMRDPGWHDWRASGGGGSGGGGS

SEQ ID NO: 494 - protein, 39 aa, Artificial Sequence
  Description of Artificial Sequence Synthetic polypeptide
MLSTKKTKTHDIYPSGRMRDPGWHDWRASGGGGSGGGGS

SEQ ID NO: 495 - protein, 39 aa, Artificial Sequence
  Description of Artificial Sequence Synthetic polypeptide
MLSTKKTKTHDSYPSGRMRDPGWHDWRASGGGGSGGGGS

SEQ ID NO: 496 - protein, 39 aa, Artificial Sequence
  Description of Artificial Sequence Synthetic polypeptide
MLSTKKTKTHDYYPSGRMRDPGWHDWRASGGGGSGGGGS

SEQ ID NO: 497 - protein, 39 aa, Artificial Sequence
  Description of Artificial Sequence Synthetic polypeptide
MLSTKKTKTHDPYPSGRMRDPGWHDWRASGGGGSGGGGS

SEQ ID NO: 498 - protein, 39 aa, Artificial Sequence
  Description of Artificial Sequence Synthetic polypeptide
MLSTKKTKTHDDYPSGRMRDPGWHDWRASGGGGSGGGGS

SEQ ID NO: 499 - protein, 39 aa, Artificial Sequence
  Description of Artificial Sequence Synthetic polypeptide
MLSTKKTKTHDVYPSGRMRDPGWHDWRASGGGGSGGGGS

SEQ ID NO: 500 - protein, 39 aa, Artificial Sequence
  Description of Artificial Sequence Synthetic polypeptide
MLSTKKTKTHDEYPSGRMRDPGWHDWRASGGGGSGGGGS

SEQ ID NO: 501 - protein, 39 aa, Artificial Sequence
  Description of Artificial Sequence Synthetic polypeptide
MLSTKKTKTHDHHPSGRMRDPGWHDWRASGGGGSGGGGS

SEQ ID NO: 502 - protein, 39 aa, Artificial Sequence
  Description of Artificial Sequence Synthetic polypeptide
MLSTKKTKTHDHSPSGRMRDPGWHDWRASGGGGSGGGGS

SEQ ID NO: 503 - protein, 39 aa, Artificial Sequence
  Description of Artificial Sequence Synthetic polypeptide
MLSTKKTKTHDHVPSGRMRDPGWHDWRASGGGGSGGGGS

SEQ ID NO: 504 - protein, 39 aa, Artificial Sequence
  Description of Artificial Sequence Synthetic polypeptide
MLSTKKTKTHDHQPSGRMRDPGWHDWRASGGGGSGGGGS

SEQ ID NO: 505 - protein, 39 aa, Artificial Sequence
  Description of Artificial Sequence Synthetic polypeptide
MLSTKKTKTHDHAPSGRMRDPGWHDWRASGGGGSGGGGS

SEQ ID NO: 506 - protein, 39 aa, Artificial Sequence
  Description of Artificial Sequence Synthetic polypeptide
MLSTKKTKTHDHDPSGRMRDPGWHDWRASGGGGSGGGGS

SEQ ID NO: 507 - protein, 39 aa, Artificial Sequence
  Description of Artificial Sequence Synthetic polypeptide
MLSTKKTKTHDHIPSGRMRDPGWHDWRASGGGGSGGGGS

SEQ ID NO: 508 - protein, 39 aa, Artificial Sequence
  Description of Artificial Sequence Synthetic polypeptide
MLSTKKTKTHDHTPSGRMRDPGWHDWRASGGGGSGGGGS

SEQ ID NO: 509 - protein, 39 aa, Artificial Sequence
  Description of Artificial Sequence Synthetic polypeptide
MLSTKKTKTHDHEPSGRMRDPGWHDWRASGGGGSGGGGS

SEQ ID NO: 510 - protein, 39 aa, Artificial Sequence
  Description of Artificial Sequence Synthetic polypeptide
MLSTKKTKTHDHYNSGRMRDPGWHDWRASGGGGSGGGGS

SEQ ID NO: 511 - protein, 39 aa, Artificial Sequence
  Description of Artificial Sequence Synthetic polypeptide
MLSTKKTKTHDHYRSGRMRDPGWHDWRASGGGGSGGGGS

SEQ ID NO: 512 - protein, 39 aa, Artificial Sequence
  Description of Artificial Sequence Synthetic polypeptide
MLSTKKTKTHDHYSSGRMRDPGWHDWRASGGGGSGGGGS

SEQ ID NO: 513 - protein, 39 aa, Artificial Sequence
  Description of Artificial Sequence Synthetic polypeptide
MLSTKKTKTHDHYLSGRMRDPGWHDWRASGGGGSGGGGS

SEQ ID NO: 514 - protein, 39 aa, Artificial Sequence
  Description of Artificial Sequence Synthetic polypeptide
MLSTKKTKTHDHYVSGRMRDPGWHDWRASGGGGSGGGGS

SEQ ID NO: 515 - protein, 39 aa, Artificial Sequence
  Description of Artificial Sequence Synthetic polypeptide
MLSTKKTKTHDHYTSGRMRDPGWHDWRASGGGGSGGGGS

SEQ ID NO: 516 - protein, 39 aa, Artificial Sequence
  Description of Artificial Sequence Synthetic polypeptide
MLSTKKTKTHDHYASGRMRDPGWHDWRASGGGGSGGGGS

SEQ ID NO: 517 - protein, 39 aa, Artificial Sequence
  Description of Artificial Sequence Synthetic polypeptide
MLSTKKTKTHDHYHSGRMRDPGWHDWRASGGGGSGGGGS

SEQ ID NO: 518 - protein, 39 aa, Artificial Sequence
  Description of Artificial Sequence Synthetic polypeptide
MLSTKKTKTHDHYQSGRMRDPGWHDWRASGGGGSGGGGS

SEQ ID NO: 519 - protein, 39 aa, Artificial Sequence
  Description of Artificial Sequence Synthetic polypeptide
MLSTKKTKTHDHYDSGRMRDPGWHDWRASGGGGSGGGGS

SEQ ID NO: 520 - protein, 39 aa, Artificial Sequence
  Description of Artificial Sequence Synthetic polypeptide
MLSTKKTKTHDHYKSGRMRDPGWHDWRASGGGGSGGGGS

SEQ ID NO: 521 - protein, 39 aa, Artificial Sequence
  Description of Artificial Sequence Synthetic polypeptide
MLSTKKTKTHDHYESGRMRDPGWHDWRASGGGGSGGGGS

SEQ ID NO: 522 - protein, 39 aa, Artificial Sequence
  Description of Artificial Sequence Synthetic polypeptide
MLSTKKTKTHDHYPTGRMRDPGWHDWRASGGGGSGGGGS

SEQ ID NO: 523 - protein, 39 aa, Artificial Sequence
  Description of Artificial Sequence Synthetic polypeptide
MLSTKKTKTHDHYPDGRMRDPGWHDWRASGGGGSGGGGS

SEQ ID NO: 524 - protein, 39 aa, Artificial Sequence
  Description of Artificial Sequence Synthetic polypeptide
MLSTKKTKTHDHYPWGRMRDPGWHDWRASGGGGSGGGGS

SEQ ID NO: 525 - protein, 39 aa, Artificial Sequence
  Description of Artificial Sequence Synthetic polypeptide
MLSTKKTKTHDHYPNGRMRDPGWHDWRASGGGGSGGGGS

SEQ ID NO: 526 - protein, 39 aa, Artificial Sequence
  Description of Artificial Sequence Synthetic polypeptide
MLSTKKTKTHDHYPHGRMRDPGWHDWRASGGGGSGGGGS

SEQ ID NO: 527 - protein, 39 aa, Artificial Sequence
  Description of Artificial Sequence Synthetic polypeptide
MLSTKKTKTHDHYPFGRMRDPGWHDWRASGGGGSGGGGS

SEQ ID NO: 528 - protein, 39 aa, Artificial Sequence
  Description of Artificial Sequence Synthetic polypeptide
MLSTKKTKTHDHYPQGRMRDPGWHDWRASGGGGSGGGGS

SEQ ID NO: 529 - protein, 39 aa, Artificial Sequence
  Description of Artificial Sequence Synthetic polypeptide
MLSTKKTKTHDHYPLGRMRDPGWHDWRASGGGGSGGGGS

SEQ ID NO: 530 - protein, 39 aa, Artificial Sequence
  Description of Artificial Sequence Synthetic polypeptide
MLSTKKTKTHDHYPEGRMRDPGWHDWRASGGGGSGGGGS

SEQ ID NO: 531 - protein, 39 aa, Artificial Sequence
  Description of Artificial Sequence Synthetic polypeptide
MLSTKKTKTHDHYPAGRMRDPGWHDWRASGGGGSGGGGS

SEQ ID NO: 532 - protein, 39 aa, Artificial Sequence
  Description of Artificial Sequence Synthetic polypeptide
MLSTKKTKTHDHYPYGRMRDPGWHDWRASGGGGSGGGGS

SEQ ID NO: 533 - protein, 39 aa, Artificial Sequence
  Description of Artificial Sequence Synthetic polypeptide
MLSTKKTKTHDHYPVGRMRDPGWHDWRASGGGGSGGGGS

SEQ ID NO: 534 - protein, 39 aa, Artificial Sequence
  Description of Artificial Sequence Synthetic polypeptide
MLSTKKTKTHDHYPIGRMRDPGWHDWRASGGGGSGGGGS

SEQ ID NO: 535 - protein, 39 aa, Artificial Sequence
  Description of Artificial Sequence Synthetic polypeptide
MLSTKKTKTHDHYPSPRMRDPGWHDWRASGGGGSGGGGS

SEQ ID NO: 536 - protein, 39 aa, Artificial Sequence
  Description of Artificial Sequence Synthetic polypeptide
MLSTKKTKTHDHYPSKRMRDPGWHDWRASGGGGSGGGGS

SEQ ID NO: 537 - protein, 39 aa, Artificial Sequence
  Description of Artificial Sequence Synthetic polypeptide
MLSTKKTKTHDHYPSNRMRDPGWHDWRASGGGGSGGGGS

SEQ ID NO: 538 - protein, 39 aa, Artificial Sequence
  Description of Artificial Sequence Synthetic polypeptide
MLSTKKTKTHDHYPSRRMRDPGWHDWRASGGGGSGGGGS

SEQ ID NO: 539 - protein, 39 aa, Artificial Sequence
  Description of Artificial Sequence Synthetic polypeptide
MLSTKKTKTHDHYPSERMRDPGWHDWRASGGGGSGGGGS

SEQ ID NO: 540 - protein, 39 aa, Artificial Sequence
  Description of Artificial Sequence Synthetic polypeptide
MLSTKKTKTHDHYPSWRMRDPGWHDWRASGGGGSGGGGS

SEQ ID NO: 541 - protein, 39 aa, Artificial Sequence
  Description of Artificial Sequence Synthetic polypeptide
MLSTKKTKTHDHYPSLRMRDPGWHDWRASGGGGSGGGGS

SEQ ID NO: 542 - protein, 39 aa, Artificial Sequence
  Description of Artificial Sequence Synthetic polypeptide
MLSTKKTKTHDHYPSDRMRDPGWHDWRASGGGGSGGGGS

SEQ ID NO: 543 - protein, 39 aa, Artificial Sequence
  Description of Artificial Sequence Synthetic polypeptide
MLSTKKTKTHDHYPSARMRDPGWHDWRASGGGGSGGGGS

SEQ ID NO: 544 - protein, 39 aa, Artificial Sequence
  Description of Artificial Sequence Synthetic polypeptide
MLSTKKTKTHDHYPSHRMRDPGWHDWRASGGGGSGGGGS

SEQ ID NO: 545 - protein, 39 aa, Artificial Sequence
  Description of Artificial Sequence Synthetic polypeptide
MLSTKKTKTHDHYPSQRMRDPGWHDWRASGGGGSGGGGS

SEQ ID NO: 546 - protein, 39 aa, Artificial Sequence
  Description of Artificial Sequence Synthetic polypeptide
MLSTKKTKTHDHYPSIRMRDPGWHDWRASGGGGSGGGGS

SEQ ID NO: 547 - protein, 39 aa, Artificial Sequence
  Description of Artificial Sequence Synthetic polypeptide
MLSTKKTKTHDHYPSTRMRDPGWHDWRASGGGGSGGGGS

SEQ ID NO: 548 - protein, 39 aa, Artificial Sequence
  Description of Artificial Sequence Synthetic polypeptide
MLSTKKTKTHDHYPSVRMRDPGWHDWRASGGGGSGGGGS

SEQ ID NO: 549 - protein, 39 aa, Artificial Sequence
  Description of Artificial Sequence Synthetic polypeptide
MLSTKKTKTHDHYPSSRMRDPGWHDWRASGGGGSGGGGS

SEQ ID NO: 550 - protein, 39 aa, Artificial Sequence
  Description of Artificial Sequence Synthetic polypeptide
MLSTKKTKTHDHYPSFRMRDPGWHDWRASGGGGSGGGGS

SEQ ID NO: 551 - protein, 39 aa, Artificial Sequence
  Description of Artificial Sequence Synthetic polypeptide
MLSTKKTKTHDHYPSYRMRDPGWHDWRASGGGGSGGGGS

SEQ ID NO: 552 - protein, 39 aa, Artificial Sequence
  Description of Artificial Sequence Synthetic polypeptide
MLSTKKTKTHDHYPSGSMRDPGWHDWRASGGGGSGGGGS

SEQ ID NO: 553 - protein, 39 aa, Artificial Sequence
  Description of Artificial Sequence Synthetic polypeptide
MLSTKKTKTHDHYPSGKMRDPGWHDWRASGGGGSGGGGS

SEQ ID NO: 554 - protein, 39 aa, Artificial Sequence
  Description of Artificial Sequence Synthetic polypeptide
MLSTKKTKTHDHYPSGFMRDPGWHDWRASGGGGSGGGGS

SEQ ID NO: 555 - protein, 39 aa, Artificial Sequence
  Description of Artificial Sequence Synthetic polypeptide
MLSTKKTKTHDHYPSGWMRDPGWHDWRASGGGGSGGGGS

SEQ ID NO: 556 - protein, 39 aa, Artificial Sequence
  Description of Artificial Sequence Synthetic polypeptide
MLSTKKTKTHDHYPSGIMRDPGWHDWRASGGGGSGGGGS

SEQ ID NO: 557 - protein, 39 aa, Artificial Sequence
  Description of Artificial Sequence Synthetic polypeptide
MLSTKKTKTHDHYPSGHMRDPGWHDWRASGGGGSGGGGS

SEQ ID NO: 558 - protein, 39 aa, Artificial Sequence
  Description of Artificial Sequence Synthetic polypeptide
MLSTKKTKTHDHYPSGPMRDPGWHDWRASGGGGSGGGGS

SEQ ID NO: 559 - protein, 39 aa, Artificial Sequence
  Description of Artificial Sequence Synthetic polypeptide
MLSTKKTKTHDHYPSGGMRDPGWHDWRASGGGGSGGGGS

SEQ ID NO: 560 - protein, 39 aa, Artificial Sequence
  Description of Artificial Sequence Synthetic polypeptide
MLSTKKTKTHDHYPSGNMRDPGWHDWRASGGGGSGGGGS

SEQ ID NO: 561 - protein, 39 aa, Artificial Sequence
  Description of Artificial Sequence Synthetic polypeptide
MLSTKKTKTHDHYPSGLMRDPGWHDWRASGGGGSGGGGS

SEQ ID NO: 562 - protein, 39 aa, Artificial Sequence
  Description of Artificial Sequence Synthetic polypeptide
MLSTKKTKTHDHYPSGYMRDPGWHDWRASGGGGSGGGGS

SEQ ID NO: 563 - protein, 39 aa, Artificial Sequence
  Description of Artificial Sequence Synthetic polypeptide
MLSTKKTKTHDHYPSGQMRDPGWHDWRASGGGGSGGGGS

SEQ ID NO: 564 - protein, 39 aa, Artificial Sequence
  Description of Artificial Sequence Synthetic polypeptide
MLSTKKTKTHDHYPSGVMRDPGWHDWRASGGGGSGGGGS

SEQ ID NO: 565 - protein, 39 aa, Artificial Sequence
  Description of Artificial Sequence Synthetic polypeptide
MLSTKKTKTHDHYPSGTMRDPGWHDWRASGGGGSGGGGS

SEQ ID NO: 566 - protein, 39 aa, Artificial Sequence
  Description of Artificial Sequence Synthetic polypeptide
MLSTKKTKTHDHYPSGAMRDPGWHDWRASGGGGSGGGGS

SEQ ID NO: 567 - protein, 39 aa, Artificial Sequence
  Description of Artificial Sequence Synthetic polypeptide
MLSTKKTKTHDHYPSGDMRDPGWHDWRASGGGGSGGGGS

SEQ ID NO: 568 - protein, 39 aa, Artificial Sequence
  Description of Artificial Sequence Synthetic polypeptide
MLSTKKTKTHDHYPSGEMRDPGWHDWRASGGGGSGGGGS

SEQ ID NO: 569 - protein, 39 aa, Artificial Sequence
  Description of Artificial Sequence Synthetic polypeptide
MLSTKKTKTHDHYPSGRIRDPGWHDWRASGGGGSGGGGS

SEQ ID NO: 570 - protein, 39 aa, Artificial Sequence
  Description of Artificial Sequence Synthetic polypeptide
MLSTKKTKTHDHYPSGRLRDPGWHDWRASGGGGSGGGGS

SEQ ID NO: 571 - protein, 39 aa, Artificial Sequence
  Description of Artificial Sequence Synthetic polypeptide
MLSTKKTKTHDHYPSGRVRDPGWHDWRASGGGGSGGGGS

SEQ ID NO: 572 - protein, 39 aa, Artificial Sequence
  Description of Artificial Sequence Synthetic polypeptide
MLSTKKTKTHDHYPSGRERDPGWHDWRASGGGGSGGGGS

SEQ ID NO: 573 - protein, 39 aa, Artificial Sequence
  Description of Artificial Sequence Synthetic polypeptide
MLSTKKTKTHDHYPSGRDRDPGWHDWRASGGGGSGGGGS

SEQ ID NO: 574 - protein, 39 aa, Artificial Sequence
  Description of Artificial Sequence Synthetic polypeptide
MLSTKKTKTHDHYPSGRMPDPGWHDWRASGGGGSGGGGS

SEQ ID NO: 575 - protein, 39 aa, Artificial Sequence
  Description of Artificial Sequence Synthetic polypeptide
MLSTKKTKTHDHYPSGRMNDPGWHDWRASGGGGSGGGGS

SEQ ID NO: 576 - protein, 39 aa, Artificial Sequence
  Description of Artificial Sequence Synthetic polypeptide
MLSTKKTKTHDHYPSGRMHDPGWHDWRASGGGGSGGGGS

SEQ ID NO: 577 - protein, 39 aa, Artificial Sequence
  Description of Artificial Sequence Synthetic polypeptide
MLSTKKTKTHDHYPSGRMWDPGWHDWRASGGGGSGGGGS

SEQ ID NO: 578 - protein, 39 aa, Artificial Sequence
  Description of Artificial Sequence Synthetic polypeptide
MLSTKKTKTHDHYPSGRMQDPGWHDWRASGGGGSGGGGS

SEQ ID NO: 579 - protein, 39 aa, Artificial Sequence
  Description of Artificial Sequence Synthetic polypeptide
MLSTKKTKTHDHYPSGRMGDPGWHDWRASGGGGSGGGGS

SEQ ID NO: 580 - protein, 39 aa, Artificial Sequence
  Description of Artificial Sequence Synthetic polypeptide
MLSTKKTKTHDHYPSGRMKDPGWHDWRASGGGGSGGGGS

SEQ ID NO: 581 - protein, 39 aa, Artificial Sequence
  Description of Artificial Sequence Synthetic polypeptide
MLSTKKTKTHDHYPSGRMVDPGWHDWRASGGGGSGGGGS

SEQ ID NO: 582 - protein, 39 aa, Artificial Sequence
  Description of Artificial Sequence Synthetic polypeptide
MLSTKKTKTHDHYPSGRMSDPGWHDWRASGGGGSGGGGS

SEQ ID NO: 583 - protein, 39 aa, Artificial Sequence
  Description of Artificial Sequence Synthetic polypeptide
MLSTKKTKTHDHYPSGRMDDPGWHDWRASGGGGSGGGGS

SEQ ID NO: 584 - protein, 39 aa, Artificial Sequence
  Description of Artificial Sequence Synthetic polypeptide
MLSTKKTKTHDHYPSGRMFDPGWHDWRASGGGGSGGGGS

SEQ ID NO: 585 - protein, 39 aa, Artificial Sequence
  Description of Artificial Sequence Synthetic polypeptide
MLSTKKTKTHDHYPSGRMTDPGWHDWRASGGGGSGGGGS

SEQ ID NO: 586 - protein, 39 aa, Artificial Sequence
  Description of Artificial Sequence Synthetic polypeptide
MLSTKKTKTHDHYPSGRMLDPGWHDWRASGGGGSGGGGS

SEQ ID NO: 587 - protein, 39 aa, Artificial Sequence
  Description of Artificial Sequence Synthetic polypeptide
MLSTKKTKTHDHYPSGRMYDPGWHDWRASGGGGSGGGGS

SEQ ID NO: 588 - protein, 39 aa, Artificial Sequence
  Description of Artificial Sequence Synthetic polypeptide
MLSTKKTKTHDHYPSGRMEDPGWHDWRASGGGGSGGGGS

SEQ ID NO: 589 - protein, 39 aa, Artificial Sequence
  Description of Artificial Sequence Synthetic polypeptide
MLSTKKTKTHDHYPSGRMIDPGWHDWRASGGGGSGGGGS

SEQ ID NO: 590 - protein, 39 aa, Artificial Sequence
  Description of Artificial Sequence Synthetic polypeptide
MLSTKKTKTHDHYPSGRMADPGWHDWRASGGGGSGGGGS

SEQ ID NO: 591 - protein, 39 aa, Artificial Sequence
  Description of Artificial Sequence Synthetic polypeptide
MLSTKKTKTHDHYPSGRMRFPGWHDWRASGGGGSGGGGS

SEQ ID NO: 592 - protein, 39 aa, Artificial Sequence
  Description of Artificial Sequence Synthetic polypeptide
MLSTKKTKTHDHYPSGRMRHPGWHDWRASGGGGSGGGGS

SEQ ID NO: 593 - protein, 39 aa, Artificial Sequence
  Description of Artificial Sequence Synthetic polypeptide
MLSTKKTKTHDHYPSGRMRWPGWHDWRASGGGGSGGGGS

SEQ ID NO: 594 - protein, 39 aa, Artificial Sequence
  Description of Artificial Sequence Synthetic polypeptide
MLSTKKTKTHDHYPSGRMRIPGWHDWRASGGGGSGGGGS

SEQ ID NO: 595 - protein, 39 aa, Artificial Sequence
  Description of Artificial Sequence Synthetic polypeptide
MLSTKKTKTHDHYPSGRMRNPGWHDWRASGGGGSGGGGS

SEQ ID NO: 596 - protein, 39 aa, Artificial Sequence
  Description of Artificial Sequence Synthetic polypeptide
MLSTKKTKTHDHYPSGRMRAPGWHDWRASGGGGSGGGGS

SEQ ID NO: 597 - protein, 39 aa, Artificial Sequence
  Description of Artificial Sequence Synthetic polypeptide
MLSTKKTKTHDHYPSGRMRSPGWHDWRASGGGGSGGGGS

SEQ ID NO: 598 - protein, 39 aa, Artificial Sequence
  Description of Artificial Sequence Synthetic polypeptide
MLSTKKTKTHDHYPSGRMREPGWHDWRASGGGGSGGGGS

SEQ ID NO: 599 - protein, 39 aa, Artificial Sequence
  Description of Artificial Sequence Synthetic polypeptide
MLSTKKTKTHDHYPSGRMRTPGWHDWRASGGGGSGGGGS

SEQ ID NO: 600 - protein, 39 aa, Artificial Sequence
  Description of Artificial Sequence Synthetic polypeptide
MLSTKKTKTHDHYPSGRMRYPGWHDWRASGGGGSGGGGS

SEQ ID NO: 601 - protein, 39 aa, Artificial Sequence
  Description of Artificial Sequence Synthetic polypeptide
MLSTKKTKTHDHYPSGRMRVPGWHDWRASGGGGSGGGGS

SEQ ID NO: 602 - protein, 39 aa, Artificial Sequence
  Description of Artificial Sequence Synthetic polypeptide
MLSTKKTKTHDHYPSGRMRQPGWHDWRASGGGGSGGGGS

SEQ ID NO: 603 - protein, 39 aa, Artificial Sequence
  Description of Artificial Sequence Synthetic polypeptide
MLSTKKTKTHDHYPSGRMRLPGWHDWRASGGGGSGGGGS

SEQ ID NO: 604 - protein, 39 aa, Artificial Sequence
  Description of Artificial Sequence Synthetic polypeptide
MLSTKKTKTHDHYPSGRMRDQGWHDWRASGGGGSGGGGS

SEQ ID NO: 605 - protein, 39 aa, Artificial Sequence
  Description of Artificial Sequence Synthetic polypeptide
MLSTKKTKTHDHYPSGRMRDWGWHDWRASGGGGSGGGGS

SEQ ID NO: 606 - protein, 39 aa, Artificial Sequence
  Description of Artificial Sequence Synthetic polypeptide
MLSTKKTKTHDHYPSGRMRDKGWHDWRASGGGGSGGGGS

SEQ ID NO: 607 - protein, 39 aa, Artificial Sequence
  Description of Artificial Sequence Synthetic polypeptide
MLSTKKTKTHDHYPSGRMRDLGWHDWRASGGGGSGGGGS

SEQ ID NO: 608 - protein, 39 aa, Artificial Sequence
  Description of Artificial Sequence Synthetic polypeptide
MLSTKKTKTHDHYPSGRMRDYGWHDWRASGGGGSGGGGS

SEQ ID NO: 609 - protein, 39 aa, Artificial Sequence
  Description of Artificial Sequence Synthetic polypeptide
MLSTKKTKTHDHYPSGRMRDHGWHDWRASGGGGSGGGGS

SEQ ID NO: 610 - protein, 39 aa, Artificial Sequence
  Description of Artificial Sequence Synthetic polypeptide
MLSTKKTKTHDHYPSGRMRDGGWHDWRASGGGGSGGGGS

SEQ ID NO: 611 - protein, 39 aa, Artificial Sequence
  Description of Artificial Sequence Synthetic polypeptide
MLSTKKTKTHDHYPSGRMRDNGWHDWRASGGGGSGGGGS

SEQ ID NO: 612 - protein, 39 aa, Artificial Sequence
  Description of Artificial Sequence Synthetic polypeptide
MLSTKKTKTHDHYPSGRMRDTGWHDWRASGGGGSGGGGS

SEQ ID NO: 613 - protein, 39 aa, Artificial Sequence
  Description of Artificial Sequence Synthetic polypeptide
MLSTKKTKTHDHYPSGRMRDSGWHDWRASGGGGSGGGGS

SEQ ID NO: 614 - protein, 39 aa, Artificial Sequence
  Description of Artificial Sequence Synthetic polypeptide
MLSTKKTKTHDHYPSGRMRDEGWHDWRASGGGGSGGGGS

SEQ ID NO: 615 - protein, 39 aa, Artificial Sequence
  Description of Artificial Sequence Synthetic polypeptide
MLSTKKTKTHDHYPSGRMRDDGWHDWRASGGGGSGGGGS

SEQ ID NO: 616 - protein, 39 aa, Artificial Sequence
  Description of Artificial Sequence Synthetic polypeptide
MLSTKKTKTHDHYPSGRMRDIGWHDWRASGGGGSGGGGS

SEQ ID NO: 617 - protein, 39 aa, Artificial Sequence
  Description of Artificial Sequence Synthetic polypeptide
MLSTKKTKTHDHYPSGRMRDAGWHDWRASGGGGSGGGGS

SEQ ID NO: 618 - protein, 39 aa, Artificial Sequence
  Description of Artificial Sequence Synthetic polypeptide
MLSTKKTKTHDHYPSGRMRDVGWHDWRASGGGGSGGGGS

SEQ ID NO: 619 - protein, 39 aa, Artificial Sequence
  Description of Artificial Sequence Synthetic polypeptide
MLSTKKTKTHDHYPSGRMRDPFWHDWRASGGGGSGGGGS

SEQ ID NO: 620 - protein, 39 aa, Artificial Sequence
  Description of Artificial Sequence Synthetic polypeptide
MLSTKKTKTHDHYPSGRMRDPQWHDWRASGGGGSGGGGS

SEQ ID NO: 621 - protein, 39 aa, Artificial Sequence
  Description of Artificial Sequence Synthetic polypeptide
MLSTKKTKTHDHYPSGRMRDPTWHDWRASGGGGSGGGGS

SEQ ID NO: 622 - protein, 39 aa, Artificial Sequence
  Description of Artificial Sequence Synthetic polypeptide
MLSTKKTKTHDHYPSGRMRDPYWHDWRASGGGGSGGGGS

SEQ ID NO: 623 - protein, 39 aa, Artificial Sequence
  Description of Artificial Sequence Synthetic polypeptide
MLSTKKTKTHDHYPSGRMRDPNWHDWRASGGGGSGGGGS

SEQ ID NO: 624 - protein, 39 aa, Artificial Sequence
  Description of Artificial Sequence Synthetic polypeptide
MLSTKKTKTHDHYPSGRMRDPHWHDWRASGGGGSGGGGS

SEQ ID NO: 625 - protein, 39 aa, Artificial Sequence
  Description of Artificial Sequence Synthetic polypeptide
MLSTKKTKTHDHYPSGRMRDPLWHDWRASGGGGSGGGGS

SEQ ID NO: 626 - protein, 39 aa, Artificial Sequence
  Description of Artificial Sequence Synthetic polypeptide
MLSTKKTKTHDHYPSGRMRDPIWHDWRASGGGGSGGGGS

SEQ ID NO: 627 - protein, 39 aa, Artificial Sequence
  Description of Artificial Sequence Synthetic polypeptide
MLSTKKTKTHDHYPSGRMRDPVWHDWRASGGGGSGGGGS

SEQ ID NO: 628 - protein, 39 aa, Artificial Sequence
  Description of Artificial Sequence Synthetic polypeptide
MLSTKKTKTHDHYPSGRMRDPSWHDWRASGGGGSGGGGS

SEQ ID NO: 629 - protein, 39 aa, Artificial Sequence
  Description of Artificial Sequence Synthetic polypeptide
MLSTKKTKTHDHYPSGRMRDPDWHDWRASGGGGSGGGGS

SEQ ID NO: 630 - protein, 39 aa, Artificial Sequence
  Description of Artificial Sequence Synthetic polypeptide
MLSTKKTKTHDHYPSGRMRDPEWHDWRASGGGGSGGGGS

SEQ ID NO: 631 - protein, 39 aa, Artificial Sequence
  Description of Artificial Sequence Synthetic polypeptide
MLSTKKTKTHDHYPSGRMRDPGPHDWRASGGGGSGGGGS

SEQ ID NO: 632 - protein, 39 aa, Artificial Sequence
  Description of Artificial Sequence Synthetic polypeptide
MLSTKKTKTHDHYPSGRMRDPGYHDWRASGGGGSGGGGS

SEQ ID NO: 633 - protein, 39 aa, Artificial Sequence
  Description of Artificial Sequence Synthetic polypeptide
MLSTKKTKTHDHYPSGRMRDPGRHDWRASGGGGSGGGGS

SEQ ID NO: 634 - protein, 39 aa, Artificial Sequence
  Description of Artificial Sequence Synthetic polypeptide
MLSTKKTKTHDHYPSGRMRDPGKHDWRASGGGGSGGGGS

SEQ ID NO: 635 - protein, 39 aa, Artificial Sequence
  Description of Artificial Sequence Synthetic polypeptide
MLSTKKTKTHDHYPSGRMRDPGHHDWRASGGGGSGGGGS

SEQ ID NO: 636 - protein, 39 aa, Artificial Sequence
  Description of Artificial Sequence Synthetic polypeptide
MLSTKKTKTHDHYPSGRMRDPGFHDWRASGGGGSGGGGS

SEQ ID NO: 637 - protein, 39 aa, Artificial Sequence
  Description of Artificial Sequence Synthetic polypeptide
MLSTKKTKTHDHYPSGRMRDPGNHDWRASGGGGSGGGGS

SEQ ID NO: 638 - protein, 39 aa, Artificial Sequence
  Description of Artificial Sequence Synthetic polypeptide
MLSTKKTKTHDHYPSGRMRDPGDHDWRASGGGGSGGGGS

SEQ ID NO: 639 - protein, 39 aa, Artificial Sequence
  Description of Artificial Sequence Synthetic polypeptide
MLSTKKTKTHDHYPSGRMRDPGSHDWRASGGGGSGGGGS

SEQ ID NO: 640 - protein, 39 aa, Artificial Sequence
  Description of Artificial Sequence Synthetic polypeptide
MLSTKKTKTHDHYPSGRMRDPGAHDWRASGGGGSGGGGS

SEQ ID NO: 641 - protein, 39 aa, Artificial Sequence
  Description of Artificial Sequence Synthetic polypeptide
MLSTKKTKTHDHYPSGRMRDPGEHDWRASGGGGSGGGGS

SEQ ID NO: 642 - protein, 39 aa, Artificial Sequence
  Description of Artificial Sequence Synthetic polypeptide
MLSTKKTKTHDHYPSGRMRDPGIHDWRASGGGGSGGGGS

SEQ ID NO: 643 - protein, 39 aa, Artificial Sequence
  Description of Artificial Sequence Synthetic polypeptide
MLSTKKTKTHDHYPSGRMRDPGGHDWRASGGGGSGGGGS

SEQ ID NO: 644 - protein, 39 aa, Artificial Sequence
  Description of Artificial Sequence Synthetic polypeptide
MLSTKKTKTHDHYPSGRMRDPGQHDWRASGGGGSGGGGS

SEQ ID NO: 645 - protein, 39 aa, Artificial Sequence
  Description of Artificial Sequence Synthetic polypeptide
MLSTKKTKTHDHYPSGRMRDPGTHDWRASGGGGSGGGGS

SEQ ID NO: 646 - protein, 39 aa, Artificial Sequence
  Description of Artificial Sequence Synthetic polypeptide
MLSTKKTKTHDHYPSGRMRDPGVHDWRASGGGGSGGGGS

SEQ ID NO: 647 - protein, 39 aa, Artificial Sequence
  Description of Artificial Sequence Synthetic polypeptide
MLSTKKTKTHDHYPSGRMRDPGWIDWRASGGGGSGGGGS

SEQ ID NO: 648 - protein, 39 aa, Artificial Sequence
  Description of Artificial Sequence Synthetic polypeptide
MLSTKKTKTHDHYPSGRMRDPGWRDWRASGGGGSGGGGS

SEQ ID NO: 649 - protein, 39 aa, Artificial Sequence
  Description of Artificial Sequence Synthetic polypeptide
MLSTKKTKTHDHYPSGRMRDPGWGDWRASGGGGSGGGGS

SEQ ID NO: 650 - protein, 39 aa, Artificial Sequence
  Description of Artificial Sequence Synthetic polypeptide
MLSTKKTKTHDHYPSGRMRDPGWFDWRASGGGGSGGGGS

SEQ ID NO: 651 - protein, 39 aa, Artificial Sequence
  Description of Artificial Sequence Synthetic polypeptide
MLSTKKTKTHDHYPSGRMRDPGWLDWRASGGGGSGGGGS

SEQ ID NO: 652 - protein, 39 aa, Artificial Sequence
  Description of Artificial Sequence Synthetic polypeptide
MLSTKKTKTHDHYPSGRMRDPGWADWRASGGGGSGGGGS

SEQ ID NO: 653 - protein, 39 aa, Artificial Sequence
  Description of Artificial Sequence Synthetic polypeptide
MLSTKKTKTHDHYPSGRMRDPGWYDWRASGGGGSGGGGS

SEQ ID NO: 654 - protein, 39 aa, Artificial Sequence
  Description of Artificial Sequence Synthetic polypeptide
MLSTKKTKTHDHYPSGRMRDPGWPDWRASGGGGSGGGGS

SEQ ID NO: 655 - protein, 39 aa, Artificial Sequence
  Description of Artificial Sequence Synthetic polypeptide
MLSTKKTKTHDHYPSGRMRDPGWQDWRASGGGGSGGGGS

SEQ ID NO: 656 - protein, 39 aa, Artificial Sequence
  Description of Artificial Sequence Synthetic polypeptide
MLSTKKTKTHDHYPSGRMRDPGWSDWRASGGGGSGGGGS

SEQ ID NO: 657 - protein, 39 aa, Artificial Sequence
  Description of Artificial Sequence Synthetic polypeptide
MLSTKKTKTHDHYPSGRMRDPGWTDWRASGGGGSGGGGS

SEQ ID NO: 658 - protein, 39 aa, Artificial Sequence
  Description of Artificial Sequence Synthetic polypeptide
MLSTKKTKTHDHYPSGRMRDPGWNDWRASGGGGSGGGGS

SEQ ID NO: 659 - protein, 39 aa, Artificial Sequence
  Description of Artificial Sequence Synthetic polypeptide
MLSTKKTKTHDHYPSGRMRDPGWDDWRASGGGGSGGGGS

SEQ ID NO: 660 - protein, 39 aa, Artificial Sequence
  Description of Artificial Sequence Synthetic polypeptide
MLSTKKTKTHDHYPSGRMRDPGWVDWRASGGGGSGGGGS

SEQ ID NO: 661 - protein, 39 aa, Artificial Sequence
  Description of Artificial Sequence Synthetic polypeptide
MLSTKKTKTHDHYPSGRMRDPGWEDWRASGGGGSGGGGS

SEQ ID NO: 662 - protein, 39 aa, Artificial Sequence
  Description of Artificial Sequence Synthetic polypeptide
MLSTKKTKTHDHYPSGRMRDPGWHEWRASGGGGSGGGGS

SEQ ID NO: 663 - protein, 39 aa, Artificial Sequence
  Description of Artificial Sequence Synthetic polypeptide
MLSTKKTKTHDHYPSGRMRDPGWHYWRASGGGGSGGGGS

SEQ ID NO: 664 - protein, 39 aa, Artificial Sequence
  Description of Artificial Sequence Synthetic polypeptide
MLSTKKTKTHDHYPSGRMRDPGWHSWRASGGGGSGGGGS

SEQ ID NO: 665 - protein, 39 aa, Artificial Sequence
  Description of Artificial Sequence Synthetic polypeptide
MLSTKKTKTHDHYPSGRMRDPGWHGWRASGGGGSGGGGS

SEQ ID NO: 666 - protein, 39 aa, Artificial Sequence
  Description of Artificial Sequence Synthetic polypeptide
MLSTKKTKTHDHYPSGRMRDPGWHTWRASGGGGSGGGGS

SEQ ID NO: 667 - protein, 39 aa, Artificial Sequence
  Description of Artificial Sequence Synthetic polypeptide
MLSTKKTKTHDHYPSGRMRDPGWHVWRASGGGGSGGGGS

SEQ ID NO: 668 - protein, 39 aa, Artificial Sequence
  Description of Artificial Sequence Synthetic polypeptide
MLSTKKTKTHDHYPSGRMRDPGWHHWRASGGGGSGGGGS

SEQ ID NO: 669 - protein, 39 aa, Artificial Sequence
  Description of Artificial Sequence Synthetic polypeptide
MLSTKKTKTHDHYPSGRMRDPGWHLWRASGGGGSGGGGS

SEQ ID NO: 670 - protein, 39 aa, Artificial Sequence
  Description of Artificial Sequence Synthetic polypeptide
MLSTKKTKTHDHYPSGRMRDPGWHIWRASGGGGSGGGGS

SEQ ID NO: 671 - protein, 39 aa, Artificial Sequence
  Description of Artificial Sequence Synthetic polypeptide
MLSTKKTKTHDHYPSGRMRDPGWHNWRASGGGGSGGGGS

SEQ ID NO: 672 - protein, 39 aa, Artificial Sequence
  Description of Artificial Sequence Synthetic polypeptide
MLSTKKTKTHDHYPSGRMRDPGWHWWRASGGGGSGGGGS

SEQ ID NO: 673 - protein, 39 aa, Artificial Sequence
  Description of Artificial Sequence Synthetic polypeptide
MLSTKKTKTHDHYPSGRMRDPGWHFWRASGGGGSGGGGS

SEQ ID NO: 674 - protein, 39 aa, Artificial Sequence
  Description of Artificial Sequence Synthetic polypeptide
MLSTKKTKTHDHYPSGRMRDPGWHDHRASGGGGSGGGGS

SEQ ID NO: 675 - protein, 39 aa, Artificial Sequence
  Description of Artificial Sequence Synthetic polypeptide
MLSTKKTKTHDHYPSGRMRDPGWHDPRASGGGGSGGGGS

SEQ ID NO: 676 - protein, 39 aa, Artificial Sequence
  Description of Artificial Sequence Synthetic polypeptide
MLSTKKTKTHDHYPSGRMRDPGWHDNRASGGGGSGGGGS

SEQ ID NO: 677 - protein, 39 aa, Artificial Sequence
  Description of Artificial Sequence Synthetic polypeptide
MLSTKKTKTHDHYPSGRMRDPGWHDSRASGGGGSGGGGS

SEQ ID NO: 678 - protein, 39 aa, Artificial Sequence
  Description of Artificial Sequence Synthetic polypeptide
MLSTKKTKTHDHYPSGRMRDPGWHDQRASGGGGSGGGGS

SEQ ID NO: 679 - protein, 39 aa, Artificial Sequence
  Description of Artificial Sequence Synthetic polypeptide
MLSTKKTKTHDHYPSGRMRDPGWHDTRASGGGGSGGGGS

SEQ ID NO: 680 - protein, 39 aa, Artificial Sequence
  Description of Artificial Sequence Synthetic polypeptide
MLSTKKTKTHDHYPSGRMRDPGWHDARASGGGGSGGGGS

SEQ ID NO: 681 - protein, 39 aa, Artificial Sequence
  Description of Artificial Sequence Synthetic polypeptide
MLSTKKTKTHDHYPSGRMRDPGWHDDRASGGGGSGGGGS

SEQ ID NO: 682 - protein, 39 aa, Artificial Sequence
  Description of Artificial Sequence Synthetic polypeptide
MLSTKKTKTHDHYPSGRMRDPGWHDGRASGGGGSGGGGS

SEQ ID NO: 683 - protein, 39 aa, Artificial Sequence
  Description of Artificial Sequence Synthetic polypeptide
MLSTKKTKTHDHYPSGRMRDPGWHDYRASGGGGSGGGGS

SEQ ID NO: 684 - protein, 39 aa, Artificial Sequence
  Description of Artificial Sequence Synthetic polypeptide
MLSTKKTKTHDHYPSGRMRDPGWHDLRASGGGGSGGGGS

SEQ ID NO: 685 - protein, 39 aa, Artificial Sequence
  Description of Artificial Sequence Synthetic polypeptide
MLSTKKTKTHDHYPSGRMRDPGWHDVRASGGGGSGGGGS

SEQ ID NO: 686 - protein, 39 aa, Artificial Sequence
  Description of Artificial Sequence Synthetic polypeptide
MLSTKKTKTHDHYPSGRMRDPGWHDERASGGGGSGGGGS

SEQ ID NO: 687 - protein, 39 aa, Artificial Sequence
  Description of Artificial Sequence Synthetic polypeptide
MLSTKKTKTHDHYPSGRMRDPGWHDFRASGGGGSGGGGS

SEQ ID NO: 688 - protein, 39 aa, Artificial Sequence
  Description of Artificial Sequence Synthetic polypeptide
MLSTKKTKTHDHYPSGRMRDPGWHDRRASGGGGSGGGGS

SEQ ID NO: 689 - protein, 39 aa, Artificial Sequence
  Description of Artificial Sequence Synthetic polypeptide
MLSTKKTKTHDHYPSGRMRDPGWHDKRASGGGGSGGGGS

SEQ ID NO: 690 - protein, 39 aa, Artificial Sequence
  Description of Artificial Sequence Synthetic polypeptide
MLSTKKTKTHDHYPSGRMRDPGWHDWAASGGGGSGGGGS

SEQ ID NO: 691 - protein, 39 aa, Artificial Sequence
  Description of Artificial Sequence Synthetic polypeptide
MLSTKKTKTHDHYPSGRMRDPGWHDWDASGGGGSGGGGS

SEQ ID NO: 692 - protein, 39 aa, Artificial Sequence
  Description of Artificial Sequence Synthetic polypeptide
MLSTKKTKTHDHYPSGRMRDPGWHDWEASGGGGSGGGGS

SEQ ID NO: 693 - protein, 39 aa, Artificial Sequence
  Description of Artificial Sequence Synthetic polypeptide
MLSTKKTKTHDHYPSGRMRDPGWHDWFASGGGGSGGGGS

SEQ ID NO: 694 - protein, 39 aa, Artificial Sequence
  Description of Artificial Sequence Synthetic polypeptide
MLSTKKTKTHDHYPSGRMRDPGWHDWGASGGGGSGGGGS

SEQ ID NO: 695 - protein, 39 aa, Artificial Sequence
  Description of Artificial Sequence Synthetic polypeptide
MLSTKKTKTHDHYPSGRMRDPGWHDWHASGGGGSGGGGS

SEQ ID NO: 696 - protein, 39 aa, Artificial Sequence
  Description of Artificial Sequence Synthetic polypeptide
MLSTKKTKTHDHYPSGRMRDPGWHDWIASGGGGSGGGGS

SEQ ID NO: 697 - protein, 39 aa, Artificial Sequence
  Description of Artificial Sequence Synthetic polypeptide
MLSTKKTKTHDHYPSGRMRDPGWHDWKASGGGGSGGGGS

SEQ ID NO: 698 - protein, 39 aa, Artificial Sequence
  Description of Artificial Sequence Synthetic polypeptide
MLSTKKTKTHDHYPSGRMRDPGWHDWNASGGGGSGGGGS

SEQ ID NO: 699 - protein, 39 aa, Artificial Sequence
  Description of Artificial Sequence Synthetic polypeptide
MLSTKKTKTHDHYPSGRMRDPGWHDWPASGGGGSGGGGS

SEQ ID NO: 700 - protein, 39 aa, Artificial Sequence
  Description of Artificial Sequence Synthetic polypeptide
MLSTKKTKTHDHYPSGRMRDPGWHDWQASGGGGSGGGGS

SEQ ID NO: 701 - protein, 39 aa, Artificial Sequence
  Description of Artificial Sequence Synthetic polypeptide
MLSTKKTKTHDHYPSGRMRDPGWHDWSASGGGGSGGGGS

SEQ ID NO: 702 - protein, 39 aa, Artificial Sequence
  Description of Artificial Sequence Synthetic polypeptide
MLSTKKTKTHDHYPSGRMRDPGWHDWTASGGGGSGGGGS

SEQ ID NO: 703 - protein, 39 aa, Artificial Sequence
  Description of Artificial Sequence Synthetic polypeptide
MLSTKKTKTHDHYPSGRMRDPGWHDWVASGGGGSGGGGS

SEQ ID NO: 704 - protein, 39 aa, Artificial Sequence
  Description of Artificial Sequence Synthetic polypeptide
MLSTKKTKTHDHYPSGRMRDPGWHDWYASGGGGSGGGGS

SEQ ID NO: 705 - protein, 39 aa, Artificial Sequence
  Description of Artificial Sequence Synthetic polypeptide
MLSTKKTKTHDHYPSGRMRDPGWHDWRTSGGGGSGGGGS

SEQ ID NO: 706 - protein, 39 aa, Artificial Sequence
  Description of Artificial Sequence Synthetic polypeptide
MLSTKKTKTHDHYPSGRMRDPGWHDWRSSGGGGSGGGGS

SEQ ID NO: 707 - protein, 39 aa, Artificial Sequence
  Description of Artificial Sequence Synthetic polypeptide
MLSTKKTKTHDHYPSGRMRDPGWHDWRRSGGGGSGGGGS

SEQ ID NO: 708 - protein, 39 aa, Artificial Sequence
  Description of Artificial Sequence Synthetic polypeptide
MLSTKKTKTHDHYPSGRMRDPGWHDWRQSGGGGSGGGGS

SEQ ID NO: 709 - protein, 39 aa, Artificial Sequence
  Description of Artificial Sequence Synthetic polypeptide
MLSTKKTKTHDHYPSGRMRDPGWHDWRNSGGGGSGGGGS

SEQ ID NO: 710 - protein, 39 aa, Artificial Sequence
  Description of Artificial Sequence Synthetic polypeptide
MLSTKKTKTHDHYPSGRMRDPGWHDWRLSGGGGSGGGGS

SEQ ID NO: 711 - protein, 39 aa, Artificial Sequence
  Description of Artificial Sequence Synthetic polypeptide
MLSTKKTKTHDHYPSGRMRDPGWHDWRKSGGGGSGGGGS

SEQ ID NO: 712 - protein, 39 aa, Artificial Sequence
  Description of Artificial Sequence Synthetic polypeptide
MLSTKKTKTHDHYPSGRMRDPGWHDWRISGGGGSGGGGS

SEQ ID NO: 713 - protein, 39 aa, Artificial Sequence
  Description of Artificial Sequence Synthetic polypeptide
MLSTKKTKTHDHYPSGRMRDPGWHDWRHSGGGGSGGGGS

SEQ ID NO: 714 - protein, 39 aa, Artificial Sequence
  Description of Artificial Sequence Synthetic polypeptide
MLSTKKTKTHDHYPSGRMRDPGWHDWRESGGGGSGGGGS

SEQ ID NO: 715 - protein, 39 aa, Artificial Sequence
  Description of Artificial Sequence Synthetic polypeptide
MLSTKKTKTHDHYPSGRMRDPGWHDWRDSGGGGSGGGGS

SEQ ID NO: 716 - protein, 39 aa, Artificial Sequence
  Description of Artificial Sequence Synthetic polypeptide
MLSTKKTKTHDHYPSGRMRDPGWHDWRAAGGGGSGGGGS

SEQ ID NO: 717 - protein, 39 aa, Artificial Sequence
  Description of Artificial Sequence Synthetic polypeptide
MLSTKKTKTHDHYPSGRMRDPGWHDWRADGGGGSGGGGS

SEQ ID NO: 718 - protein, 39 aa, Artificial Sequence
  Description of Artificial Sequence Synthetic polypeptide
MLSTKKTKTHDHYPSGRMRDPGWHDWRAEGGGGSGGGGS

SEQ ID NO: 719 - protein, 39 aa, Artificial Sequence
  Description of Artificial Sequence Synthetic polypeptide
MLSTKKTKTHDHYPSGRMRDPGWHDWRAFGGGGSGGGGS

SEQ ID NO: 720 - protein, 39 aa, Artificial Sequence
  Description of Artificial Sequence Synthetic polypeptide
MLSTKKTKTHDHYPSGRMRDPGWHDWRAGGGGGSGGGGS

SEQ ID NO: 721 - protein, 39 aa, Artificial Sequence
  Description of Artificial Sequence Synthetic polypeptide
MLSTKKTKTHDHYPSGRMRDPGWHDWRAHGGGGSGGGGS

SEQ ID NO: 722 - protein, 39 aa, Artificial Sequence
  Description of Artificial Sequence Synthetic polypeptide
MLSTKKTKTHDHYPSGRMRDPGWHDWRAIGGGGSGGGGS

SEQ ID NO: 723 - protein, 39 aa, Artificial Sequence
  Description of Artificial Sequence Synthetic polypeptide
MLSTKKTKTHDHYPSGRMRDPGWHDWRAKGGGGSGGGGS

SEQ ID NO: 724 - protein, 39 aa, Artificial Sequence
  Description of Artificial Sequence Synthetic polypeptide
MLSTKKTKTHDHYPSGRMRDPGWHDWRALGGGGSGGGGS

SEQ ID NO: 725 - protein, 39 aa, Artificial Sequence
  Description of Artificial Sequence Synthetic polypeptide
MLSTKKTKTHDHYPSGRMRDPGWHDWRANGGGGSGGGGS

SEQ ID NO: 726 - protein, 39 aa, Artificial Sequence
  Description of Artificial Sequence Synthetic polypeptide
MLSTKKTKTHDHYPSGRMRDPGWHDWRAPGGGGSGGGGS

SEQ ID NO: 727 - protein, 39 aa, Artificial Sequence
  Description of Artificial Sequence Synthetic polypeptide
MLSTKKTKTHDHYPSGRMRDPGWHDWRAQGGGGSGGGGS

SEQ ID NO: 728 - protein, 39 aa, Artificial Sequence
  Description of Artificial Sequence Synthetic polypeptide
MLSTKKTKTHDHYPSGRMRDPGWHDWRATGGGGSGGGGS

SEQ ID NO: 729 - protein, 39 aa, Artificial Sequence
  Description of Artificial Sequence Synthetic polypeptide
MLSTKKTKTHDHYPSGRMRDPGWHDWRAVGGGGSGGGGS

SEQ ID NO: 730 - protein, 39 aa, Artificial Sequence
  Description of Artificial Sequence Synthetic polypeptide
MLSTKKTKTHDHYPSGRMRDPGWHDWRAYGGGGSGGGGS

SEQ ID NO: 731 - protein, 27 aa, Artificial Sequence
  Description of Artificial Sequence Synthetic peptide
LGKTDWIEKYFKVKKEKIDKMQRFLQG

SEQ ID NO: 732 - protein, 42 aa, Artificial Sequence
  Description of Artificial Sequence Synthetic polypeptide
MLSTKKTKTHDHYPCGRMRDPGWHDWRACLTHQGIEEDEWPV

SEQ ID NO: 733 - protein, 29 aa, Artificial Sequence
  Description of Artificial Sequence Synthetic peptide
FASAAKGKWHDHNGGGRHRPFKWHDWWFR

SEQ ID NO: 734 - protein, 29 aa, Artificial Sequence
  Description of Artificial Sequence Synthetic peptide
PGSGKKTKTHDHWWSGRWRDPPWHDWRGS

SEQ ID NO: 735 - protein, 29 aa, Artificial Sequence
  Description of Artificial Sequence Synthetic peptide
SHSTKKTKTHDHYPSGRYRDPWWHDWRVS

SEQ ID NO: 736 - protein, 29 aa, Artificial Sequence
  Description of Artificial Sequence Synthetic peptide
VNSTKKTKTHDHYPSGRMRDPGWHDWRWS

SEQ ID NO: 737 - protein, 29 aa, Artificial Sequence
  Description of Artificial Sequence Synthetic peptide
WPSTKKTKTHDHYPSGRMRDPGWHDWRAS

SEQ ID NO: 738 - protein, 29 aa, Artificial Sequence
  Description of Artificial Sequence Synthetic peptide
DLSTKKTKTHDHYPSGRMRDPGWHDWRAS

SEQ ID NO: 739 - protein, 29 aa, Artificial Sequence
  Description of Artificial Sequence Synthetic peptide
ELSTKKTKTHDHYPSGRMRDPGWHDWRAS

SEQ ID NO: 740 - protein, 29 aa, Artificial Sequence
  Description of Artificial Sequence Synthetic peptide
LLSTKKTKTHDHYPSGRMRDPGWHDWRAS

SEQ ID NO: 741 - protein, 29 aa, Artificial Sequence
  Description of Artificial Sequence Synthetic peptide
NLSTKKTKTHDHYPSGRMRDPGWHDWRAS

SEQ ID NO: 742 - protein, 29 aa, Artificial Sequence
  Description of Artificial Sequence Synthetic peptide
TLSTKKTKTHDHYPSGRMRDPGWHDWRAS

SEQ ID NO: 743 - protein, 29 aa, Artificial Sequence
  Description of Artificial Sequence Synthetic peptide
MESTKKTKTHDHYPSGRMRDPGWHDWRAS

SEQ ID NO: 744 - protein, 29 aa, Artificial Sequence
  Description of Artificial Sequence Synthetic peptide
MDSTKKTKTHDHYPSGRMRDPGWHDWRAS

SEQ ID NO: 745 - protein, 29 aa, Artificial Sequence
  Description of Artificial Sequence Synthetic peptide
MLYTKKTKTHDHYPSGRMRDPGWHDWRAS

SEQ ID NO: 746 - protein, 29 aa, Artificial Sequence
  Description of Artificial Sequence Synthetic peptide
MLWTKKTKTHDHYPSGRMRDPGWHDWRAS

SEQ ID NO: 747 - protein, 29 aa, Artificial Sequence
  Description of Artificial Sequence Synthetic peptide
MLTTKKTKTHDHYPSGRMRDPGWHDWRAS

SEQ ID NO: 748 - protein, 29 aa, Artificial Sequence
  Description of Artificial Sequence Synthetic peptide
MLETKKTKTHDHYPSGRMRDPGWHDWRAS

SEQ ID NO: 749 - protein, 29 aa, Artificial Sequence
  Description of Artificial Sequence Synthetic peptide
MLKTKKTKTHDHYPSGRMRDPGWHDWRAS

SEQ ID NO: 750 - protein, 29 aa, Artificial Sequence
  Description of Artificial Sequence Synthetic peptide
MLHTKKTKTHDHYPSGRMRDPGWHDWRAS

SEQ ID NO: 751 - protein, 29 aa, Artificial Sequence
  Description of Artificial Sequence Synthetic peptide
MLLTKKTKTHDHYPSGRMRDPGWHDWRAS

SEQ ID NO: 752 - protein, 29 aa, Artificial Sequence
  Description of Artificial Sequence Synthetic peptide
MLNTKKTKTHDHYPSGRMRDPGWHDWRAS

SEQ ID NO: 753 - protein, 29 aa, Artificial Sequence
  Description of Artificial Sequence Synthetic peptide
MLATKKTKTHDHYPSGRMRDPGWHDWRAS

SEQ ID NO: 754 - protein, 29 aa, Artificial Sequence
  Description of Artificial Sequence Synthetic peptide
MLDTKKTKTHDHYPSGRMRDPGWHDWRAS

SEQ ID NO: 755 - protein, 29 aa, Artificial Sequence
  Description of Artificial Sequence Synthetic peptide
MLSYKKTKTHDHYPSGRMRDPGWHDWRAS

SEQ ID NO: 756 - protein, 29 aa, Artificial Sequence
  Description of Artificial Sequence Synthetic peptide
MLSNKKTKTHDHYPSGRMRDPGWHDWRAS

SEQ ID NO: 757 - protein, 29 aa, Artificial Sequence
  Description of Artificial Sequence Synthetic peptide
MLSHKKTKTHDHYPSGRMRDPGWHDWRAS

SEQ ID NO: 758 - protein, 29 aa, Artificial Sequence
  Description of Artificial Sequence Synthetic peptide
MLSPKKTKTHDHYPSGRMRDPGWHDWRAS

SEQ ID NO: 759 - protein, 29 aa, Artificial Sequence
  Description of Artificial Sequence Synthetic peptide
MLSSKKTKTHDHYPSGRMRDPGWHDWRAS

SEQ ID NO: 760 - protein, 29 aa, Artificial Sequence
  Description of Artificial Sequence Synthetic peptide
MLSQKKTKTHDHYPSGRMRDPGWHDWRAS

SEQ ID NO: 761 - protein, 29 aa, Artificial Sequence
  Description of Artificial Sequence Synthetic peptide
MLSVKKTKTHDHYPSGRMRDPGWHDWRAS

SEQ ID NO: 762 - protein, 29 aa, Artificial Sequence
  Description of Artificial Sequence Synthetic peptide
MLSFKKTKTHDHYPSGRMRDPGWHDWRAS

SEQ ID NO: 763 - protein, 29 aa, Artificial Sequence
  Description of Artificial Sequence Synthetic peptide
MLSLKKTKTHDHYPSGRMRDPGWHDWRAS

SEQ ID NO: 764 - protein, 29 aa, Artificial Sequence
  Description of Artificial Sequence Synthetic peptide
MLSIKKTKTHDHYPSGRMRDPGWHDWRAS

SEQ ID NO: 765 - protein, 29 aa, Artificial Sequence
  Description of Artificial Sequence Synthetic peptide
MLSDKKTKTHDHYPSGRMRDPGWHDWRAS

SEQ ID NO: 766 - protein, 29 aa, Artificial Sequence
  Description of Artificial Sequence Synthetic peptide
MLSEKKTKTHDHYPSGRMRDPGWHDWRAS

SEQ ID NO: 767 - protein, 29 aa, Artificial Sequence
  Description of Artificial Sequence Synthetic peptide
MLSTVKTKTHDHYPSGRMRDPGWHDWRAS

SEQ ID NO: 768 - protein, 29 aa, Artificial Sequence
  Description of Artificial Sequence Synthetic peptide
MLSTTKTKTHDHYPSGRMRDPGWHDWRAS

SEQ ID NO: 769 - protein, 29 aa, Artificial Sequence
  Description of Artificial Sequence Synthetic peptide
MLSTLKTKTHDHYPSGRMRDPGWHDWRAS

SEQ ID NO: 770 - protein, 29 aa, Artificial Sequence
  Description of Artificial Sequence Synthetic peptide
MLSTSKTKTHDHYPSGRMRDPGWHDWRAS

SEQ ID NO: 771 - protein, 29 aa, Artificial Sequence
  Description of Artificial Sequence Synthetic peptide
MLSTHKTKTHDHYPSGRMRDPGWHDWRAS

SEQ ID NO: 772 - protein, 29 aa, Artificial Sequence
  Description of Artificial Sequence Synthetic peptide
MLSTFKTKTHDHYPSGRMRDPGWHDWRAS

SEQ ID NO: 773 - protein, 29 aa, Artificial Sequence
  Description of Artificial Sequence Synthetic peptide
MLSTQKTKTHDHYPSGRMRDPGWHDWRAS

SEQ ID NO: 774 - protein, 29 aa, Artificial Sequence
  Description of Artificial Sequence Synthetic peptide
MLSTNKTKTHDHYPSGRMRDPGWHDWRAS

SEQ ID NO: 775 - protein, 29 aa, Artificial Sequence
  Description of Artificial Sequence Synthetic peptide
MLSTPKTKTHDHYPSGRMRDPGWHDWRAS

SEQ ID NO: 776 - protein, 29 aa, Artificial Sequence
  Description of Artificial Sequence Synthetic peptide
MLSTYKTKTHDHYPSGRMRDPGWHDWRAS

SEQ ID NO: 777 - protein, 29 aa, Artificial Sequence
  Description of Artificial Sequence Synthetic peptide
MLSTGKTKTHDHYPSGRMRDPGWHDWRAS

SEQ ID NO: 778 - protein, 29 aa, Artificial Sequence
  Description of Artificial Sequence Synthetic peptide
MLSTIKTKTHDHYPSGRMRDPGWHDWRAS

SEQ ID NO: 779 - protein, 29 aa, Artificial Sequence
  Description of Artificial Sequence Synthetic peptide
MLSTWKTKTHDHYPSGRMRDPGWHDWRAS

SEQ ID NO: 780 - protein, 29 aa, Artificial Sequence
  Description of Artificial Sequence Synthetic peptide
MLSTEKTKTHDHYPSGRMRDPGWHDWRAS

SEQ ID NO: 781 - protein, 29 aa, Artificial Sequence
  Description of Artificial Sequence Synthetic peptide
MLSTDKTKTHDHYPSGRMRDPGWHDWRAS

SEQ ID NO: 782 - protein, 29 aa, Artificial Sequence
  Description of Artificial Sequence Synthetic peptide
MLSTKITKTHDHYPSGRMRDPGWHDWRAS

SEQ ID NO: 783 - protein, 29 aa, Artificial Sequence
  Description of Artificial Sequence Synthetic peptide
MLSTKLTKTHDHYPSGRMRDPGWHDWRAS

SEQ ID NO: 784 - protein, 29 aa, Artificial Sequence
  Description of Artificial Sequence Synthetic peptide
MLSTKWTKTHDHYPSGRMRDPGWHDWRAS

SEQ ID NO: 785 - protein, 29 aa, Artificial Sequence
  Description of Artificial Sequence Synthetic peptide
MLSTKYTKTHDHYPSGRMRDPGWHDWRAS

SEQ ID NO: 786 - protein, 29 aa, Artificial Sequence
  Description of Artificial Sequence Synthetic peptide
MLSTKFTKTHDHYPSGRMRDPGWHDWRAS

SEQ ID NO: 787 - protein, 29 aa, Artificial Sequence
  Description of Artificial Sequence Synthetic peptide
MLSTKVTKTHDHYPSGRMRDPGWHDWRAS

SEQ ID NO: 788 - protein, 29 aa, Artificial Sequence
  Description of Artificial Sequence Synthetic peptide
MLSTKSTKTHDHYPSGRMRDPGWHDWRAS

SEQ ID NO: 789 - protein, 29 aa, Artificial Sequence
  Description of Artificial Sequence Synthetic peptide
MLSTKNTKTHDHYPSGRMRDPGWHDWRAS

SEQ ID NO: 790 - protein, 29 aa, Artificial Sequence
  Description of Artificial Sequence Synthetic peptide
MLSTKGTKTHDHYPSGRMRDPGWHDWRAS

SEQ ID NO: 791 - protein, 29 aa, Artificial Sequence
  Description of Artificial Sequence Synthetic peptide
MLSTKHTKTHDHYPSGRMRDPGWHDWRAS

SEQ ID NO: 792 - protein, 29 aa, Artificial Sequence
  Description of Artificial Sequence Synthetic peptide
MLSTKQTKTHDHYPSGRMRDPGWHDWRAS

SEQ ID NO: 793 - protein, 29 aa, Artificial Sequence
  Description of Artificial Sequence Synthetic peptide
MLSTKATKTHDHYPSGRMRDPGWHDWRAS

SEQ ID NO: 794 - protein, 29 aa, Artificial Sequence
  Description of Artificial Sequence Synthetic peptide
MLSTKPTKTHDHYPSGRMRDPGWHDWRAS

SEQ ID NO: 795 - protein, 29 aa, Artificial Sequence
  Description of Artificial Sequence Synthetic peptide
MLSTKETKTHDHYPSGRMRDPGWHDWRAS

SEQ ID NO: 796 - protein, 29 aa, Artificial Sequence
  Description of Artificial Sequence Synthetic peptide
MLSTKDTKTHDHYPSGRMRDPGWHDWRAS

SEQ ID NO: 797 - protein, 29 aa, Artificial Sequence
  Description of Artificial Sequence Synthetic peptide
MLSTKKSKTHDHYPSGRMRDPGWHDWRAS

SEQ ID NO: 798 - protein, 29 aa, Artificial Sequence
  Description of Artificial Sequence Synthetic peptide
MLSTKKLKTHDHYPSGRMRDPGWHDWRAS

SEQ ID NO: 799 - protein, 29 aa, Artificial Sequence
  Description of Artificial Sequence Synthetic peptide
MLSTKKWKTHDHYPSGRMRDPGWHDWRAS

SEQ ID NO: 800 - protein, 29 aa, Artificial Sequence
  Description of Artificial Sequence Synthetic peptide
MLSTKKFKTHDHYPSGRMRDPGWHDWRAS

SEQ ID NO: 801 - protein, 29 aa, Artificial Sequence
  Description of Artificial Sequence Synthetic peptide
MLSTKKAKTHDHYPSGRMRDPGWHDWRAS

SEQ ID NO: 802 - protein, 29 aa, Artificial Sequence
  Description of Artificial Sequence Synthetic peptide
MLSTKKIKTHDHYPSGRMRDPGWHDWRAS

SEQ ID NO: 803 - protein, 29 aa, Artificial Sequence
  Description of Artificial Sequence Synthetic peptide
MLSTKKHKTHDHYPSGRMRDPGWHDWRAS

SEQ ID NO: 804 - protein, 29 aa, Artificial Sequence
  Description of Artificial Sequence Synthetic peptide
MLSTKKPKTHDHYPSGRMRDPGWHDWRAS

SEQ ID NO: 805 - protein, 29 aa, Artificial Sequence
  Description of Artificial Sequence Synthetic peptide
MLSTKKVKTHDHYPSGRMRDPGWHDWRAS

SEQ ID NO: 806 - protein, 29 aa, Artificial Sequence
  Description of Artificial Sequence Synthetic peptide
MLSTKKYKTHDHYPSGRMRDPGWHDWRAS

SEQ ID NO: 807 - protein, 29 aa, Artificial Sequence
  Description of Artificial Sequence Synthetic peptide
MLSTKKEKTHDHYPSGRMRDPGWHDWRAS

SEQ ID NO: 808 - protein, 29 aa, Artificial Sequence
  Description of Artificial Sequence Synthetic peptide
MLSTKKDKTHDHYPSGRMRDPGWHDWRAS

SEQ ID NO: 809 - protein, 29 aa, Artificial Sequence
  Description of Artificial Sequence Synthetic peptide
MLSTKKTHTHDHYPSGRMRDPGWHDWRAS

SEQ ID NO: 810 - protein, 29 aa, Artificial Sequence
  Description of Artificial Sequence Synthetic peptide
MLSTKKTPTHDHYPSGRMRDPGWHDWRAS

SEQ ID NO: 811 - protein, 29 aa, Artificial Sequence
  Description of Artificial Sequence Synthetic peptide
MLSTKKTFTHDHYPSGRMRDPGWHDWRAS

SEQ ID NO: 812 - protein, 29 aa, Artificial Sequence
  Description of Artificial Sequence Synthetic peptide
MLSTKKTGTHDHYPSGRMRDPGWHDWRAS

SEQ ID NO: 813 - protein, 29 aa, Artificial Sequence
  Description of Artificial Sequence Synthetic peptide
MLSTKKTATHDHYPSGRMRDPGWHDWRAS

SEQ ID NO: 814 - protein, 29 aa, Artificial Sequence
  Description of Artificial Sequence Synthetic peptide
MLSTKKTLTHDHYPSGRMRDPGWHDWRAS

SEQ ID NO: 815 - protein, 29 aa, Artificial Sequence
  Description of Artificial Sequence Synthetic peptide
MLSTKKTQTHDHYPSGRMRDPGWHDWRAS

SEQ ID NO: 816 - protein, 29 aa, Artificial Sequence
  Description of Artificial Sequence Synthetic peptide
MLSTKKTYTHDHYPSGRMRDPGWHDWRAS

SEQ ID NO: 817 - protein, 29 aa, Artificial Sequence
  Description of Artificial Sequence Synthetic peptide
MLSTKKTVTHDHYPSGRMRDPGWHDWRAS

SEQ ID NO: 818 - protein, 29 aa, Artificial Sequence
  Description of Artificial Sequence Synthetic peptide
MLSTKKTSTHDHYPSGRMRDPGWHDWRAS

SEQ ID NO: 819 - protein, 29 aa, Artificial Sequence
  Description of Artificial Sequence Synthetic peptide
MLSTKKTTTHDHYPSGRMRDPGWHDWRAS

SEQ ID NO: 820 - protein, 29 aa, Artificial Sequence
  Description of Artificial Sequence Synthetic peptide
MLSTKKTDTHDHYPSGRMRDPGWHDWRAS

SEQ ID NO: 821 - protein, 29 aa, Artificial Sequence
  Description of Artificial Sequence Synthetic peptide
MLSTKKTITHDHYPSGRMRDPGWHDWRAS

SEQ ID NO: 822 - protein, 29 aa, Artificial Sequence
  Description of Artificial Sequence Synthetic peptide
MLSTKKTNTHDHYPSGRMRDPGWHDWRAS

SEQ ID NO: 823 - protein, 29 aa, Artificial Sequence
  Description of Artificial Sequence Synthetic peptide
MLSTKKTETHDHYPSGRMRDPGWHDWRAS

SEQ ID NO: 824 - protein, 29 aa, Artificial Sequence
  Description of Artificial Sequence Synthetic peptide
MLSTKKTKLHDHYPSGRMRDPGWHDWRAS

SEQ ID NO: 825 - protein, 29 aa, Artificial Sequence
  Description of Artificial Sequence Synthetic peptide
MLSTKKTKYHDHYPSGRMRDPGWHDWRAS

SEQ ID NO: 826 - protein, 29 aa, Artificial Sequence
  Description of Artificial Sequence Synthetic peptide
MLSTKKTKPHDHYPSGRMRDPGWHDWRAS

SEQ ID NO: 827 - protein, 29 aa, Artificial Sequence
  Description of Artificial Sequence Synthetic peptide
MLSTKKTKKHDHYPSGRMRDPGWHDWRAS

SEQ ID NO: 828 - protein, 29 aa, Artificial Sequence
  Description of Artificial Sequence Synthetic peptide
MLSTKKTKNHDHYPSGRMRDPGWHDWRAS

SEQ ID NO: 829 - protein, 29 aa, Artificial Sequence
  Description of Artificial Sequence Synthetic peptide
MLSTKKTKFHDHYPSGRMRDPGWHDWRAS

SEQ ID NO: 830 - protein, 29 aa, Artificial Sequence
  Description of Artificial Sequence Synthetic peptide
MLSTKKTKSHDHYPSGRMRDPGWHDWRAS

SEQ ID NO: 831 - protein, 29 aa, Artificial Sequence
  Description of Artificial Sequence Synthetic peptide
MLSTKKTKHHDHYPSGRMRDPGWHDWRAS

SEQ ID NO: 832 - protein, 29 aa, Artificial Sequence
  Description of Artificial Sequence Synthetic peptide
MLSTKKTKQHDHYPSGRMRDPGWHDWRAS

SEQ ID NO: 833 - protein, 29 aa, Artificial Sequence
  Description of Artificial Sequence Synthetic peptide
MLSTKKTKAHDHYPSGRMRDPGWHDWRAS

SEQ ID NO: 834 - protein, 29 aa, Artificial Sequence
  Description of Artificial Sequence Synthetic peptide
MLSTKKTKVHDHYPSGRMRDPGWHDWRAS

SEQ ID NO: 835 - protein, 29 aa, Artificial Sequence
  Description of Artificial Sequence Synthetic peptide
MLSTKKTKEHDHYPSGRMRDPGWHDWRAS

SEQ ID NO: 836 - protein, 29 aa, Artificial Sequence
  Description of Artificial Sequence Synthetic peptide
MLSTKKTKIHDHYPSGRMRDPGWHDWRAS

SEQ ID NO: 837 - protein, 29 aa, Artificial Sequence
  Description of Artificial Sequence Synthetic peptide
MLSTKKTKDHDHYPSGRMRDPGWHDWRAS

SEQ ID NO: 838 - protein, 29 aa, Artificial Sequence
  Description of Artificial Sequence Synthetic peptide
MLSTKKTKTWDHYPSGRMRDPGWHDWRAS

SEQ ID NO: 839 - protein, 29 aa, Artificial Sequence
  Description of Artificial Sequence Synthetic peptide
MLSTKKTKTFDHYPSGRMRDPGWHDWRAS

SEQ ID NO: 840 - protein, 29 aa, Artificial Sequence
  Description of Artificial Sequence Synthetic peptide
MLSTKKTKTPDHYPSGRMRDPGWHDWRAS

SEQ ID NO: 841 - protein, 29 aa, Artificial Sequence
  Description of Artificial Sequence Synthetic peptide
MLSTKKTKTQDHYPSGRMRDPGWHDWRAS

SEQ ID NO: 842 - protein, 29 aa, Artificial Sequence
  Description of Artificial Sequence Synthetic peptide
MLSTKKTKTLDHYPSGRMRDPGWHDWRAS

SEQ ID NO: 843 - protein, 29 aa, Artificial Sequence
  Description of Artificial Sequence Synthetic peptide
MLSTKKTKTTDHYPSGRMRDPGWHDWRAS

SEQ ID NO: 844 - protein, 29 aa, Artificial Sequence
  Description of Artificial Sequence Synthetic peptide
MLSTKKTKTYDHYPSGRMRDPGWHDWRAS

SEQ ID NO: 845 - protein, 29 aa, Artificial Sequence
  Description of Artificial Sequence Synthetic peptide
MLSTKKTKTDDHYPSGRMRDPGWHDWRAS

SEQ ID NO: 846 - protein, 29 aa, Artificial Sequence
  Description of Artificial Sequence Synthetic peptide
MLSTKKTKTEDHYPSGRMRDPGWHDWRAS

SEQ ID NO: 847 - protein, 29 aa, Artificial Sequence
  Description of Artificial Sequence Synthetic peptide
MLSTKKTKTVDHYPSGRMRDPGWHDWRAS

SEQ ID NO: 848 - protein, 29 aa, Artificial Sequence
  Description of Artificial Sequence Synthetic peptide
MLSTKKTKTADHYPSGRMRDPGWHDWRAS

SEQ ID NO: 849 - protein, 29 aa, Artificial Sequence
  Description of Artificial Sequence Synthetic peptide
MLSTKKTKTIDHYPSGRMRDPGWHDWRAS

SEQ ID NO: 850 - protein, 29 aa, Artificial Sequence
  Description of Artificial Sequence Synthetic peptide
MLSTKKTKTHEHYPSGRMRDPGWHDWRAS

SEQ ID NO: 851 - protein, 29 aa, Artificial Sequence
  Description of Artificial Sequence Synthetic peptide
MLSTKKTKTHDNYPSGRMRDPGWHDWRAS

SEQ ID NO: 852 - protein, 29 aa, Artificial Sequence
  Description of Artificial Sequence Synthetic peptide
MLSTKKTKTHDWYPSGRMRDPGWHDWRAS

SEQ ID NO: 853 - protein, 29 aa, Artificial Sequence
  Description of Artificial Sequence Synthetic peptide
MLSTKKTKTHDLYPSGRMRDPGWHDWRAS

SEQ ID NO: 854 - protein, 29 aa, Artificial Sequence
  Description of Artificial Sequence Synthetic peptide
MLSTKKTKTHDFYPSGRMRDPGWHDWRAS

SEQ ID NO: 855 - protein, 29 aa, Artificial Sequence
  Description of Artificial Sequence Synthetic peptide
MLSTKKTKTHDTYPSGRMRDPGWHDWRAS

SEQ ID NO: 856 - protein, 29 aa, Artificial Sequence
  Description of Artificial Sequence Synthetic peptide
MLSTKKTKTHDQYPSGRMRDPGWHDWRAS

SEQ ID NO: 857 - protein, 29 aa, Artificial Sequence
  Description of Artificial Sequence Synthetic peptide
MLSTKKTKTHDGYPSGRMRDPGWHDWRAS

SEQ ID NO: 858 - protein, 29 aa, Artificial Sequence
  Description of Artificial Sequence Synthetic peptide
MLSTKKTKTHDAYPSGRMRDPGWHDWRAS

SEQ ID NO: 859 - protein, 29 aa, Artificial Sequence
  Description of Artificial Sequence Synthetic peptide
MLSTKKTKTHDIYPSGRMRDPGWHDWRAS

SEQ ID NO: 860 - protein, 29 aa, Artificial Sequence
  Description of Artificial Sequence Synthetic peptide
MLSTKKTKTHDSYPSGRMRDPGWHDWRAS

SEQ ID NO: 861 - protein, 29 aa, Artificial Sequence
  Description of Artificial Sequence Synthetic peptide
MLSTKKTKTHDYYPSGRMRDPGWHDWRAS

SEQ ID NO: 862 - protein, 29 aa, Artificial Sequence
  Description of Artificial Sequence Synthetic peptide
MLSTKKTKTHDPYPSGRMRDPGWHDWRAS

SEQ ID NO: 863 - protein, 29 aa, Artificial Sequence
  Description of Artificial Sequence Synthetic peptide
MLSTKKTKTHDDYPSGRMRDPGWHDWRAS

SEQ ID NO: 864 - protein, 29 aa, Artificial Sequence
  Description of Artificial Sequence Synthetic peptide
MLSTKKTKTHDVYPSGRMRDPGWHDWRAS

SEQ ID NO: 865 - protein, 29 aa, Artificial Sequence
  Description of Artificial Sequence Synthetic peptide
MLSTKKTKTHDEYPSGRMRDPGWHDWRAS

SEQ ID NO: 866 - protein, 29 aa, Artificial Sequence
  Description of Artificial Sequence Synthetic peptide
MLSTKKTKTHDHHPSGRMRDPGWHDWRAS

SEQ ID NO: 867 - protein, 29 aa, Artificial Sequence
  Description of Artificial Sequence Synthetic peptide
MLSTKKTKTHDHSPSGRMRDPGWHDWRAS

SEQ ID NO: 868 - protein, 29 aa, Artificial Sequence
  Description of Artificial Sequence Synthetic peptide
MLSTKKTKTHDHVPSGRMRDPGWHDWRAS

SEQ ID NO: 869 - protein, 29 aa, Artificial Sequence
  Description of Artificial Sequence Synthetic peptide
MLSTKKTKTHDHQPSGRMRDPGWHDWRAS

SEQ ID NO: 870 - protein, 29 aa, Artificial Sequence
  Description of Artificial Sequence Synthetic peptide
MLSTKKTKTHDHAPSGRMRDPGWHDWRAS

SEQ ID NO: 871 - protein, 29 aa, Artificial Sequence
  Description of Artificial Sequence Synthetic peptide
MLSTKKTKTHDHDPSGRMRDPGWHDWRAS

SEQ ID NO: 872 - protein, 29 aa, Artificial Sequence
  Description of Artificial Sequence Synthetic peptide
MLSTKKTKTHDHIPSGRMRDPGWHDWRAS

SEQ ID NO: 873 - protein, 29 aa, Artificial Sequence
  Description of Artificial Sequence Synthetic peptide
MLSTKKTKTHDHTPSGRMRDPGWHDWRAS

SEQ ID NO: 874 - protein, 29 aa, Artificial Sequence
  Description of Artificial Sequence Synthetic peptide
MLSTKKTKTHDHEPSGRMRDPGWHDWRAS

SEQ ID NO: 875 - protein, 29 aa, Artificial Sequence
  Description of Artificial Sequence Synthetic peptide
MLSTKKTKTHDHYNSGRMRDPGWHDWRAS

SEQ ID NO: 876 - protein, 29 aa, Artificial Sequence
  Description of Artificial Sequence Synthetic peptide
MLSTKKTKTHDHYRSGRMRDPGWHDWRAS

SEQ ID NO: 877 - protein, 29 aa, Artificial Sequence
  Description of Artificial Sequence Synthetic peptide
MLSTKKTKTHDHYSSGRMRDPGWHDWRAS

SEQ ID NO: 878 - protein, 29 aa, Artificial Sequence
  Description of Artificial Sequence Synthetic peptide
MLSTKKTKTHDHYLSGRMRDPGWHDWRAS

SEQ ID NO: 879 - protein, 29 aa, Artificial Sequence
  Description of Artificial Sequence Synthetic peptide
MLSTKKTKTHDHYVSGRMRDPGWHDWRAS

SEQ ID NO: 880 - protein, 29 aa, Artificial Sequence
  Description of Artificial Sequence Synthetic peptide
MLSTKKTKTHDHYTSGRMRDPGWHDWRAS

SEQ ID NO: 881 - protein, 29 aa, Artificial Sequence
  Description of Artificial Sequence Synthetic peptide
MLSTKKTKTHDHYASGRMRDPGWHDWRAS

SEQ ID NO: 882 - protein, 29 aa, Artificial Sequence
  Description of Artificial Sequence Synthetic peptide
MLSTKKTKTHDHYHSGRMRDPGWHDWRAS

SEQ ID NO: 883 - protein, 29 aa, Artificial Sequence
  Description of Artificial Sequence Synthetic peptide
MLSTKKTKTHDHYQSGRMRDPGWHDWRAS

SEQ ID NO: 884 - protein, 29 aa, Artificial Sequence
  Description of Artificial Sequence Synthetic peptide
MLSTKKTKTHDHYDSGRMRDPGWHDWRAS

SEQ ID NO: 885 - protein, 29 aa, Artificial Sequence
  Description of Artificial Sequence Synthetic peptide
MLSTKKTKTHDHYKSGRMRDPGWHDWRAS

SEQ ID NO: 886 - protein, 29 aa, Artificial Sequence
  Description of Artificial Sequence Synthetic peptide
MLSTKKTKTHDHYESGRMRDPGWHDWRAS

SEQ ID NO: 887 - protein, 29 aa, Artificial Sequence
  Description of Artificial Sequence Synthetic peptide
MLSTKKTKTHDHYPTGRMRDPGWHDWRAS

SEQ ID NO: 888 - protein, 29 aa, Artificial Sequence
  Description of Artificial Sequence Synthetic peptide
MLSTKKTKTHDHYPDGRMRDPGWHDWRAS

SEQ ID NO: 889 - protein, 29 aa, Artificial Sequence
  Description of Artificial Sequence Synthetic peptide
MLSTKKTKTHDHYPWGRMRDPGWHDWRAS

SEQ ID NO: 890 - protein, 29 aa, Artificial Sequence
  Description of Artificial Sequence Synthetic peptide
MLSTKKTKTHDHYPNGRMRDPGWHDWRAS

SEQ ID NO: 891 - protein, 29 aa, Artificial Sequence
  Description of Artificial Sequence Synthetic peptide
MLSTKKTKTHDHYPHGRMRDPGWHDWRAS

SEQ ID NO: 892 - protein, 29 aa, Artificial Sequence
  Description of Artificial Sequence Synthetic peptide
MLSTKKTKTHDHYPFGRMRDPGWHDWRAS

SEQ ID NO: 893 - protein, 29 aa, Artificial Sequence
  Description of Artificial Sequence Synthetic peptide
MLSTKKTKTHDHYPQGRMRDPGWHDWRAS

SEQ ID NO: 894 - protein, 29 aa, Artificial Sequence
  Description of Artificial Sequence Synthetic peptide
MLSTKKTKTHDHYPLGRMRDPGWHDWRAS

SEQ ID NO: 895 - protein, 29 aa, Artificial Sequence
  Description of Artificial Sequence Synthetic peptide
MLSTKKTKTHDHYPEGRMRDPGWHDWRAS

SEQ ID NO: 896 - protein, 29 aa, Artificial Sequence
  Description of Artificial Sequence Synthetic peptide
MLSTKKTKTHDHYPAGRMRDPGWHDWRAS

SEQ ID NO: 897 - protein, 29 aa, Artificial Sequence
  Description of Artificial Sequence Synthetic peptide
MLSTKKTKTHDHYPYGRMRDPGWHDWRAS

SEQ ID NO: 898 - protein, 29 aa, Artificial Sequence
  Description of Artificial Sequence Synthetic peptide
MLSTKKTKTHDHYPVGRMRDPGWHDWRAS

SEQ ID NO: 899 - protein, 29 aa, Artificial Sequence
  Description of Artificial Sequence Synthetic peptide
MLSTKKTKTHDHYPIGRMRDPGWHDWRAS

SEQ ID NO: 900 - protein, 29 aa, Artificial Sequence
  Description of Artificial Sequence Synthetic peptide
MLSTKKTKTHDHYPSPRMRDPGWHDWRAS

SEQ ID NO: 901 - protein, 29 aa, Artificial Sequence
  Description of Artificial Sequence Synthetic peptide
MLSTKKTKTHDHYPSKRMRDPGWHDWRAS

SEQ ID NO: 902 - protein, 29 aa, Artificial Sequence
  Description of Artificial Sequence Synthetic peptide
MLSTKKTKTHDHYPSNRMRDPGWHDWRAS

SEQ ID NO: 903 - protein, 29 aa, Artificial Sequence
  Description of Artificial Sequence Synthetic peptide
MLSTKKTKTHDHYPSRRMRDPGWHDWRAS

SEQ ID NO: 904 - protein, 29 aa, Artificial Sequence
  Description of Artificial Sequence Synthetic peptide
MLSTKKTKTHDHYPSERMRDPGWHDWRAS

SEQ ID NO: 905 - protein, 29 aa, Artificial Sequence
  Description of Artificial Sequence Synthetic peptide
MLSTKKTKTHDHYPSWRMRDPGWHDWRAS

SEQ ID NO: 906 - protein, 29 aa, Artificial Sequence
  Description of Artificial Sequence Synthetic peptide
MLSTKKTKTHDHYPSLRMRDPGWHDWRAS

SEQ ID NO: 907 - protein, 29 aa, Artificial Sequence
  Description of Artificial Sequence Synthetic peptide
MLSTKKTKTHDHYPSDRMRDPGWHDWRAS

SEQ ID NO: 908 - protein, 29 aa, Artificial Sequence
  Description of Artificial Sequence Synthetic peptide
MLSTKKTKTHDHYPSARMRDPGWHDWRAS

SEQ ID NO: 909 - protein, 29 aa, Artificial Sequence
  Description of Artificial Sequence Synthetic peptide
MLSTKKTKTHDHYPSHRMRDPGWHDWRAS

SEQ ID NO: 910 - protein, 29 aa, Artificial Sequence
  Description of Artificial Sequence Synthetic peptide
MLSTKKTKTHDHYPSQRMRDPGWHDWRAS

SEQ ID NO: 911 - protein, 29 aa, Artificial Sequence
  Description of Artificial Sequence Synthetic peptide
MLSTKKTKTHDHYPSIRMRDPGWHDWRAS

SEQ ID NO: 912 - protein, 29 aa, Artificial Sequence
  Description of Artificial Sequence Synthetic peptide
MLSTKKTKTHDHYPSTRMRDPGWHDWRAS

SEQ ID NO: 913 - protein, 29 aa, Artificial Sequence
  Description of Artificial Sequence Synthetic peptide
MLSTKKTKTHDHYPSVRMRDPGWHDWRAS

SEQ ID NO: 914 - protein, 29 aa, Artificial Sequence
  Description of Artificial Sequence Synthetic peptide
MLSTKKTKTHDHYPSSRMRDPGWHDWRAS

SEQ ID NO: 915 - protein, 29 aa, Artificial Sequence
  Description of Artificial Sequence Synthetic peptide
MLSTKKTKTHDHYPSFRMRDPGWHDWRAS

SEQ ID NO: 916 - protein, 29 aa, Artificial Sequence
  Description of Artificial Sequence Synthetic peptide
MLSTKKTKTHDHYPSYRMRDPGWHDWRAS

SEQ ID NO: 917 - protein, 29 aa, Artificial Sequence
  Description of Artificial Sequence Synthetic peptide
MLSTKKTKTHDHYPSGSMRDPGWHDWRAS

SEQ ID NO: 918 - protein, 29 aa, Artificial Sequence
  Description of Artificial Sequence Synthetic peptide
MLSTKKTKTHDHYPSGKMRDPGWHDWRAS

SEQ ID NO: 919 - protein, 29 aa, Artificial Sequence
  Description of Artificial Sequence Synthetic peptide
MLSTKKTKTHDHYPSGFMRDPGWHDWRAS

SEQ ID NO: 920 - protein, 29 aa, Artificial Sequence
  Description of Artificial Sequence Synthetic peptide
MLSTKKTKTHDHYPSGWMRDPGWHDWRAS

SEQ ID NO: 921 - protein, 29 aa, Artificial Sequence
  Description of Artificial Sequence Synthetic peptide
MLSTKKTKTHDHYPSGIMRDPGWHDWRAS

SEQ ID NO: 922 - protein, 29 aa, Artificial Sequence
  Description of Artificial Sequence Synthetic peptide
MLSTKKTKTHDHYPSGHMRDPGWHDWRAS

SEQ ID NO: 923 - protein, 29 aa, Artificial Sequence
  Description of Artificial Sequence Synthetic peptide
MLSTKKTKTHDHYPSGPMRDPGWHDWRAS

SEQ ID NO: 924 - protein, 29 aa, Artificial Sequence
  Description of Artificial Sequence Synthetic peptide
MLSTKKTKTHDHYPSGGMRDPGWHDWRAS

SEQ ID NO: 925 - protein, 29 aa, Artificial Sequence
  Description of Artificial Sequence Synthetic peptide
MLSTKKTKTHDHYPSGNMRDPGWHDWRAS

SEQ ID NO: 926 - protein, 29 aa, Artificial Sequence
  Description of Artificial Sequence Synthetic peptide
MLSTKKTKTHDHYPSGLMRDPGWHDWRAS

SEQ ID NO: 927 - protein, 29 aa, Artificial Sequence
  Description of Artificial Sequence Synthetic peptide
MLSTKKTKTHDHYPSGYMRDPGWHDWRAS

SEQ ID NO: 928 - protein, 29 aa, Artificial Sequence
  Description of Artificial Sequence Synthetic peptide
MLSTKKTKTHDHYPSGQMRDPGWHDWRAS

SEQ ID NO: 929 - protein, 29 aa, Artificial Sequence
  Description of Artificial Sequence Synthetic peptide
MLSTKKTKTHDHYPSGVMRDPGWHDWRAS

SEQ ID NO: 930 - protein, 29 aa, Artificial Sequence
  Description of Artificial Sequence Synthetic peptide
MLSTKKTKTHDHYPSGTMRDPGWHDWRAS

SEQ ID NO: 931 - protein, 29 aa, Artificial Sequence
  Description of Artificial Sequence Synthetic peptide
MLSTKKTKTHDHYPSGAMRDPGWHDWRAS

SEQ ID NO: 932 - protein, 29 aa, Artificial Sequence
  Description of Artificial Sequence Synthetic peptide
MLSTKKTKTHDHYPSGDMRDPGWHDWRAS

SEQ ID NO: 933 - protein, 29 aa, Artificial Sequence
  Description of Artificial Sequence Synthetic peptide
MLSTKKTKTHDHYPSGEMRDPGWHDWRAS

SEQ ID NO: 934 - protein, 29 aa, Artificial Sequence
  Description of Artificial Sequence Synthetic peptide
MLSTKKTKTHDHYPSGRIRDPGWHDWRAS

SEQ ID NO: 935 - protein, 29 aa, Artificial Sequence
  Description of Artificial Sequence Synthetic peptide
MLSTKKTKTHDHYPSGRLRDPGWHDWRAS

SEQ ID NO: 936 - protein, 29 aa, Artificial Sequence
  Description of Artificial Sequence Synthetic peptide
MLSTKKTKTHDHYPSGRVRDPGWHDWRAS

SEQ ID NO: 937 - protein, 29 aa, Artificial Sequence
  Description of Artificial Sequence Synthetic peptide
MLSTKKTKTHDHYPSGRERDPGWHDWRAS

SEQ ID NO: 938 - protein, 29 aa, Artificial Sequence
  Description of Artificial Sequence Synthetic peptide
MLSTKKTKTHDHYPSGRDRDPGWHDWRAS

SEQ ID NO: 939 - protein, 29 aa, Artificial Sequence
  Description of Artificial Sequence Synthetic peptide
MLSTKKTKTHDHYPSGRMPDPGWHDWRAS

SEQ ID NO: 940 - protein, 29 aa, Artificial Sequence
  Description of Artificial Sequence Synthetic peptide
MLSTKKTKTHDHYPSGRMNDPGWHDWRAS

SEQ ID NO: 941 - protein, 29 aa, Artificial Sequence
  Description of Artificial Sequence Synthetic peptide
MLSTKKTKTHDHYPSGRMHDPGWHDWRAS

SEQ ID NO: 942 - protein, 29 aa, Artificial Sequence
  Description of Artificial Sequence Synthetic peptide
MLSTKKTKTHDHYPSGRMWDPGWHDWRAS

SEQ ID NO: 943 - protein, 29 aa, Artificial Sequence
  Description of Artificial Sequence Synthetic peptide
MLSTKKTKTHDHYPSGRMQDPGWHDWRAS

SEQ ID NO: 944 - protein, 29 aa, Artificial Sequence
  Description of Artificial Sequence Synthetic peptide
MLSTKKTKTHDHYPSGRMGDPGWHDWRAS

SEQ ID NO: 945 - protein, 29 aa, Artificial Sequence
  Description of Artificial Sequence Synthetic peptide
MLSTKKTKTHDHYPSGRMKDPGWHDWRAS

SEQ ID NO: 946 - protein, 29 aa, Artificial Sequence
  Description of Artificial Sequence Synthetic peptide
MLSTKKTKTHDHYPSGRMVDPGWHDWRAS

SEQ ID NO: 947 - protein, 29 aa, Artificial Sequence
  Description of Artificial Sequence Synthetic peptide
MLSTKKTKTHDHYPSGRMSDPGWHDWRAS

SEQ ID NO: 948 - protein, 29 aa, Artificial Sequence
  Description of Artificial Sequence Synthetic peptide
MLSTKKTKTHDHYPSGRMDDPGWHDWRAS

SEQ ID NO: 949 - protein, 29 aa, Artificial Sequence
  Description of Artificial Sequence Synthetic peptide
MLSTKKTKTHDHYPSGRMFDPGWHDWRAS

SEQ ID NO: 950 - protein, 29 aa, Artificial Sequence
  Description of Artificial Sequence Synthetic peptide
MLSTKKTKTHDHYPSGRMTDPGWHDWRAS

SEQ ID NO: 951 - protein, 29 aa, Artificial Sequence
  Description of Artificial Sequence Synthetic peptide
MLSTKKTKTHDHYPSGRMLDPGWHDWRAS

SEQ ID NO: 952 - protein, 29 aa, Artificial Sequence
  Description of Artificial Sequence Synthetic peptide
MLSTKKTKTHDHYPSGRMYDPGWHDWRAS

SEQ ID NO: 953 - protein, 29 aa, Artificial Sequence
  Description of Artificial Sequence Synthetic peptide
MLSTKKTKTHDHYPSGRMEDPGWHDWRAS

SEQ ID NO: 954 - protein, 29 aa, Artificial Sequence
  Description of Artificial Sequence Synthetic peptide
MLSTKKTKTHDHYPSGRMIDPGWHDWRAS

SEQ ID NO: 955 - protein, 29 aa, Artificial Sequence
  Description of Artificial Sequence Synthetic peptide
MLSTKKTKTHDHYPSGRMADPGWHDWRAS

SEQ ID NO: 956 - protein, 29 aa, Artificial Sequence
  Description of Artificial Sequence Synthetic peptide
MLSTKKTKTHDHYPSGRMRFPGWHDWRAS

SEQ ID NO: 957 - protein, 29 aa, Artificial Sequence
  Description of Artificial Sequence Synthetic peptide
MLSTKKTKTHDHYPSGRMRHPGWHDWRAS

SEQ ID NO: 958 - protein, 29 aa, Artificial Sequence
  Description of Artificial Sequence Synthetic peptide
MLSTKKTKTHDHYPSGRMRWPGWHDWRAS

SEQ ID NO: 959 - protein, 29 aa, Artificial Sequence
  Description of Artificial Sequence Synthetic peptide
MLSTKKTKTHDHYPSGRMRIPGWHDWRAS

SEQ ID NO: 960 - protein, 29 aa, Artificial Sequence
  Description of Artificial Sequence Synthetic peptide
MLSTKKTKTHDHYPSGRMRNPGWHDWRAS

SEQ ID NO: 961 - protein, 29 aa, Artificial Sequence
  Description of Artificial Sequence Synthetic peptide
MLSTKKTKTHDHYPSGRMRAPGWHDWRAS

SEQ ID NO: 962 - protein, 29 aa, Artificial Sequence
  Description of Artificial Sequence Synthetic peptide
MLSTKKTKTHDHYPSGRMRSPGWHDWRAS

SEQ ID NO: 963 - protein, 29 aa, Artificial Sequence
  Description of Artificial Sequence Synthetic peptide
MLSTKKTKTHDHYPSGRMREPGWHDWRAS

SEQ ID NO: 964 - protein, 29 aa, Artificial Sequence
  Description of Artificial Sequence Synthetic peptide
MLSTKKTKTHDHYPSGRMRTPGWHDWRAS

SEQ ID NO: 965 - protein, 29 aa, Artificial Sequence
  Description of Artificial Sequence Synthetic peptide
MLSTKKTKTHDHYPSGRMRYPGWHDWRAS

SEQ ID NO: 966 - protein, 29 aa, Artificial Sequence
  Description of Artificial Sequence Synthetic peptide
MLSTKKTKTHDHYPSGRMRVPGWHDWRAS

SEQ ID NO: 967 - protein, 29 aa, Artificial Sequence
  Description of Artificial Sequence Synthetic peptide
MLSTKKTKTHDHYPSGRMRQPGWHDWRAS

SEQ ID NO: 968 - protein, 29 aa, Artificial Sequence
  Description of Artificial Sequence Synthetic peptide
MLSTKKTKTHDHYPSGRMRLPGWHDWRAS

SEQ ID NO: 969 - protein, 29 aa, Artificial Sequence
  Description of Artificial Sequence Synthetic peptide
MLSTKKTKTHDHYPSGRMRDQGWHDWRAS

SEQ ID NO: 970 - protein, 29 aa, Artificial Sequence
  Description of Artificial Sequence Synthetic peptide
MLSTKKTKTHDHYPSGRMRDWGWHDWRAS

SEQ ID NO: 971 - protein, 29 aa, Artificial Sequence
  Description of Artificial Sequence Synthetic peptide
MLSTKKTKTHDHYPSGRMRDKGWHDWRAS

SEQ ID NO: 972 - protein, 29 aa, Artificial Sequence
  Description of Artificial Sequence Synthetic peptide
MLSTKKTKTHDHYPSGRMRDLGWHDWRAS

SEQ ID NO: 973 - protein, 29 aa, Artificial Sequence
  Description of Artificial Sequence Synthetic peptide
MLSTKKTKTHDHYPSGRMRDYGWHDWRAS

SEQ ID NO: 974 - protein, 29 aa, Artificial Sequence
  Description of Artificial Sequence Synthetic peptide
MLSTKKTKTHDHYPSGRMRDHGWHDWRAS

SEQ ID NO: 975 - protein, 29 aa, Artificial Sequence
  Description of Artificial Sequence Synthetic peptide
MLSTKKTKTHDHYPSGRMRDGGWHDWRAS

SEQ ID NO: 976 - protein, 29 aa, Artificial Sequence
  Description of Artificial Sequence Synthetic peptide
MLSTKKTKTHDHYPSGRMRDNGWHDWRAS

SEQ ID NO: 977 - protein, 29 aa, Artificial Sequence
  Description of Artificial Sequence Synthetic peptide
MLSTKKTKTHDHYPSGRMRDTGWHDWRAS

SEQ ID NO: 978 - protein, 29 aa, Artificial Sequence
  Description of Artificial Sequence Synthetic peptide
MLSTKKTKTHDHYPSGRMRDSGWHDWRAS

SEQ ID NO: 979 - protein, 29 aa, Artificial Sequence
  Description of Artificial Sequence Synthetic peptide
MLSTKKTKTHDHYPSGRMRDEGWHDWRAS

SEQ ID NO: 980 - protein, 29 aa, Artificial Sequence
  Description of Artificial Sequence Synthetic peptide
MLSTKKTKTHDHYPSGRMRDDGWHDWRAS

SEQ ID NO: 981 - protein, 29 aa, Artificial Sequence
  Description of Artificial Sequence Synthetic peptide
MLSTKKTKTHDHYPSGRMRDIGWHDWRAS

SEQ ID NO: 982 - protein, 29 aa, Artificial Sequence
  Description of Artificial Sequence Synthetic peptide
MLSTKKTKTHDHYPSGRMRDAGWHDWRAS

SEQ ID NO: 983 - protein, 29 aa, Artificial Sequence
  Description of Artificial Sequence Synthetic peptide
MLSTKKTKTHDHYPSGRMRDVGWHDWRAS

SEQ ID NO: 984 - protein, 29 aa, Artificial Sequence
  Description of Artificial Sequence Synthetic peptide
MLSTKKTKTHDHYPSGRMRDPFWHDWRAS

SEQ ID NO: 985 - protein, 29 aa, Artificial Sequence
  Description of Artificial Sequence Synthetic peptide
MLSTKKTKTHDHYPSGRMRDPQWHDWRAS

SEQ ID NO: 986 - protein, 29 aa, Artificial Sequence
  Description of Artificial Sequence Synthetic peptide
MLSTKKTKTHDHYPSGRMRDPTWHDWRAS

SEQ ID NO: 987 - protein, 29 aa, Artificial Sequence
  Description of Artificial Sequence Synthetic peptide
MLSTKKTKTHDHYPSGRMRDPYWHDWRAS

SEQ ID NO: 988 - protein, 29 aa, Artificial Sequence
  Description of Artificial Sequence Synthetic peptide
MLSTKKTKTHDHYPSGRMRDPNWHDWRAS

SEQ ID NO: 989 - protein, 29 aa, Artificial Sequence
  Description of Artificial Sequence Synthetic peptide
MLSTKKTKTHDHYPSGRMRDPHWHDWRAS

SEQ ID NO: 990 - protein, 29 aa, Artificial Sequence
  Description of Artificial Sequence Synthetic peptide
MLSTKKTKTHDHYPSGRMRDPLWHDWRAS

SEQ ID NO: 991 - protein, 29 aa, Artificial Sequence
  Description of Artificial Sequence Synthetic peptide
MLSTKKTKTHDHYPSGRMRDPIWHDWRAS

SEQ ID NO: 992 - protein, 29 aa, Artificial Sequence
  Description of Artificial Sequence Synthetic peptide
MLSTKKTKTHDHYPSGRMRDPVWHDWRAS

SEQ ID NO: 993 - protein, 29 aa, Artificial Sequence
  Description of Artificial Sequence Synthetic peptide
MLSTKKTKTHDHYPSGRMRDPSWHDWRAS

SEQ ID NO: 994 - protein, 29 aa, Artificial Sequence
  Description of Artificial Sequence Synthetic peptide
MLSTKKTKTHDHYPSGRMRDPDWHDWRAS

SEQ ID NO: 995 - protein, 29 aa, Artificial Sequence
  Description of Artificial Sequence Synthetic peptide
MLSTKKTKTHDHYPSGRMRDPEWHDWRAS

SEQ ID NO: 996 - protein, 29 aa, Artificial Sequence
  Description of Artificial Sequence Synthetic peptide
MLSTKKTKTHDHYPSGRMRDPGPHDWRAS

SEQ ID NO: 997 - protein, 29 aa, Artificial Sequence
  Description of Artificial Sequence Synthetic peptide
MLSTKKTKTHDHYPSGRMRDPGYHDWRAS

SEQ ID NO: 998 - protein, 29 aa, Artificial Sequence
  Description of Artificial Sequence Synthetic peptide
MLSTKKTKTHDHYPSGRMRDPGRHDWRAS

SEQ ID NO: 999 - protein, 29 aa, Artificial Sequence
  Description of Artificial Sequence Synthetic peptide
MLSTKKTKTHDHYPSGRMRDPGKHDWRAS

SEQ ID NO: 1000 - protein, 29 aa, Artificial Sequence
  Description of Artificial Sequence Synthetic peptide
MLSTKKTKTHDHYPSGRMRDPGHHDWRAS

SEQ ID NO: 1001 - protein, 29 aa, Artificial Sequence
  Description of Artificial Sequence Synthetic peptide
MLSTKKTKTHDHYPSGRMRDPGFHDWRAS

SEQ ID NO: 1002 - protein, 29 aa, Artificial Sequence
  Description of Artificial Sequence Synthetic peptide
MLSTKKTKTHDHYPSGRMRDPGNHDWRAS

SEQ ID NO: 1003 - protein, 29 aa, Artificial Sequence
  Description of Artificial Sequence Synthetic peptide
MLSTKKTKTHDHYPSGRMRDPGDHDWRAS

SEQ ID NO: 1004 - protein, 29 aa, Artificial Sequence
  Description of Artificial Sequence Synthetic peptide
MLSTKKTKTHDHYPSGRMRDPGSHDWRAS

SEQ ID NO: 1005 - protein, 29 aa, Artificial Sequence
  Description of Artificial Sequence Synthetic peptide
MLSTKKTKTHDHYPSGRMRDPGAHDWRAS

SEQ ID NO: 1006 - protein, 29 aa, Artificial Sequence
  Description of Artificial Sequence Synthetic peptide
MLSTKKTKTHDHYPSGRMRDPGEHDWRAS

SEQ ID NO: 1007 - protein, 29 aa, Artificial Sequence
  Description of Artificial Sequence Synthetic peptide
MLSTKKTKTHDHYPSGRMRDPGIHDWRAS

SEQ ID NO: 1008 - protein, 29 aa, Artificial Sequence
  Description of Artificial Sequence Synthetic peptide
MLSTKKTKTHDHYPSGRMRDPGGHDWRAS

SEQ ID NO: 1009 - protein, 29 aa, Artificial Sequence
  Description of Artificial Sequence Synthetic peptide
MLSTKKTKTHDHYPSGRMRDPGQHDWRAS

SEQ ID NO: 1010 - protein, 29 aa, Artificial Sequence
  Description of Artificial Sequence Synthetic peptide
MLSTKKTKTHDHYPSGRMRDPGTHDWRAS

SEQ ID NO: 1011 - protein, 29 aa, Artificial Sequence
  Description of Artificial Sequence Synthetic peptide
MLSTKKTKTHDHYPSGRMRDPGVHDWRAS

SEQ ID NO: 1012 - protein, 29 aa, Artificial Sequence
  Description of Artificial Sequence Synthetic peptide
MLSTKKTKTHDHYPSGRMRDPGWIDWRAS

SEQ ID NO: 1013 - protein, 29 aa, Artificial Sequence
  Description of Artificial Sequence Synthetic peptide
MLSTKKTKTHDHYPSGRMRDPGWRDWRAS

SEQ ID NO: 1014 - protein, 29 aa, Artificial Sequence
  Description of Artificial Sequence Synthetic peptide
MLSTKKTKTHDHYPSGRMRDPGWGDWRAS

SEQ ID NO: 1015 - protein, 29 aa, Artificial Sequence
  Description of Artificial Sequence Synthetic peptide
MLSTKKTKTHDHYPSGRMRDPGWFDWRAS

SEQ ID NO: 1016 - protein, 29 aa, Artificial Sequence
  Description of Artificial Sequence Synthetic peptide
MLSTKKTKTHDHYPSGRMRDPGWLDWRAS

SEQ ID NO: 1017 - protein, 29 aa, Artificial Sequence
  Description of Artificial Sequence Synthetic peptide
MLSTKKTKTHDHYPSGRMRDPGWADWRAS

SEQ ID NO: 1018 - protein, 29 aa, Artificial Sequence
  Description of Artificial Sequence Synthetic peptide
MLSTKKTKTHDHYPSGRMRDPGWYDWRAS

SEQ ID NO: 1019 - protein, 29 aa, Artificial Sequence
  Description of Artificial Sequence Synthetic peptide
MLSTKKTKTHDHYPSGRMRDPGWPDWRAS

SEQ ID NO: 1020 - protein, 29 aa, Artificial Sequence
  Description of Artificial Sequence Synthetic peptide
MLSTKKTKTHDHYPSGRMRDPGWQDWRAS

SEQ ID NO: 1021 - protein, 29 aa, Artificial Sequence
  Description of Artificial Sequence Synthetic peptide
MLSTKKTKTHDHYPSGRMRDPGWSDWRAS

SEQ ID NO: 1022 - protein, 29 aa, Artificial Sequence
  Description of Artificial Sequence Synthetic peptide
MLSTKKTKTHDHYPSGRMRDPGWTDWRAS

SEQ ID NO: 1023 - protein, 29 aa, Artificial Sequence
  Description of Artificial Sequence Synthetic peptide
MLSTKKTKTHDHYPSGRMRDPGWNDWRAS

SEQ ID NO: 1024 - protein, 29 aa, Artificial Sequence
  Description of Artificial Sequence Synthetic peptide
MLSTKKTKTHDHYPSGRMRDPGWDDWRAS

SEQ ID NO: 1025 - protein, 29 aa, Artificial Sequence
  Description of Artificial Sequence Synthetic peptide
MLSTKKTKTHDHYPSGRMRDPGWVDWRAS

SEQ ID NO: 1026 - protein, 29 aa, Artificial Sequence
  Description of Artificial Sequence Synthetic peptide
MLSTKKTKTHDHYPSGRMRDPGWEDWRAS

SEQ ID NO: 1027 - protein, 29 aa, Artificial Sequence
  Description of Artificial Sequence Synthetic peptide
MLSTKKTKTHDHYPSGRMRDPGWHEWRAS

SEQ ID NO: 1028 - protein, 29 aa, Artificial Sequence
  Description of Artificial Sequence Synthetic peptide
MLSTKKTKTHDHYPSGRMRDPGWHYWRAS

SEQ ID NO: 1029 - protein, 29 aa, Artificial Sequence
  Description of Artificial Sequence Synthetic peptide
MLSTKKTKTHDHYPSGRMRDPGWHSWRAS

SEQ ID NO: 1030 - protein, 29 aa, Artificial Sequence
  Description of Artificial Sequence Synthetic peptide
MLSTKKTKTHDHYPSGRMRDPGWHGWRAS

SEQ ID NO: 1031 - protein, 29 aa, Artificial Sequence
  Description of Artificial Sequence Synthetic peptide
MLSTKKTKTHDHYPSGRMRDPGWHTWRAS

SEQ ID NO: 1032 - protein, 29 aa, Artificial Sequence
  Description of Artificial Sequence Synthetic peptide
MLSTKKTKTHDHYPSGRMRDPGWHVWRAS

SEQ ID NO: 1033 - protein, 29 aa, Artificial Sequence
  Description of Artificial Sequence Synthetic peptide
MLSTKKTKTHDHYPSGRMRDPGWHHWRAS

SEQ ID NO: 1034 - protein, 29 aa, Artificial Sequence
  Description of Artificial Sequence Synthetic peptide
MLSTKKTKTHDHYPSGRMRDPGWHLWRAS

SEQ ID NO: 1035 - protein, 29 aa, Artificial Sequence
  Description of Artificial Sequence Synthetic peptide
MLSTKKTKTHDHYPSGRMRDPGWHIWRAS

SEQ ID NO: 1036 - protein, 29 aa, Artificial Sequence
  Description of Artificial Sequence Synthetic peptide
MLSTKKTKTHDHYPSGRMRDPGWHNWRAS

SEQ ID NO: 1037 - protein, 29 aa, Artificial Sequence
  Description of Artificial Sequence Synthetic peptide
MLSTKKTKTHDHYPSGRMRDPGWHWWRAS

SEQ ID NO: 1038 - protein, 29 aa, Artificial Sequence
  Description of Artificial Sequence Synthetic peptide
MLSTKKTKTHDHYPSGRMRDPGWHFWRAS

SEQ ID NO: 1039 - protein, 29 aa, Artificial Sequence
  Description of Artificial Sequence Synthetic peptide
MLSTKKTKTHDHYPSGRMRDPGWHDHRAS

SEQ ID NO: 1040 - protein, 29 aa, Artificial Sequence
  Description of Artificial Sequence Synthetic peptide
MLSTKKTKTHDHYPSGRMRDPGWHDPRAS

SEQ ID NO: 1041 - protein, 29 aa, Artificial Sequence
  Description of Artificial Sequence Synthetic peptide
MLSTKKTKTHDHYPSGRMRDPGWHDNRAS

SEQ ID NO: 1042 - protein, 29 aa, Artificial Sequence
  Description of Artificial Sequence Synthetic peptide
MLSTKKTKTHDHYPSGRMRDPGWHDSRAS

SEQ ID NO: 1043 - protein, 29 aa, Artificial Sequence
  Description of Artificial Sequence Synthetic peptide
MLSTKKTKTHDHYPSGRMRDPGWHDQRAS

SEQ ID NO: 1044 - protein, 29 aa, Artificial Sequence
  Description of Artificial Sequence Synthetic peptide
MLSTKKTKTHDHYPSGRMRDPGWHDTRAS

SEQ ID NO: 1045 - protein, 29 aa, Artificial Sequence
  Description of Artificial Sequence Synthetic peptide
MLSTKKTKTHDHYPSGRMRDPGWHDARAS

SEQ ID NO: 1046 - protein, 29 aa, Artificial Sequence
  Description of Artificial Sequence Synthetic peptide
MLSTKKTKTHDHYPSGRMRDPGWHDDRAS

SEQ ID NO: 1047 - protein, 29 aa, Artificial Sequence
  Description of Artificial Sequence Synthetic peptide
MLSTKKTKTHDHYPSGRMRDPGWHDGRAS

SEQ ID NO: 1048 - protein, 29 aa, Artificial Sequence
  Description of Artificial Sequence Synthetic peptide
MLSTKKTKTHDHYPSGRMRDPGWHDYRAS

SEQ ID NO: 1049 - protein, 29 aa, Artificial Sequence
  Description of Artificial Sequence Synthetic peptide
MLSTKKTKTHDHYPSGRMRDPGWHDLRAS

SEQ ID NO: 1050 - protein, 29 aa, Artificial Sequence
  Description of Artificial Sequence Synthetic peptide
MLSTKKTKTHDHYPSGRMRDPGWHDVRAS

SEQ ID NO: 1051 - protein, 29 aa, Artificial Sequence
  Description of Artificial Sequence Synthetic peptide
MLSTKKTKTHDHYPSGRMRDPGWHDERAS

SEQ ID NO: 1052 - protein, 29 aa, Artificial Sequence
  Description of Artificial Sequence Synthetic peptide
MLSTKKTKTHDHYPSGRMRDPGWHDFRAS

SEQ ID NO: 1053 - protein, 29 aa, Artificial Sequence
  Description of Artificial Sequence Synthetic peptide
MLSTKKTKTHDHYPSGRMRDPGWHDRRAS

SEQ ID NO: 1054 - protein, 29 aa, Artificial Sequence
  Description of Artificial Sequence Synthetic peptide
MLSTKKTKTHDHYPSGRMRDPGWHDKRAS

SEQ ID NO: 1055 - protein, 29 aa, Artificial Sequence
  Description of Artificial Sequence Synthetic peptide
MLSTKKTKTHDHYPSGRMRDPGWHDWAAS

SEQ ID NO: 1056 - protein, 29 aa, Artificial Sequence
  Description of Artificial Sequence Synthetic peptide
MLSTKKTKTHDHYPSGRMRDPGWHDWDAS

SEQ ID NO: 1057 - protein, 29 aa, Artificial Sequence
  Description of Artificial Sequence Synthetic peptide
MLSTKKTKTHDHYPSGRMRDPGWHDWEAS

SEQ ID NO: 1058 - protein, 29 aa, Artificial Sequence
  Description of Artificial Sequence Synthetic peptide
MLSTKKTKTHDHYPSGRMRDPGWHDWFAS

SEQ ID NO: 1059 - protein, 29 aa, Artificial Sequence
  Description of Artificial Sequence Synthetic peptide
MLSTKKTKTHDHYPSGRMRDPGWHDWGAS

SEQ ID NO: 1060 - protein, 29 aa, Artificial Sequence
  Description of Artificial Sequence Synthetic peptide
MLSTKKTKTHDHYPSGRMRDPGWHDWHAS

SEQ ID NO: 1061 - protein, 29 aa, Artificial Sequence
  Description of Artificial Sequence Synthetic peptide
MLSTKKTKTHDHYPSGRMRDPGWHDWIAS

SEQ ID NO: 1062 - protein, 29 aa, Artificial Sequence
  Description of Artificial Sequence Synthetic peptide
MLSTKKTKTHDHYPSGRMRDPGWHDWKAS

SEQ ID NO: 1063 - protein, 29 aa, Artificial Sequence
  Description of Artificial Sequence Synthetic peptide
MLSTKKTKTHDHYPSGRMRDPGWHDWNAS

SEQ ID NO: 1064 - protein, 29 aa, Artificial Sequence
  Description of Artificial Sequence Synthetic peptide
MLSTKKTKTHDHYPSGRMRDPGWHDWPAS

SEQ ID NO: 1065 - protein, 29 aa, Artificial Sequence
  Description of Artificial Sequence Synthetic peptide
MLSTKKTKTHDHYPSGRMRDPGWHDWQAS

SEQ ID NO: 1066 - protein, 29 aa, Artificial Sequence
  Description of Artificial Sequence Synthetic peptide
MLSTKKTKTHDHYPSGRMRDPGWHDWSAS

SEQ ID NO: 1067 - protein, 29 aa, Artificial Sequence
  Description of Artificial Sequence Synthetic peptide
MLSTKKTKTHDHYPSGRMRDPGWHDWTAS

SEQ ID NO: 1068 - protein, 29 aa, Artificial Sequence
  Description of Artificial Sequence Synthetic peptide
MLSTKKTKTHDHYPSGRMRDPGWHDWVAS

SEQ ID NO: 1069 - protein, 29 aa, Artificial Sequence
  Description of Artificial Sequence Synthetic peptide
MLSTKKTKTHDHYPSGRMRDPGWHDWYAS

SEQ ID NO: 1070 - protein, 29 aa, Artificial Sequence
  Description of Artificial Sequence Synthetic peptide
MLSTKKTKTHDHYPSGRMRDPGWHDWRTS

SEQ ID NO: 1071 - protein, 29 aa, Artificial Sequence
  Description of Artificial Sequence Synthetic peptide
MLSTKKTKTHDHYPSGRMRDPGWHDWRSS

SEQ ID NO: 1072 - protein, 29 aa, Artificial Sequence
  Description of Artificial Sequence Synthetic peptide
MLSTKKTKTHDHYPSGRMRDPGWHDWRRS

SEQ ID NO: 1073 - protein, 29 aa, Artificial Sequence
  Description of Artificial Sequence Synthetic peptide
MLSTKKTKTHDHYPSGRMRDPGWHDWRQS

SEQ ID NO: 1074 - protein, 29 aa, Artificial Sequence
  Description of Artificial Sequence Synthetic peptide
MLSTKKTKTHDHYPSGRMRDPGWHDWRNS

SEQ ID NO: 1075 - protein, 29 aa, Artificial Sequence
  Description of Artificial Sequence Synthetic peptide
MLSTKKTKTHDHYPSGRMRDPGWHDWRLS

SEQ ID NO: 1076 - protein, 29 aa, Artificial Sequence
  Description of Artificial Sequence Synthetic peptide
MLSTKKTKTHDHYPSGRMRDPGWHDWRKS

SEQ ID NO: 1077 - protein, 29 aa, Artificial Sequence
  Description of Artificial Sequence Synthetic peptide
MLSTKKTKTHDHYPSGRMRDPGWHDWRIS

SEQ ID NO: 1078 - protein, 29 aa, Artificial Sequence
  Description of Artificial Sequence Synthetic peptide
MLSTKKTKTHDHYPSGRMRDPGWHDWRHS

SEQ ID NO: 1079 - protein, 29 aa, Artificial Sequence
  Description of Artificial Sequence Synthetic peptide
MLSTKKTKTHDHYPSGRMRDPGWHDWRES

SEQ ID NO: 1080 - protein, 29 aa, Artificial Sequence
  Description of Artificial Sequence Synthetic peptide
MLSTKKTKTHDHYPSGRMRDPGWHDWRDS

SEQ ID NO: 1081 - protein, 29 aa, Artificial Sequence
  Description of Artificial Sequence Synthetic peptide
MLSTKKTKTHDHYPSGRMRDPGWHDWRAA

SEQ ID NO: 1082 - protein, 29 aa, Artificial Sequence
  Description of Artificial Sequence Synthetic peptide
MLSTKKTKTHDHYPSGRMRDPGWHDWRAD

SEQ ID NO: 1083 - protein, 29 aa, Artificial Sequence
  Description of Artificial Sequence Synthetic peptide
MLSTKKTKTHDHYPSGRMRDPGWHDWRAE

SEQ ID NO: 1084 - protein, 29 aa, Artificial Sequence
  Description of Artificial Sequence Synthetic peptide
MLSTKKTKTHDHYPSGRMRDPGWHDWRAF

SEQ ID NO: 1085 - protein, 29 aa, Artificial Sequence
  Description of Artificial Sequence Synthetic peptide
MLSTKKTKTHDHYPSGRMRDPGWHDWRAG

SEQ ID NO: 1086 - protein, 29 aa, Artificial Sequence
  Description of Artificial Sequence Synthetic peptide
MLSTKKTKTHDHYPSGRMRDPGWHDWRAH

SEQ ID NO: 1087 - protein, 29 aa, Artificial Sequence
  Description of Artificial Sequence Synthetic peptide
MLSTKKTKTHDHYPSGRMRDPGWHDWRAI

SEQ ID NO: 1088 - protein, 29 aa, Artificial Sequence
  Description of Artificial Sequence Synthetic peptide
MLSTKKTKTHDHYPSGRMRDPGWHDWRAK

SEQ ID NO: 1089 - protein, 29 aa, Artificial Sequence
  Description of Artificial Sequence Synthetic peptide
MLSTKKTKTHDHYPSGRMRDPGWHDWRAL

SEQ ID NO: 1090 - protein, 29 aa, Artificial Sequence
  Description of Artificial Sequence Synthetic peptide
MLSTKKTKTHDHYPSGRMRDPGWHDWRAN

SEQ ID NO: 1091 - protein, 29 aa, Artificial Sequence
  Description of Artificial Sequence Synthetic peptide
MLSTKKTKTHDHYPSGRMRDPGWHDWRAP

SEQ ID NO: 1092 - protein, 29 aa, Artificial Sequence
  Description of Artificial Sequence Synthetic peptide
MLSTKKTKTHDHYPSGRMRDPGWHDWRAQ

SEQ ID NO: 1093 - protein, 29 aa, Artificial Sequence
  Description of Artificial Sequence Synthetic peptide
MLSTKKTKTHDHYPSGRMRDPGWHDWRAT

SEQ ID NO: 1094 - protein, 29 aa, Artificial Sequence
  Description of Artificial Sequence Synthetic peptide
MLSTKKTKTHDHYPSGRMRDPGWHDWRAV

SEQ ID NO: 1095 - protein, 29 aa, Artificial Sequence
  Description of Artificial Sequence Synthetic peptide
MLSTKKTKTHDHYPSGRMRDPGWHDWRAY

(19) United States
(12) Patent Application Publication
(10) Pub. No.: US 2023/0272016 A1
(43) Pub. Date: Aug. 31, 2023

(54) PEPTIDES FOR IMMUNOTHERAPY

(71) Applicant: Second Genome, Inc., Brisbane, CA (US)

(72) Inventors: Bum-Yeol Hwang, Brisbane, CA (US); Michi Izumi Willcoxon, Brisbane, CA (US); Helena Kiefel, Brisbane, CA (US); Toshihiko Takeuchi, Brisbane, CA (US); Dhwani Haria, Brisbane, CA (US); Michelle Lin, Brisbane, CA (US); Roberta L. Hannibal, Brisbane, CA (US); Joanna Catherine Ceolane Dreux, Brisbane, CA (US); Jayamary Divya Ravichandar, Brisbane, CA (US)

(21) Appl. No.: 18/009,045

(22) PCT Filed: Jun. 4, 2021
(86) PCT No.: PCT/US2021/035983

§ 371 Date: Dec. 8, 2022

Related U.S. Application Data
(60) Provisional application No. 63/036,359, filed on Jun. 8, 2020.

(51) Int. Cl. C07K 14/195; G01N 33/569; A61P 35/00

(52) U.S. Cl. CPC C07K 14/195; G01N 33/56911; A61P 35/00; G01N 2333/90206; G01N 2333/988; G01N 2800/52; C12N 2510/02

(57) ABSTRACT

The disclosure provides peptides and pharmaceutical compositions thereof. Such peptides can be useful, for example, in treating various human diseases such as immunological diseases. In some embodiments, the peptides are useful as immunotherapeutics for modulating regulatory and effector molecules of the mammalian immune system.

### CROSS REFERENCE TO RELATED APPLICATION

This application claims priority to U.S. Provisional Patent Application Ser. No. 63/036,359, filed on Jun. 8, 2020, which is incorporated herein by reference in its entirety.

### TECHNICAL FIELD

The present disclosure is related to peptides and compositions thereof, and using such peptides and compositions thereof for treating a disease in a subject associated with the adaptive and innate immune system and immunology-associated disorders.

### BACKGROUND

Inflammatory and immune-related diseases are the manifestation or consequence of complex and often multiple interconnected biological pathways, which in normal physiology are critical to respond to injury or insult, initiate repair from injury or insult, and mount an innate and/or acquired defense against foreign organisms. Disease or pathology can occur when these normal physiological pathways cause additional injury or insult that can be directly related to the intensity of the response, as a consequence of abnormal regulation or excessive stimulation, as a reaction to self, or as a combination thereof.

While the genesis of these diseases often involves multistep pathways and often multiple biological systems or pathways, intervention at critical points in one or more of these pathways can have an ameliorative or therapeutic effect. Therapeutic intervention can occur by either antagonism of a detrimental process/pathway or stimulation of a beneficial process/pathway. Many immune-related diseases are known and have been extensively studied. Such diseases include inflammatory diseases, infectious diseases, immunodeficiency diseases, neoplastic diseases, etc.

Cancer is the second leading cause of death, resulting in one out of every four deaths in the United States. More than one million people in the U.S. get cancer each year, and in 2016, it was estimated that 595,690 cancer deaths occurred. Due to the ever-increasing aging population in the U.S., it is reasonable to expect that rates of cancer incidence will continue to grow. See American Cancer Society.

Cancer is a disease which involves the uncontrolled growth (i.e., division) of cells. Some of the known mechanisms which contribute to the uncontrolled proliferation of cancer cells include growth factor independence, failure to detect genomic mutation, and inappropriate cell signaling. The ability of cancer cells to ignore normal growth controls may result in an increased rate of proliferation. Although the causes of cancer have not been firmly established, there are some factors known to contribute, or at least predispose a subject, to cancer. Such factors include particular genetic mutations (e.g., BRCA gene mutation for breast cancer, APC for colon cancer), exposure to suspected cancer-causing agents, or carcinogens (e.g., asbestos, UV radiation) and familial disposition for particular cancers such as breast cancer.

Cancer is currently treated using a variety of modalities including surgery, radiation therapy and chemotherapy. The choice of treatment will depend upon the type, location and dissemination of the cancer. For example, surgery and radiation therapy may be used to treat non-solid tumor cancers such as leukemia and lymphoma. One of the advantages of surgery and radiation therapy is the ability to control to some extent the impact of the therapy, and thus to limit the toxicity to normal tissues in the body. However, surgery and radiation therapy are often followed by chemotherapy to guard against any remaining or radio-resistant cancer cells. Chemotherapy is also the most appropriate treatment for disseminated cancers such as leukemia and lymphoma, as well as metastases.

Because many chemotherapy agents target cancer cells based on their proliferative profiles, tissues such as the gastrointestinal tract and the bone marrow which are normally proliferative are also susceptible to the effects of the chemotherapy.

Many chemotherapeutic agents have been developed for the treatment of cancer. Not all tumors, however, respond to chemotherapeutic agents and others, although initially responsive to chemotherapeutic agents, may develop resistance. As a result, the search for effective anti-cancer drugs has intensified in an effort to find even more effective agents with less non-specific toxicity.

Thus, there is a great need to develop additional and safer cancer therapeutics that leverage aspects of the mammalian immune system to aid in treating and preventing cancer. Despite new cancer treatments coming to market each year, these treatments are further accompanied by problematic side effects.

### SUMMARY

This document is based, at least in part, on the identification of peptides that can, for example, bind to T cells and modulate the production of cytokines (e.g., an anti-inflammatory cytokine and/or a pro-inflammatory cytokine in a subject). Provided herein are peptides comprising an amino acid sequence having at least 60% sequence identity to SEQ ID NO: 1, 117, or 162 (e.g., an amino acid sequence having at least 70%, at least 80%, at least 85%, at least 90%, at least 95%, or at least 99% sequence identity to SEQ ID NO: 1, 117, or 162). In some embodiments, the peptide comprises the amino acid sequence of SEQ ID NO: 1, 117, or 162.

Provided herein are methods for identifying a subject as having a decreased likelihood of positively responding to treatment with an immunomodulator, the method can include identifying a subject having a sample that has one or more of:
(i) a decreased level of the expression of dgoD, graR, or both relative to the same in a reference sample;
(ii) a decreased level of activity of a trans-2-enoyl-CoA reductase, an Acinetobacter tetrose transporter, or both relative to the same in a reference sample; and/or
(iii) a decreased flux through the β-ureidopropionase reaction relative to the same in a reference sample,
   as having a decreased likelihood of having a positive response to treatment with an immunomodulator. In some embodiments, the method further can include identifying, in the sample from the subject, an increased level of one or more bacterial species selected from the group consisting of: Clostridium clostridioforme, Prevotella sp., Streptococcus parasanguinis, Anaerostipes hadrus, Parasutterella excrementihominis, and Eisenbergiella massiliensis relative to the same in a reference sample. In some embodiments, the method further can include identifying, in the sample from the subject, a decreased level of one or more bacterial species selected from the group consisting of: Bifidobacterium sp., Collinsella sp., Methanobrevibacter smithii, Oscillibacter sp., Faecalibacterium prausnitzii C, Faecalibacterium prausnitzii I, Intestinimonas timonensis, Faecalibacterium prausnitzii, Bacteroides caccae, Barnesiella intestinihominis, Clostridiaceae bacterium, Clostridium sp., and Bifidobacterium adolescentis relative to the same in a reference sample.

Also provided herein are methods for identifying a subject as having an increased likelihood of having a positive response to treatment with an immunomodulatory. The method can include identifying a subject having a sample that has one or more of:
(i) an increased level of the expression of dgoD, graR, or both relative to the same in a reference sample;
(ii) an increased level of activity of a trans-2-enoyl-CoA reductase, an Acinetobacter tetrose transporter, or both relative to the same in a reference sample;
(iii) an increased flux through the β-ureidopropionase reaction relative to the same in a reference sample,

as having an increased likelihood of having a positive response to treatment with an immunomodulator. In some embodiments, the method can include identifying, in the sample from the subject, a decreased level of one or more bacterial species selected from the group consisting of: Clostridium clostridioforme, Prevotella sp., Streptococcus parasanguinis, Anaerostipes hadrus, Parasutterella excrementihominis, and Eisenbergiella massiliensis relative to the same in a reference sample. In some embodiments, the method can include identifying, in the sample from the subject, an increased level of one or more bacterial species selected from the group consisting of: Bifidobacterium sp., Collinsella sp., Methanobrevibacter smithii, Oscillibacter sp., Faecalibacterium prausnitzii C, Faecalibacterium prausnitzii I, Intestinimonas timonensis, Faecalibacterium prausnitzii, Bacteroides caccae, Barnesiella intestinihominis, Clostridiaceae bacterium, Clostridium sp., and Bifidobacterium adolescentis relative to the same in a reference sample.

In any of the methods described herein, the immunomodulator can be an immune checkpoint inhibitor selected from the group consisting of: ipilimumab, nivolumab, pembrolizumab, atezolizumab, avelumab, durvalumab, cemiplimab, and a combination thereof.

In any of the methods described herein, the immunomodulator can be a co-stimulatory immune checkpoint agent selected from the group consisting of: IBI101, utomilumab, MEDI1873, and a combination thereof.

In any of the methods described herein, the cell therapy can be a CAR T cell therapy.

In any of the methods described herein, the immunomodulator can target one or more of: CTLA-4, PD-1, PD-L1, BTLA, LAG-3, A2AR, TIM-3, B7-H3, VISTA, IDO, OX40, 4-1BB, and GITR.

Also provided herein are peptides that may include an amino acid sequence having at least 60% sequence identity to SEQ ID NO: 1. For example, the peptide can comprise an amino acid sequence having at least 70%, at least 80%, at least 85%, at least 90%, at least 95%, or at least 99% sequence identity to SEQ ID NO: 1. In some embodiments, the peptide comprises the amino acid sequence of SEQ ID NO: 1.

Also provided herein are peptides that can include the amino acid sequence of SEQ ID NO: 1, or a variant thereof comprising one to 15 amino acid substitutions. For example, the methionine at position at 1 of SEQ ID NO:1 can be substituted with an amino acid selected from the group consisting of: W, F, V, P, K, R, and S. For example, the leucine at position at 2 of SEQ ID NO:1 can be substituted with an amino acid selected from the group consisting of: S, P, G, T, V, A, K, Q, R, W, Y, F, and N. For example, the serine at position at 3 of SEQ ID NO:1 can be substituted with an amino acid selected from the group consisting of: Q and R. For example, the threonine at position at 4 of SEQ ID NO:1 can be substituted with an amino acid selected from the group consisting of: G, A, and R. For example, the lysine at position at 5 of SEQ ID NO:1 can be substituted with an amino acid selected from the group consisting of: A and R. For example, the lysine at position at 6 of SEQ ID NO:1 can be substituted with an amino acid selected from the group consisting of: R, T, and A. For example, the threonine at position at 7 of SEQ ID NO:1 can be substituted with an amino acid selected from the group consisting of: G, K, and R. For example, the threonine at position at 9 of SEQ ID NO:1 is substituted with an amino acid selected from the group consisting of: W and R. For example, the histidine at position at 10 of SEQ ID NO:1 can be substituted with an amino acid selected from the group consisting of: K and R. For example, the aspartic acid at position at 11 of SEQ ID NO:1 can be substituted with an amino acid selected from the group consisting of: F, G, H, I, K, P, R, T, V, W, and Y. For example, the histidine at position at 12 of SEQ ID NO:1 can be substituted with an amino acid selected from the group consisting of: K and R. For example, the tyrosine at position at 13 of SEQ ID NO:1 can be substituted with an amino acid selected from the group consisting of: W, N, G, K, R, and W. For example, the proline at position at 14 of SEQ ID NO:1 can be substituted with an amino acid selected from the group consisting of: G and W. For example, the serine at position at 15 of SEQ ID NO:1 can be substituted with an amino acid selected from the group consisting of: G and R. For example, the methionine at position at 18 of SEQ ID NO:1 can be substituted with an amino acid selected from the group consisting of: W, H, Y, G, and R. For example, the aspartic acid at position at 20 of SEQ ID NO:1 can be substituted with an amino acid selected from the group consisting of: P and R. For example, the proline at position at 21 of SEQ ID NO:1 can be an F. For example, the glycine at position at 22 of SEQ ID NO:1 can be substituted with an amino acid selected from the group consisting of: P, K, and W. For example, the aspartic acid at position at 25 of SEQ ID NO:1 can be substituted with an amino acid selected from the group consisting of: P and R. For example, the arginine at position at 27 of SEQ ID NO:1 can be a W. For example, the alanine at position at 28 of SEQ ID NO:1 can be substituted with an amino acid selected from the group consisting of: F, G, V, Y, and W. For example, the serine at position at 29 of SEQ ID NO:1 can be substituted with an R.

In some embodiments, the peptide comprises an amino acid sequence selected from the group consisting of: SEQ ID NOs: 2-35.

In some embodiments, the peptide comprises an amino acid sequence selected from the group consisting of: SEQ ID NOs: 36-93.

In some embodiments, the peptide comprises an amino acid sequence selected from the group consisting of: SEQ ID NOs: 94-114.

Also provided herein are peptides that include the amino acid sequence set forth in X₁X₂SX₄AKX₇KX₈HDHX₁₂X₁₃X₁₄GRX₁₅RX₁₆PX₁₈WHDWX₂₀X₂₁X₂₂ (SEQ ID NO:115), where each of X₁-X₂₂ is independently selected from any naturally occurring amino acid. For example, X₁ can be an amino acid selected from the group consisting of: M, W, F, V, and P. For example, X₂ can be an amino acid selected from the group consisting of: L, S, P, G, T, V, and A. For example, X₄ can be an amino acid selected from the group consisting of: T, G, and A. For example, X₇ can be an amino acid selected from the group consisting of: G and T. For example, X₈ can be an amino acid selected from the group consisting of: T and W. For example, X₁₂ can be an amino acid selected from the group consisting of: Y, W, and N. For example, X₁₃ can be an amino acid selected from the group consisting of: P, G, and W. For example, X₁₄ can be an amino acid selected from the group consisting of: S and G. For example, X₁₅ can be an amino acid selected from the group consisting of: M, W, H, and Y. For example, X₁₆ can be an amino acid selected from the group consisting of: D and P. For example, X₁₈ can be an amino acid selected from the group consisting of: G, P, and K. For example, X₂₀ can be an amino acid selected from the group consisting of: R and W. For example, X₂₁ can be an amino acid selected from the group consisting of: A, F, G, and V. For example, X₂₂ can be an amino acid selected from the group consisting of: R and W.

In some embodiments, the peptide increases activity of a CD2 protein, a BST2 protein, or a TNF protein.

In some embodiments, the peptide binds to a CD2 protein, a BST2 protein, or a TNF protein.

Also provided herein are peptides that include an amino acid sequence having at least 60% sequence identity to SEQ ID NO: 117 or SEQ ID NO: 162. In some embodiments, the peptide comprises an amino acid sequence having at least 70%, at least 80%, at least 85%, at least 90%, at least 95%, or at least 99% sequence identity to SEQ ID NO: 117 or SEQ ID NO: 162.

In some embodiments, the peptide comprises the amino acid sequence of SEQ ID NO: 117 or SEQ ID NO: 162.

In some embodiments, the peptide comprises an amino acid sequence selected from the group consisting of: SEQ ID NOs: 117-160.

Also provided herein are peptides that may include the amino acid sequence set forth in:

X₁KX₃X₄X₅SVKX₉X₁₀CX₁₂X₁₃CX₁₄X₁₅X₁₆IX₁₈RX₂₀GX₂₂X₂₃X₂₄X₂₅IX₂₇X₂₈X₂₉PX₃₁HKQX₃₅QX₃₇ (SEQ ID NO: 161), wherein X₁ is optional, each of X₂-X₂₅ and X₂₈-X₃₅ is independently a naturally occurring amino acid, X₂₇ is selected from the group consisting of C and CP; and X₃₇ is selected from the group consisting of: G, GN, and DRH. For example, X₁ can be the amino acid M. For example, X₃ can be an amino acid selected from the group consisting of: V, I, and T. For example, X₄ can be an amino acid selected from the group consisting of: R, K, and Q. For example, X₅ can be an amino acid selected from the group consisting of: P, S, and A. For example, X₉ can be an amino acid selected from the group consisting of: P, T, and K. For example, X₁₀ can be an amino acid selected from the group consisting of: M and I. For example, X₁₂ can be an amino acid selected from the group consisting of: E and D. For example, X₁₃ can be an amino acid selected from the group consisting of: K and Y. For example, X₁₅ can be an amino acid selected from the group consisting of: K and R. For example, X₁₆ can be an amino acid selected from the group consisting of: V and I. For example, X₁₈ can be an amino acid selected from the group consisting of: K and R. For example, X₂₀ can be an amino acid selected from the group consisting of: K, N, and H. For example, X₂₂ can be an amino acid selected from the group consisting of: R, K, H, S, and I. For example, X₂₃ can be an amino acid selected from the group consisting of: V and I. For example, X₂₄ can be an amino acid selected from the group consisting of: M, R, A, and L. For example, X₂₅ can be an amino acid selected from the group consisting of: V and I. For example, X₂₈ can be an amino acid selected from the group consisting of: E, Q, A, and T. For example, X₂₉ can be an amino acid selected from the group consisting of: N and E. For example, X₃₁ can be an amino acid selected from the group consisting of: K and R. For example, X₃₅ can be an amino acid selected from the group consisting of: K and R.

In some embodiments, a peptide modulates activity of a CCR9 protein, a CHRM5 protein, a CXCR3 protein, a CXCR4 protein, a HCRTR2 protein, a MRGPRX2 protein, a SSTR1 protein, or a TSHR(L) protein.

In some embodiments, a peptide binds to a CCR9 protein, a CHRM5 protein, a CXCR3 protein, a CXCR4 protein, a HCRTR2 protein, a MRGPRX2 protein, a SSTR1 protein, or a TSHR(L) protein.

Also provided herein are recombinant host cells (e.g., a prokaryotic cell, a eukaryotic cell, or a fungal cell) that include an exogenous polynucleotide, wherein the polynucleotide encodes any of the peptides described herein.

In some embodiments, the exogenous polynucleotide further can encode a host cell specific signal sequence. In some embodiments, the exogenous polynucleotide further encodes a heterologous promoter. In some embodiments, the heterologous promoter is a constitutive promoter. In some embodiments, the heterologous promoter is an inducible promoter. In some embodiments, the host cell is selected from the group consisting of: an Escherichia coli cell, a Lactococcus lactis cell, a Streptomyces coelicolor cell, a Streptomyces lividans cell, a Streptomyces albus cell, a Streptomyces venezuelae cell, or a Bacillus subtilis cell. In some embodiments, the host cell is a Saccharomyces cerevisiae cell, a Pichia pastoris cell, a Yarrowia lipolytica cell, an Aspergillus niger cell, or a Hansenula polymorpha cell. In some embodiments, the host cell is a Chinese Hamster Ovary cell.

Also provided herein are pharmaceutical compositions that include any of the peptides described herein or a plurality of recombinant host cells described herein, and a pharmaceutically acceptable carrier. The pharmaceutical composition can be formulated for oral administration.

In some embodiments, the pharmaceutical composition can include a therapeutically effective amount of a bacterial species selected from the group consisting of: Bifidobacterium sp., Collinsella sp., Methanobrevibacter smithii, Oscillibacter sp., Barnesiella intestinihominis, Faecalibacterium prausnitzii C, Faecalibacterium prausnitzii I, Ruminococcaceae bacterium, Intestinimonas timonensis, Faecalibacterium prausnitzii, Bacteroides caccae, Clostridiaceae bacterium, Clostridium sp., Bifidobacterium adolescentis, and a combination thereof.

Also provided herein are nucleic acid constructs that include a polynucleotide, wherein the polynucleotide encodes any of peptides described herein.

Also provided here are methods of producing a peptide that include culturing any recombinant host cell described herein, under conditions sufficient for expression of the encoded peptide.

Also provided herein are methods for treating a disease in a subject in need thereof, the method comprising administering, to the subject, any of the peptides, recombinant hosts, pharmaceutical compositions, or nucleic acid constructs described herein.

In some embodiments, the peptide modulates the production of at least one cytokine in the subject. For example, the peptide can modulate the production of a cytokine selected from the group consisting of TNF-α, IL-17, IL-1β, IL-2, IFN-γ, IL-6, IL-12, IL-25, IL-33, IL-8, MCP-1, MIP-3α, CXCL1, IL-23, IL-4, IL-10, IL-13, IFN-α, and TGF-β. In some embodiments, the peptide induces the production of at least one pro-inflammatory cytokine in the subject. For example, the peptide can induce the production of at least one pro-inflammatory cytokine selected from the group consisting of TNF-α, IL-17, IL-1β, IL-2, IFN-γ, IL-6, IL-12, IL-25, IL-33, IL-8, MCP-1, MIP-3α, CXCL1, and IL-23.

In some embodiments, the peptide suppresses the production of at least one anti-inflammatory cytokine in the subject. For example, the peptide can suppress the production of at least one anti-inflammatory cytokine of IL-4, IL-10, IL-13, IFN-α, or TGF-β in the subject.

In some embodiments, the peptide increases Th1 activation in the subject. In some embodiments, the peptide increases dendritic cell maturation in the subject. In some embodiments, the peptide increases CD70 expression in the subject. In some embodiments, the peptide increases the clonal expansion of Teff in the subject.

In some embodiments, the peptide increases activity of a CD2 protein, a BST2 protein, or a TNF protein. In some embodiments, the peptide increases activity of a CXCL3 protein. In some embodiments, the peptide binds to a CD2 protein, a BST2 protein, or a TNF protein. In some embodiments, the peptide binds to a CXCL3 protein.

In some embodiments, the disease is a neoplasm or is a cancer. For example, the disease can be at least one of basal cell carcinoma, biliary tract cancer, bladder cancer, bone cancer, brain and central nervous system cancer, breast cancer, cervical cancer, choriocarcinoma, colon and rectum cancer, connective tissue cancer, cancer of the digestive system, endometrial cancer, esophageal cancer, eye cancer, cancer of the head and neck, gastric cancer, intra-epithelial neoplasm, kidney cancer, larynx cancer, leukemia, liver cancer, small-cell lung cancer, non-small-cell lung cancer, Hodgkin's lymphoma, non-Hodgkins lymphoma, melanoma, myeloma, neuroblastoma, oral cavity cancer, ovarian cancer, pancreatic cancer, prostate cancer, retinoblastoma, rhabdomyosarcoma, rectal cancer, renal cancer, cancer of the respiratory system, sarcoma, skin cancer, stomach cancer, testicular cancer, thyroid cancer, uterine cancer, or cancer of the urinary system.

In some embodiments, the method can include administering a treatment for cancer.

Also provided herein are methods of treating cancer in a subject that can include:
(a) identifying a subject having a sample that has one or more of:
   (i) a decreased level of the expression of dgoD, graR, or both relative to the same in a reference sample;
   (ii) a decreased level of activity of a trans-2-enoyl-CoA reductase, an Acinetobacter tetrose transporter, or both relative to the same in a reference sample;
   (iii) a decreased flux through the β-ureidopropionase reaction relative to the same in a reference sample;
   (iv) an increased level of one or more bacterial species selected from the group consisting of: Clostridium clostridioforme, Prevotella sp., Streptococcus parasanguinis, Anaerostipes hadrus, Parasutterella excrementihominis, and Eisenbergiella massiliensis relative to the same in a reference sample; and
   (v) a decreased level of one or more bacterial species selected from the group consisting of: Bifidobacterium sp., Collinsella sp., Methanobrevibacter smithii, Oscillibacter sp., Faecalibacterium prausnitzii C, Faecalibacterium prausnitzii Intestinimonas timonensis, Faecalibacterium prausnitzii, Bacteroides caccae, Barnesiella intestinihominis, Clostridiaceae bacterium, Clostridium sp., and Bifidobacterium adolescentis relative to the same in a reference sample; and
(b) administering a therapy to the identified subject that includes any peptide described herein, any pharmaceutical composition described herein, or any recombinant host described herein.

Also provided herein are methods of treating cancer in a subject that can include administering to a subject identified as having one or more of:
(i) a decreased level of the expression of dgoD, graR, or both relative to the same in a reference sample;
(ii) a decreased level of activity of a trans-2-enoyl-CoA reductase, an Acinetobacter tetrose transporter, or both relative to the same in a reference sample;
(iii) a decreased flux through the β-ureidopropionase reaction relative to the same in a reference sample;
(iv) an increased level of one or more bacterial species selected from the group consisting of: Clostridium clostridioforme, Prevotella sp., Streptococcus parasanguinis, Anaerostipes hadrus, Parasutterella excrementihominis, and Eisenbergiella massiliensis relative to the same in a reference sample; and
(v) a decreased level of one or more bacterial species selected from the group consisting of: Bifidobacterium sp., Collinsella sp., Methanobrevibacter smithii, Oscillibacter sp., Faecalibacterium prausnitzii C, Faecalibacterium prausnitzii Intestinimonas timonensis, Faecalibacterium prausnitzii, Bacteroides caccae, Barnesiella intestinihominis, Clostridiaceae bacterium, Clostridium sp., and Bifidobacterium adolescentis relative to the same in a reference sample a therapy comprising any peptide described herein, any pharmaceutical composition described herein, or any recombinant host described herein.

Also, provided herein are methods of treating a cancer in a subject that has previously received one or more doses of an immunomodulator that may include administering to a subject identified as having one or more of:
(i) a decreased level of the expression of dgoD, graR, or both relative to the same in a reference sample;
(ii) a decreased level of activity of a trans-2-enoyl-CoA reductase, an Acinetobacter tetrose transporter, or both relative to the same in a reference sample;
(iii) a decreased flux through the β-ureidopropionase reaction relative to the same in a reference sample;
(iv) an increased level of one or more bacterial species selected from the group consisting of: Clostridium clostridioforme, Prevotella sp., Streptococcus parasanguinis, Anaerostipes hadrus, Parasutterella excrementihominis, and Eisenbergiella massiliensis relative to the same in a reference sample; and
(v) a decreased level of one or more bacterial species selected from the group consisting of: Bifidobacterium sp., Collinsella sp., Methanobrevibacter smithii, Oscillibacter sp., Faecalibacterium prausnitzii C, Faecalibacterium prausnitzii Intestinimonas timonensis, Faecalibacterium prausnitzii, Bacteroides caccae, Barnesiella intestinihominis, Clostridiaceae bacterium, Clostridium sp., and Bifidobacterium adolescentis relative to the same in a reference sample a therapy comprising any peptide described herein, any pharmaceutical composition described herein, or any recombinant host described herein.

Also provided herein are methods of treating cancer in a subject that can include:
(a) administering to the subject one or more doses of an immunomodulator for a period of time;
(b) after (a), determining if a sample obtained from the subject has one or more of:
   (i) a decreased level of the expression of dgoD, graR, or both relative to the same in a reference sample;
   (ii) a decreased level of activity of a trans-2-enoyl-CoA reductase, an Acinetobacter tetrose transporter, or both relative to the same in a reference sample;
   (iii) a decreased flux through the β-ureidopropionase reaction relative to the same in a reference sample;
   (iv) an increased level of one or more bacterial species selected from the group consisting of: Clostridium clostridioforme, Prevotella sp., Streptococcus parasanguinis, Anaerostipes hadrus, Parasutterella excrementihominis, and Eisenbergiella massiliensis relative to the same in a reference sample; and
   (v) a decreased level of one or more bacterial species selected from the group consisting of: Bifidobacterium sp., Collinsella sp., Methanobrevibacter smithii, Oscillibacter sp., Faecalibacterium prausnitzii C, Faecalibacterium prausnitzii Intestinimonas timonensis, Faecalibacterium prausnitzii, Bacteroides caccae, Barnesiella intestinihominis, Clostridiaceae bacterium, Clostridium sp., and Bifidobacterium adolescentis relative to the same in a reference sample; and
(c) administering a therapy to the identified subject that can include any peptide described herein, any pharmaceutical composition described herein, or any recombinant host described herein.

Any of the methods can include a treatment for cancer.

Also provided herein are methods of treating cancer in a subject that may include:
(a) administering to the subject one or more doses of an immunomodulator for a period of time;
(b) after (a), identifying a subject having a sample that has one or more of:
   (i) an increased level of the expression of dgoD, graR, or both relative to the same in a reference sample;
   (ii) an increased level of activity of a trans-2-enoyl-CoA reductase, an Acinetobacter tetrose transporter, or both relative to the same in a reference sample;
   (iii) an increased flux through the β-ureidopropionase reaction relative to the same in a reference sample;
   (iv) a decreased level of one or more bacterial species selected from the group consisting of: Clostridium clostridioforme, Prevotella sp., Streptococcus parasanguinis, Anaerostipes hadrus, Parasutterella excrementihominis, and Eisenbergiella massiliensis relative to the same in a reference sample; and
   (v) an increased level of one or more bacterial species selected from the group consisting of: Bifidobacterium sp., Collinsella sp., Methanobrevibacter smithii, Oscillibacter sp., Faecalibacterium prausnitzii C, Faecalibacterium prausnitzii Intestinimonas timonensis, Faecalibacterium prausnitzii, Bacteroides caccae, Barnesiella intestinihominis, Clostridiaceae bacterium, Clostridium sp., and Bifidobacterium adolescentis relative to the same in a reference sample; and
(c) administering a therapy to the identified subject that can include any peptide described herein, any pharmaceutical composition described herein, or any recombinant host described herein.

Also provided herein are methods of treating cancer in a subject that can include administering, to a subject identified as having one or more of:
(i) an increased level of the expression of dgoD, graR, or both relative to the same in a reference sample;
(ii) an increased level of activity of a trans-2-enoyl-CoA reductase, an Acinetobacter tetrose transporter, or both relative to the same in a reference sample;
(iii) an increased flux through the β-ureidopropionase reaction relative to the same in a reference sample;
(iv) a decreased level of one or more bacterial species selected from the group consisting of: Clostridium clostridioforme, Prevotella sp., Streptococcus parasanguinis, Anaerostipes hadrus, Parasutterella excrementihominis, and Eisenbergiella massiliensis relative to the same in a reference sample; and
(v) an increased level of one or more bacterial species selected from the group consisting of: Bifidobacterium sp., Collinsella sp., Methanobrevibacter smithii, Oscillibacter sp., Faecalibacterium prausnitzii C, Faecalibacterium prausnitzii Intestinimonas timonensis, Faecalibacterium prausnitzii, Bacteroides caccae, Barnesiella intestinihominis, Clostridiaceae bacterium, Clostridium sp., and Bifidobacterium adolescentis relative to the same in a reference sample,
   a therapy comprising any peptide described herein, any pharmaceutical composition described herein, or any recombinant host described herein; where the subject has received a therapeutically effective amount of an immunomodulator.

In some embodiments, the therapy can include one or more of:
a) a therapeutically effective amount of an immunomodulator;
b) an effective amount of one or more bacterial species selected from the group consisting of: Bifidobacterium sp., Collinsella sp., Methanobrevibacter smithii, Oscillibacter sp., Faecalibacterium prausnitzii C, Faecalibacterium prausnitzii Ruminococcaceae bacterium, Intestinimonas timonensis, Faecalibacterium prausnitzii, Bacteroides caccae, Barnesiella intestinihominis, Clostridiaceae bacterium, Ruminococcaceae bacterium, Clostridium sp., and Bifidobacterium adolescentis; and/or
c) an additional treatment of cancer excluding an immunomodulator.

Also provided herein are methods of modulating the activity of one or more target proteins in a subject that can include administering to the subject any peptide described herein; or a plurality of any of the recombinant host cells described herein; where the one or more target proteins is a CD2 protein, a BST2 protein, a TNF protein, a CXCL3 protein, a ADRA2A protein, a ADRB2 protein, a CCR6 protein, a CCR9 protein, a CHRM5 protein, a CXCR3 protein, a CXCR4 protein, a EDGE protein, a HCRTR2 protein, a HRH4 protein, a MRGPRX2 protein, a MTNR1A protein, a NPFFR1 protein, a SSTR1 protein, a SSTR3 protein, a TRHR protein, or a TSHR(L) protein. For example, the one or more target proteins can be a CD2 protein, a BST2 protein, or a TNF protein. For example, the one or more target proteins is a CXCL3 protein. For example, the one or more target proteins can be a CCR9 protein, a CHRM5 protein, a CXCR3 protein, a CXCR4 protein, a HCRTR2 protein, a MRGPRX2 protein, a SSTR1 protein, and/or a TSHR(L) protein.

In some embodiments, any of the methods described may include detecting the level of one or more bacterial species, RNA transcripts, protein activity, or flux though a metabolic pathway in a sample from the subject.

Also provided herein are methods for increasing the response to an immunomodulator in a subject in need thereof that can include administering to the subject a composition that includes any peptide described herein, any pharmaceutical composition described herein, or any recombinant host described herein. In some embodiments, the subject has cancer.

Also provided herein are methods for treating cancer in a subject that may include:
(a) detecting a dysbiosis associated with response to therapy with an immunomodulator in a sample from the subject; and
(b) administering to the subject a composition that includes any peptide described herein, any pharmaceutical composition described herein, or any recombinant host described herein. The sample can be a fecal sample or a tumor biopsy sample.

In some embodiments, detecting the dysbiosis associated with response to therapy with an immunomodulator can include determining bacterial gene expression in the sample from the subject.

In some embodiments, detecting the dysbiosis associated with response to therapy with an immunomodulator can include determining bacterial composition in the sample from the subject.

In some embodiments, detecting the dysbiosis associated with response to therapy with an immunomodulator can include determining bacterial protein activity in the sample from the subject.

In any of the methods described herein, the immunomodulator can target one or more of: CTLA-4, PD-1, PD-L1, BTLA, LAG-3, A2AR, TIM-3, B7-H3, VISTA, IDO, OX40, 4-1BB, and GITR.

In any of the methods described herein, the subject can have a solid tumor. For example, the subject can have a solid tumor selected from the group consisting of: melanoma, lung cancer, kidney cancer, bladder cancer, a head and neck cancer, Merkel cell carcinoma, urothelial cancer, breast cancer, glioblastoma, gastric cancer, a nasopharyngeal neoplasm, colorectal cancer, hepatocellular carcinoma, ovarian cancer, and pancreatic cancer.

In any of the methods described herein, the subject can have a hematological malignancy. For example, the subject can have a hematological malignancy that is multiple myeloma, non-Hodgkin lymphoma, Hodgkin lymphoma, diffuse large B-cell lymphoma, or chronic lymphocytic leukemia/small lymphocytic lymphoma (CLL/SLL).

In any of the methods described herein, the subject can have a cancer that is melanoma, non-small cell lung cancer (NSCLC), small cell lung cancer, squamous cell lung carcinoma, kidney cancer, bladder cancer, a head and neck cancer, Hodgkin lymphoma, Merkel cell carcinoma, urothelial cancer, breast cancer, glioblastoma, gastric adenocarcinoma, transitional cell carcinoma, a biliary tract neoplasm, a nasopharyngeal neoplasm, colorectal cancer, hepatocellular carcinoma, renal cell carcinoma, ovarian cancer, and/or pancreatic cancer.

In any of the methods described herein, the melanoma can be unresectable or metastatic melanoma.

In any of the methods described herein, the method can include administering the composition to the subject once, twice, or three times per day.

In any of the methods described herein, the composition can be formulated for oral administration, rectal administration, intravenous administration, or intratumoral administration.

In any of the methods described herein, the composition can be formulated as a tablet, a capsule, a powder, or a liquid. For example, the composition can be formulated as a tablet (e.g., a coated tablet). In some embodiments, the coating comprises an enteric coating.

In any of the methods described herein, the method can include administering a treatment for cancer, an additional treatment for cancer, and/or other adjunct therapy to the subject.

In some embodiments, the bacterial strain treatment and the treatment for cancer and/or adjunct therapy are administered simultaneously.

In some embodiments, the composition may include the bacterial strain treatment and the treatment for cancer and/or adjunct therapy are administered sequentially.

In some embodiments, the treatment for cancer and/or adjunct therapy may include a probiotic.

In some embodiments, the treatment for cancer and/or adjunct therapy may include surgery, radiation therapy, or a combination thereof.

In some embodiments of any of the methods described herein, the treatment for cancer and/or adjunct therapy may include a therapeutic agent. For example, the therapeutic agent can include a chemotherapeutic agent, a targeted therapy, an immunotherapy, or a combination thereof. For example, the chemotherapeutic agent can be carboplatin, cisplatin, gemcitabine, methotrexate, paclitaxel, pemetrexed, lomustine, temozolomide, dacarbazine, or a combination thereof. The targeted therapy can be afatinib dimaleate, bevacizumab, cetuximab, crizotinib, erlotinib, gefitinib, sorafenib, sunitinib, pazopanib, everolimus, dabrafenib, aldesleukin, interferon alfa-2b, ipilimumab, peginterferon alfa-2b, trametinib, vemurafenib, or a combination thereof. The immunotherapy can include a cell therapy, a therapy with an immunomodulator, or a combination thereof. For example, the immunomodulator can be an immune checkpoint inhibitor that is ipilimumab, nivolumab, pembrolizumab, atezolizumab, avelumab, durvalumab, cemiplimab, or a combination thereof.

In some embodiments, the immunomodulator is a co-stimulatory immune checkpoint agent that is IBI101, utomilumab, MEDI1873, or a combination thereof.

In some embodiments, the cell therapy is a CAR T cell therapy

In any of the methods described herein, the subject is a human.

The term “subject” refers to a mammal such as a human, a non-human primate, a livestock animal (e.g., bovine, porcine), a companion animal (e.g., canine, feline) and a rodent (e.g., a mouse and a rat). In some embodiments, the term refers to a human subject.

As used herein, unless otherwise noted, the terms “treating,” “treatment,” and the like, shall include the management and care of a subject (e.g., a mammal such as a human) for the purpose of combating a disease, condition, or disorder and includes the administration of a disclosed peptide to alleviate the symptoms or complications, or reduce the rate of progression of the disease, condition, or disorder. In some embodiments, treatment can be of a subject who has been diagnosed as suffering from the relevant disease, disorder, and/or condition. In some embodiments, treatment can be of a subject known to have one or more susceptibility factors that are statistically correlated with increased risk of development of the relevant disease, disorder, and/or condition.

The term “T cell-mediated disease” means a disease in which T cells directly or indirectly mediate. The T cell-mediated disease may be associated with, but not limited to, cell-mediated effects, lymphokine-mediated effects, and/or effects associated with B cells if the B cells are stimulated, for example, by the lymphokines secreted by T cells.

A “peptide” as used herein refers to a polypeptide having 3 to 50 amino acids. For example, a peptide can have 10 to 50 amino acids, 20 to 40 amino acids, 20 to 30 amino acids, 25 to 35 amino acids, or 30 to 40 amino acids. In some embodiments, a peptide can be produced recombinantly. In some embodiments, a peptide can be produced by chemical synthesis.

The term “pharmaceutically acceptable salt,” as used herein, represents salts or zwitterionic forms of the peptides, proteins, or compounds of the present disclosure, which are water or oil-soluble or dispersible, which are suitable for treatment of diseases without undue toxicity, irritation, and allergic response; which are commensurate with a reasonable benefit/risk ratio, and which are effective for their intended use. The salts can be prepared during the final isolation and purification of the compounds or separately by reacting an amino group with a suitable acid. Representative acid addition salts include acetate, adipate, alginate, citrate, aspartic acid, benzoate, benzenesulfonate, bisulfate, butyrate, camphorate, camphorsulfonate, digluconate, glycerophosphate, hemisulfate, heptanoate, hexanoate, formate, fumarate, hydrochloride, hydrobromide, hydroiodide, 2-hydroxyethansulfonate (isethionate), lactate, maleate, mesitylenesulfonate, methanesulfonate, naphthylenesulfonate, nicotinate, 2-naphthalenesulfonate, oxalate, pamoate, pectinate, persulfate, 3-phenylproprionate, picrate, pivalate, propionate, succinate, tartrate, trichloroacetate, trifluoroacetate, phosphate, glutamic acid, bicarbonate, para-toluenesulfonate, and undecanoate. Also, amino groups in the compounds of the present disclosure can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. A pharmaceutically acceptable salt can suitably be a salt chosen, e.g., among acid addition salts and basic salts. Examples of acid addition salts include chloride salts, citrate salts and acetate salts. Examples of basic salts include salts where the cation is selected among alkali metal cations, such as sodium or potassium ions, alkaline earth metal cations, such as calcium or magnesium ions, as well as substituted ammonium ions, such as ions of the type N(R¹)(R²)(R³)(R⁴)⁺, where R¹, R², R³ and R⁴ independently will typically designate hydrogen, optionally substituted C₁₋₆-alkyl or optionally substituted C₂₋₆-alkenyl. Examples of relevant C₁₋₆-alkyl groups include methyl, ethyl, 1-propyl and 2-propyl groups. Examples of C₂₋₆-alkenyl groups of possible relevance include ethenyl, 1-propenyl and 2-propenyl. Other examples of pharmaceutically acceptable salts are described in “Remington's Pharmaceutical Sciences”, 17th edition, Alfonso R. Gennaro (Ed.), Mark Publishing Company, Easton, Pa., USA, 1985 (and more recent editions thereof), in the “Encyclopedia of Pharmaceutical Technology”, 3rd edition, James Swarbrick (Ed.), Informa Healthcare USA (Inc.), NY, USA, 2007, and in J. Pharm. Sci. 66: 2 (1977). Also, for a review on suitable salts, see Handbook of Pharmaceutical Salts: Properties, Selection, and Use by Stahl and Wermuth (Wiley-VCH, 2002). Other suitable base salts are formed from bases which form non-toxic salts. Representative examples include the aluminum, arginine, benzathine, calcium, choline, diethylamine, diolamine, glycine, lysine, magnesium, meglumine, olamine, potassium, sodium, tromethamine, and zinc salts. Hemisalts of acids and bases can also be formed, e.g., hemisulphate and hemicalcium salts.

As used herein, the term “therapeutically effective amount” refers to an amount of a therapeutic agent (e.g., a peptide, polypeptide, or protein of the disclosure), which confers a therapeutic effect on the treated subject. Such a therapeutic effect can be objective (i.e., measurable by some test or marker) or subjective (i.e., subject gives an indication of, or feels an effect). In some embodiments, “therapeutically effective amount” refers to an amount of a therapeutic agent or composition effective to treat or ameliorate a relevant disease or condition, and/or to exhibit a detectable therapeutic or preventative effect, such as by ameliorating symptoms associated with the disease and/or also lessening severity or frequency of symptoms of the disease. For any particular therapeutic agent, a therapeutically effective amount (and/or an appropriate unit dose within an effective dosing regimen) can vary, for example, depending on route of administration or on combination with other therapeutic agents. Alternatively or additionally, a specific therapeutically effective amount (and/or unit dose) for any particular subject can depend upon a variety of factors including the particular form of disease being treated; the severity of the condition or pre-condition; the activity of the specific therapeutic agent employed; the specific composition employed; the age, body weight, general health, sex and diet of the subject; the time of administration, route of administration, and/or rate of excretion or metabolism of the specific therapeutic agent employed; the duration of the treatment; and like factors as is well known in the medical arts. The current disclosure utilizes therapeutically effective amounts of peptides and compositions comprising the same, to treat a variety of diseases, such as a variety of parasitic worm infections. The therapeutically effective amounts of the administered peptide, or compositions comprising the same, will in some embodiments modulate a circadian rhythm.

“Pharmaceutical” implies that a composition, reagent, method, and the like, are capable of a pharmaceutical effect, and also that the composition is capable of being administered to a subject safely. “Pharmaceutical effect,” without limitation, can imply that the composition, reagent, or method, is capable of stimulating a desired biochemical, genetic, cellular, physiological, or clinical effect, in at least one subject, such as a mammalian subject, for example, a human, in at least 5% of a population of subjects, in at least 10%, in at least 20%, in at least 30%, in at least 50% of subjects, and the like.

The phrases “pharmaceutical” or “pharmacologically acceptable” or “pharmaceutically acceptable” refer to molecular entities and compositions suitable for administration to a subject, such as, for example, a human, as appropriate. For example, “pharmaceutical” or “pharmacologically acceptable” or “pharmaceutically acceptable” can refer to agents approved by a regulatory agency of the Federal or a state government or listed in the U.S. Pharmacopoeia or other generally recognized pharmacopoeia for safe use in animals, and more particularly safe use in humans.

“Pharmaceutically acceptable vehicle” or “pharmaceutically acceptable carrier” refers to a diluent, adjuvant, excipient or carrier with which a peptide as described herein is administered. For example, a “pharmaceutically acceptable carrier” can include any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

“Prophylaxis” means a measure taken for the prevention of a disease or condition or at least one symptom thereof.

“Preventing” or “prevention” refers to a reduction in risk of acquiring a disease or disorder (i.e., causing at least one of the clinical symptoms of the disease not to develop in a subject that can be exposed to or predisposed to the disease but does not yet experience or display symptoms of the disease, or causing the symptom to develop with less severity than in absence of the treatment). “Prevention” or “prophylaxis” can also refer to delaying the onset of the disease or disorder.

“Prophylactically effective amount” means the amount of a compound, i.e., a peptide as described herein, that when administered to a subject for prevention of a disease or condition, is sufficient to effect such prevention of the disease or condition or to prevent development of at least one symptom of the disease or condition or effect development of the symptom at a lower level of severity than in the absence of administration of the compound. The “prophylactically effective amount” can vary depending on the compound, the disease and its severity, and the age, weight, etc., of the subject to be treated.

The term “amino acid” or “any amino acid” refers to any and all amino acids, including naturally occurring amino acids (e.g., alpha-amino acids), unnatural amino acids, and modified amino acids. It includes both D- and L-amino acids. Non-limiting examples of unnatural amino acids include beta-amino acids, homo-amino acids, proline and pyruvic acid derivatives, 3-substituted alanine derivatives, glycine derivatives, ring substituted phenylalanine and tyrosine derivatives, linear core amino acids, and N-methyl amino acids. A modified amino acid can be an amino acid resulting from a reaction at an amino group, carboxy group, side-chain functional group, or from the replacement of any hydrogen by a heteroatom. Amino acids are referred to herein by their full name and/or by their IUPAC one-letter abbreviation.

The recitations “sequence identity,” “percent identity,” “percent homology,” or for example, “comprising a sequence 50% identical to,” as used herein, refer to the extent that sequences are identical on an amino acid-by-amino acid basis, or a nucleotide-by-nucleotide basis, or over a window of comparison. Thus, a “percentage of sequence identity” can be calculated by comparing two optimally aligned sequences over the window of comparison, determining the number of positions at which the identical nucleic acid base (e.g., A, T, C, G, U) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

Calculations of sequence similarity or sequence identity between sequences (the terms are used interchangeably herein) can be performed as follows. To determine the percent identity of two amino acid sequences, or of two nucleic acid sequences, the sequences can be aligned for optimal comparison purposes (e.g., gaps can be introduced in one or both of a first and a second amino acid or nucleic acid sequence for optimal alignment and non-homologous sequences can be disregarded for comparison purposes. The comparison of sequences and determination of percent identity between two sequences can be accomplished using a mathematical algorithm. In some embodiments, the percent identity between two amino acid sequences is determined using the Needleman and Wunsch, (1970, J. Mol. Biol. 48: 444-453) algorithm which has been incorporated into the GAP program in the GCG software package, using either a BLOSUM 62 matrix or a PAM250 matrix, and a gap weight of 16, 14, 12, 10, 8, 6, or 4 and a length weight of 1, 2, 3, 4, 5, or 6. 0), using a PAM120 weight residue table, a gap length penalty of 12 and a gap penalty of 4.

Related (and variant) peptides encompass “variant” peptides. Variant peptides differ from another (i.e., parental) peptide and/or from one another by a small number of amino acid residues. A variant can include one or more amino acid modifications (e.g., amino acid deletion, insertion, or substitution) as compared to the parental protein/peptide from which it is derived. In some embodiments, the number of different amino acid residues is any of about 1, 2, 3, 4, 5, 10, or 20. In some embodiments, variants differ by about 1 to about 15 amino acids (e.g., 1 to 5, 1 to 10, 5 to 10, 5 to 15, or 10 to 15). Alternatively or additionally, variants can have a specified degree of sequence identity with a reference protein/peptide or nucleic acid, e.g., as determined using a sequence alignment tool, such as the previously discussed BLAST, ALIGN, and CLUSTAL. For example, variant proteins/peptides or nucleic acid can have at least about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or even 99.5% amino acid sequence identity with a reference sequence. In some embodiments, variant proteins/peptides or nucleic acids are not 100% identical to a reference sequence.

As used herein, the term “amino acid modification” refers to, e.g., an amino acid substitution, deletion, and/or insertion, as is well understood in the art.

The term “bacterium” or “bacterial cell” means any cell from or derived from any bacterium (e.g., a Gram positive bacterium, or a Gram negative bacterium). Non-limiting examples of bacteria are described herein. Additional examples of bacteria are known in the art.

The term “recombinant bacterium” means a bacterium that contains a nucleic acid that is not naturally present in the bacterium. For example, the nucleic acid that is not naturally present in the bacterium can encode a recombinant polypeptide (e.g., any of the exemplary recombinant peptides described herein) and/or can encode a selectable marker (e.g., any of the exemplary selectable markers described herein). The nucleic acid that is not naturally present in the cell can, e.g., be integrated into the genome of the bacterium. In other examples, the nucleic acid that is not naturally present in the cell is not integrated into the genome of the bacterium. For example, a nucleic acid that is not naturally present in the bacterium can be episomal.

The term “promoter” is a nucleic acid sequence that is operably linked to a nucleic acid sequence encoding a polypeptide (e.g., a recombinant peptide) that can increase the transcription of the nucleic acid sequence encoding the polypeptide (e.g., a peptide described herein). In some aspects, a promoter is constitutive. In other aspects, a promoter is inducible. Non-limiting examples of promoters are described herein. Additional examples of promoters are known in the art.

The term “culturing” refers to growing a population of cells, e.g., microbial cells, under suitable conditions for growth, in a liquid or solid medium.

The term “purifying” means a step performed to isolate a recombinant peptide from one or more other impurities (e.g., bulk impurities) or components present in a fluid containing a recombinant peptide (e.g., liquid culture medium polypeptides or one or more other components (e.g., DNA, RNA, other polypeptides, endotoxins, viruses, etc.) present in or secreted from a mammalian cell).

The terms “isolated,” “purified,” “separated,” and “recovered” as used herein refer to a material (e.g., a polypeptide, nucleic acid, or cell) that is removed from at least one component with which it is naturally associated, for example, at a concentration of at least 90% by weight, or at least 95% by weight, or at least 98% by weight of the sample in which it is contained. For example, these terms can refer to a material which is substantially or essentially free from components which normally accompany it as found in its native state, such as, for example, an intact biological system, or is substantially or essentially free from other proteins in the system from which it is expressed.

As used herein, the term “host cell” refers to a cell or cell line into which a recombinant expression vector (e.g., a nucleic acid construct) can be introduced for expression of the peptide in the host cell. A host cell comprising a recombinant vector can be referred to as a “recombinant host cell.”

As used herein, “inhibiting and suppressing” and like terms should not be construed to require complete inhibition or suppression, although this can be desired in some embodiments.

Thus, as used herein, the terms “increase” or “reduce,” or grammatical equivalents, indicate values that are relative to a reference (e.g., baseline) measurement, such as a measurement taken under comparable conditions (e.g., in the same subject prior to initiation of treatment described herein, or a measurement in a control subject (or multiple control subjects) in the absence of treatment) described herein. In some embodiments, a suitable control is a baseline measurement, such as a measurement in the same subject prior to initiation of the treatment described herein, or a measurement in a control subject (or multiple control subjects) in the absence of the treatment described herein.

Reference to the term “about” has its usual meaning in the context of compositions to allow for reasonable variations in amounts that can achieve the same effect and also refers herein to a value of plus or minus 10% of the provided value. For example, “about 20” means or includes amounts from 18 to and including 22.

Unless otherwise required by context, singular terms shall include pluralities and plural terms shall include the singular. As used herein, the singular form “a”, “an”, and “the” include plural references unless indicated otherwise. For example, “an” excipient includes one or more excipients. It is understood that aspects and variations of the invention described herein include “consisting of” and/or “consisting essentially of” aspects and variations,

The details of one or more embodiments of the invention are set forth in the accompanying drawings and the description below. Other features, objects, and advantages of the invention will be apparent from the description and drawings, and from the claims. Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### DESCRIPTION OF DRAWINGS

FIG. 1 is a schematic for a human T cell activation assay.

FIG. 2A shows a protocol for in vitro activation of the human T cells and FIG. 2B shows a protocol used for flow cytometry analysis.

FIG. 3 shows effects of a peptide such as SG-3-0020_{C→S} on activation of human T cells.

FIG. 4 shows effects of a peptide such as SG-3-0020_{C→S} on activation of human T cells.

FIG. 5 shows effects of a peptide such as SG-3-0020_{C→S} on activation of human T cells.

FIG. 6A provides sequences (SEQ ID NOs: 358-372) used in Example 3 and FIG. 6B shows the effects of alanine shaving on T-cell binding.

FIGS. 7A and 7B show effects of a peptide such as SG-3-0020_{C→S} on activation of human T cells.

FIG. 8 shows effects of SG-3-0020 1-29 aa N-terminal+middle regions on activation of human T cells.

FIG. 9 shows effects of SG-3-0020 1-29 aa N-terminal+middle regions on activation of human T cells.

FIG. 10 shows peptide sequences from the combinatorial library.

FIGS. 11 and 12 show 150 peptide variants of the SG-3-0020 1-29 aa N-terminal+middle region, derived from a combinatorial phage display library, were tested for activation potential of human T cells. FIG. 11 is a plot showing that 136 peptides showed an increase in IFN-g secretion (hit) by at least 2 fold when compared to the wild type 29 aa form. FIG. 12 is a plot showing that 34 peptides showed an increase in IFN-g secretion (hit) by at least 2 fold when compared to the wild type full length SG-3-0020 form, which was tested a 10 fold higher concentration than the novel variants. Increase in cytokine secretion was concordant in 3 donors.

FIG. 13 is an alignment of peptides identified as low binders in Example 4. The top reference peptide is SEQ ID NO: 1.

FIG. 14 is a list of peptides synthesized from the combinatorial library of Example 5.

FIG. 15 is a flow chart depicting the steps to identify target binding partners of peptides.

FIG. 16 is a flow chart depicting the steps of CRISPR KO in T cells and dendritic cells to confirm putative human targets.

FIGS. 17A-17B are plots showing INF-γ cytokine release and relative sizes of cell populations relative to the parent population.

FIG. 18 is a heat map of relative cytokine levels in the tumor microenvironment following treatment with various peptides.

FIG. 19 shows plots of cytokine concentrations normalized to 30 μM peptide for various peptides.

FIG. 20 is a plot of mean fluorescence intensity (MFI) of human monocyte-derived dendritic cells stimulated with various peptides and cytokines.

FIGS. 21A-21E are plots of activation of chemokine receptors when incubated with peptides.

FIGS. 22A-22E are plots showing the activation of chemokine receptors when incubated with various peptides.

FIG. 23A is a predicted structure of SG-3-00802 peptide with a zinc ion. FIG. 23B is a plot of binding of peptides to human dendritic cells tested by flow cytometry.

FIG. 24 is a plot of CXCL10 cytokine release after treatment with SG-3-00802 peptide.

FIG. 25 is a plot of CXCL10 cytokine release after treatment with SG-3-05021 peptide.

FIGS. 26A-26B are plots of cytokine release after treatment with SG-3-00802 alone or in combination with anti-CD40 and anti-PD-L1.

FIGS. 27A-27B are plots of cytokine release after treatment with SG-3-05021 alone or in combination with anti-CD40 and anti-PD-L1.

FIG. 28A is a plot of tumor volume after various treatments. FIG. 28B is a plot of tumor volume after various treatments.

FIG. 29 is a plot of a survival curve of mice after various treatments.

FIGS. 30A-30B are plots of survival curves of mice after various treatments.

FIGS. 31A-31B are plots of survival curves of mice after various treatments.

FIGS. 32-32B are plots of tumor volume after various treatments.

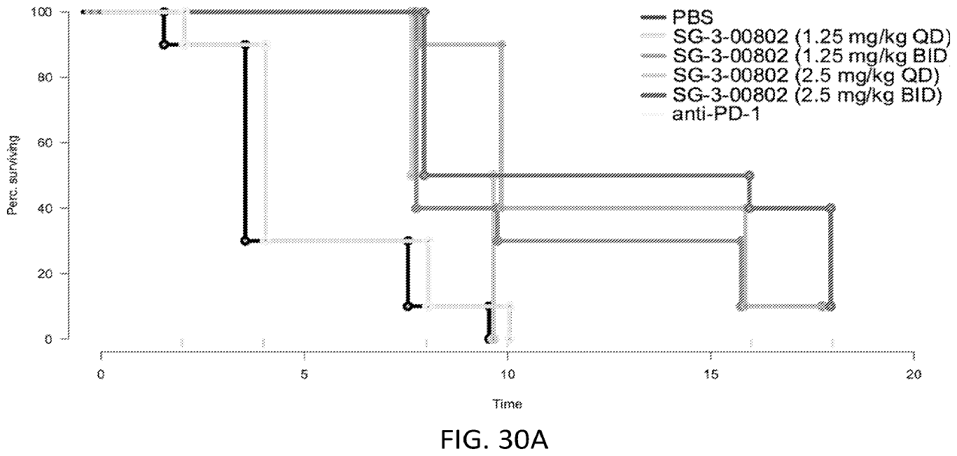

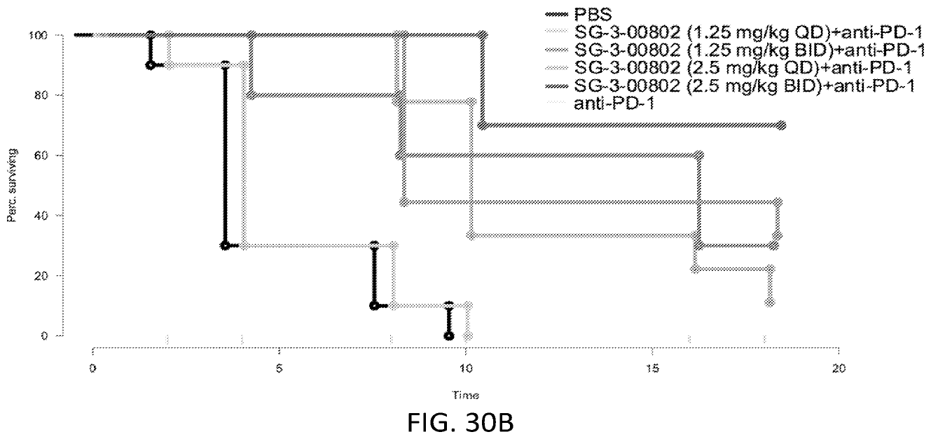

FIG. 30A is a plot showing the number of responder, partial responder, and non-responder patients in study cohorts. FIG. 30B is a plot showing the number of patients in each immune checkpoint inhibitor therapy group.

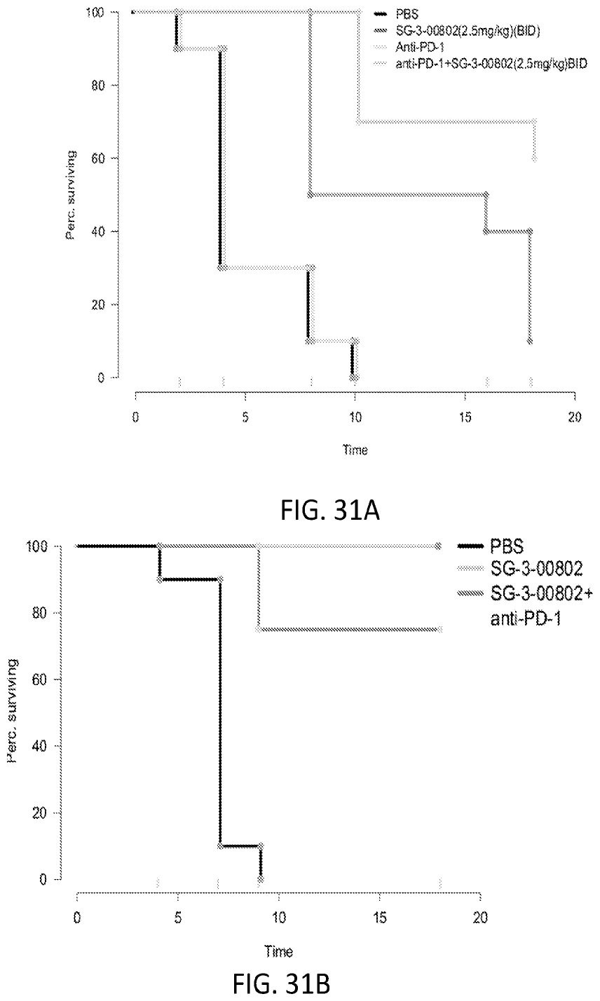

FIGS. 31A-31B are plots of survival curves of mice after various treatments.

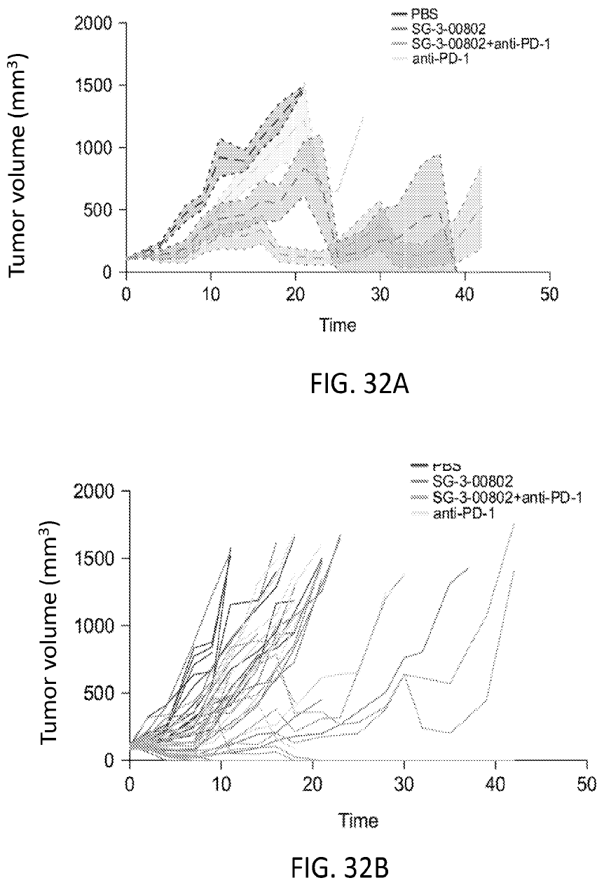

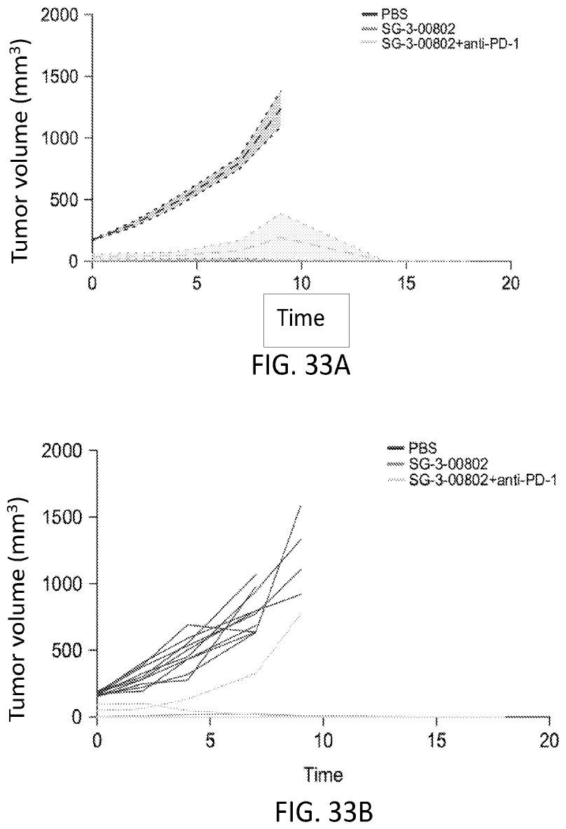

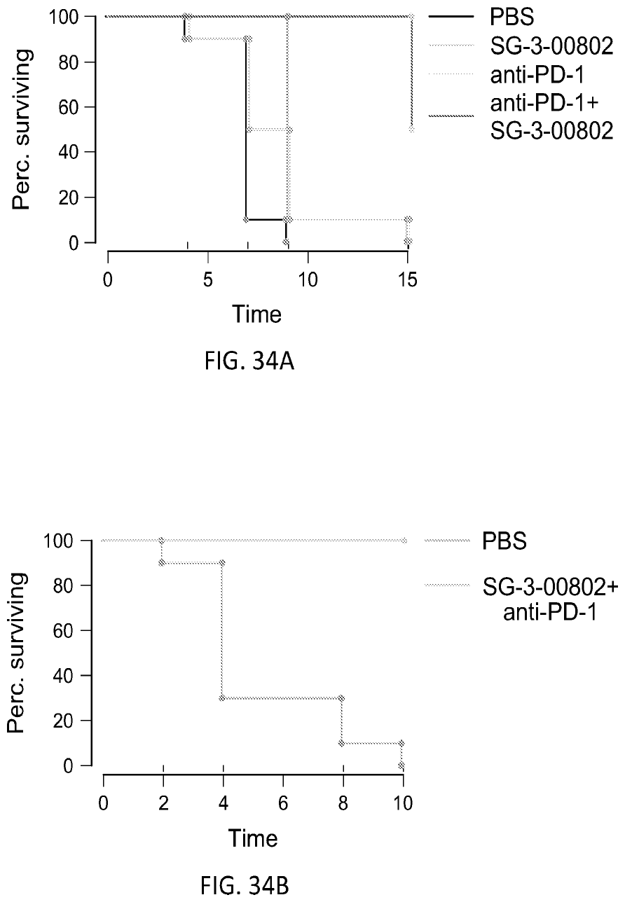

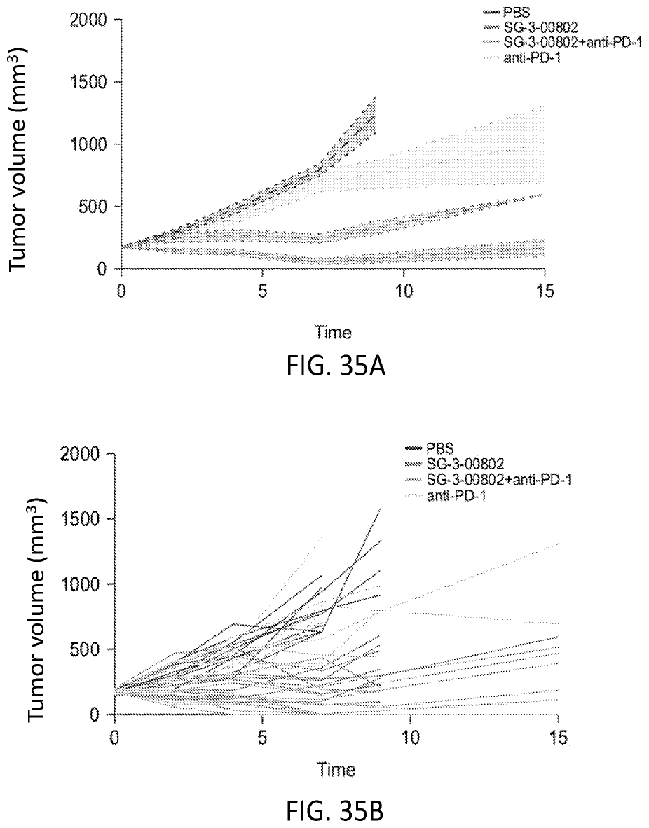

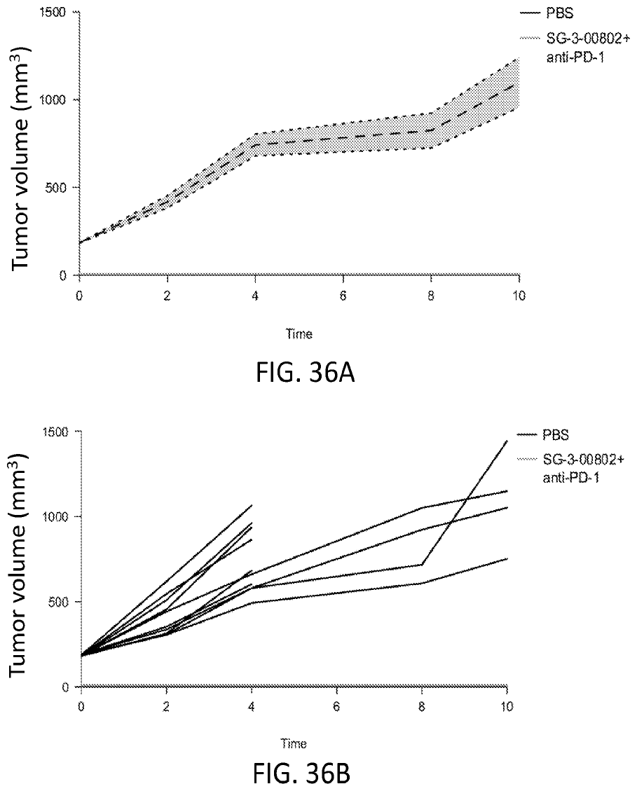

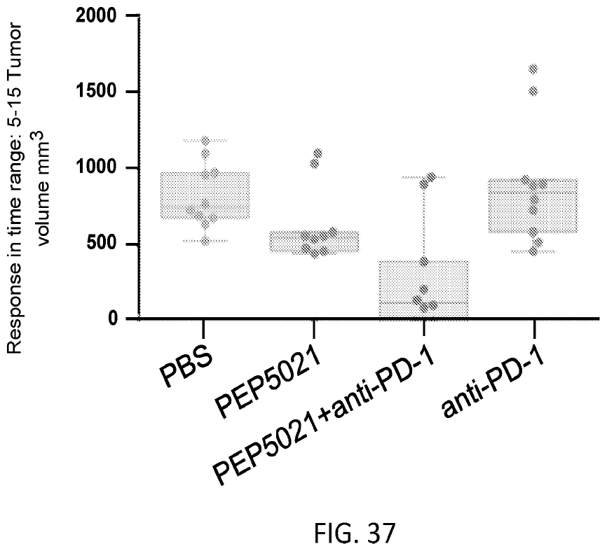

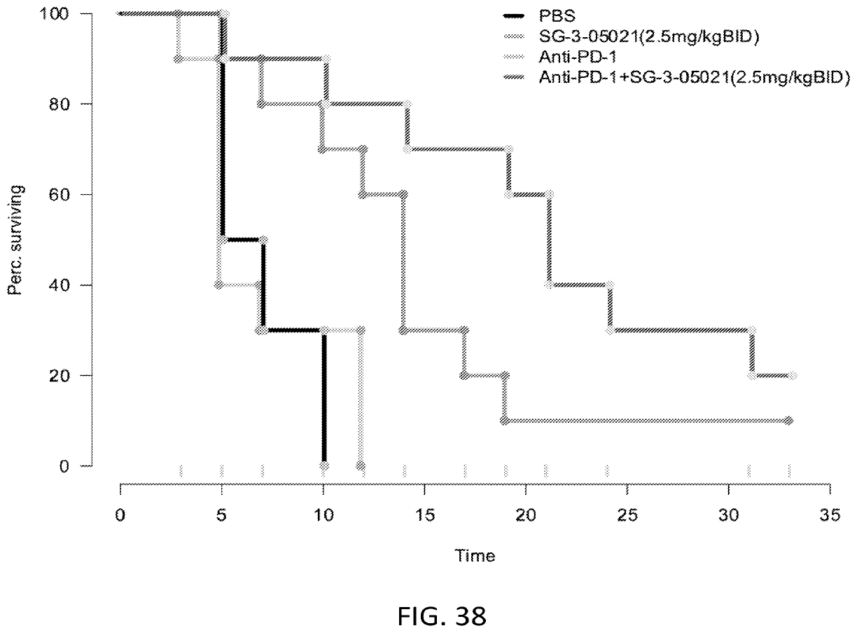

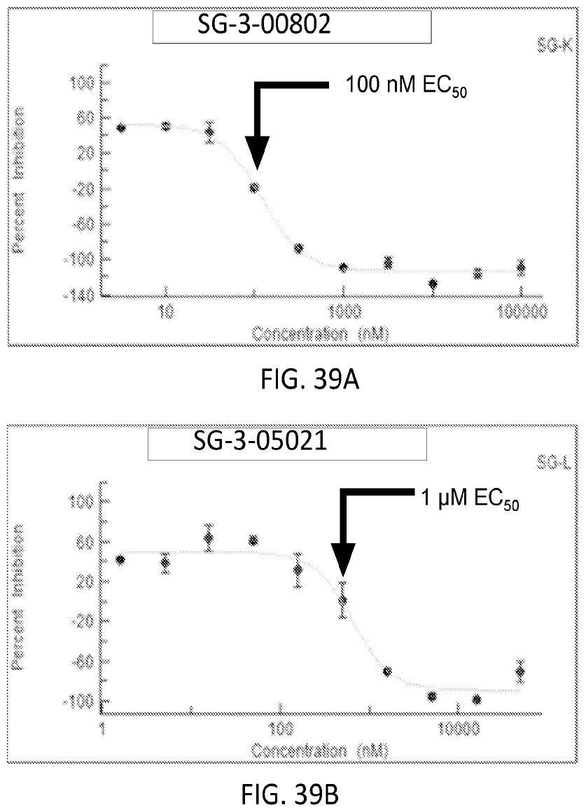

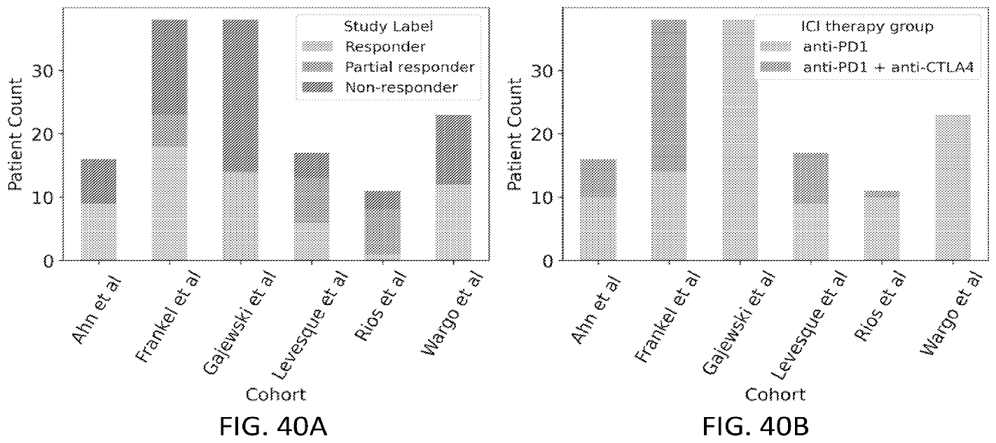

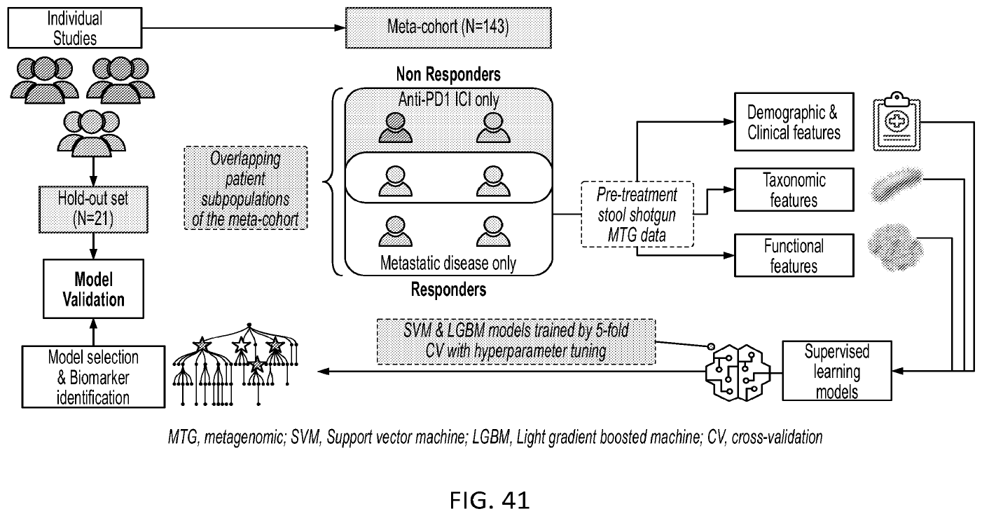

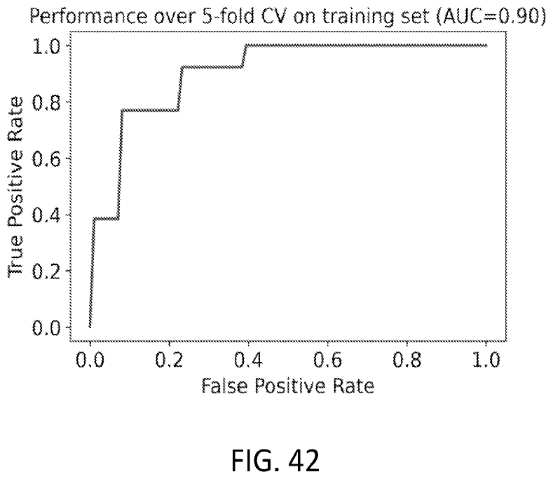

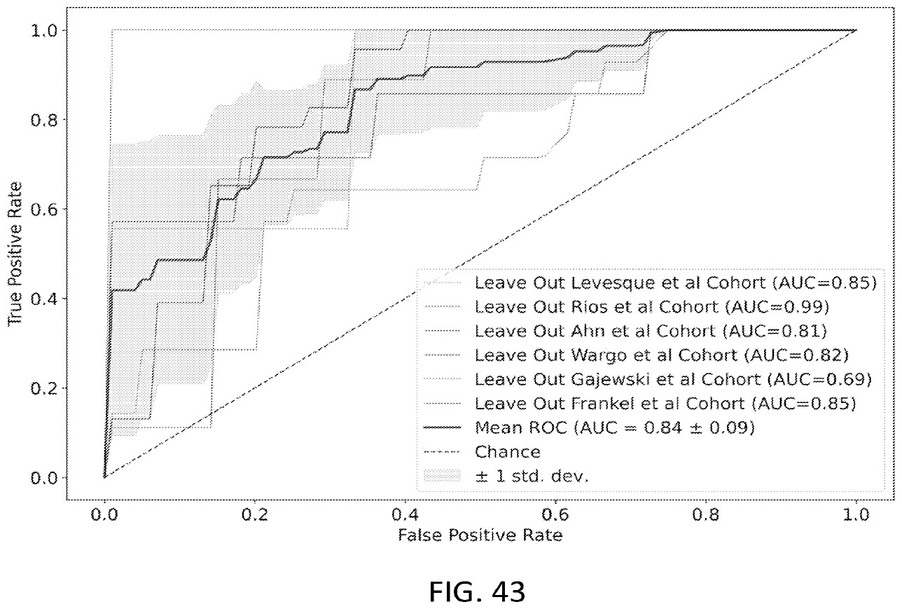

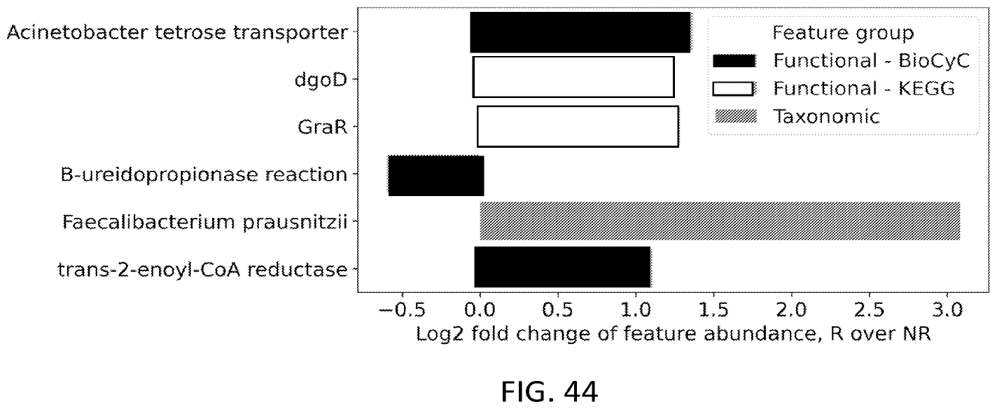

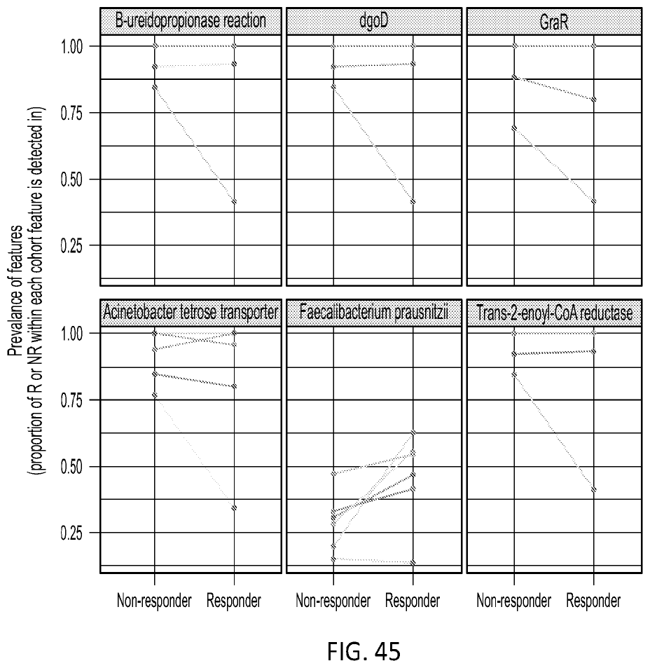

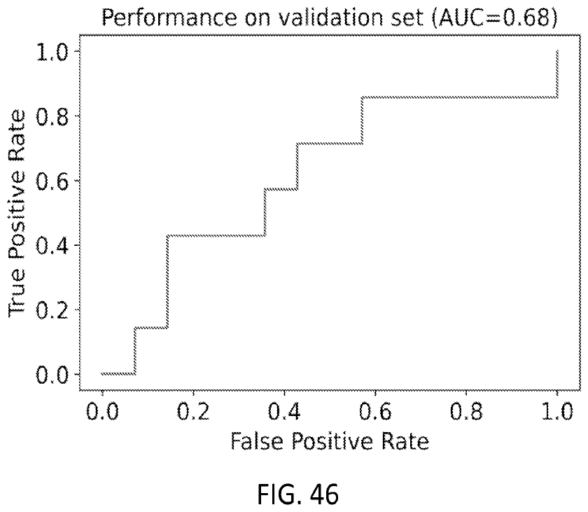

FIGS. 32A-32B are plots of tumor volume after various treatments.

FIGS. 33A-33B are plots of tumor volume after various treatments.

FIGS. 34A-34B are plots of a survival curve of mice after various treatments.

FIGS. 35A-35B are plots of tumor volume after various treatments.

FIGS. 36A-36B are plots of tumor volume after various treatments.

FIG. 37 is a plot of tumor volume after various treatments.

FIG. 38 is a plot of a survival curve of mice after various treatments.

FIGS. 39A-39B are plots inhibition after treatment with peptides.

FIGS. 40A-40B are plots of the number of patients (patient count) in various studies according to their responder category and checkpoint inhibitor therapy group.

FIG. 41 depicts a flow chart of the model training and validation.

FIG. 42 is a graph of biomarker model performance.

FIG. 43 is a graph of biomarker model performance with each cohort.

FIG. 44 is a graph of model feature importance.

FIG. 45 is a panel of graphs of biomarker model feature prevalence in non-responder and responder patients.

FIG. 46 is a graph of biomarker model validation.

### DETAILED DESCRIPTION

Bifidobacterium, a commensal bacterium, has been shown to promote anti-tumor immunity and facilitate anti-programmed cell death protein ligand 1 (PD-L1) efficacy. In fact, the bacterium elicits an additive effect with anti-PD-L1 and increases MHC-II on dendritic cells. See Sivan et al. (2015. Science. 350(6264):1084-1089). Polypeptides or fragments thereof expressed by Bifidobacterium can promote activation of immune cells which can facilitate destruction of tumor cells. Increasing the ratio of T_{eff}:T_{reg} cells may prevent tumor cells from escaping immune surveillance, leading to the destruction of tumor cells by the subject immune system.

This document provides compositions and methods for treating subjects in need thereof (e.g., subjects having an immunological disease or cancer) using one or more peptides described herein. Immunological diseases and cancers that can be treated using a peptide as described herein can include diseases that are associated with inflammatory immune responses. In some embodiments, peptides described herein modulate immunoregulatory cells, including but not limited to T cells, effector T cells and dendritic cells.

### Peptides

Polypeptides encoded by the Bifidobacterium genome or fragments thereof have been tested for their ability to stimulate differentiation of naïve CD4+ and CD8+ T cells (Th0) to CD4+ and CD8+ activated T cells (T_{act}). Incubation of a naïve T cell with a Bifidobacterium polypeptide, SG-3-0020, resulted in an increase in the number of CD4+ and/or CD8+ T cells having a CD25+/FoxP3− phenotype suggesting that the Bifidobacterium polypeptide has increased the ratio of T_{eff}:T_{reg}. See International Application No. PCT/US2020/012431.

In some embodiments, peptides described herein include a peptide of SEQ ID NO:1 or a variant thereof (see Table 1).

TABLE 1

| Sequence | |
|---|---|
| Identifier | Amino Acid Sequence |
| SEQ ID NO: 1 | MLSTKKTKTHDHYPSGRMRDPGWHDWRAS |

A variant of SEQ ID NO:1 according to the present disclosure can be a peptide with 1, 2, 3, 4, 5, 6, 7, 8, 9, 10, 11, 12, 13, 14, or 15 amino acid modifications relative to SEQ ID NO:1. In some embodiments, the variant can be at least 50%, 55%, 60%, 65%, 70%, 75%, 80%, 85%, 90%, 91%, 92%, 93%, 94%, 95%, 96%, 97%, or 98% identical to SEQ ID NO:1. Examples of amino acid modifications with respect to the amino acid sequence set forth in SEQ ID NO:1, include, without limitation, amino acid substitutions, amino acid deletions, and amino acid insertions. In some embodiments, a peptide of the present disclosure has a deletion of 1, 2, 3, 4 or 5 N- or C-terminal residues of SEQ ID NO:1. Alternatively or additionally, there is an internal deletion of 1, 2, 3, 4, or 5 amino acids relative to SEQ ID NO:1.

An amino acid substitution of SEQ ID NO:1 can be a conservative amino acid substitution. For example, conservative amino acid substitutions can be made by substituting one amino acid residue for another amino acid residue having a similar side chain. Families of amino acid residues having similar side chains can include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine).

In some embodiments, an amino acid substitution of SEQ ID NO:1 is a non-conservative amino acid substitution. Non-conservative amino acid substitutions can be made by substituting one amino acid residue for another amino acid residue having a dissimilar side chain. Examples of non-conservative substitutions include, without limitation, substituting (a) a hydrophilic residue (e.g., serine or threonine) for a hydrophobic residue (e.g., leucine, isoleucine, phenylalanine, valine, or alanine); (b) a cysteine or proline for any other residue; (c) a residue having a basic side chain (e.g., lysine, arginine, or histidine) for a residue having an acidic side chain (e.g., aspartic acid or glutamic acid); and (d) a residue having a bulky side chain (e.g., phenylalanine) for glycine or other residue having a small side chain.

In some embodiments, the methionine at position at 1 of SEQ ID NO:1 is substituted with another amino acid (e.g., any of the other naturally-occurring amino acids). In some embodiments, the methionine at position at 1 of SEQ ID NO:1 is substituted with an amino acid selected from the group consisting of: W, F, V, P, K, R, and S.

In some embodiments, the leucine at position at 2 of SEQ ID NO:1 is substituted with another amino acid (e.g., any of the other naturally-occurring amino acids). In some embodiments, the leucine at position at 2 of SEQ ID NO:1 is substituted with an amino acid selected from the group consisting of: S, P, G, T, V, A, K, Q, R, W, Y, F, and N.

In some embodiments, the serine at position at 3 of SEQ ID NO:1 is substituted with another amino acid (e.g., any of the other naturally-occurring amino acids). In some embodiments, the serine at position at 3 of SEQ ID NO:1 is substituted with an amino acid selected from the group consisting of: Q and R.

In some embodiments, the threonine at position at 4 of SEQ ID NO:1 is substituted with another amino acid (e.g., any of the other naturally-occurring amino acids). In some embodiments, the threonine at position at 4 of SEQ ID NO:1 is substituted with an amino acid selected from the group consisting of: G, A, and R.

In some embodiments, the lysine at position at 5 of SEQ ID NO:1 is substituted with another amino acid (e.g., any of the other naturally-occurring amino acids). In some embodiments, the lysine at position at 5 of SEQ ID NO:1 is substituted with an amino acid selected from the group consisting of: A and R.

In some embodiments, the lysine at position at 6 of SEQ ID NO:1 substituted with another amino acid (e.g., any of the other naturally-occurring amino acids). In some embodiments, the lysine at position at 6 of SEQ ID NO:1 is substituted with an amino acid selected from the group consisting of: R, T, and A.

In some embodiments, the threonine at position at 7 of SEQ ID NO:1 substituted with another amino acid (e.g., any of the other naturally-occurring amino acids). In some embodiments, the threonine at position at 7 of SEQ ID NO:1 is substituted with an amino acid selected from the group consisting of: G, K, and R.

In some embodiments, the threonine at position at 9 of SEQ ID NO:1 is substituted with another amino acid (e.g., any of the other naturally-occurring amino acids). In some embodiments, the threonine at position at 9 of SEQ ID NO:1 is substituted with an amino acid selected from the group consisting of: W and R.

In some embodiments, the histidine at position at 10 of SEQ ID NO:1 is substituted with another amino acid (e.g., any of the other naturally-occurring amino acids). In some embodiments, the histidine at position at 10 of SEQ ID NO:1 is substituted with an amino acid selected from the group consisting of: K and R.

In some embodiments, the aspartic acid at position at 11 of SEQ ID NO:1 is substituted with another amino acid (e.g., any of the other naturally-occurring amino acids). In some embodiments, the aspartic acid at position at 11 of SEQ ID NO:1 is substituted with an amino acid selected from the group consisting of: F, G, H, I, K, P, R, T, V, W, and Y.

In some embodiments, the histidine at position at 12 of SEQ ID NO:1 is substituted with another amino acid (e.g., any of the other naturally-occurring amino acids). In some embodiments, the histidine at position at 12 of SEQ ID NO:1 is substituted with an amino acid selected from the group consisting of: K and R.

In some embodiments, the tyrosine at position at 13 of SEQ ID NO:1 is substituted with another amino acid (e.g., any of the other naturally-occurring amino acids). In some embodiments, the tyrosine at position at 13 of SEQ ID NO:1 is substituted with an amino acid selected from the group consisting of: W, N, G, K, R, and W.

In some embodiments, the proline at position at 14 of SEQ ID NO:1 is substituted with another amino acid (e.g., any of the other naturally-occurring amino acids). In some embodiments, the proline at position at 14 of SEQ ID NO:1 is substituted with an amino acid selected from the group consisting of: G and W.

In some embodiments, the serine at position at 15 of SEQ ID NO:1 is substituted with another amino acid (e.g., any of the other naturally-occurring amino acids). In some embodiments, the serine at position at 15 of SEQ ID NO:1 is substituted with an amino acid selected from the group consisting of: G and R.

In some embodiments, the methionine at position at 18 of SEQ ID NO:1 is substituted with another amino acid (e.g., any of the other naturally-occurring amino acids). In some embodiments, the methionine at position at 18 of SEQ ID NO:1 is substituted with an amino acid selected from the group consisting of: W, H, Y, G, and R.

In some embodiments, the aspartic acid at position at 20 of SEQ ID NO:1 is substituted with another amino acid (e.g., any of the other naturally-occurring amino acids). In some embodiments, the aspartic acid at position at 20 of SEQ ID NO:1 is substituted with an amino acid selected from the group consisting of: P and R.

In some embodiments, the proline at position at 21 of SEQ ID NO:1 is substituted with another amino acid (e.g., any of the other naturally-occurring amino acids). In some embodiments, the proline at position at 21 of SEQ ID NO:1 is F.

In some embodiments, the glycine at position at 22 of SEQ ID NO:1 is substituted with another amino acid (e.g., any of the other naturally-occurring amino acids). In some embodiments, the glycine at position at 22 of SEQ ID NO:1 is substituted with an amino acid selected from the group consisting of: P, K, and W.

In some embodiments, the aspartic acid at position at 25 of SEQ ID NO:1 is substituted with another amino acid (e.g., any of the other naturally-occurring amino acids). In some embodiments, the aspartic acid at position at 25 of SEQ ID NO:1 is substituted with an amino acid selected from the group consisting of: P and R.

In some embodiments, the arginine at position at 27 of SEQ ID NO:1 is substituted with another amino acid (e.g., any of the other naturally-occurring amino acids). In some embodiments, the arginine at position at 27 of SEQ ID NO:1 is W.

In some embodiments, the alanine at position at 28 of SEQ ID NO:1 is substituted with another amino acid (e.g., any of the other naturally-occurring amino acids). In some embodiments, the alanine at position at 28 of SEQ ID NO:1 is substituted with an amino acid selected from the group consisting of: F, G, V, Y, and W.

In some embodiments, the serine at position at 29 of SEQ ID NO:1 is substituted with another amino acid (e.g., any of the other naturally-occurring amino acids). In some embodiments, the serine at position at 29 of SEQ ID NO:1 is substituted with R.

In some embodiments, none of the amino acids of SEQ ID NO:1 are substituted with cysteine.

In some embodiments, a peptide described herein has an amino acid sequence selected from Table 2.

TABLE 2

| | |
|---|---|
| SEQ ID NO: 2 | MLSTAKGKTHDHWGGGRMRPPPWHDWRFS |
| SEQ ID NO: 3 | MSSGAKGKWHDHYPSGRMRPPPWHDWRFS |
| SEQ ID NO: 4 | WPSGAKGKWHDHYPGGRMRPPPWHDWRFS |
| SEQ ID NO: 5 | WGSGAKGKTHDHWGSGRWRDPGWHDWRGS |
| SEQ ID NO: 6 | FGSGAKGKTHDHWGGGRHRPPPWHDWRGS |
| SEQ ID NO: 7 | FGSGAKGKTHDHYPSGRMRPPPWHDWRVR |
| SEQ ID NO: 8 | MPSAAKGKWHDHYPGGRMRDPKWHDWRFS |
| SEQ ID NO: 9 | VTSTAKGKTHDHNWSGRYRPPPWHDWRAR |
| SEQ ID NO: 10 | FVSGAKGKWHDHWPSGRWRDPPWHDWRAS |
| SEQ ID NO: 11 | MASAAKGKWHDHWGSGRHRPPPWHDWRVS |
| SEQ ID NO: 12 | MPSAAKGKWHDHYPSGRMRPPPWHDWRAR |
| SEQ ID NO: 13 | MPSGAKGKTHDHYGGGRMRPPPWHDWRFS |
| SEQ ID NO: 14 | FSSAAKGKWHDHWPGGRYRDPPWHDWRVS |
| SEQ ID NO: 15 | FLSGAKGKTHDHYPSGRYRPPPWHDWRAR |
| SEQ ID NO: 16 | MPSAAKTKWHDHYPGGRMRPPPWHDWRGR |
| SEQ ID NO: 17 | WSSAAKGKTHDHWGGGRYRPPPWHDWRAS |
| SEQ ID NO: 18 | PVSAAKGKWHDHNWSGRMRPPPWHDWRAR |
| SEQ ID NO: 19 | WSSGAKTKWHDHYGSGRHRDPKWHDWRGS |
| SEQ ID NO: 20 | WASGAKGKWHDHYGGGRYRDPGWHDWRGS |
| SEQ ID NO: 21 | WGSAAKGKTHDHWPSGRYRDPKWHDWRVS |
| SEQ ID NO: 22 | MLSGAKGKTHDHYWGGRMRPPPWHDWRGS |
| SEQ ID NO: 23 | FGSAAKGKTHDHWPSGRHRDPGWHDWRFS |
| SEQ ID NO: 24 | WVSAAKGKWHDHYGSGRMRDPPWHDWWGS |
| SEQ ID NO: 25 | FVSAAKGKTHDHYPSGRYRPPPWHDWRAR |
| SEQ ID NO: 26 | WPSAAKGKTHDHNPSGRMRPPPWHDWRFS |
| SEQ ID NO: 27 | MASAAKTKWHDHYPSGRMRPPPWHDWRAR |
| SEQ ID NO: 28 | MPSAAKTKWHDHYPSGRMRPPPWHDWRGR |
| SEQ ID NO: 29 | MVSTAKGKTHDHYPSGRYRPPPWHDWRFS |
| SEQ ID NO: 30 | WVSAAKGKTHDHWGSGRHRDPPWHDWRAR |
| SEQ ID NO: 31 | WGSAAKGKTHDHWPSGRHRDPPWHDWRVR |
| SEQ ID NO: 32 | WGSAAKGKTHDHYGSGRYRDPPWHDWRAR |
| SEQ ID NO: 33 | FASAAKGKTHDHYWSGRYRPPPWHDWRAS |
| SEQ ID NO: 34 | FGSAAKGKTHDHYWSGRMRPPPWHDWRGS |
| SEQ ID NO: 35 | WSSTAKGKTHDHWPSGRYRPPPWHDWRAR |
| SEQ ID NO: 36 | FLSTKKTKTHDHYPSGRMRDPGWHDWRAS |
| SEQ ID NO: 37 | KLSTKKTKTHDHYPSGRMRDPGWHDWRAS |
| SEQ ID NO: 38 | PLSTKKTKTHDHYPSGRMRDPGWHDWRAS |
| SEQ ID NO: 39 | RLSTKKTKTHDHYPSGRMRDPGWHDWRAS |
| SEQ ID NO: 40 | MASTKKTKTHDHYPSGRMRDPGWHDWRAS |
| SEQ ID NO: 41 | MGSTKKTKTHDHYPSGRMRDPGWHDWRAS |
| SEQ ID NO: 42 | MKSTKKTKTHDHYPSGRMRDPGWHDWRAS |
| SEQ ID NO: 43 | MPSTKKTKTHDHYPSGRMRDPGWHDWRAS |
| SEQ ID NO: 44 | MQSTKKTKTHDHYPSGRMRDPGWHDWRAS |
| SEQ ID NO: 45 | MRSTKKTKTHDHYPSGRMRDPGWHDWRAS |
| SEQ ID NO: 46 | MSSTKKTKTHDHYPSGRMRDPGWHDWRAS |
| SEQ ID NO: 47 | MTSTKKTKTHDHYPSGRMRDPGWHDWRAS |
| SEQ ID NO: 48 | MWSTKKTKTHDHYPSGRMRDPGWHDWRAS |
| SEQ ID NO: 49 | MYSTKKTKTHDHYPSGRMRDPGWHDWRAS |
| SEQ ID NO: 50 | MLQTKKTKTHDHYPSGRMRDPGWHDWRAS |
| SEQ ID NO: 51 | MLRTKKTKTHDHYPSGRMRDPGWHDWRAS |
| SEQ ID NO: 52 | MLSRKKTKTHDHYPSGRMRDPGWHDWRAS |
| SEQ ID NO: 53 | MLSTRKTKTHDHYPSGRMRDPGWHDWRAS |
| SEQ ID NO: 54 | MLSTKRTKTHDHYPSGRMRDPGWHDWRAS |
| SEQ ID NO: 55 | MLSTKTTKTHDHYPSGRMRDPGWHDWRAS |
| SEQ ID NO: 56 | MLSTKKKKTHDHYPSGRMRDPGWHDWRAS |
| SEQ ID NO: 57 | MLSTKKRKTHDHYPSGRMRDPGWHDWRAS |
| SEQ ID NO: 58 | MLSTKKTKRHDHYPSGRMRDPGWHDWRAS |
| SEQ ID NO: 59 | MLSTKKTKTKDHYPSGRMRDPGWHDWRAS |
| SEQ ID NO: 60 | MLSTKKTKTRDHYPSGRMRDPGWHDWRAS |
| SEQ ID NO: 61 | MLSTKKTKTHFHYPSGRMRDPGWHDWRAS |
| SEQ ID NO: 62 | MLSTKKTKTHGHYPSGRMRDPGWHDWRAS |
| SEQ ID NO: 63 | MLSTKKTKTHHHYPSGRMRDPGWHDWRAS |
| SEQ ID NO: 64 | MLSTKKTKTHIHYPSGRMRDPGWHDWRAS |
| SEQ ID NO: 65 | MLSTKKTKTHKHYPSGRMRDPGWHDWRAS |
| SEQ ID NO: 66 | MLSTKKTKTHPHYPSGRMRDPGWHDWRAS |
| SEQ ID NO: 67 | MLSTKKTKTHRHYPSGRMRDPGWHDWRAS |
| SEQ ID NO: 68 | MLSTKKTKTHTHYPSGRMRDPGWHDWRAS |
| SEQ ID NO: 69 | MLSTKKTKTHVHYPSGRMRDPGWHDWRAS |
| SEQ ID NO: 70 | MLSTKKTKTHWHYPSGRMRDPGWHDWRAS |
| SEQ ID NO: 71 | MLSTKKTKTHYHYPSGRMRDPGWHDWRAS |
| SEQ ID NO: 72 | MLSTKKTKTHDKYPSGRMRDPGWHDWRAS |
| SEQ ID NO: 73 | MLSTKKTKTHDRYPSGRMRDPGWHDWRAS |
| SEQ ID NO: 74 | MLSTKKTKTHDHGPSGRMRDPGWHDWRAS |
| SEQ ID NO: 75 | MLSTKKTKTHDHKPSGRMRDPGWHDWRAS |
| SEQ ID NO: 76 | MLSTKKTKTHDHRPSGRMRDPGWHDWRAS |
| SEQ ID NO: 77 | MLSTKKTKTHDHWPSGRMRDPGWHDWRAS |
| SEQ ID NO: 78 | MLSTKKTKTHDHYPRGRMRDPGWHDWRAS |
| SEQ ID NO: 79 | MLSTKKTKTHDHYPSGRGRDPGWHDWRAS |
| SEQ ID NO: 80 | MLSTKKTKTHDHYPSGRHRDPGWHDWRAS |
| SEQ ID NO: 81 | MLSTKKTKTHDHYPSGRKRDPGWHDWRAS |
| SEQ ID NO: 82 | MLSTKKTKTHDHYPSGRRRDPGWHDWRAS |
| SEQ ID NO: 83 | MLSTKKTKTHDHYPSGRWRDPGWHDWRAS |
| SEQ ID NO: 84 | MLSTKKTKTHDHYPSGRMRPPGWHDWRAS |
| SEQ ID NO: 85 | MLSTKKTKTHDHYPSGRMRRPGWHDWRAS |
| SEQ ID NO: 86 | MLSTKKTKTHDHYPSGRMRDPPWHDWRAS |
| SEQ ID NO: 87 | MLSTKKTKTHDHYPSGRMRDPGWHPWRAS |
| SEQ ID NO: 88 | MLSTKKTKTHDHYPSGRMRDPGWHRWRAS |
| SEQ ID NO: 89 | MLSTKKTKTHDHYPSGRMRDPGWHDWWAS |
| SEQ ID NO: 90 | MLSTKKTKTHDHYPSGRMRDPGWHDWRFS |
| SEQ ID NO: 91 | MLSTKKTKTHDHYPSGRMRDPGWHDWRGS |
| SEQ ID NO: 92 | MLSTKKTKTHDHYPSGRMRDPGWHDWRVS |
| SEQ ID NO: 93 | MLSTKKTKTHDHYPSGRMRDPGWHDWRYS |
| SEQ ID NO: 94 | SLSTKKTKTHDHYPSGRMRDPGWHDWRAS |
| SEQ ID NO: 95 | VLSTKKTKTHDHYPSGRMRDPGWHDWRAS |
| SEQ ID NO: 96 | WLSTKKTKTHDHYPSGRMRDPGWHDWRAS |
| SEQ ID NO: 97 | MFSTKKTKTHDHYPSGRMRDPGWHDWRAS |
| SEQ ID NO: 98 | MNSTKKTKTHDHYPSGRMRDPGWHDWRAS |
| SEQ ID NO: 99 | MVSTKKTKTHDHYPSGRMRDPGWHDWRAS |
| SEQ ID NO: 100 | MLSAKKTKTHDHYPSGRMRDPGWHDWRAS |
| SEQ ID NO: 101 | MLSGKKTKTHDHYPSGRMRDPGWHDWRAS |
| SEQ ID NO: 102 | MLSTAKTKTHDHYPSGRMRDPGWHDWRAS |
| SEQ ID NO: 103 | MLSTKKGKTHDHYPSGRMRDPGWHDWRAS |
| SEQ ID NO: 104 | MLSTKKTKWHDHYPSGRMRDPGWHDWRAS |
| SEQ ID NO: 105 | MLSTKKTKTHDHNPSGRMRDPGWHDWRAS |
| SEQ ID NO: 106 | MLSTKKTKTHDHYGSGRMRDPGWHDWRAS |
| SEQ ID NO: 107 | MLSTKKTKTHDHYWSGRMRDPGWHDWRAS |
| SEQ ID NO: 108 | MLSTKKTKTHDHYPGGRMRDPGWHDWRAS |
| SEQ ID NO: 109 | MLSTKKTKTHDHYPSGRYRDPGWHDWRAS |
| SEQ ID NO: 110 | MLSTKKTKTHDHYPSGRMRDFGWHDWRAS |
| SEQ ID NO: 111 | MLSTKKTKTHDHYPSGRMRDPKWHDWRAS |
| SEQ ID NO: 112 | MLSTKKTKTHDHYPSGRMRDPWWHDWRAS |
| SEQ ID NO: 113 | MLSTKKTKTHDHYPSGRMRDPGWHDWRWS |
| SEQ ID NO: 114 | MLSTKKTKTHDHYPSGRMRDPGWHDWRAR |

In some embodiments, variants of any one of SEQ ID NOs:2-114 according to the present disclosure can be a peptide with 1, 2, 3, 4, 5, 6, 7, 8, 9, 10, 11, 12, 13, 14, or 15 amino acid modifications relative to any one of SEQ ID NOs:2-114. In some embodiments, the variant can be at least 50%, 55%, 60%, 65%, 70%, 75%, 80%, 85%, 90%, 91%, 92%, 93%, 94%, 95%, 96%, 97%, or 98% identical to any one of SEQ ID NOs:2-114

Also provided herein are peptides comprising the amino acid sequence set forth in SEQ ID NO:115, where Xₓ represents any naturally-occurring amino acid (See Table 3).

TABLE 3

| Sequence | |
|---|---|
| Identifier | Amino Acid Sequence |
| SEQ ID | X₁X₂X₃X₄X₅X₆X₇KX₈X₉X₁₀X₁₁X₁₂X₁₃X₁₄GRX₁₅R |
| NO: 115 | X₁₆X₁₇X₁₈WHX₁₉WX₂₀X₂₁X₂₂ |

In some embodiments, X₁ is an amino acid selected from the group consisting of: M, W, F, V, P, K, R, S, and V. In some embodiments, X₁ is an amino acid selected from the group consisting of: M, F, K, P, and R. In some embodiments, X₁ is an amino acid selected from the group consisting of: M, S, V, and W. In some embodiments, X₁ is an amino acid selected from the group consisting of: M, W, F, V, and P.

In some embodiments, X₂ is an amino acid selected from the group consisting of: L, S, P, G, T, V, A, K, Q, R, W, Y, F, and N. In some embodiments, X₂ is an amino acid selected from the group consisting of: M, F, K, P, and R. In some embodiments, X₂ is an amino acid selected from the group consisting of: M, S, V, and W. In some embodiments, X₂ is an amino acid selected from the group consisting of: L, S, P, G, T, V, and A.

In some embodiments, X₃ is an amino acid selected from the group consisting of: S, Q, and R. In some embodiments, X₃ is S.

In some embodiments, X₄ is an amino acid selected from the group consisting of: T, G, R, and A. In some embodiments, X₄ is an amino acid selected from the group consisting of: T and R. In some embodiments, X₄ is an amino acid selected from the group consisting of: T, G, and A.

In some embodiments, X₅ is an amino acid selected from the group consisting of: K, A, and R. In some embodiments, X₅ is an amino acid selected from the group consisting of: K and R. In some embodiments, X₅ is A.

In some embodiments, X₆ is an amino acid selected from the group consisting of: K, R, T, and G. In some embodiments, X₆ is an amino acid selected from the group consisting of: K, R, and T. In some embodiments, X₆ is an amino acid selected from the group consisting of: K and A. In some embodiments, X₆ is K.

In some embodiments, X₇ is an amino acid selected from the group consisting of: T, G, K, and R. In some embodiments, X₇ is an amino acid selected from the group consisting of: K, R, and T. In some embodiments, X₇ is an amino acid selected from the group consisting of: T and G.

In some embodiments, X₈ is an amino acid selected from the group consisting of: T, W, and R. In some embodiments, X₈ is an amino acid selected from the group consisting of: T and W. In some embodiments, X₈ is an amino acid selected from the group consisting of: T and R.

In some embodiments, X₉ is an amino acid selected from the group consisting of: H, K, and R. In some embodiments, X₉ is H.

In some embodiments, X₁₀ is an amino acid selected from the group consisting of: D, F, G, H, I, K, P, R, T, V, W, and Y. In some embodiments, X₁₀ is D.

In some embodiments, X₁₁ is an amino acid selected from the group consisting of: H, K, and R. In some embodiments, X₁₁ is H.

In some embodiments, X₁₂ is an amino acid selected from the group consisting of: Y, W, N, G, K, and R. In some embodiments, X₁₂ is an amino acid selected from the group consisting of: Y, G, K, R, and W. In some embodiments, X₁₂ is an amino acid selected from the group consisting of: Y, W, and N. In some embodiments, X₁₂ is an amino acid selected from the group consisting of: Y and N.

In some embodiments, X₁₃ is an amino acid selected from the group consisting of: P, G, and W. In some embodiments, X₁₃ is P.

In some embodiments, X₁₄ is an amino acid selected from the group consisting of: S, T, and R. In some embodiments, X₁₄ is an amino acid selected from the group consisting of: S and R. In some embodiments, X₁₄ is an amino acid selected from the group consisting of: S and G.

In some embodiments, X₁₅ is an amino acid selected from the group consisting of: M, W, H, Y, G, and R. In some embodiments, X₁₅ is an amino acid selected from the group consisting of: M, G, H, K, R, and W. In some embodiments, X₁₅ is an amino acid selected from the group consisting of: M, W, H, and Y. In some embodiments, X₁₅ is an amino acid selected from the group consisting of: M and Y.

In some embodiments, X₁₆ is an amino acid selected from the group consisting of: D, P, and R. In some embodiments, X₁₆ is an amino acid selected from the group consisting of: D and P. In some embodiments, X₁₆ is D.

In some embodiments, X₁₇ is an amino acid selected from the group consisting of: P and F. In some embodiments, X₁₇ is P.

In some embodiments, X₁₈ is an amino acid selected from the group consisting of: G, P, K, and W. In some embodiments, X₁₈ is an amino acid selected from the group consisting of: G, K, and W. In some embodiments, X₁₈ is an amino acid selected from the group consisting of: P and G. In some embodiments, X₁₈ is an amino acid selected from the group consisting of: G, P, and K.

In some embodiments, X₁₉ is an amino acid selected from the group consisting of: D, P, and R. In some embodiments, X₁₉ is D.

In some embodiments, X₂₀ is an amino acid selected from the group consisting of: R and W. In some embodiments, X₂₀ is R.

In some embodiments, X₂₁ is an amino acid selected from the group consisting of: F, G, V, A, Y, and W. In some embodiments, X₂₁ is an amino acid selected from the group consisting of: A, F, G, V, and Y. In some embodiments, X₂₁ is an amino acid selected from the group consisting of: A and W. In some embodiments, X₂₁ is an amino acid selected from the group consisting of: F, G, V, and A.

In some embodiments, X₂₂ is an amino acid selected from the group consisting of: S and R. In some embodiments, X₂₂ is S.

In some embodiments, provided herein are peptides comprising the amino acid sequence set forth in SEQ ID NO:116, where Xₓ represents any naturally-occurring amino acid (See Table 4).

TABLE 4

| Sequence | |
|---|---|
| Identifier | Amino Acid Sequence |
| SEQ ID NO: 116 | X₁X₂SX₄AKX₇KX₈HDHX₁₂X₁₃X₁₄GR |
| | X₁₅RX₁₆PX₁₈WHDWX₂₀X₂₁X₂₂ |

In some embodiments, X₁ is an amino acid selected from the group consisting of: M, W, F, V, and P.

In some embodiments, X₂ is an amino acid selected from the group consisting of: L, S, P, G, T, V, and A.

In some embodiments, X₄ is an amino acid selected from the group consisting of: T, G, and A.

In some embodiments, X₇ is an amino acid selected from the group consisting of: T and G.

In some embodiments, X₈ is an amino acid selected from the group consisting of: T and W.

In some embodiments, X₁₂ is an amino acid selected from the group consisting of: Y, W, and N.

In some embodiments, X₁₃ is an amino acid selected from the group consisting of: P, G, and W.

In some embodiments, X₁₄ is an amino acid selected from the group consisting of: S and G.

In some embodiments, X₁₅ is an amino acid selected from the group consisting of: M, W, H, and Y.

In some embodiments, X₁₆ is an amino acid selected from the group consisting of: D and P.

In some embodiments, X₁₈ is an amino acid selected from the group consisting of: G, P, and K.

In some embodiments, X₂₀ is an amino acid selected from the group consisting of: R and W.

In some embodiments, X₂₁ is an amino acid selected from the group consisting of: F, G, V, and A.

In some embodiments, X₂₂ is an amino acid selected from the group consisting of: S and R.

In some embodiments, a peptide (e.g., peptide having the amino acid sequence of SEQ ID NO:1 or a variant thereof) modulates the activity of one or more of a CD2 protein, a BST2 protein, or a TNF protein relative to the activity of the protein in a patient or cell not treated with the peptide. In some embodiments, a peptide (e.g., peptide having the amino acid sequence of SEQ ID NO:1 or a variant thereof) increases activity of one or more of a CD2 protein, a BST2 protein, or a TNF protein relative to the activity of the protein in a patient or cell not treated with the peptide.

In some embodiments, a peptide (e.g., peptide having the amino acid sequence of SEQ ID NO:1 or a variant thereof) binds to one or more of a CD2 protein, a BST2 protein, or a TNF protein. Binding may help modulate (e.g. increase or decrease) the activity or function of the protein.

In some embodiments, provided herein are peptides comprising the amino acid sequence set forth in SEQ ID NO:117 (See Table 5).

TABLE 5

| Sequence | |
|---|---|
| Identifier | Amino Acid Sequence |
| SEQ ID NO: 117 | MKVRPSVKPMCEKCKIIRRKGRVMVICE |
| | NPKHKQRQG |

A variant of SEQ ID NO:117 according to the present disclosure can be a peptide with 1, 2, 3, 4, 5, 6, 7, 8, 9, 10, 11, 12, 13, 14, or 15 amino acid modifications relative to SEQ ID NO:117. In some embodiments, the variant can be at least 50%, 55%, 60%, 65%, 70%, 75%, 80%, 85%, 90%, 91%, 92%, 93%, 94%, 95%, 96%, 97%, or 98% identical to SEQ ID NO:117. Examples of amino acid modifications with respect to the amino acid sequence set forth in SEQ ID NO:117, include, without limitation, amino acid substitutions, amino acid deletions, and amino acid insertions. In some embodiments, a peptide of the present disclosure has a deletion of 1, 2, 3, 4 or 5 N- or C-terminal residues of SEQ ID NO:117. Alternatively or additionally, there is an internal deletion of 1, 2, 3, 4, or 5 amino acids relative to SEQ ID NO:117.

An amino acid substitution of SEQ ID NO:117 can be a conservative amino acid substitution. For example, conservative amino acid substitutions can be made by substituting one amino acid residue for another amino acid residue having a similar side chain.

In some embodiments, an amino acid substitution of SEQ ID NO:117 is a non-conservative amino acid substitution. Non-conservative amino acid substitutions can be made by substituting one amino acid residue for another amino acid residue having a dissimilar side chain.

In some embodiments, the methionine at position at 1 of SEQ ID NO:117 is substituted with another amino acid (e.g., any of the other naturally-occurring amino acids).

In some embodiments, the valine at position at 3 of SEQ ID NO:117 is substituted with another amino acid (e.g., any of the other naturally-occurring amino acids). In some embodiments, the valine at position at 3 of SEQ ID NO:117 is substituted with an amino acid selected from the group consisting of: I and T.

In some embodiments, the arginine at position at 4 of SEQ ID NO:117 is substituted with another amino acid (e.g., any of the other naturally-occurring amino acids). In some embodiments, the arginine at position at 4 of SEQ ID NO:117 is substituted with an amino acid selected from the group consisting of: K and Q.

In some embodiments, the proline at position at 5 of SEQ ID NO:117 is substituted with another amino acid (e.g., any of the other naturally-occurring amino acids). In some embodiments, the proline at position at 5 of SEQ ID NO:117 is substituted with an amino acid selected from the group consisting of: S and A.

In some embodiments, the proline at position at 9 of SEQ ID NO:117 is substituted with another amino acid (e.g., any of the other naturally-occurring amino acids). In some embodiments, the proline at position at 9 of SEQ ID NO:117 is substituted with an amino acid selected from the group consisting of: T and K.

In some embodiments, the methionine at position at 10 of SEQ ID NO:117 is substituted with another amino acid (e.g., any of the other naturally-occurring amino acids). In some embodiments, the methionine at position at 10 of SEQ ID NO:117 is substituted with the amino acid I.

In some embodiments, the glutamic acid at position at 12 of SEQ ID NO:117 is substituted with another amino acid (e.g., any of the other naturally-occurring amino acids). In some embodiments, the glutamic acid at position at 12 of SEQ ID NO:117 is substituted with the amino acid D.

In some embodiments, the lysine at position at 13 of SEQ ID NO:117 is substituted with another amino acid (e.g., any of the other naturally-occurring amino acids). In some embodiments, the lysine at position at 13 of SEQ ID NO:117 is substituted with the amino acid Y.

In some embodiments, the lysine at position at 15 of SEQ ID NO:117 is substituted with another amino acid (e.g., any of the other naturally-occurring amino acids). In some embodiments, the lysine at position at 15 of SEQ ID NO:117 is substituted with the amino acid R.

In some embodiments, the valine at position at 16 of SEQ ID NO:117 is substituted with another amino acid (e.g., any of the other naturally-occurring amino acids). In some embodiments, the valine at position at 16 of SEQ ID NO:117 is substituted with the amino acid I.

In some embodiments, the lysine at position at 18 of SEQ ID NO:117 is substituted with another amino acid (e.g., any of the other naturally-occurring amino acids). In some embodiments, the lysine at position at 18 of SEQ ID NO:117 is substituted with the amino acid R.

In some embodiments, the lysine at position at 20 of SEQ ID NO:117 is substituted with another amino acid (e.g., any of the other naturally-occurring amino acids). In some embodiments, the lysine at position at 20 of SEQ ID NO:117 is substituted with an amino acid selected from the group consisting of: N and H.

In some embodiments, the arginine at position at 22 of SEQ ID NO:117 is substituted with another amino acid (e.g., any of the other naturally-occurring amino acids). In some embodiments, the arginine at position at 22 of SEQ ID NO:117 is substituted with an amino acid selected from the group consisting of: K, H, S, and I.

In some embodiments, the valine at position at 23 of SEQ ID NO:117 is substituted with another amino acid (e.g., any of the other naturally-occurring amino acids). In some embodiments, the valine at position at 23 of SEQ ID NO:117 is substituted with the amino acid I.

In some embodiments, the methionine at position at 24 of SEQ ID NO:117 is substituted with another amino acid (e.g., any of the other naturally-occurring amino acids). In some embodiments, the methionine at position at 24 of SEQ ID NO:117 is substituted with an amino acid selected from the group consisting of: R, A, and L.

In some embodiments, the valine at position at 25 of SEQ ID NO:117 is substituted with another amino acid (e.g., any of the other naturally-occurring amino acids). In some embodiments, the valine at position at 25 of SEQ ID NO:117 is substituted with the amino acid I.

In some embodiments, the glutamic acid at position at 28 of SEQ ID NO:117 is substituted with another amino acid (e.g., any of the other naturally-occurring amino acids). In some embodiments, the glutamic acid at position at 28 of SEQ ID NO:117 is substituted with an amino acid selected from the group consisting of: Q, A, and T.

In some embodiments, the asparagine at position at 29 of SEQ ID NO:117 is substituted with another amino acid (e.g., any of the other naturally-occurring amino acids). In some embodiments, the asparagine at position at 29 of SEQ ID NO:117 is substituted with the amino acid E.

In some embodiments, the lysine at position at 31 of SEQ ID NO:117 is substituted with another amino acid (e.g., any of the other naturally-occurring amino acids). In some embodiments, the lysine at position at 31 of SEQ ID NO:117 is substituted with the amino acid R.

In some embodiments, the lysine at position at 35 of SEQ ID NO:117 is substituted with another amino acid (e.g., any of the other naturally-occurring amino acids). In some embodiments, the lysine at position at 35 of SEQ ID NO:117 is substituted with the amino acid R.

In some embodiments, none of the amino acids of SEQ ID NO:117 are substituted with cysteine.

In some embodiments, a peptide described herein (e.g., peptide having the amino acid sequence of SEQ ID NO:117 or 162 or a variant thereof) modulates activity of a CXCR3 protein. In some cases, a peptide described herein (e.g., peptide having the amino acid sequence of SEQ ID NO:117 or 162 or a variant thereof) increases activity of a CXCR3 protein. In some embodiments, a peptide modulates activity of a CCR9 protein, a CHRM5 protein, a CXCR3 protein, a CXCR4 protein, a HCRTR2 protein, a MRGPRX2 protein, a SSTR1 protein, or a TSHR(L) protein.

In some embodiments, a peptide described herein (e.g., a peptide having the amino acid sequence of SEQ ID NO:117 or 162 or a variant thereof) binds to of a CCR9 protein, a CHRM5 protein, a CXCR3 protein, a CXCR4 protein, a HCRTR2 protein, a MRGPRX2 protein, a SSTR1 protein, or a TSHR(L) protein. In some embodiments, a peptide described herein (e.g., a peptide having the amino acid sequence of SEQ ID NO:117 or 162 or a variant thereof) binds to a CXCR3 protein.

In some embodiments, a peptide described herein has an amino acid sequence selected from Table 6, where Xₓ represents any naturally-occurring amino acid.

TABLE 6

| Sequence | |
|---|---|
| Identifier | Amino Acid Sequence |
| SEQ ID NO: 117 | MKVRPSVKPMCEKCKIIRRKGRVMVICENPKHKQRQG |
| SEQ ID NO: 118 | MKVRPSVKPICEKCKIIRRKGRVMVICENPKHKQKQG |
| SEQ ID NO: 119 | MKVRPSVKPMCEKCKIIKRKGKVMVICENPKHKQRQG |
| SEQ ID NO: 120 | MKVRPSVKPMCEKCKIIKRKGKVMVICENPKHKQKQG |
| SEQ ID NO: 121 | MKVRPSVKPICEKCKIIRRKGRVMVICQNPKHKQKQG |
| SEQ ID NO: 122 | MKVRPSVKPICEKCKIIKRKGRVMVICENPKHKQKQG |
| SEQ ID NO: 123 | MKVRPSVKPMCEKCKVIKRKGKVMVICENPKHKQRQG |
| SEQ ID NO: 124 | MKVRPSVKPMCEKCKVIKRKGRVMVICENPKHKQKQG |
| SEQ ID NO: 125 | MKVRPSVKPICEKCKVIRRKGRVMVICENPKHKQKQG |
| SEQ ID NO: 126 | MKVKPSVKPICEKCKIIKRKGRVMVICENPKHKQKQG |
| SEQ ID NO: 127 | MKVRPSVKPMCEKCKVIKRKGKVMVICENPKHKQKQG |
| SEQ ID NO: 128 | MKVRPSVKKICEKCKIIKRKGRVMVICENPKHKQKQG |
| SEQ ID NO: 129 | MKIRPSVKPMCEKCRIIKRKGRVMVICENPKHKQKQG |
| SEQ ID NO: 130 | MKVRPSVKPICEKCKVIRRKGKVMVICENPKHKQKQG |
| SEQ ID NO: 131 | MKVRPSVKPICEKCKIIKRKGKVMVICENPKHKQKQG |
| SEQ ID NO: 132 | MKVRPSVKPICEKCKIIRRKGKVMVICQNPKHKQKQG |
| SEQ ID NO: 133 | MKVRPSVKPICEKCKVIKRKGRVMVICENPKHKQKQG |
| SEQ ID NO: 134 | MKVKPSVKTICEKCKIIRRKGRVMVICENPKHKQKQG |
| SEQ ID NO: 135 | MKVRPSVKPICEKCKVIKRKGKVMVICENPKHKQRQG |
| SEQ ID NO: 136 | MKVRPSVKPICDKCRIIKRKGRVMVICENPKHKQRQGN |
| SEQ ID NO: 137 | MKVKPSVKPICEKCKVIRRKGRVMVICQNPKHKQRQG |
| SEQ ID NO: 138 | MKVKPSVKTICEKCKIIKRKGRVMVICENPKHKQKQG |
| SEQ ID NO: 139 | MKVQPSVKKICEKCKIIKRKGRVMVICENPKHKQKQG |
| SEQ ID NO: 140 | MKVRSSVKPICEKCKIIRRKGSIRVICENPKHKQRQG |
| SEQ ID NO: 141 | MKVKPSVKPICEKCKVIKRKGRVMVICQNPKHKQRQG |
| SEQ ID NO: 142 | MKVRSSVKPICEKCKIIKRKGRIRVICENPKHKQRQG |
| SEQ ID NO: 143 | MKVRPSVKPICEKCKVIKRKGKVMVICENPKHKQKQG |
| SEQ ID NO: 144 | MKVKPSVKKICEKCKIIKRKGRVMVICENPKHKQKQG |
| SEQ ID NO: 145 | MKVRPSVKPICEKCKVIKRKGKVMVICQNPKHKQRQG |
| SEQ ID NO: 146 | MKVKPSVKTICEKCKIIRRKGRVMIICENPKHKQKQG |
| SEQ ID NO: 147 | MKVRPSVKPICEKCKVIKRKGHVMVICENPKHKQKQG |
| SEQ ID NO: 148 | MKVRPSVKPICDKCRVIKRKGRVMVICENPKHKQRQG |
| SEQ ID NO: 149 | MKVRSSVKPICEKCKIIRRKGSIRVICENPKHKQRQDRH |
| SEQ ID NO: 150 | MKVRPSVKPICEYCKVIRRNGRVMVICPTNPKHKQRQG |
| SEQ ID NO: 151 | MKVRSSVKPICEKCKIIKRKGSIRVICENPKHKQRQG |
| SEQ ID NO: 152 | MKVKPSVKPICEKCKVIKRKGRVMIICANPKHKQRQG |
| SEQ ID NO: 153 | MKVKPSVKTICEKCKIIKRKGRVMIICENPKHKQKQG |
| SEQ ID NO: 154 | MKTRSSVKPMCDKCKVIKRKGRVAVICENPKHKQRQG |
| SEQ ID NO: 155 | MKVRPSVKPMCDKCKVIKRKGKVMVICQEPKHKQRQG |
| SEQ ID NO: 156 | KVRSSVKPICEKCKVIKRKGIVRVICENPKHKQRQG |
| SEQ ID NO: 157 | MKVRASVKPICDKCKVIKRKGIVRVICENPKHKQRQG |
| SEQ ID NO: 158 | MKVRPSVKKMCDKCKIIRRHGKILVICENPRHKQRQG |
| SEQ ID NO: 159 | MKVRPSVKKMCDKCKVIKRKGKILVICENPKHKQRQG |
| SEQ ID NO: 160 | MKVRSSVKPICEKCKVIKRKGSVRIICENPKHKQRQG |
| SEQ ID NO: 161 | X₁KX₃X₄X₅SVKX₉X₁₀CX₁₂X₁₃CX₁₄X₁₅X₁₆IX₁₈RX₂₀GX₂₂X₂₃X₂₄X₂₅IX₂₇ |
| | X₂₈X₂₉P X₃₁HKQX₃₅QX₃₇ |

In some embodiments, X₁ of SEQ ID NO:161 is the amino acid M.

In some embodiments, X₃ of SEQ ID NO:161 is an amino acid selected from the group consisting of: V, I, and T.

In some embodiments, X₄ of SEQ ID NO:161 is an amino acid selected from the group consisting of: R, K, and Q.

In some embodiments, X₅ of SEQ ID NO:161 is an amino acid selected from the group consisting of: P, S, and A.

In some embodiments, X₉ of SEQ ID NO:161 is an amino acid selected from the group consisting of: P, T, and K.

In some embodiments, X₁₀ of SEQ ID NO:161 is an amino acid selected from the group consisting of: M and I.

In some embodiments, X₁₂ of SEQ ID NO:161 is an amino acid selected from the group consisting of: E and D.

In some embodiments, X₁₃ of SEQ ID NO:161 is an amino acid selected from the group consisting of: K and Y.

In some embodiments, X₁₅ of SEQ ID NO:161 is an amino acid selected from the group consisting of: K and R.

In some embodiments, X₁₆ of SEQ ID NO:161 is an amino acid selected from the group consisting of: V and I.

In some embodiments, X₁₈ of SEQ ID NO:161 is an amino acid selected from the group consisting of: K and R.

In some embodiments, X₂₀ of SEQ ID NO:161 is an amino acid selected from the group consisting of: K, N, and H.

In some embodiments, X₂₂ of SEQ ID NO:161 is an amino acid selected from the group consisting of: R, K, H, S, and I.

In some embodiments, X₂₃ of SEQ ID NO:161 is an amino acid selected from the group consisting of: V and I.

In some embodiments, X₂₄ of SEQ ID NO:161 is an amino acid selected from the group consisting of: M, R, A, and L.

In some embodiments, X₂₅ of SEQ ID NO:161 is an amino acid selected from the group consisting of: V and I.

In some embodiments, X₂₈ of SEQ ID NO:161 is substituted with an amino acid selected from the group consisting of: E, Q, A, and T.

In some embodiments, X₂₉ of SEQ ID NO:161 is substituted with an amino acid selected from the group consisting of: N and E.

In some embodiments, X₃₁ of SEQ ID NO:161 is substituted with an amino acid selected from the group consisting of: K and R.

In some embodiments, X₃₅ of SEQ ID NO:161 is substituted with an amino acid selected from the group consisting of: K and R.

In some embodiments, provided herein are peptides comprising the amino acid sequence set forth in SEQ ID NO:162 (See Table 7).

TABLE 7

| Sequence | |
|---|---|
| Identifier | Amino Acid Sequence |
| SEQ ID NO: 162 | MKVRPSVKPMCEKCKIIRRKGRVMVICENPKH |
| | KQRQG |

A variant of SEQ ID NO:162 according to the present disclosure can be a peptide with 1, 2, 3, 4, 5, 6, 7, 8, 9, 10, 11, 12, 13, 14, or 15 amino acid modifications relative to SEQ ID NO:162. In some embodiments, the variant can be at least 50%, 55%, 60%, 65%, 70%, 75%, 80%, 85%, 90%, 91%, 92%, 93%, 94%, 95%, 96%, 97%, or 98% identical to SEQ ID NO:162. Examples of amino acid modifications with respect to the amino acid sequence set forth in SEQ ID NO:162, include, without limitation, amino acid substitutions, amino acid deletions, and amino acid insertions. In some embodiments, a peptide of the present disclosure has a deletion of 1, 2, 3, 4 or 5 N- or C-terminal residues of SEQ ID NO:162. Alternatively or additionally, there is an internal deletion of 1, 2, 3, 4, or 5 amino acids relative to SEQ ID NO:162.

An amino acid substitution of SEQ ID NO:162 can be a conservative amino acid substitution. For example, conservative amino acid substitutions can be made by substituting one amino acid residue for another amino acid residue having a similar side chain.

In some embodiments, an amino acid substitution of SEQ ID NO:162 is a non-conservative amino acid substitution. Non-conservative amino acid substitutions can be made by substituting one amino acid residue for another amino acid residue having a dissimilar side chain.

In some embodiments, a peptide described herein (e.g., a peptide having the amino acid sequence of SEQ ID NO:1, 117 or 162 or a variant thereof) can be modified. For example, an acetyl (Ac) and/or or an amide group can be added at the C- and/or N-terminus of the peptide. In some embodiments, the peptide is “cyclized,” referring to a reaction in which one part of a polypeptide or peptide molecule becomes linked to another part of the polypeptide or peptide molecule to form a closed ring, such as by forming a disulfide bridge or other similar bond. In some embodiments, a peptide described herein (e.g., a peptide having the amino acid sequence of SEQ ID NO:1, 117 or 162 or a variant thereof) is linked to another molecule, e.g., protein (e.g., BSA or Fc domain) or stabilizing group such as a PEG molecule, by a linker. A “linker moiety,” as used herein, refers broadly to a chemical structure that is capable of linking or joining together two peptide monomer subunits to form a dimer.

In some embodiments, a peptide of the present disclosure can be modified to increase the solubility of the peptide in an aqueous solution, relative to the unmodified peptide. In some embodiments, a peptide of the present disclosure can be modified to decrease the solubility of the peptide in an aqueous solution, relative to the unmodified peptide. In some embodiments, a peptide of the present disclosure can be modified to increase the solubility of the peptide in a polar solvent, relative to the unmodified peptide. In some embodiments, a peptide of the present disclosure can be modified to decrease the solubility of the peptide in a polar solvent, relative to the unmodified peptide. In some embodiments, a peptide of the present disclosure can be modified to increase the solubility of the peptide in a non-polar solvent, relative to the unmodified peptide. In some embodiments, a peptide of the present disclosure can be modified to decrease the solubility of the peptide in a non-polar solvent, relative to the unmodified peptide.

In some embodiments, a peptide of the present disclosure can be modified to increase the net charge of the peptide at human physiological pH, relative to the unmodified peptide at human physiological pH. In some embodiments, a peptide of the present disclosure can be modified to decrease the net charge of the peptide at human physiological pH, relative to the unmodified peptide at human physiological pH.

In some embodiments, a peptide described herein can have a post-translational modification (PTM). Protein PTMs (e.g., peptide PTMs) occur in vivo and can increase the functional diversity of the proteome by the covalent addition of functional groups or proteins, proteolytic cleavage of regulatory subunits or degradation of entire proteins. Isolated peptides prepared according to the present disclosure can undergo one or more PTMs in vivo or in vitro. The type of modification(s) depends on host cell in which the peptide is expressed and includes but is not limited to phosphorylation, glycosylation, ubiquitination, nitrosylation (e.g., S-nitrosylation), methylation, acetylation (e.g., N-acetylation), lipidation (myristoylation, N-myristoylation, S-palmitoylation, farnesylation, S-prenylation, S-palmitoylation) and proteolysis can influence aspects of normal cell biology and pathogenesis. The peptides as disclosed herein can comprise one or more the above recited post-translational modifications.

### Methods of Use

Provided herein are methods for identifying a subject as having a decreased likelihood of positively responding to treatment with an immunomodulator (e.g., an immune checkpoint inhibitor therapy and/or a co-stimulatory immune checkpoint therapy). For example, the subject may have a sample (e.g., a fecal sample) that has a decreased level of the expression of dgoD, graR, or both relative to the same in a reference sample; a decreased level of activity of a trans-2-enoyl-CoA reductase, a tetrose transporter (e.g., an Acinetobacter tetrose transporter), or both relative to the same in a reference sample; a decreased flux through the B-ureidopropionase reaction relative to the same in a reference sample; an increased level of one or more bacterial species selected from the group consisting of: Clostridium clostridioforme, Prevotella sp., Streptococcus parasanguinis, Anaerostipes hadrus, Parasutterella excrementihominis, and Eisenbergiella massiliensis relative to the same in a reference sample; or a decreased level of one or more bacterial species selected from the group consisting of: Bifidobacterium sp., Collinsella sp., Methanobrevibacter smithii, Oscillibacter sp., Faecalibacterium prausnitzii C, Faecalibacterium prausnitzii I, Intestinimonas timonensis, Faecalibacterium prausnitzii, Bacteroides caccae, Barnesiella intestinihominis, Clostridiaceae bacterium, Clostridium sp., and Bifidobacterium adolescentis relative to the same in a reference sample is identified as having a decreased likelihood of having a positive response, or is less likely to respond to treatment with an immunomodulator.

In any of the methods provided herein the subject may have or may previously been identified as having a sample (e.g., a fecal sample) can include a subject that has a decreased level of the expression of dgoD, graR, or both relative to the same in a reference sample; a decreased level of activity of a trans-2-enoyl-CoA reductase, a tetrose transporter (e.g., an Acinetobacter tetrose transporter), or both relative to the same in a reference sample; a decreased flux through the B-ureidopropionase reaction relative to the same in a reference sample; or a decreased level of Faecalibacterium prausnitzii relative to the same in a reference sample.

In any of the methods provided herein, the subject may have or may previously been identified as having a sample (e.g., a fecal sample) that has a decreased level of the expression of dgoD relative to the same in a reference sample. In some embodiments, the subject may have or may previously been identified as having a sample (e.g., a fecal sample) that has a decreased level of the expression of graR relative to the same in a reference sample. In some embodiments, the subject may have or may previously been identified as having a sample (e.g., a fecal sample) that has a decreased level of activity of a trans-2-enoyl-CoA reductase relative to the same in a reference sample. In some embodiments, the subject may have or may previously been identified as having a sample (e.g., a fecal sample) that has a decreased level of activity of a tetrose transporter (e.g., an Acinetobacter tetrose transporter) relative to the same in a reference sample. In some embodiments, the subject may have or may previously been identified as having a sample (e.g., a fecal sample) that has a decreased flux through the B-ureidopropionase reaction relative to the same in a reference sample. In some embodiments, the subject may have or may previously been identified as having a sample (e.g., a fecal sample) that has a decreased level of Faecalibacterium prausnitzii relative to the same in a reference sample.

Also provided herein are methods for identifying a subject likely to respond to therapy with an immunomodulator (e.g., an immune checkpoint inhibitor therapy and/or a co-stimulatory immune checkpoint therapy). For example, a subject with a sample (e.g., a fecal sample) with an increased level of expression of genes, for example, the dgoD or graR genes, relative to the same in a reference sample, an increased level of activity of a trans-2-enoyl-CoA reductase, a tetrose transporter (e.g., an Acinetobacter tetrose transporter), or both relative to the same in a reference sample, an increased flux through the B-ureidopropionase reaction relative to the same in a reference sample, a decreased level of one or more bacterial species selected from the group consisting of: Clostridium clostridioforme, Prevotella sp., Streptococcus parasanguinis, Anaerostipes hadrus, Parasutterella excrementihominis, or Eisenbergiella massiliensis relative to the same in a reference sample, or an increased level of one or more bacterial species selected from the group consisting of: Bifidobacterium sp., Collinsella sp., Methanobrevibacter smithii, Oscillibacter sp., Faecalibacterium prausnitzii C, Faecalibacterium prausnitzii I, Intestinimonas timonensis, Faecalibacterium prausnitzii, Bacteroides caccae, Barnesiella intestinihominis, Clostridiaceae bacterium, Clostridium sp., and Bifidobacterium adolescentis relative to the same in a reference sample may be identified as having an increased likelihood of having a positive response to treatment with an immunomodulator.

In any of the methods provided herein, the subject can have or may previously been identified as having a sample (e.g., a fecal sample) with an increased level of expression of genes, for example, the dgoD or graR genes, relative to the same in a reference sample, an increased level of activity of a trans-2-enoyl-CoA reductase, a tetrose transporter (e.g., an Acinetobacter tetrose transporter), or both relative to the same in a reference sample, an increased flux through the B-ureidopropionase reaction relative to the same in a reference sample, or an increased level of Faecalibacterium prausnitzii relative to the same in a reference sample.

In any of the methods provided herein, the subject may have or may previously been identified as having a sample (e.g., a fecal sample) that has an increased level of expression of dgoD relative to the same in a reference sample. In some embodiments, the subject may have or may previously been identified as having a sample (e.g., a fecal sample) that has an increased level of expression of graR relative to the same in a reference sample. In some embodiments, the subject may have or may previously been identified as having a sample (e.g., a fecal sample) that has an increased level of activity of a trans-2-enoyl-CoA reductase relative to the same in a reference sample. In some embodiments, the subject may have or may previously been identified as having a sample (e.g., a fecal sample) that has an increased level of activity of a tetrose transporter (e.g., an Acinetobacter tetrose transporter) relative to the same in a reference sample. In some embodiments, the subject may have or may previously been identified as having a sample (e.g., a fecal sample) that has an increased flux through the B-ureidopropionase reaction relative to the same in a reference sample. In some embodiments, the subject may have or may previously been identified as having a sample (e.g., a fecal sample) that has an increased level of Faecalibacterium prausnitzii relative to the same in a reference sample.

Also provided herein are methods for treating subjects in need thereof (e.g., subjects having an immunological disease and/or cancer) using one or more peptides described herein (e.g., a peptide having the amino acid sequence of SEQ ID NO:1, 117 or 162 or a variant thereof). Immunological diseases and cancers that can be treated using a peptide as described herein (e.g., a peptide having the amino acid sequence of SEQ ID NO:1, 117 or 162 or a variant thereof) can include diseases that are associated with inflammatory immune responses. In some embodiments, a peptide described herein (e.g., a peptide having the amino acid sequence of SEQ ID NO:1, 117 or 162 or a variant thereof) modulates immunoregulatory cells, including but not limited to T cells, effector T cells and dendritic cells. In some embodiments, the subject has a T-cell mediated disease. In some embodiments, the immunological disease and/or cancer includes those that are characterized by infiltration of inflammatory cells into a tissue, stimulation of T cell proliferation, inhibition of T cell proliferation, increased or decreased vascular permeability, or the inhibition thereof.

In some embodiments, the method uses one or more recombinant hosts. In some embodiments, the method uses one or more pharmaceutical compositions. In some embodiments, the method uses one or more nucleic acid constructs.

In some embodiments, administration of a peptide described herein (e.g., a peptide having the amino acid sequence of SEQ ID NO:1, 117 or 162 or a variant thereof) can prevent, reduce the severity of, or eliminate at least one symptom, of the disease or condition in the subject. The subject may be an animal. The subject may be a mammal. The subject may be a human subject.

In some embodiments, the disease or condition is cancer, e.g., any of the cancers described herein. In some embodiments, the disease or condition is autoimmune thyroiditis. In some embodiments, the disease or conditions is Hodgkin's lymphoma.

In some embodiments of the methods provided herein, a peptide described herein (e.g., a peptide having the amino acid sequence of SEQ ID NO:1, 117 or 162 or a variant thereof) can modulate the production of at least one cytokine in the subject. In some embodiments of the methods provided herein, a peptide described herein (e.g., a peptide having the amino acid sequence of SEQ ID NO:1, 117 or 162 or a variant thereof) can induce or increase the production of at least one pro-inflammatory cytokine (e.g., TNF-α and/or IL-23) by an immune cell (e.g., in an immune cell in a subject administered the peptide). In some embodiments of the methods provided herein, the peptide can suppress (e.g., prevent, inhibit, or decrease the production of) at least one anti-inflammatory cytokine (e.g., IL-10) by an immune cell (e.g., in an immune cell in a subject administered the peptide). Pro-inflammatory cytokines can include TNF-α, IL-17, IL-1β, IL-2, IFN-γ, IL-6, IL-12, IL-25, IL-33, IL-8, MCP-1, MIP-3α, CXCL1, and IL-23. Anti-inflammatory cytokines can include IL-4, IL-10, IL-13, IFN-α, and TGF-β.

In some embodiments, treatment with a peptide described herein can induce naïve T cells to differentiate. In some embodiments, treatment with a peptide described herein (e.g., a peptide having the amino acid sequence of SEQ ID NO:1, 117 or 162 or a variant thereof) can induce production of IFN-γ and IL-4 from T cells. In some embodiments, treatment with a peptide described herein (e.g., a peptide having the amino acid sequence of SEQ ID NO:1, 117 or 162 or a variant thereof) can induce maintained IFN-γ production from a variety of T cell subsets. In some embodiments, a peptide described herein (e.g., a peptide having the amino acid sequence of SEQ ID NO:1, 117 or 162 or a variant thereof) increases Th1 activation in a subject administered the peptide. In some embodiments, a peptide described herein (e.g., a peptide having the amino acid sequence of SEQ ID NO:1, 117 or 162 or a variant thereof) increases dendritic cell maturation, in a subject administered the peptide. In some embodiments, a peptide described herein increases the number of antigen-presenting T cell-priming cells. In some embodiments, a peptide described herein (e.g., a peptide having the amino acid sequence of SEQ ID NO:1, 117 or 162 or a variant thereof) can increase conversion of antigens into immunogens. In some embodiments, a peptide described herein (e.g., a peptide having the amino acid sequence of SEQ ID NO:1, 117 or 162 or a variant thereof) increases CD70 expression in a subject administered the peptide. In some embodiments, a peptide described herein (e.g., a peptide having the amino acid sequence of SEQ ID NO:1, 117 or 162 or a variant thereof) increases the clonal expansion of T_{eff}, in a subject administered the peptide.

In some embodiments, a peptide described herein increases one or more T cells selected from the group consisting of CD4+CD25+, CD4+PD-1+, CD4+ICOS+, CD4+OX40+, CD8+CD25+, CD8+PD-1+, CD8+ICOS+, and CD8+OX40+ in the subject. In some embodiments, a peptide described herein increases secretion of one or more cytokines selected from the group consisting of IFN-γ, IL-2, IL-10 and TNF-α in the subject.

In some embodiments of the methods provided herein, treatment with a peptide described herein (e.g., a peptide having the amino acid sequence of SEQ ID NO:1, 117 or 162 or a variant thereof) increases activity of a CD2 protein, a BST2 protein, or a TNF protein relative to the activity of the protein in a patient or cell not treated with the peptide. In some embodiments of the methods provided herein, treatment with a peptide described herein results in the peptide binding to a CD2 protein, a BST2 protein, or a TNF protein.

In some embodiments of the methods provided herein, treatment with a peptide described herein (e.g., a peptide having the amino acid sequence of SEQ ID NO:1, 117 or 162 or a variant thereof) modulates activity of a CXCR3 protein. In some embodiments of the methods provided herein, treatment with a peptide described herein increases activity of a CXCR3 protein. In some embodiments of the methods provided herein, treatment with a peptide described herein modulates activity of a CCR9 protein, a CHRM5 protein, a CXCR3 protein, a CXCR4 protein, a HCRTR2 protein, a MRGPRX2 protein, a SSTR1 protein, or a TSHR(L) protein. In some embodiments of the methods provided herein, treatment with a peptide described herein results in the peptide binding to a CCR9 protein, a CHRM5 protein, a CXCR3 protein, a CXCR4 protein, a HCRTR2 protein, a MRGPRX2 protein, a SSTR1 protein, or a TSHR(L) protein. In some embodiments of the methods provided herein, treatment with a peptide described herein results in the peptide binding to a CXCR3 protein.

Also described herein are methods of treating cancer in a subject that includes administering to a subject identified as having a decreased level of the expression of dgoD, graR, or both relative to the same in a reference sample; a decreased level of activity of a trans-2-enoyl-CoA reductase, an Acinetobacter tetrose transporter, or both relative to the same in a reference sample; a decreased flux through the B-ureidopropionase reaction relative to the same in a reference sample; an increased level of one or more bacterial species selected from the group consisting of: Clostridium clostridioforme, Prevotella sp., Streptococcus parasanguinis, Anaerostipes hadrus, Parasutterella excrementihominis, and Eisenbergiella massiliensis relative to the same in a reference sample; or a decreased level of one or more bacterial species selected from the group consisting of: Bifidobacterium sp., Collinsella sp., Methanobrevibacter smithii, Oscillibacter sp., Faecalibacterium prausnitzii C, Faecalibacterium prausnitzii I, Intestinimonas timonensis, Faecalibacterium prausnitzii, Bacteroides caccae, Barnesiella intestinihominis, Clostridiaceae bacterium, Clostridium sp., and Bifidobacterium adolescentis relative to the same in a reference sample a therapy.

Also provided herein are methods of treating cancer in a subject that has previously received one or more doses of an immunomodulator that include administering to a subject identified as having a decreased level of the expression of dgoD, graR, or both relative to the same in a reference sample; a decreased level of activity of a trans-2-enoyl-CoA reductase, an Acinetobacter tetrose transporter, or both relative to the same in a reference sample; a decreased flux through the B-ureidopropionase reaction relative to the same in a reference sample; an increased level of one or more bacterial species selected from the group consisting of: Clostridium clostridioforme, Prevotella sp., Streptococcus parasanguinis, Anaerostipes hadrus, Parasutterella excrementihominis, and Eisenbergiella massiliensis relative to the same in a reference sample; or a decreased level of one or more bacterial species selected from the group consisting of: Bifidobacterium sp., Collinsella sp., Methanobrevibacter smithii, Oscillibacter sp., Faecalibacterium prausnitzii C, Faecalibacterium prausnitzii I, Intestinimonas timonensis, Faecalibacterium prausnitzii, Bacteroides caccae, Barnesiella intestinihominis, Clostridiaceae bacterium, Clostridium sp., and Bifidobacterium adolescentis relative to the same in a reference sample a therapy.

Also provided herein are methods of treating cancer in a subject that include administering to the subject one or more doses of an immunomodulator for a period of time, determining, following administration of the one or more doses, if a sample obtained (e.g. a fecal sample) from the subject has a decreased level of the expression of dgoD, graR, or both relative to the same in a reference sample; a decreased level of activity of a trans-2-enoyl-CoA reductase, an Acinetobacter tetrose transporter, or both relative to the same in a reference sample; a decreased flux through the B-ureidopropionase reaction relative to the same in a reference sample; an increased level of one or more bacterial species selected from the group consisting of: Clostridium clostridioforme, Prevotella sp., Streptococcus parasanguinis, Anaerostipes hadrus, Parasutterella excrementihominis, and Eisenbergiella massiliensis relative to the same in a reference sample; or a decreased level of one or more bacterial species selected from the group consisting of: Bifidobacterium sp., Collinsella sp., Methanobrevibacter smithii, Oscillibacter sp., Faecalibacterium prausnitzii C, Faecalibacterium prausnitzii I, Intestinimonas timonensis, Faecalibacterium prausnitzii, Bacteroides caccae, Barnesiella intestinihominis, Clostridiaceae bacterium, Clostridium sp., and Bifidobacterium adolescentis relative to the same in a reference sample, and administering a therapy to the identified subject. In some embodiments, the determining if a sample from the subject has a decreased level of the expression of dgoD, graR, or both relative to the same in a reference sample; a decreased level of activity of a trans-2-enoyl-CoA reductase, an Acinetobacter tetrose transporter, or both relative to the same in a reference sample; a decreased flux through the B-ureidopropionase reaction relative to the same in a reference sample; an increased level of one or more bacterial species selected from the group consisting of: Clostridium clostridioforme, Prevotella sp., Streptococcus parasanguinis, Anaerostipes hadrus, Parasutterella excrementihominis, and Eisenbergiella massiliensis relative to the same in a reference sample; or a decreased level of one or more bacterial species selected from the group consisting of: Bifidobacterium sp., Collinsella sp., Methanobrevibacter smithii, Oscillibacter sp., Faecalibacterium prausnitzii C, Faecalibacterium prausnitzii I, Intestinimonas timonensis, Faecalibacterium prausnitzii, Bacteroides caccae, Barnesiella intestinihominis, Clostridiaceae bacterium, Clostridium sp., and Bifidobacterium adolescentis relative to the same in a reference sample occurs after the subject has received one or more doses of an immunomodulator.

Also provided herein are methods of treating cancer in a subject that include administering to the subject one or more doses of an immunomodulator for a period of time, determining if a sample obtained (e.g. a fecal sample) from the subject has an increased level of the expression of dgoD, graR, or both relative to the same in a reference sample, an increased level of activity of a trans-2-enoyl-CoA reductase, an Acinetobacter tetrose transporter, or both relative to the same in a reference sample, an increased flux through the B-ureidopropionase reaction relative to the same in a reference sample, a decreased level of one or more bacterial species selected from the group consisting of: Clostridium clostridioforme, Prevotella sp., Streptococcus parasanguinis, Anaerostipes hadrus, Parasutterella excrementihominis, and Eisenbergiella massiliensis relative to the same in a reference sample, and/or an increased level of one or more bacterial species selected from the group consisting of: Bifidobacterium sp., Collinsella sp., Methanobrevibacter smithii, Oscillibacter sp., Faecalibacterium prausnitzii C, Faecalibacterium prausnitzii I, Intestinimonas timonensis, Faecalibacterium prausnitzii, Bacteroides caccae, Barnesiella intestinihominis, Clostridiaceae bacterium, Clostridium sp., and Bifidobacterium adolescentis relative to the same in a reference sample, and administering a therapy to the identified subject. In some embodiments, the determining if a sample from the subject an increased level of the expression of dgoD, graR, or both relative to the same in a reference sample, an increased level of activity of a trans-2-enoyl-CoA reductase, an Acinetobacter tetrose transporter, or both relative to the same in a reference sample, an increased flux through the B-ureidopropionase reaction relative to the same in a reference sample, a decreased level of one or more bacterial species selected from the group consisting of: Clostridium clostridioforme, Prevotella sp., Streptococcus parasanguinis, Anaerostipes hadrus, Parasutterella excrementihominis, and Eisenbergiella massiliensis relative to the same in a reference sample, and/or an increased level of one or more bacterial species selected from the group consisting of: Bifidobacterium sp., Collinsella sp., Methanobrevibacter smithii, Oscillibacter sp., Faecalibacterium prausnitzii C, Faecalibacterium prausnitzii I, Intestinimonas timonensis, Faecalibacterium prausnitzii, Bacteroides caccae, Barnesiella intestinihominis, Clostridiaceae bacterium, Clostridium sp., and Bifidobacterium adolescentis relative to the same in a reference sample occurs after the subject has received one or more doses of an immunomodulator.

Also provided herein are methods of treating cancer that include administering in a subject identified as having an increased level of the expression of dgoD, graR, or both relative to the same in a reference sample, an increased level of activity of a trans-2-enoyl-CoA reductase, an Acinetobacter tetrose transporter, or both relative to the same in a reference sample, an increased flux through the B-ureidopropionase reaction relative to the same in a reference sample, a decreased level of one or more bacterial species selected from the group consisting of: Clostridium clostridioforme, Prevotella sp., Streptococcus parasanguinis, Anaerostipes hadrus, Parasutterella excrementihominis, and Eisenbergiella massiliensis relative to the same in a reference sample, and/or an increased level of one or more bacterial species selected from the group consisting of: Bifidobacterium sp., Collinsella sp., Methanobrevibacter smithii, Oscillibacter sp., Faecalibacterium prausnitzii C, Faecalibacterium prausnitzii I, Intestinimonas timonensis, Faecalibacterium prausnitzii, Bacteroides caccae, Barnesiella intestinihominis, Clostridiaceae bacterium, Clostridium sp., and Bifidobacterium adolescentis relative to the same in a reference sample a therapy after the subject has received a therapeutically effective amount of an immunomodulator.

Also provided herein are method for increasing the response to an immunomodulator in a subject in need that includes administering to the subject a peptide described herein (e.g., a peptide having the amino acid sequence of SEQ ID NO:1, 117 or 162 or a variant thereof), a recombinant host described herein, or a pharmaceutical composition described herein.

“Dysbiosis” refers to a state of the microbiota or microbiome of the gut or other body area (e.g., mucosal or skin surfaces or any other microbiota niche) of a subject (i.e., the host) in which the diversity and/or function of the ecological network is disrupted, e.g., as compared to the state of the microbiota or microbiome of the gut or other body area in a control population (e.g., a reference population). A control population can include individuals that meet one or more qualifications such as individuals that have not been diagnosed with a disease (e.g., the same disease as the subject); individuals that do not have a known genetic predisposition to a disease (e.g., the same disease as the subject); or individuals that do not have a known environmental predisposition to a disease (e.g., the same disease as the subject); or individuals that do not have a known predisposition that would prevent treatment of and/or recovery from a disease (e.g., the same disease as the subject). In some embodiments, the individuals in the control population meet one of the above control population qualifications. In some embodiments, the individuals in the control population meet two of the above control population qualifications. In some embodiments, the individuals in the control population meet three of the above control population qualifications. In some embodiments, the individuals in the control population meet four of the above control population qualifications. In some embodiments, the control population is homogenous with respect to at least one of the qualifications. Any disruption in the microbiota or microbiome of a subject (i.e., host) compared to the microbiota or microbiome of a control population can be considered a dysbiosis, even if such dysbiosis does not result in a detectable decrease in health of the subject. Dysbiosis in a subject may be unhealthy for the subject (e.g., result in a diseased state in the subject), it may be unhealthy for the subject under only certain conditions (e.g., result in diseased state under only certain conditions), or it may prevent the subject from becoming healthier (e.g., may prevent a subject from responding to treatment or recovering from a disease or disorder). Dysbiosis may be due to a decrease in diversity of the microbiota population composition (e.g., a depletion of one or more bacterial strains, an overgrowth of one or more bacterial strains, or a combination thereof), the overgrowth of one or more population of pathogens (e.g., a population of pathogenic bacteria) or pathobionts, the presence of and/or overgrowth of a symbiotic organism able to cause disease only when certain genetic and/or environmental conditions are present in a subject, or a shift to an ecological network that no longer provides a beneficial function to the host and therefore no longer promotes health.

Also provided herein are methods for treating cancer in a subject that include detecting a dysbiosis associated with response to therapy with an immunomodulator in a sample from a subject. In some embodiments, a method can include detecting, in a sample from the subject, a dysbiosis associated with response to therapy with an immunomodulator, e.g., before administering to the subject an effective amount of a bacterial strain or a composition containing the bacterial strain. In some embodiments, a dysbiosis associated with response to therapy with an immunomodulator is present in the subject before treatment with an immunomodulator. In some embodiments, a dysbiosis associated with response to therapy with an immunomodulator in a subject is present during treatment with an immunomodulator. In some embodiments, a dysbiosis associated with response to therapy with an immunomodulator decreases the efficacy of the therapy with an immunomodulator. The sample can be a biopsy sample such as a biopsy sample (e.g., a tumor biopsy sample) or a fecal sample.

In some embodiments, detecting the dysbiosis associated with response to therapy with an immunomodulator includes determining bacterial gene expression, bacterial composition, and/or bacterial protein activity in a sample from a subject using any of the methods described above. In some embodiments, detecting the dysbiosis associated with response to therapy with an immunomodulator can include determining bacterial gene expression in the sample from the subject. (e.g., a tumor biopsy sample). For example, the bacterial gene expression can be determined in the sample from the subject e.g., before administering to the subject an effective amount of a bacterial strain or a composition containing the bacterial strain and/or after administering to the subject an effective amount of a bacterial strain or a composition containing the bacterial strain. Determining the bacterial gene expression can include performing, for example, RNAseq and/or RT-qPCR. In some embodiments, detecting the dysbiosis associated with response to therapy with an immunomodulator comprises determining bacterial composition in the sample from the subject (e.g., fecal sample or a biopsy sample such as tumor biopsy sample). For example, the bacterial composition can be determined in a sample from the subject, e.g., before administering to the subject an effective amount of a bacterial strain or a composition containing the bacterial strain and/or after administering to the subject an effective amount of a bacterial strain or a composition containing the bacterial strain. Determining the bacterial composition can include, for example, sequencing one or more nucleic acids from the bacteria. In some embodiments, bacteria can be identified by their 16S rRNA gene sequence.

Any of the methods of treatment described herein can include administering a therapy to the identified subject. The therapy can include one or more peptides described herein, one or more pharmaceutical compositions described herein, and/or one or more pharmaceutical compositions described herein.

Any of the methods of treatment described herein can further include treatment with a therapy of a therapeutically effective amount of an immunomodulator, an effective amount of one or more bacterial species selected from the group consisting of: Bifidobacterium sp., Collinsella sp., Methanobrevibacter smithii, Oscillibacter sp., Faecalibacterium prausnitzii C, Faecalibacterium prausnitzii I, Ruminococcaceae bacterium, Intestinimonas timonensis, Faecalibacterium prausnitzii, Bacteroides caccae, Barnesiella intestinihominis, Clostridiaceae bacterium, Ruminococcaceae bacterium, Clostridium sp., and Bifidobacterium adolescentis, and/or an additional treatment of cancer excluding an immunomodulator.

In some embodiments, additional treatment(s) of cancer can include chemotherapeutic agents. Non-limiting examples of chemotherapeutic agents include carboplatin, cisplatin, gemcitabine, methotrexate, paclitaxel, pemetrexed, lomustine, temozolomide, dacarbazine, and combinations thereof. Non-limiting examples of targeted therapies include afatinib dimaleate, bevacizumab, cetuximab, crizotinib, erlotinib, gefitinib, sorafenib, sunitinib, pazopanib, everolimus, dabrafenib, aldesleukin, interferon alfa-2b, peginterferon alfa-2b, trametinib, vemurafenib, and combinations thereof. Non-limiting examples of an immunotherapy include an immune checkpoint inhibitor (e.g., ipilimumab, nivolumab, pembrolizumab, atezolizumab, avelumab, durvalumab, cemiplimab, and combinations thereof); co-stimulatory immune checkpoint agent (e.g., IBI101, utomilumab, MEDI1873, and combinations thereof); and a cell therapy (e.g., a CAR T cell therapy). In some examples, the therapy is a CAR T cell therapy.

In some embodiments, a prebiotic and/or probiotic can be administered in combination with a composition comprising a peptide as described herein. Non-limiting examples of a probiotic include one of more of Bifidobacteria (e.g., B. animalis, B. breve, B. lactis, B. longum, or B. infantis), Lactobacillus (e.g., L. acidophilus, L. reuteri, L. bulgaricus, L. lactis, L. casei, L. rhamnosus, L. plantarum, L. paracasei, or L. delbreuckii/bulgaricus), Saccharomyces boulardii, E. coli Nissle 1917, and Streptococcus thermophiles. Non-limiting examples of a prebiotic include a fructooligosaccharide (e.g., oligofructose, inulin, or an inulin-type fructan), a galactooligosaccharide, an amino acid, or an alcohol. See, for example, Ramirez-Farias et al. (2008. Br. J Nutr. 4:1-10) and Pool-Zobel and Sauer (2007. J Nut. 137:2580-2584).

In some embodiments, methods provided herein can include administering a peptide or pharmaceutical composition thereof described herein to the subject at least once per day. For example, the peptide or pharmaceutical composition thereof can be administered two, three, four, or more times per day. In some embodiments, an effective amount of the peptide or pharmaceutical composition thereof is administered in one dose, e.g., once per day. In some embodiments, an effective amount of the peptide or pharmaceutical composition thereof is administered in more than one dose, e.g., more than once per day. In some embodiments, the method comprises administering the peptide, or pharmaceutical composition thereof to the subject daily, every other day, every three days, or once a week.

An immunomodulator can include an immune checkpoint inhibitor and/or a co-stimulatory immune checkpoint agent. Non-limiting examples of immune checkpoint inhibitors include inhibitors that target CTLA-4 (cytotoxic T-lymphocyte-associated protein 4) such as ipilimumab (YERVOY®); PD-1 (Programmed cell death protein 1) such as pembrolizumab (KEYTRUDA®), nivolumab (OPDIVO®), or cemiplimab (LIBTAYO®); PD-L1 (Programmed death-ligand 1) such as atezolizumab (TECENTRIQ®), avelumab (BAVENCIO®), or durvalumab (IMFINZI®); BTLA (B and T lymphocyte attenuator); LAG-3 (Lymphocyte Activation Gene 3) such as IMP701 (LAG525); A2AR (Adenosine A2a receptor) such as CPI-444; TIM-3 (T-cell immunoglobulin and mucin domain-3) such as MBG453; B7-H3 (B7 homolog 3; also known as CD276) such as enoblituzumab; VISTA (V-domain Ig suppressor of T cell activation) such as JNJ-61610588; and IDO (Indole amine 2,3-dioxygenase) such as indoximod. See, for example, Marin-Acevedo, et al., J Hematol Oncol. 11: 39 (2018). Non-limiting examples of co-stimulatory immune checkpoint agents include agents that target OX40 such as IBI101; 4-1BB such as utomilumab (PF-05082566); and GITR such as MEDI1873.

Immunomodulators can modulate (e.g., increase or decrease) expression or activity of CTLA-4, PD-1, PD-L1, BTLA, LAG-3, A2AR, TIM-3, B7-H3, VISTA, IDO, OX40, 4-1BB, or GITR.

While immunotherapy with immunomodulators such as those described herein has largely been effective, many subjects do not respond to immune checkpoint inhibitors (see, e.g., Humphries and Daud. Hum Vaccin Immunother. 2018; 14(9): 2178-2182). The gut microbiome may be one of the factors that determines the efficacy of immune checkpoint treatment and whether a subject responds to such treatment.

The level of expression of genes, for example a dgoD or graR gene, can be measured with a variety of RNA-based techniques that are known in the art including reverse-transcription polymerase chain reaction (RT-PCR), quantitative RT-PCR (qRT-PCR), global transcriptomics, or RNA-seq.

The level of activity of an enzyme, for example a trans-2-enoyl-CoA reductase or a tetrose transporter, can be measured with a variety of protein-based methods that are known in the art including Western blots, ELISA assays, mass spectrometry, or global proteomic analysis.

The flux of a reaction, or metabolic flux, is the rate of turnover of metabolites through a metabolic pathway. The flux through a reaction, for example, a B-ureidopropionase reaction, can be measured by, for example, comparing the ratio of products to reactants. If the ratio has increased, then there are more products compared to the reactants, indicating that reactants have been converted into products by, for example, an enzyme that catalyzes the reaction.

Any of the methods described herein can include detecting the level of one or more bacterial species, RNA transcripts, protein activity, or flux though a metabolic pathway in a sample from the subject. Detecting levels of bacterial species, RNA transcripts, protein activity or flux can include any of the methods of detection described above.

### T Cells

A critical step in mounting an immune response in mammals is the activation of an appropriate set of T cells which can recognize an antigen associated with a disease or disorder. T cells can differentiate into helper, regulatory, cytotoxic or memory T cells. CD4+ and CD8+ T cells make up the majority of T cells. CD4+ helper T cells recognize MHC-II restricted exogenous antigens that have been captured and processed in the cellular endosomal pathway in antigen presenting cells, such as dendritic cells (DCs), then complexed onto the MHC-II in the Golgi compartment to form an antigen-MHC-II complex. This complex, expressed on the cell surface, can induce activation of CD4+ effector T cells.

CD4+ T cells can be activated and differentiated into distinct effector subtypes including T-helper 1 (Th1), T-helper 2 (Th2), T-helper 17 (Th17), follicular helper T cell (Tfh), induced T-regulatory cells (iTreg) and regulatory type 1 cells (Trl). CD4+ T cells (Th cells) produce interleukins which in turn help to activate other arms of the immune system. For example, Th cells produce interleukin-4 (IL-4) and IL-5, which aid B cells in producing antibodies; IL-2 which activates CD4+ and CD8+ T cells. Interleukin-12 (IL12) and interferon γ (IFNγ) are critical cytokines which initiate the downstream signaling cascade to develop Th1 cells. The IL12, in turn, induces natural killer cells (NK) to produce IFNγ. Since Th cells that recognize MHC-II restricted antigens play a central role in the activation and clonal expansion of cytotoxic T cells, macrophages, natural killer (NK) cells, and B cells, the initial event of activating the helper T cells in response to an antigen is crucial for the induction of an effective immune response directed against that antigen.

CD8+ T cells (cytotoxic T lymphocytes) are important for immune defense against intracellular pathogens and for tumor surveillance. CD8+ cells are activated when the desired protein/peptide is routed through the cell in such a manner so as to be presented on the cell surface as a processed protein/peptide, which is complexed with MHC-I antigens. CD8+ cytotoxic T cells destroy infected target cells though the release of perforin, granzymes, and granulysis.

Regulatory T cells (Tregs) function primarily to suppress potentially deleterious activities of Th cells. Tregs may express a member of the FOX protein family, forkhead box P3 (FOXP3), which functions as a master regulator of the regulatory pathway in the development and function of regulatory T cells. Tregs are often involved in dialing down the immune response. In the instance of cancer, an excess of T_{reg} activity can prevent the immune system from destroying cancer cells.

### Dendritic Cells

In addition to the critical roles that T cells play in the immune response, dendritic cells (DCs) are equally important. DCs are professional antigen-presenting cells that process antigen material and present it on the cell surface to T cells. DCs having a key regulatory role in the maintenance of tolerance to self-antigens and in the activation of innate and adaptive immunity (Banchereau et al., 1998, Nature 392:245-52; Steinman et al., 2003, Annu. Rev. Immunol. 21:685-711).

DCs are derived from hematopoietic bone marrow progenitor cells, which initially transform into immature dendritic cells. Immature dendritic cells constantly sample the surrounding environment for pathogens, which is down through pattern recognition receptors such as the toll-like receptors (TLRs). Antigen-presenting cells (APCs), such as DCs and macrophages, play important roles in the activation of innate and adaptive immunity as well as in the maintenance of immunological tolerance.

When DCs encounter pro-inflammatory stimuli such as microbial products, the maturation process of the cell is initiated by up-regulating cell surface expressed antigenic peptide-loaded MHC molecules and co-stimulatory molecules. Following maturation and homing to local lymph nodes, DCs establish contact with T cells by forming an immunological synapse, where the T cell receptor (TCR) and co-stimulatory molecules congregate in a central area surrounded by adhesion molecules (Dustin et al., 2000, Nat. Immunol. 1:23-9). Once activated in the presence of DCs, e.g., CD8+ T cells can autonomously proliferate for several generations and acquire cytotoxic function without further antigenic stimulation (Kaech et al., 2001, Nat. Immunol. 2:415-22; van Stipdonk et al., 2001, Nat. Immunol. 2:423-9). It has therefore been proposed that the level and duration of peptide-MHC complexes (signal 1) and co-stimulatory molecules (signal 2) provided by DCs are essential for determining the magnitude and fate of an antigen-specific T cell response (Lanzavecchia et al., 2001, Nat. Immunol. 2:487-92; Gett et al., 2003, Nat. Immunol. 4:355-60).

DCs use TLRs, which recognize conserved microbial structures such as lipopolysaccharide (LPS), to promote DC maturation by activating the nuclear factor-κB (NF-κB) signaling pathway (Akira et al., 2004, Nat. Rev. Immunol. 4:499-51 1). Efforts to induce immunization to tumors have attempted to promote DC maturation and co-stimulation as a means of enhancing antitumor immunity.

Much attention has also been focused on pro-inflammatory signaling but less is known about the mechanisms that suppress and resolve inflammation. The magnitude and duration of TLR-initiated immune responses is dictated by the strength and duration of proinflammatory signaling and by the regulation of signal transduction pathways. Since TLR-induced activation of the transcription factor NF-κB is essential for the transcription of a large number of proinflammatory genes, multiple mechanisms are utilized to negatively regulate TLR signaling at multiple levels for the protection of subjects from excessive immune responses such as septic shock and for maintaining immune homeostasis in situations of chronic microbial exposure such as the intestinal microenvironment.

### Cytokines

Cytokines are small secreted proteins released by cells that have a specific effect on the interactions and communications between cells. Cytokine is a general name; other names include lymphokine (cytokines made by lymphocytes), monokine (cytokines made by monocytes), chemokine (cytokines with chemotactic activities), and interleukin (cytokines made by one leukocyte and acting on other leukocytes). Cytokines may act on the cells that secrete them (autocrine action), on nearby cells (paracrine action), or in some instances on distant cells (endocrine action). There are both pro-inflammatory cytokines and anti-inflammatory cytokines. Zhang et al., “Cytokines, Inflammation and Pain,” Int. Anesthesiol. Clin., Vol. 45(2):27-37 (Spring 2007). Cytokines generally stimulate proliferation or differentiation of cells of the hematopoietic lineage or participate in the immune and inflammatory response mechanisms of the body.

Cytokines are critically involved in the regulation of multiple immune cell functions (Curtsinger et al., 2003, J. Exp. Med. 197:1141-51; Valenzuela et al., 2002, J. Immunol. 169:6842-9). As noted above, various immune cell phenotypes are characterized in terms of cytokines which they secrete. Cytokines are often classified as either pro- or anti-inflammatory.

The interleukins are a family of cytokines that mediate immunological responses. Central to an immune response is the T cell, which produces many cytokines and plays a role in adaptive immunity to antigens. Cytokines produced by the T cell have been classified as type 1 and type 2 (Kelso et al., 1998. Immun. Cell Biol. 76:00-317). The type 1 cytokines include IL-2, IFN-γ, LT-α, and are involved in inflammatory responses, viral immunity, intracellular parasite immunity, and allograft rejection. Type 2 cytokines include IL-4, IL-5, IL-6, IL-10, and IL-13, and are involved in humoral responses, helminth immunity, and allergic response.

### Pro-Inflammatory Cytokines

Pro-inflammatory cytokines are cytokines that are important in cell signaling and promote systemic inflammation. They are produced predominantly by activated macrophages and are involved in the upregulation of inflammatory reactions. Pro-inflammatory cytokines arise from genes that code for the translation of small mediator molecules that induce a response after upregulation. Interleukin-1 (IL-1), IL-2, IL-6, IL-12, IL-17, IL-18, IL-23, CD40L, tumor necrosis factor (TNF) such as TNF-α, gamma-interferon (IFN-gamma), granulocyte-macrophage colony stimulating factor, MCP-1, TNF-related apoptosis-inducing ligand, RANK-ligand, and TALL-1/BAFF are well characterized as pro-inflammatory cytokines. Inflammation is characterized by an interplay between pro- and anti-inflammatory cytokines. In some embodiments, administration of the peptides of the present disclosure is accompanied by an increase in pro-inflammatory cytokines.

### Anti-Inflammatory Cytokines

Anti-inflammatory cytokines are a series of immunoregulatory molecules that control the pro-inflammatory cytokine response. These molecules thus modulate and help to decrease the pro-inflammatory response created by pro-inflammatory cytokines. IL-4, IL-10, IL-13, IFN-α (IFN), and transforming growth factor-beta (TGF-β) are recognized as anti-inflammatory cytokines. In some embodiments, administration of the peptides of the present disclosure is accompanied by a decrease of anti-inflammatory cytokines.

It is understood that there is a strong interplay with respect to the effects of cytokines. For example, the pro-inflammatory activity of one cytokine can be attenuated or eliminated by the anti-inflammatory activity of another.

### Target Proteins

Provided herein are method of modulating the activity of one or more target proteins. In some embodiments of the methods provided herein, a peptide is administered that has a target protein. A “target protein” is defined as a protein that the peptide modulated, changes, increases, decreases, or alters the activity, function, or binding partners of. Target proteins of a peptide described herein (e.g., a peptide having a sequence of SEQ ID NO: 1-162) or of a recombinant host cell described herein can include a CD2 protein, a BST2 protein, a TNF protein, a CXCL3 protein, a ADRA2A protein, a ADRB2 protein, a CCR6 protein, a CCR9 protein, a CHRM5 protein, a CXCR3 protein, a CXCR4 protein, a EDGE protein, a HCRTR2 protein, a HRH4 protein, a MRGPRX2 protein, a MTNR1A protein, a NPFFR1 protein, a SSTR1 protein, a SSTR3 protein, a TRHR protein, or a TSHR(L) protein.

In some embodiments, the one or more target proteins is selected from the group consisting of a CD2 protein, a BST2 protein, and a TNF protein (e.g., as a human target for a peptide having a sequence of SEQ ID NO: 1-116). In some embodiments, wherein the one or more target proteins is a CXCL3 protein (e.g., as a human target for a peptide having a sequence of SEQ ID NO: 117-162). In some embodiments, the one or more target proteins is selected from the group consisting of a CCR9 protein, a CHRM5 protein, a CXCR3 protein, a CXCR4 protein, a HCRTR2 protein, a MRGPRX2 protein, a SSTR1 protein, and a TSHR(L) protein (e.g., as a human target for a peptide having a sequence of SEQ ID NO: 117-161). In some embodiments, the one or more target proteins is selected from the group consisting of a CCR9 protein, a CHRM5 protein, a CXCR3 protein, a CXCR4 protein, a HCRTR2 protein, a MRGPRX2 protein, a SSTR1 protein, or a TSHR(L) protein (e.g., as a human target for a peptide having a sequence of SEQ ID NO: 162).

In some embodiments of methods of modulating the activity of one or more target proteins, the method includes identifying a subject as having a decreased likelihood of positively responding to treatment with an immunomodulator. For example, the subject may have a sample (e.g., a fecal sample) that has a decreased level of the expression of dgoD, graR, or both relative to the same in a reference sample; a decreased level of activity of a trans-2-enoyl-CoA reductase, an Acinetobacter tetrose transporter, or both relative to the same in a reference sample; a decreased flux through the B-ureidopropionase reaction relative to the same in a reference sample; an increased level of one or more bacterial species selected from the group consisting of: Clostridium clostridioforme, Prevotella sp., Streptococcus parasanguinis, Anaerostipes hadrus, Parasutterella excrementihominis, and Eisenbergiella massiliensis relative to the same in a reference sample; or a decreased level of one or more bacterial species selected from the group consisting of: Bifidobacterium sp., Collinsella sp., Methanobrevibacter smithii, Oscillibacter sp., Faecalibacterium prausnitzii C, Faecalibacterium prausnitzii I, Intestinimonas timonensis, Faecalibacterium prausnitzii, Bacteroides caccae, Barnesiella intestinihominis, Clostridiaceae bacterium, Clostridium sp., and Bifidobacterium adolescentis relative to the same in a reference sample as having a decreased likelihood of having a positive response, or is less likely to respond to treatment with an immunomodulator.

In some embodiments of method of modulating the activity of one or more target proteins, the method includes identifying a subject as having an increased likelihood of having a positive response to treatment with an immunomodulator. For example, a subject with a sample (e.g., a fecal sample) with an increased level of expression of genes, for example, the dgoD or graR genes, relative to the same in a reference sample, an increased level of activity of a trans-2-enoyl-CoA reductase, a tetrose transporter (e.g., an Acinetobacter tetrose transporter), or both relative to the same in a reference sample, an increased flux through the B-ureidopropionase reaction relative to the same in a reference sample, a decreased level of one or more bacterial species selected from the group consisting of: Clostridium clostridioforme, Prevotella sp., Streptococcus parasanguinis, Anaerostipes hadrus, Parasutterella excrementihominis, or Eisenbergiella massiliensis relative to the same in a reference sample, or a increased level of one or more bacterial species selected from the group consisting of: Bifidobacterium sp., Collinsella sp., Methanobrevibacter smithii, Oscillibacter sp., Faecalibacterium prausnitzii C, Faecalibacterium prausnitzii I, Intestinimonas timonensis, Faecalibacterium prausnitzii, Bacteroides caccae, Barnesiella intestinihominis, Clostridiaceae bacterium, Clostridium sp., and Bifidobacterium adolescentis relative to the same in a reference sample may be identified as having an increased likelihood of having a positive response to treatment with an immunomodulator.

### Cancer

Globally suppressed T cell function has been described in many subjects with cancer to be a major hurdle for the development of clinically efficient cancer immunotherapies. The inhibition of antitumor immune responses has largely been linked to inhibitory factors present in subjects presenting with cancer. A “neoplastic disorder” is any disorder associated with cell proliferation, specifically with a neoplasm. A “neoplasm” or “neoplasia” is an abnormal mass of tissue that may be benign or malignant. Nearly all benign tumors are encapsulated and are non-invasive. In contrast, malignant tumors are almost never encapsulated and invade adjacent tissue by infiltrative destructive growth. This infiltrative growth can be followed by tumor cells implanting at sites discontinuous with the original tumor.

A neoplasm or a neoplastic disorder can be a cancer. “Cancer” as used herein refers to an uncontrolled growth of cells which interfere with the normal functioning of the bodily organs and systems. Hemopoietic cancers, such as leukemia, are able to outcompete the normal hemopoietic compartments in a subject, thereby leading to hemopoietic failure in the form of anemia, thrombocytopenia and neutropenia; ultimately causing death.

Cancers which migrate from their original location and seed vital organs can eventually lead to the death of the subject through the functional deterioration of the affected organ(s). A metastasis is a region of cancer cells, distinct from the primary tumor location resulting from the dissemination of cancer cells from the primary tumor to other parts of the body. At the time of diagnosis of the primary tumor mass, the subject may be monitored for the presence of metastases. Metastases are most often detected through the sole or combined use of magnetic resonance imaging (MRI) scans, computed tomography (CT) scans, blood and platelet counts, liver function assays, chest X-rays and bone scan, in addition to the monitoring of specific symptoms.

Methods of the present disclosure may be utilized to treat or prevent neoplastic disorders in humans, including but not limited to cancers such as sarcoma, carcinoma, fibroma, leukemia, lymphoma, melanoma, myeloma, neuroblastoma, rhabdomyosarcoma, retinoblastoma, and glioma. Cancers include but are not limited to basal cell carcinoma, biliary tract cancer, bladder cancer, bone cancer, brain and central nervous system (CNS) cancer, breast cancer, cervical cancer, choriocarcinoma, colon and rectum cancer, connective tissue cancer, cancer of the digestive system, endometrial cancer, esophageal cancer, eye cancer, cancer of the head and neck, gastric cancer, intra-epithelial neoplasm, kidney cancer, larynx cancer, leukemia, liver cancer, lung cancer (small cell and non-small cell), lymphoma (including Hodgkin's and non-Hodgkin's), melanoma, myeloma, neuroblastoma, oral cavity cancer (lip, tongue, mouth, and pharynx), ovarian cancer, pancreatic cancer, prostate cancer, retinoblastoma, rhabdomyosarcoma, rectal cancer, renal cancer, cancer of the respiratory system, sarcoma, skin cancer, stomach cancer, testicular cancer, thyroid cancer, uterine cancer, cancer of the urinary system, as well as other carcinomas and sarcomas.

In some embodiments of the method described herein, cancers can include melanoma, lung cancer, kidney cancer, bladder cancer, a head and neck cancer, Merkel cell carcinoma, urothelial cancer, breast cancer, glioblastoma, gastric cancer, a nasopharyngeal neoplasm, colorectal cancer, hepatocellular carcinoma, ovarian cancer, and/or pancreatic cancer.

In some embodiments, the subject has a hematological malignancy. Hematological malignancies can include multiple myeloma, non-Hodgkin lymphoma, Hodgkin lymphoma, diffuse large B-cell lymphoma, and chronic lymphocytic leukemia/small lymphocytic lymphoma (CLL/SLL).

“A subject having cancer” is a subject that has been diagnosed with a cancer. In some embodiments, the subject has a cancer type characterized by a solid mass tumor. The solid tumor mass, if present, may be a primary tumor mass. A primary tumor mass refers to a growth of cancer cells in a tissue resulting from the transformation of a normal cell of that tissue. In most cases, the primary tumor mass is identified by the presence of a cyst, which can be found through visual or palpation methods, or by irregularity in shape, texture, or weight of the tissue.

Some primary tumors are not palpable and can be detected only through medical imaging techniques such as X-rays or by needle aspirations. The use of these latter techniques is more common in early detection. Molecular and phenotypic analysis of cancer cells within a tissue will usually confirm if the cancer is endogenous to the tissue or if the lesion is due to metastasis from another site.

It has been estimated that almost half of all currently diagnosed cancers will be treated with some form of cancer medicament. However, many forms of cancer, including melanoma, colorectal, prostate, endometrial, cervical, and bladder cancer do not respond well to treatment with cancer medicaments. In fact, only about 5-10 percent of cancers can be cured using cancer medicaments alone. These include some forms of leukemias and lymphomas, testicular cancer, choriocarcinoma, Wilm's tumor, Ewing's sarcoma, neuroblastoma, small-cell lung cancer, and ovarian cancer. Treatment of still other cancers, including breast cancer, requires a combination of therapy of surgery or radiotherapy in conjunction with a cancer medicament. See Bratzler and Peterson.

The tumor environment is often refractory to immunological attack. It is desirable in cancer immunotherapy to make the tumor environment less refractory so as to increase the activity of CTLs or other effector T cells within the tumor and to improve the overall efficacy of treatment. As used herein, “efficacy” refers to the ability of a chemotherapeutic and/or immunological composition or a combination treatment thereof to achieve a desired action or result.

It has been demonstrated that some human cancer patients develop an antibody and/or T lymphocyte response to antigens on neoplastic cells. It has also been shown in animal models of neoplasia that enhancement of the immune response can result in rejection or regression of that particular neoplasm. Molecules that enhance the T lymphocyte response in the mixed lymphocyte reaction (MLR) have utility in vivo in enhancing the immune response against neoplasia. Molecules which enhance the T lymphocyte proliferative response in the MLR (or small molecule agonists or antibodies that affected the same receptor in an agonistic fashion) can be used therapeutically to treat cancer. Molecules that inhibit the lymphocyte response in the MLR also function in vivo during neoplasia to suppress the immune response to a neoplasm; such molecules can either be expressed by the neoplastic cells themselves or their expression can be induced by the neoplasm in other cells. Antagonism of such inhibitory molecules (either with antibody, small molecule antagonists or other means) enhances immune-mediated tumor rejection.

### Clinical Parameters for Treating Neoplasia

The administration of a composition comprising a peptide of the present disclosure results in a biological response in the subject/subject's cells. In some embodiments, administration of one or more peptides of the present disclosure results in the subject or the cells isolated therefrom to exhibit one or more of a reduction in the expression of IL-10, an increase of inflammatory (pro-inflammatory) cytokines, an increase of TNF-α, a reduction in anti-inflammatory cytokines, a limiting of tolerogenic dendritic cell expansion, a reduction in the ratio of IL-10:TNF, increase in the expression of IL-12, an increase or promotion of Th1 activation, an increase in TNF, an increase or enhancement of dendritic cell maturation, an increase in CD70 expression, an increase in T-cell activation, an increase in T-cell activation along with co-stimulation via CD27, an increase in the expression of CD80 and/or CD86, an increase or the enhancement of T-cell activation, an increase in T-cell activation along with co-stimulation via CD28, an increase in the expression of MHC I and/or MHC II, an increase or enhancement of T-cell activation by means of an increase in MHC-involved antigen presentation, a decrease in the number of T_{reg} cells, preventing the clonal expansion of T_{reg} cells and/or promoting the clonal expansion of T_{eff} cells, an increase in the number of T_{act} cells, an increase in the number of CTL cells, a decrease in the size and/or volume of neoplastic tissue, preventing metastasis of neoplastic tissue or cells, induction of apoptosis in neoplastic cells, an increase in the rate of apoptosis of neoplastic cells, a reduction in the number of neoplastic masses in one or more tissues, a decrease in the size of neoplastic lesions, and an increase in the clonal expansion of T_{act}, T_{eff/mem}, and/or CTL cells. In some embodiments, administration of one or more peptides of the present disclosure to a subject results in a decrease in expression of one or more genes selected from the group consisting of: signal transducer and activator of transcription 1 (STAT1), interferon regulatory factor 1 (IRF1), cluster of differentiation 96 (CD96), mothers against decapentaplegic homolog 3 (SMAD3), C—X—C motif chemokine receptor 6 (CXCR6), transcription factor 7 (TCF7), lymphocyte antigen 9 (LY9), C—X—C motif chemokine 10 (CXCL10), granzyme K (GZMK), interferon stimulated exonuclease gene 20 (ISG20), and signaling lymphocytic activation molecule F7 (SLAMF7) in the subject (e.g., in cells such as T cells of the subject). In some embodiments, administration of one or more peptides of the present disclosure to a subject results in an increase in expression of one more genes selected from the group consisting of: dual specificity phosphatase 6 (DUSP6), cathepsin L (CTSL), IL-9, IL-2, IL-10, IL-24, IL-21, and IL-3 in the subject (e.g., in cells such as T cells of the subject).

In some embodiments, administration of one or more peptides of the present disclosure to a subject results in an increase in expression of one more genes selected from the group consisting of: DUSP6, CTSL, IL-9, IL-2, IL-10, IL-24, IL-21, and IL-3 in the plasma of the subject.

In some embodiments, administration of a composition comprising the peptide to a subject results in an increased life expectancy in the subject of at least 1, 2, 3, 4, 5, 6, 7, 8, 9, 10, 11, 12, 13, 14, 15, 16, 17, 18, 19, 20, 21, 22, 23, 24, 25, 26, 27, 2, 29, 30, 31, 32, 33, 34, 35, 36, 37, 38, 39, 40 41, 42, 43, 44, 45, 46, 47, 48, 49, 50, 51, or 52 weeks. In some embodiments, administration of a composition comprising the peptide to a subject results in an increased life expectancy in the subject of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 41, 42, 43, 44, 45, 46, 47, or 48 months. In some embodiments, administration of a composition comprising the peptide to a subject results in an increased life expectancy in the subject of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 years.

In some embodiments, administration to a subject of a composition comprising the peptide results in a reduction in the volume of one or more neoplasia by at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 98% of the volume of the one or more neoplasia.

In some embodiments, administration to a subject of a composition comprising the peptide results in a reduction of the size of one or more neoplastic lesions by 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 98% of the volume of the one or more neoplastic lesions.

In some embodiments, administration to a subject of a composition comprising the peptide results in a reduction in one or more negative side effects of the neoplasia by at least 5%, 10%, 20%, 25%, 30%, 35%, 40%, 45%, 50%, 55%, 60%, 65%, 70%, 75%, 80%, 85%, 90%, or 95%; wherein the negative side effects include nausea, pain, discomfort, vomiting, diarrhea, vertigo, loss of appetite, nerve pain, seizures, periodic loss of consciousness, loss or lack of ambulatory movement, loss or lack of physical coordination, and loss or lack of vision.

In some embodiments, administration to a subject of a composition comprising the peptide results in a shift in the clonal populations of T_{reg} and T_{eff} cells in contact with the one or more neoplasia, wherein the population of T_{eff} increases at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30-fold relative to the population of T_{reg}.

### Combination Therapies Comprising Peptides

The pharmaceutical compositions provided herein comprising a peptide may be combined with other treatment therapies (e.g., treatment(s) for cancer) and/or pharmaceutical compositions. For example, a subject suffering from an immunological associated disease or disorder, or cancer, may already be taking a pharmaceutical prescribed by their doctor to treat the condition. In embodiments, the pharmaceutical compositions provided herein, are able to be administered in conjunction with the subject's existing medicines.

For example, the peptides provided herein may be combined with one or more of: a 5-aminosalicylic acid compound, an anti-inflammatory agent, an antibiotic, an antibody (e.g. antibodies targeting an inflammatory cytokine, e.g. antibodies targeting TNF-α, such as adalimumab, pegol, golimumab, infliximab, and certolizumab), an anti-cytokine agent, an anti-inflammatory cytokine agent, a steroid, a corticosteroid, an immunosuppressant (e.g. azathioprine and mercaptopurine), vitamins, and/or specialized diet. In some embodiments, the peptide of the present disclosure is administered with a checkpoint inhibitor, such as an agent that targets PD-1, PD-L1, CTLA-4, BTLA, LAG-3, A2AR, TIM-3, B7-H3, VISTA, or IDO. Such drugs include but are not limited to pembrolizumab, nivolumab, atezolizumab, avelumab, durvalumab, cemiplimab, and ipilimumab. The peptides provided herein may be combined with an autologous cellular immunotherapy (e.g., sipuleucel-T). In some embodiments, the other treatment therapies and/or pharmaceutical compositions may be selected from cancer immunotherapies such as monoclonal antibodies that activate NK cells and enhance antibody-dependent cellular cytotoxicity; cancer vaccines with or without adjuvants that stimulate a cancer-antigen-specific humoral immune response; chemotherapeutic agents such as carboplatin and/or mitotane; hormones such as adrenocorticosteroids or fluoxymesterone; or biological response modifiers that alter a subject's response to cancer rather than by direct cytotoxicity of cancer cells, such as erythropoietin or GM-CSF. An extensive, but non-limiting list of treatment therapies, pharmaceutical compositions/medicaments are disclosed in Bratzler and Peterson.

In some embodiments, one or more tumors or neoplastic tissues are debulked prior to or during immunotherapy. In some embodiments, debulking one or more tumors prior to or during immunotherapy results in either a slowing or a halt of disease progression. In some embodiments, debulking one or more tumors prior to or during immunotherapy results in tumor regression or elimination.

In some embodiments, a slowing of disease progression comprises at least a 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% decrease in the rate of growth or expansion of the tumor.

In some embodiments, tumor regression comprises at least a 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% decrease in the tumor numbers, tumor size, or tumor volume.

Any procedure that allows an assessment of the tumor or lesion size can be used. Non-limiting examples include digital rectal exam, an endoscopy (e.g., a colonoscopy), and imaging (e.g., PET, MRI, ERUS, DRE, CT). See, for example, McKeown et al. J Cancer. 2014; 5(1): 31-43. In some embodiments, tumor burden can be assessed using RECIST (e.g., RECIST version 1 or version 1.1). See, for example, Eisenhauer et al., Eur. Cancer. J. 45(2):228-47 (2009).

Criteria for evaluating immunotherapy have also been developed. Non-limiting examples include the immune-related response criteria (irRC) (see Wolchok et al. Clin Cancer Res. 2009 Dec. 1; 15(23):7412-20) and immune response criteria in solid tumors (iRECIST) criteria (see Seymour et al. Lancet Oncol. 2017 March; 18(3): e143-e152). See also, Thallinger et al. Wien Klin Wochenschr. 2018; 130(3): 85-91.

Having an antigen-specific humoral and cell-mediated immune response, in addition to activating NK cells and endogenous dendritic cells, and increasing IFN levels, can be helpful for treating cancer. Some cancer cells are antigenic and thus can be targeted by the immune system. In one aspect, peptides of the present disclosure, or peptides of the present disclosure plus one or more cancer medicaments are particularly useful for stimulating an immune response against a cancer. A “cancer antigen” as used herein is a compound, such as a peptide, associated with a tumor or cancer cell surface and which is capable of provoking an immune response when expressed on the surface of an antigen presenting cell, such as a dendritic cell. In some aspects, the antigen is presented on an APC via an MHC molecule. Cancer antigens, such as those present in cancer vaccines or those used to prepare cancer immunotherapies, can be prepared from crude cancer cell extracts or by partially purifying the antigens, using recombinant technology or de novo synthesis of known antigens. In some aspects, proteins isolated from other organisms or synthetic proteins sharing a degree of homology to the proteins are used to prepare cancer immunotherapies.

Different types of cells that can kill tumor targets in vitro and in vivo have been identified: natural killer cells (NK cells), cytolytic T lymphocytes (CTLs), lymphokine-activated killer cells (LAKs), and activated macrophages. NK cells can kill tumor cells without having been previously sensitized to specific antigens, and the activity does not require the presence of class I antigens encoded by the major histocompatibility complex (MHC) on target cells. NK cells are thought to participate in the control of nascent tumors and in the control of metastatic growth. In contrast to NK cells, CTLs can kill tumor cells only after they have been sensitized to tumor antigens and when the target antigen is expressed on the tumor cells that also express MHC class I. CTLs are thought to be effector cells in the rejection of transplanted tumors and of tumors caused by DNA viruses. LAK cells are a subset of null lymphocytes distinct from the NK and CTL populations. Activated macrophages can kill tumor cells in a manner that is not antigen dependent nor MHC restricted once activated. Activated macrophages are thought to decrease the growth rate of the tumors they infiltrate. In vitro assays have identified other immune mechanisms such as antibody-dependent, cell-mediated cytotoxic reactions and lysis by antibody plus complement. However, these immune effector mechanisms are thought to be less important in vivo than the function of NK, CTLs, LAK, and macrophages.

The use of peptides of the present disclosure in conjunction with cancer vaccines provides an improved antigen-specific humoral and cell-mediated immune response, in addition to activating NK cells and endogenous dendritic cells, and increasing IFN levels. Such an enhancement can allow for the use of a vaccine with a reduced antigen dose to achieve the same beneficial effect.

### Pharmaceutical Compositions Comprising Peptides

Compositions are provided that comprise a peptide as described herein. In some embodiments, the compositions described herein are pharmaceutical compositions suitable for administration to as subject and that demonstrate a therapeutic effect when administered to a subject in need thereof. Pharmaceutical compositions of the present disclosure can comprise a therapeutically effective amount of a peptide in a pharmaceutically acceptable carrier. The preparation of a pharmaceutical composition or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by the FDA Office of Biological Standards.

The compositions of the disclosure can comprise different types of carriers depending on whether it is to be administered in solid, liquid or aerosol form, and whether it needs to be sterile for such routes of administration as injection. The peptides of the disclosure can be administered orally, or rectally, but can also be administered intrathecally, intranasally, subcutaneously, mucosally, by inhalation (e.g., aerosol inhalation), by injection, by infusion or continuous infusion, topically, localized perfusion bathing target cells directly, via a catheter, via a lavage, or by other method or any combination of the foregoing as would be known to one of ordinary skill in the art (see, for example, Remington's Pharmaceutical Sciences, 18^{th} Ed. Mack Printing Company, 1990, incorporated herein by reference).

The peptides of the present disclosure can be formulated into a composition in a free base, neutral, or salt form. Pharmaceutically acceptable salts, include the acid addition salts, e.g., those formed with the free amino groups of a proteinaceous composition, or which are formed with inorganic acids such as, for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium, or ferric hydroxides; or such organic bases as isopropylamine, trimethylamine, histidine, or procaine.

In some embodiments where the composition is in a liquid form, a carrier can be a solvent or dispersion medium comprising but not limited to water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In particular embodiments, the peptide compositions of the present disclosure are prepared for administration by such routes as oral ingestion (e.g. oral administration). In these embodiments, the solid composition can comprise, for example, solutions, suspensions, emulsions, tablets, pills, capsules (e.g., hard or soft shelled gelatin capsules), delayed release capsules, sustained release formulations, buccal compositions, troches, elixirs, suspensions, syrups, wafers, or combinations thereof. Oral compositions can be incorporated directly with the food of the diet. Preferred carriers for oral administration can comprise inert diluents, assimilable edible carriers, or combinations thereof. In other aspects of the disclosure, the oral composition can be prepared as a syrup or elixir. A syrup or elixir, and can comprise, for example, at least one active agent, a sweetening agent, a preservative, a flavoring agent, a dye, a preservative, or combinations thereof.

In some embodiments, an oral composition can comprise one or more binders, excipients, disintegration agents, lubricants, flavoring agents, and combinations thereof. In some embodiments, a composition can comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it can contain, in addition to materials of the above type, carriers such as a liquid carrier. Various other materials can be present as coatings or to otherwise modify the physical form of the dosage unit. For instance, tablets, pills, or capsules can be coated with shellac, sugar or both.

Additional formulations which are suitable for other modes of administration include suppositories (e.g. rectal administration). Suppositories are solid dosage forms of various weights and shapes, usually medicated, for insertion into the rectum. After insertion, suppositories soften, melt or dissolve in the cavity. In general, for suppositories, traditional carriers can include, for example, polyalkylene glycols, triglycerides or combinations thereof. In some embodiments, suppositories can be formed from mixtures containing, for example, the active ingredient in the range of about 0.5% to about 10%, and preferably about 1% to about 2%.

In some embodiments, compositions suitable of intravenous administration are provided. In some embodiments, compositions suitable for intratumoral administration are provided. In some embodiments, compositions suitable for parenteral administration are provided. Injectable formulations comprise one or more described peptides in combination with one or more pharmaceutically-acceptable sterile isotonic aqueous or nonaqueous solutions, dispersions, suspensions or emulsions, or sterile powders which can be reconstituted into sterile injectable solutions or dispersions just prior to use, which can contain sugars, alcohols, amino acids, antioxidants, buffers, bacteriostats, solutes which render the formulation isotonic with the blood of the intended recipient or suspending or thickening agents. Examples of suitable aqueous and nonaqueous carriers which can be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate.

These pharmaceutical compositions can also contain adjuvants such as preservatives, wetting agents, emulsifying agents, and dispersing agents. Prevention of the action of microorganisms upon the described compounds can be ensured by the inclusion of various antibacterial and antifungal agents, for example, paraben, chlorobutanol, phenol sorbic acid, and the like. It can also be desirable to include agents to control tonicity, such as sugars, sodium chloride, and the like into the compositions. In addition, prolonged absorption of the injectable pharmaceutical form can be brought about by the inclusion of agents which delay absorption such as aluminum monostearate and gelatin.

Sterile injectable solutions can be prepared by incorporating the active compounds, e.g., a peptide described herein, in the required amount in the appropriate solvent with various combinations of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of a peptide described herein plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition should be stable under the conditions of manufacture and storage, and preserved against the contaminating action of microorganisms, such as bacteria and fungi. It will be appreciated that endotoxin contamination should be kept minimally at a safe level, for example, less than 0.5 ng/mg protein/peptide.

In some embodiments, the composition comprises a purified peptide that comprises, consists of, or consists essentially of, any of the amino acid sequences listed in Table 1, 2, 3, 4, 5, or 6.

### Production or Synthesis of Peptides

Any of the peptides described herein can be generated using recombinant techniques. For example, a polynucleotide sequence encoding the peptide, e.g., any of the peptides described herein, or a variant thereof, can be cloned into a nucleic acid construct (e.g., an expression vector), which can be used to transform an appropriate host cell, e.g., a prokaryote (e.g., a eubacteria, an archaea) or a eukaryote (yeast). In some embodiments, the host cells can be, e.g., E. coli BL21 cells, Bacillus sp. such as B. thuringiensis, or P. fluorescens.

Numerous cell lines and cultures are available for use as a host cell, and they can be obtained for example through the American Type Culture Collection (ATCC), which is an organization that serves as an archive for living cultures and genetic materials. An appropriate host can be determined by one of skill in the art based on the vector backbone and the desired result. A plasmid or cosmid, for example, can be introduced into a prokaryote host cell for replication of many vectors. Cell types available for vector replication and/or expression include, but are not limited to, bacteria, such as E. coli (e.g., E. coli strain RR1, E. coli LE392, E. coli B, E. coli X 1776 (ATCC No. 31537) as well as E. coli W3110 (F-, lambda-, prototrophic, ATCC No. 273325), DH5α, JM109, and KC8, bacilli such as Bacillus subtilis and Bacillus thuringiensis; and other enterobacteriaceae such as Salmonella typhimurium, Serratia marcescens, various Pseudomonas species such as P. fluorescens, as well as a number of commercially available bacterial hosts such as SURE® Competent Cells and SOLOPACK™ Gold Cells (STRATAGENE®, La Jolla). In some embodiments, bacterial cells such as E. coli are particularly contemplated as host cells.

Examples of eukaryotic host cells for replication and/or expression of a vector include, but are not limited to, HeLa, NIH3T3, Jurkat, 293, Cos, Chinese Hamster Ovary (CHO), Saos, and PC12. Many host cells from various cell types and organisms are available and would be known to one of skill in the art. Similarly, a viral vector can be used in conjunction with either a eukaryotic or prokaryotic host cell, particularly one that is permissive for replication or expression of the vector.

From a given amino acid sequence (e.g. any of the sequences in Table 1-6), the nucleic acid sequence can be codon optimized (e.g., using a codon optimization algorithm) to generate the nucleotide sequence. The codon optimization algorithm chooses an appropriate codon for a given amino acid based on the expression host's codon usage bias. Many codon optimization algorithms also take into account other factors such as mRNA structure, host GC content, ribosomal entry sites. Some examples of codon optimization algorithms and gene synthesis service providers are: GenScript: genscript.com/codon-opt.html on the World Wide Web; ThermoFisher: thermofisher.com/us/en/home/life-science/cloning/gene-synthesis/geneart-gene-synthesis/geneoptimizer.html on the World Wide Web; and Integrated DNA Technologies: idtdna.com/CodonOpt on the World Wide Web. The nucleotide sequence is then synthesized and cloned into an appropriate nucleic acid construct (e.g., an appropriate expression vector).

The host cells containing the nucleic acid construct can be cultured to allow growth of the cells and expression of the peptide. In some embodiments, expressed peptide can then be purified, again using a variety of methods readily known to a person having ordinary skill in the art. Generally, “purified” will refer to a specific peptide composition that has been subjected to fractionation to remove non-proteinaceous components and various other proteins, polypeptides, or peptides, and which composition substantially retains its activity, as can be assessed, for example, by the protein assays, as described herein below, or as would be known to one of ordinary skill in the art for the desired protein, peptide or peptide.

Where the term “substantially purified” is used, this will refer to a composition in which the specific protein, peptide, or peptide forms the major component of the composition, such as constituting about 50% of the peptides in the composition or more. In preferred embodiments, a substantially purified peptide will constitute more than 60%, 70%, 80%, 90%, 95%, 99% or even more of the peptides in the composition.

A peptide, polypeptide or protein that is “purified to homogeneity,” as applied to the present disclosure, means that the peptide, polypeptide or protein has a level of purity where the peptide, polypeptide or protein is substantially purified or free from other proteins/peptides and biological components. For example, a purified peptide, polypeptide or protein will often be sufficiently free of other protein/peptide components so that degradative sequencing can be performed successfully.

Various methods for quantifying the degree of purification of proteins, peptides, or peptides will be known to those of skill in the art in light of the present disclosure. These include, for example, determining the specific polypeptide activity of a fraction, or assessing the number of peptides within a fraction by gel electrophoresis.

Although preferred for use in some embodiments, there is no general requirement that the protein, polypeptide, or peptide always be provided in their most purified state. Indeed, it is contemplated that less substantially purified protein, polypeptide or peptide, which are nonetheless enriched in the desired peptide compositions, relative to the natural state, will have utility in some embodiments.

Methods exhibiting a lower degree of relative purification can have advantages in total recovery of peptide product, or in maintaining the activity of an expressed peptide. Inactive products also have utility in some embodiments, such as, e.g., in determining antigenicity via antibody generation.

In other embodiments, a preparation enriched with the peptides can be used instead of a purified preparation. In this document, whenever purified is used, enriched can be used also. A preparation can be enriched not only by methods of purification, but also by the over-expression or over-production of the peptide by bacteria when compared to wild-type. This can be accomplished using recombinant methods, or by selecting conditions which will induce the expression of the peptide from the wild type cells.

### Expression Systems

Provided herein are compositions and methods for producing peptides of the present disclosure. Also provided are nucleic acid constructs that contain a polynucleotide sequence encoding a peptide described herein. Also provided herein are host cells which harbor the nucleic acid constructs. The peptides of the present disclosure can be prepared by routine recombinant methods, e.g., culturing cells transformed or transfected with a nucleic acid construct (e.g., an expression vector) containing a nucleic acid encoding a peptide described herein. Numerous expression systems can be used to produce a peptide as discussed above. Prokaryote- and/or eukaryote-based systems can be employed for use with the present disclosure to produce nucleic acid sequences, or their cognate peptides, polyproteins and peptides. Many such systems are commercially and widely available. Expression systems include but are not limited to insect cell/baculovirus systems and inducible mammalian expression systems, it is contemplated that the proteins, polypeptides or peptides produced by the methods of the disclosure can be “overexpressed.” For example, proteins, peptides or peptides can be expressed in increased levels relative to its natural expression in cells.

Accordingly, a method for producing any of the herein described peptides is further provided and comprises culturing host cells under conditions suitable for expression of the desired peptide and recovering the desired peptide from the cell culture. The recovered peptide can then be isolated and/or purified for use in in vitro and in vivo methods, as well as for formulation into a pharmaceutically acceptable composition. In some embodiments, the peptide is expressed in a prokaryotic cell such as E. coli, Lactococcus lactis, Streptomyces species (e.g., S. coelicolor, S. lividans, S. albus, or S. venezuelae), or Bacillus species (e.g., B. subtilis). In some embodiments, the peptide is expressed in a eukaryotic cell such as a yeast (e.g., Saccharomyces cerevisiae, Pichia pastoris, Yarrowia lipolytica, Aspergillus niger, Hansenula polymorpha) or an insect cell (e.g., sf9, sf21, Tni, and S2). In some embodiments, the isolation and purification of the peptide includes one or more steps to reduce endotoxin to levels acceptable for therapeutic use in humans or other animals.

Also provided herein are nucleic acid constructs which comprise a polynucleotide sequence which encodes a peptide of the present disclosure. Polynucleotide sequences encoding the peptides of the disclosure can be obtained using standard recombinant techniques. Desired encoding polynucleotide sequences can be amplified from the genomic DNA of the source bacterium, i.e., Bacillus thuringiensis. Alternatively, polynucleotides can be synthesized using nucleotide synthesizer. Once obtained, sequences encoding the peptides are inserted into a recombinant vector capable of replicating and expressing heterologous (exogenous) polynucleotides in a host cell. Many vectors that are available and known in the art can be used for the purpose of the present disclosure. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides. The vector components generally include, but are not limited to: an origin of replication, a selection marker gene, a promoter, a ribosome binding site (RBS), a signal sequence, the heterologous nucleic acid insert and a transcription termination sequence.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is typically transformed using a pBR322, pUC, pET or pGEX vector, a plasmid derived from an E. coli species. Such vectors can contain genes encoding ampicillin (Amp) and tetracycline (Tet) resistance and thus provides easy means for identifying transformed cells. These vectors as well as their variants or other microbial plasmids or bacteriophage can also contain, or be modified to contain, promoters which can be used by the microbial organism for expression of endogenous proteins.

A nucleic acid construct of the present disclosure can comprise a promoter, an untranslated regulatory sequence located upstream (5′) and operably linked to a polypeptide-encoding nucleotide sequence such that the promoter regulated transcription of that coding sequence. Prokaryotic promoters typically fall into two classes, inducible and constitutive. An inducible promoter is a promoter that initiates increased levels of transcription of the encoding polynucleotide under its control in response to changes in the culture condition, e.g., the presence or absence of a nutrient or a change in temperature. A large number of promoters recognized by a variety of potential host cells are well known and a skilled artisan can choose the promoter according to desired expression levels. Promoters suitable for use with prokaryotic hosts include E. coli promoters such as lac, trp, tac, trc and ara, viral promoters recognized by E. coli such as lambda and T5 promoters, and the T7 and T7lac promoters derived from T7 bacteriophage. A host cell harboring a vector comprising a T7 promoter, e.g., is engineered to express a T7 polymerase. Such host cells include E. coli BL21(DE3), Lemo21(DE3), and NiCo21(DE3) cells. Promoters suitable for use with yeast hosts include promoters such as yeast alcohol dehydrogenase 1 (ADH1) promoter, yeast phosphoglycerate kinase (PGK1) promoter, and translational elongation factor EF-1 alpha promoter. In some embodiments, wherein the host cell is a H. polymorpha cell, the promoter is a MOX promoter. In some embodiments, the promoter is an inducible promoter which is under the control of chemical or environmental factors.

Further useful plasmid vectors include pIN vectors (Inouye et al., 1985); and pGEX vectors, for use in generating glutathione S-transferase (GST) soluble fusion proteins for later purification and separation or cleavage. Other suitable fusion proteins are those with β-galactosidase, ubiquitin, and the like.

Suitable vectors for expression in both prokaryotic and eukaryotic host cells are known in the art.

Vectors of the present disclosure can further comprise a signal sequence which allows the translated recombinant peptide to be recognized and processed (i.e., cleaved by a signal peptidase) by the host cell. For prokaryotic host cells that do not recognize and process the signal sequences native to the heterologous peptides, the signal sequence is substituted by a prokaryotic signal sequence selected, for example, from the group consisting of the alkaline phosphatase, penicillinase, Ipp, or heat-stable enterotoxin II (STII) leaders, LamB, PhoE, PeIB, OmpA and MBP. Well-known signal sequences for use in eukaryotic expression systems include but are not limited to interleukin-2, CD5, the Immunoglobulin Kappa light chain, trypsinogen, serum albumin, and prolactin.

The peptides as described herein (e.g., a peptide comprising a sequence from Tables 1-6) can be expressed as a fusion protein or peptide. Commonly used fusion partners include but are not limited to human serum albumin and the crystallizable fragment, or constant domain of IgG, Fc. The histidine tag or FLAG tag can also be used to simplify purification of recombinant peptide from the expression media or recombinant cell lysate. The fusion partners can be fused to the N- and/or C-terminus of the peptide of interest.

Methods are well known for introducing recombinant DNA, i.e., a nucleic acid construct such as an expression vector, into a host cell so that the DNA is replicable, either as an extrachromosomal element or as a chromosomal integrant, thereby generating a host cell which harbors the nucleic acid construct of interest. Methods of transfection are known to the ordinarily skilled artisan, for example, by CaPO₄ and electroporation. Depending on the host cell used, transformation is performed using standard techniques appropriate to such cells. The calcium treatment employing calcium chloride, as described in Sambrook et al., supra, or electroporation is generally used for prokaryotes or other cells that contain substantial cell-wall barriers. General aspects of mammalian cell host system transformations have been described in U.S. Pat. No. 4,399,216. Transformations into yeast are can be carried out according to the method of Van Solingen et al., J. Bact, 130:946 (1977) and Hsiao et al., Proc. Natl. Acad. Sci. (USA), 76:3829 (1979). Other methods for introducing DNA into cells include nuclear microinjection, electroporation, bacterial protoplast fusion with intact cells, or introduction using polycations, e.g., polybrene, polyornithine. For various techniques for transforming mammalian cells, see Keown et al., Methods in Enzymology. 185:527-537 (1990) and Mansour et al., Nature, 336:348-352 (1988).

Accordingly, provided herein is a nucleic acid construct (e.g., a recombinant vector or expression vector) as described above and comprising a polynucleotide which encodes a peptide sequence of interest (e.g., any of the peptide described herein such as those in Tables 1-6). Moreover, the present disclosure provides a host cell harboring the vector. The host cell can be a eukaryotic or prokaryotic cell as detailed above. In some embodiments, the host cell is a prokaryotic cell. In some embodiments, the host cell is E. coli, L. lactis, S. coelicolor, S. lividans, S. albus, S. venezuelae, or B. subtilis.

### EXAMPLES

### Example 1. Effects of Cysteine to Serine Mutant of SG-3-0020 (SG-3-0020_{C→S}) on Human T Cell Activation

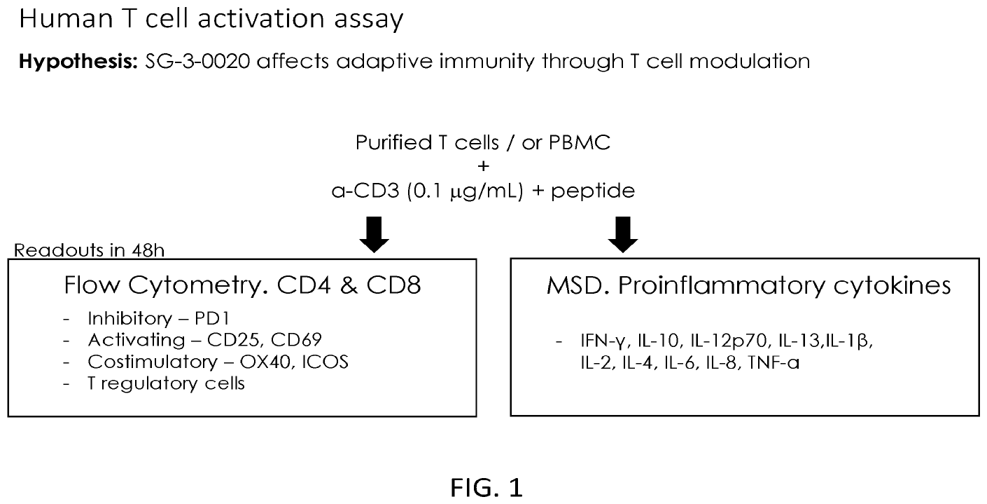

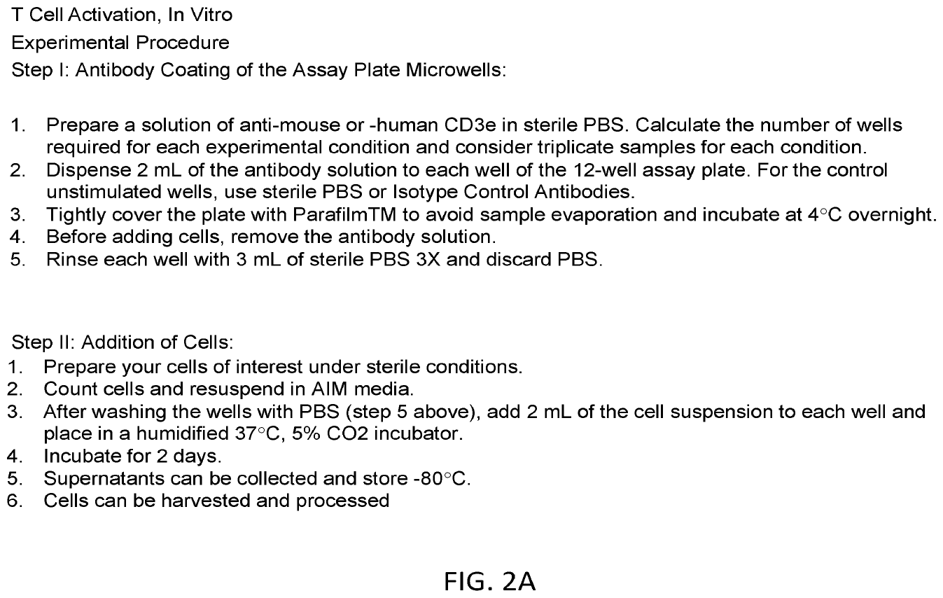

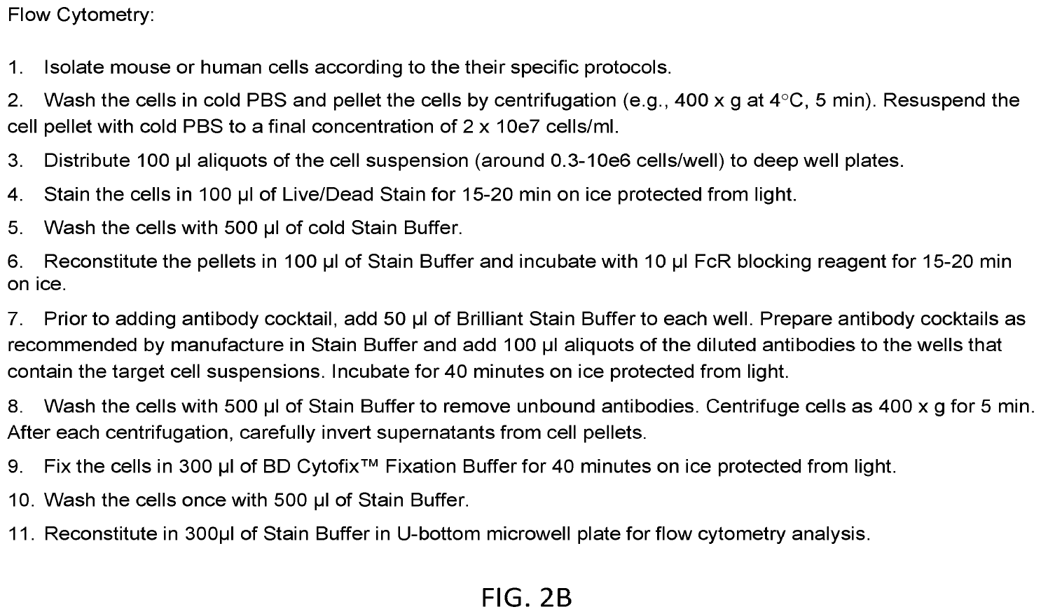

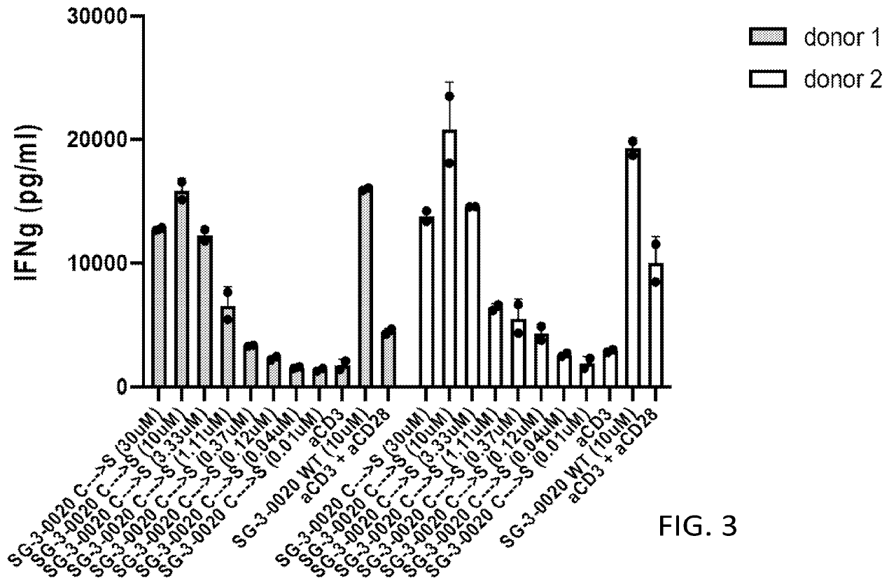

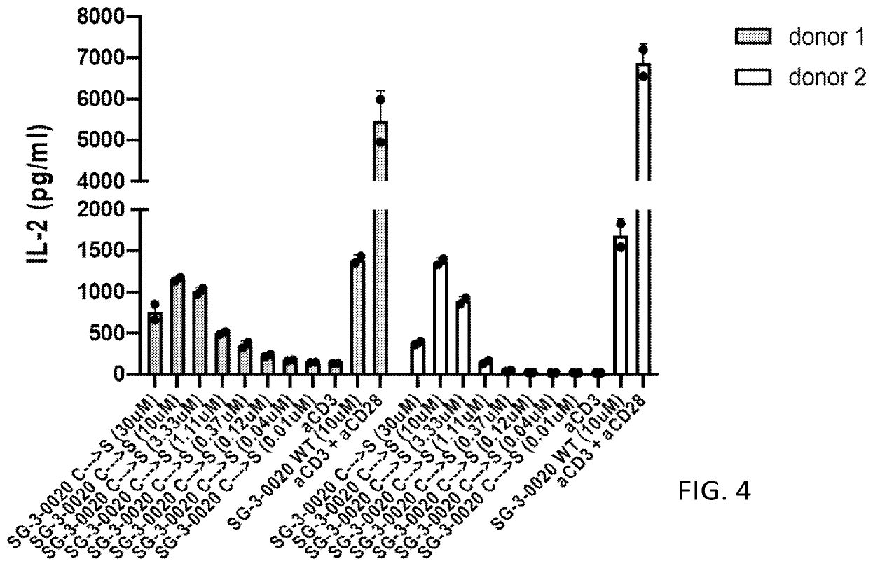

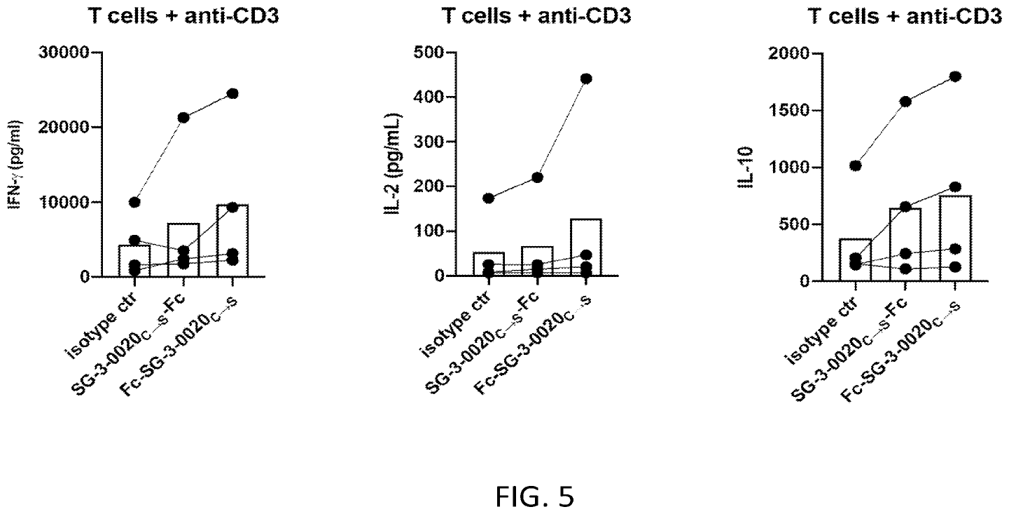

Studies were performed to demonstrate the ability of (SG-3-0020_{C→S}) to activate human T cells and to affect adaptive immunity through T cell modulation as described previously (see International Application No. PCT/US2020/012431). For example, PBMCs were obtained from individual human donors. T cells were purified from the frozen PBMCs (EasySep™ Human T Cell Isolation Kit (Cat. No. 17951, StemCell Technologies). A summary of the protocol is provided in FIG. 1. In vitro activation of the human T cells was performed according to the protocol provided in FIG. 2A. The protocol used for flow cytometry analysis is provided in FIG. 2B. In summary, purified T cells were incubated with anti-CD3 antibody with SG-3-0020_{C→S} or with anti-CD3 and/or anti-CD28 antibody as a control. The results, provided in FIGS. 3 and 4, show that SG-3-0020_{C→S} induces IL2 and IFNg secretion similar to SG-3-0020. FIG. 5 shows that SG-3-0020_mIgG1_N-fusion (SG-3-0020_{C→S}Fc) maintains IL2 and IFNg secretion in human T cells.

### Example 2. Phage Panning to Human T Cells Using SG-2-0020 Mutants Phages Library

SG-3-0020 (Table 7; see International Application No. PCT/US2020/012431) mutants with higher binding affinity to human T cells were enriched through phage panning.

TABLE 7

| SG-3- | MLSTKKTKTHDHYPCGRMRDPGWHDWRACLTHQGIEEDEWP |
|---|---|
| 0020 | V |

### Phage Panning

300 mcL of phage library (multivalent display) were mixed with 300 mcL of 1×PBS+2% BSA. The solution was rotated at room temperature for 1 hour. The cells, either naïve T-cells (about 13×10⁶ cells) or activated T-cells (about 16×10⁶ cells) were thawed. The cells were then centrifuged at 1,500 for 5 min at 4° C., and the supernatant was aspirated. The cell pellets were washed with 5 mL of 1×PBS+2% BSA by pipetting. This was repeated such that the pellets were centrifuged and washed a total of 3 times. The cell pellets were resuspended with 200 mcL of 1×PBS+2% BSA. 100 mcL of resuspended cells and 100 mcL of diluted phages were mixed in a well of 96-well plate (phage binding).

The cells were put on the rocker and incubated at 4° C. for 2 hours for binding and then centrifuged at 1,500 for 5 min at 4° C. The supernatant was discarded. The cell pellets were washed with 250 mcL of 1×PBS+2% BSA by pipetting. This was repeated such that the pellets were centrifuged and washed a total of 5 times.

### Elution of Phages

The cells were washed with 250 mcL of 1×PBS for elution then centrifuged at 1,500 for 5 min at 4° C. 140 mcL of 7.18 M triethylamine (TEA) were added to 10 mL H₂O to make 0.1 M TEA. Elution was carried out with 100 mcL of freshly prepared 0.1 M TEA. This was immediately neutralized by adding 100 mcL of 1 M TRIS pH 8.0.

### Amplification of Phages

5 mL of ice-chilled E. coli TG-1 cells (OD˜0.6) were added into 125 mL of flask. 350 mcL of eluted phages were added to E. coli TG-1 cells. The E. coli TG-1 cells were incubated at 37 C without shaking for 30 min. 45 mL of 2×YT media with 100 mcg/ml of carbenicillin was added to the cells and incubated at 37 C, 200 rpm, for 1 hr. 50 mcL of helper phage M13KO7d3 was added to the cells and incubated at 37 C, 200 rpm, for 1 hr. 50 mcL of kanamycine (50 mg/mL) was added and incubated at 30 .C°, 200 rpm, overnight.

### Example 3. SG-3-0020 Binding Core to Human T-Cell

Binding region was analyzed by replacing a stretch of amino acids to all alanine residues (alanine shaving). Polynucleotides coding sequence Ala1-12, NTF corresponding Met1 to Pro14 of SG-3-0020, MID corresponding Gly16 to Ala28 of SG-3-0020, CTF corresponding Leu32 to Val42 of SG-3-0020 with more than 25-base length flanking region at the both 5′- and 3′-ends were synthesized by Integrated DNA Technologies. Typically, synthesized DNA was dissolved in 100 μL of water and assembled to phagemid pADL-23c using NEBuilder® HiFi DNA Assembly Master Mix (New England BioLabs). Phagemid DNA and synthesized DNA were mixed with 1:3 molar ratio and up to 0.2 pmol of total amount in 5 μL total volume. 10 μL of NEBuilder HiFi DNA Assembly Master Mix was added to DNA. Assemble reaction was initiated by heating at 50° C. for 15 min and completed by heating at 75° C. for 10 min. Assembled mixture was typically transformed to E. coli and inoculated 2YT plate containing 100 μg/mL carbenicillin and phagemid was isolated for sequencing. Phage displaying Ala1-12 was amplified as mentioned above (Example 2).

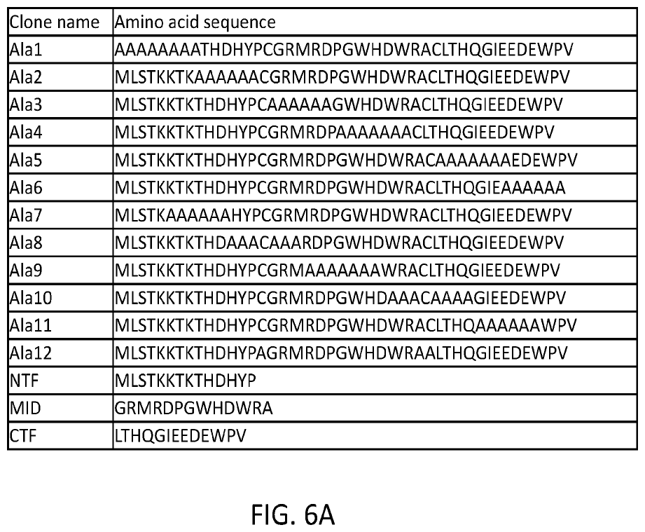

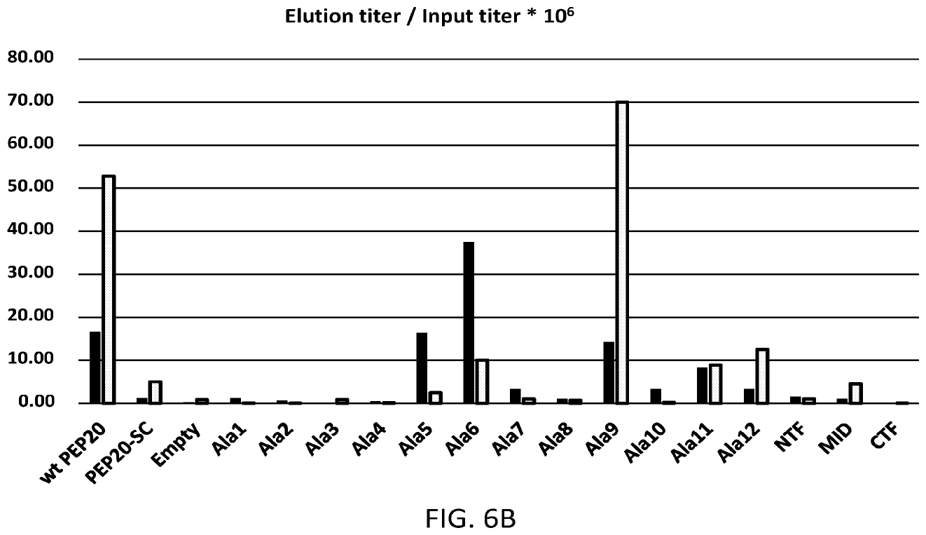

Phage binding to activated human T-cell was measured by comparing input and elution titer. Standard methods were described in Microbiological Methods (Rider J. E., et al., 1996). Alanine shaving between L32 to V42 didn't affect binding capacity. Other residues significantly reduced binding. See FIGS. 6A and 6B.

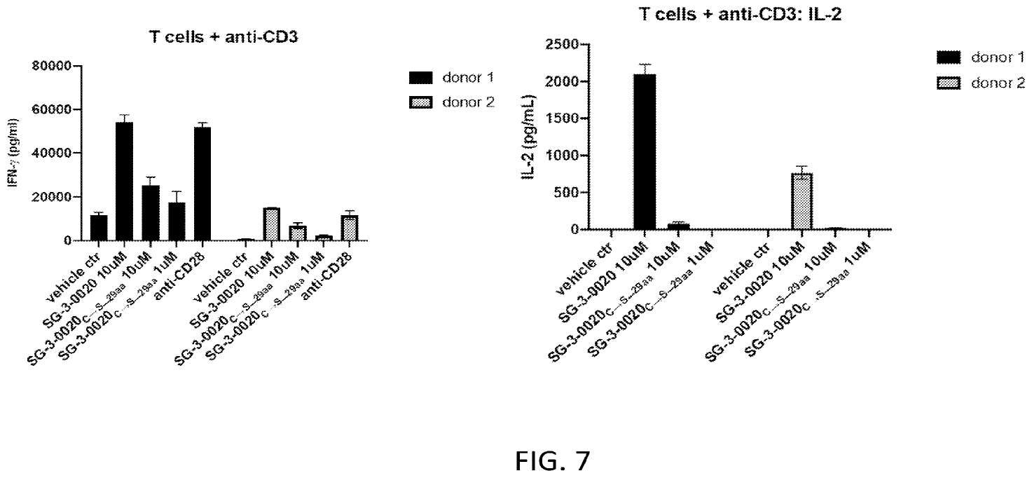

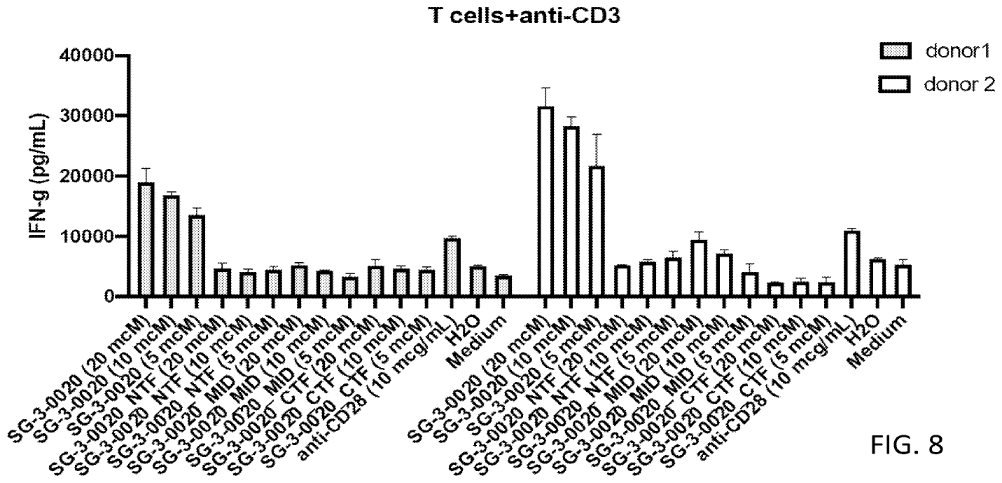

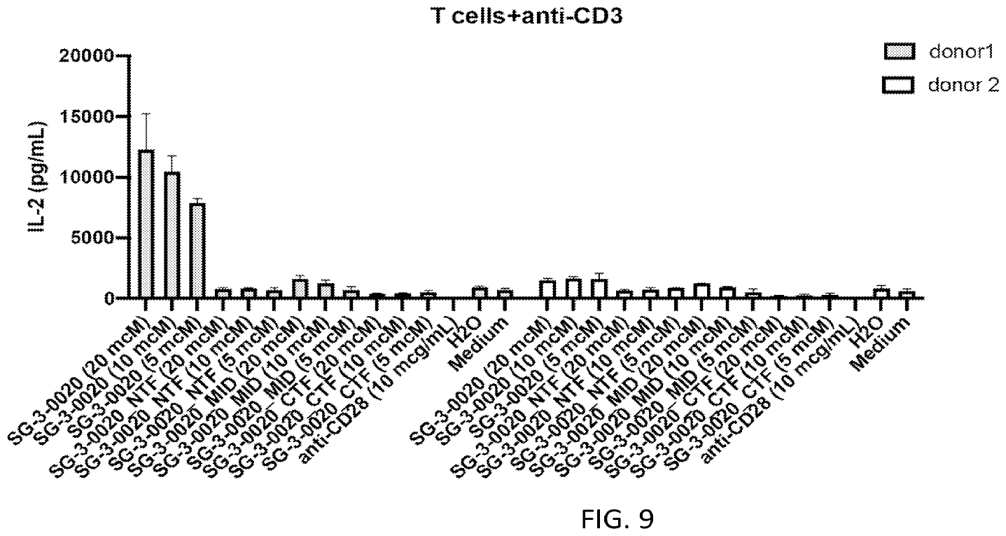

T-cell activation was also determined as described previously (see above and International Application No. PCT/US2020/012431). SG-3-0020 1-29 aa N-terminal+middle regions required for binding show partial T cell activation compared to the full length peptides. Separate N-, MID, and C-terminal fragments lose activity. The data support that both N- and middle region are minimally required for binding and activation to T cells. See FIGS. 7A, 7B, 8, and 9.

### Example 4. Saturation Mutagenesis on SG-3-05067

SEQ ID NO:1 (MLSTKKTKTHDHYPSGRMRDPGWHDWRAS) was used as a template for saturation mutagenesis at all positions. Each amino acid was replaced to 18 or 17 amino acids. Wildtype amino acid, methionine and cysteine residues were excluded from mutations. Polynucleotides with mutations were chemically synthesized by Twist Bioscience with 24-base length flanking regions at both 5′ and 3′ ends for cloning. The synthesized polynucleotides mixture was cloned into pADL-3c phagemid using NEBuilder® HiFi DNA Assembly Master Mix as described above. Assembled DNA was typically transformed to E. coli TG-1 cells with electroporation and plated onto 2-YT plates containing 100 μg/mL carbenicillin. The phage library was amplified as described above.

Phage panning was performed against naïve and activated human T-cells, respectively as described above (Example 2).

Eluted phage was amplified as described above and was analyzed by Illumina MiSeq NGS. For next generation sequencing in the Illumina MiSeq, nucleotide sequences are amplified from plasmid DNA purified from E. coli transformed with a phagemid vector produced for a Phage Display Library. The PCR amplification primers used match the common flanking regions of the nucleotide sequences inserted the phagemid vector. The PCR primers have been specifically designed to incorporate Illumina flow cell adapters with indexing barcodes. Standard methods were used as described in Ultrahighthroughput microbial community analysis on the Illumina HiSeq and MiSeq platforms (Caporaso, J. G. et al. 2012).

NGS sequence counts were compared enrichment ratio between naïve and activated human T-cell. Among 437 mutants, 58 mutants were more than 1.5-fold enriched in activated T-cell comparing the original bacteriophage pool and 21 mutants were more than 1.8-fold enriched in activated T-cell over naïve T-cells. 358 mutants weaken its binding to active T-cell, less than 0.7-fold enrichment over the original bacteriophage pool. The selection criteria were:
High binders: Activated/original pool>=1.5. See Table 8.
Activated T-cell specific binders: Medium binder, T-cell over original pool (1.0-1.5), but enriched in activated T-cell over naïve T-cell (>1.8). See Table 9.
Low binders: 0.7>Activated/original pool. See Table 10.

TABLE 8
High Binders

| Sequence | |
|---|---|
| Identifier | Sequence |
| SEQ ID NO: 163 | FLSTKKTKTHDHYPSGRMRDPGWHDWRASGGGGSGGGGS |
| SEQ ID NO: 164 | KLSTKKTKTHDHYPSGRMRDPGWHDWRASGGGGSGGGGS |
| SEQ ID NO: 165 | PLSTKKTKTHDHYPSGRMRDPGWHDWRASGGGGSGGGGS |
| SEQ ID NO: 166 | RLSTKKTKTHDHYPSGRMRDPGWHDWRASGGGGSGGGGS |
| SEQ ID NO: 167 | MASTKKTKTHDHYPSGRMRDPGWHDWRASGGGGSGGGGS |
| SEQ ID NO: 168 | MGSTKKTKTHDHYPSGRMRDPGWHDWRASGGGGSGGGGS |
| SEQ ID NO: 169 | MKSTKKTKTHDHYPSGRMRDPGWHDWRASGGGGSGGGGS |
| SEQ ID NO: 170 | MPSTKKTKTHDHYPSGRMRDPGWHDWRASGGGGSGGGGS |
| SEQ ID NO: 171 | MQSTKKTKTHDHYPSGRMRDPGWHDWRASGGGGSGGGGS |
| SEQ ID NO: 172 | MRSTKKTKTHDHYPSGRMRDPGWHDWRASGGGGSGGGGS |
| SEQ ID NO: 173 | MSSTKKTKTHDHYPSGRMRDPGWHDWRASGGGGSGGGGS |
| SEQ ID NO: 174 | MTSTKKTKTHDHYPSGRMRDPGWHDWRASGGGGSGGGGS |
| SEQ ID NO: 175 | MWSTKKTKTHDHYPSGRMRDPGWHDWRASGGGGSGGGGS |
| SEQ ID NO: 176 | MYSTKKTKTHDHYPSGRMRDPGWHDWRASGGGGSGGGGS |
| SEQ ID NO: 177 | MLQTKKTKTHDHYPSGRMRDPGWHDWRASGGGGSGGGGS |
| SEQ ID NO: 178 | MLRTKKTKTHDHYPSGRMRDPGWHDWRASGGGGSGGGGS |
| SEQ ID NO: 179 | MLSRKKTKTHDHYPSGRMRDPGWHDWRASGGGGSGGGGS |
| SEQ ID NO: 180 | MLSTRKTKTHDHYPSGRMRDPGWHDWRASGGGGSGGGGS |
| SEQ ID NO: 181 | MLSTKRTKTHDHYPSGRMRDPGWHDWRASGGGGSGGGGS |
| SEQ ID NO: 182 | MLSTKTTKTHDHYPSGRMRDPGWHDWRASGGGGSGGGGS |
| SEQ ID NO: 183 | MLSTKKKKTHDHYPSGRMRDPGWHDWRASGGGGSGGGGS |
| SEQ ID NO: 184 | MLSTKKRKTHDHYPSGRMRDPGWHDWRASGGGGSGGGGS |
| SEQ ID NO: 185 | MLSTKKTKRHDHYPSGRMRDPGWHDWRASGGGGSGGGGS |
| SEQ ID NO: 186 | MLSTKKTKTKDHYPSGRMRDPGWHDWRASGGGGSGGGGS |
| SEQ ID NO: 187 | MLSTKKTKTRDHYPSGRMRDPGWHDWRASGGGGSGGGGS |
| SEQ ID NO: 188 | MLSTKKTKTHFHYPSGRMRDPGWHDWRASGGGGSGGGGS |
| SEQ ID NO: 189 | MLSTKKTKTHGHYPSGRMRDPGWHDWRASGGGGSGGGGS |
| SEQ ID NO: 190 | MLSTKKTKTHHHYPSGRMRDPGWHDWRASGGGGSGGGGS |
| SEQ ID NO: 191 | MLSTKKTKTHIHYPSGRMRDPGWHDWRASGGGGSGGGGS |
| SEQ ID NO: 192 | MLSTKKTKTHKHYPSGRMRDPGWHDWRASGGGGSGGGGS |
| SEQ ID NO: 193 | MLSTKKTKTHPHYPSGRMRDPGWHDWRASGGGGSGGGGS |
| SEQ ID NO: 194 | MLSTKKTKTHRHYPSGRMRDPGWHDWRASGGGGSGGGGS |
| SEQ ID NO: 195 | MLSTKKTKTHTHYPSGRMRDPGWHDWRASGGGGSGGGGS |
| SEQ ID NO: 196 | MLSTKKTKTHVHYPSGRMRDPGWHDWRASGGGGSGGGGS |
| SEQ ID NO: 197 | MLSTKKTKTHWHYPSGRMRDPGWHDWRASGGGGSGGGGS |
| SEQ ID NO: 198 | MLSTKKTKTHYHYPSGRMRDPGWHDWRASGGGGSGGGGS |
| SEQ ID NO: 199 | MLSTKKTKTHDKYPSGRMRDPGWHDWRASGGGGSGGGGS |
| SEQ ID NO: 200 | MLSTKKTKTHDRYPSGRMRDPGWHDWRASGGGGSGGGGS |
| SEQ ID NO: 201 | MLSTKKTKTHDHGPSGRMRDPGWHDWRASGGGGSGGGGS |
| SEQ ID NO: 202 | MLSTKKTKTHDHKPSGRMRDPGWHDWRASGGGGSGGGGS |
| SEQ ID NO: 203 | MLSTKKTKTHDHRPSGRMRDPGWHDWRASGGGGSGGGGS |
| SEQ ID NO: 204 | MLSTKKTKTHDHWPSGRMRDPGWHDWRASGGGGSGGGGS |
| SEQ ID NO: 205 | MLSTKKTKTHDHYPRGRMRDPGWHDWRASGGGGSGGGGS |
| SEQ ID NO: 206 | MLSTKKTKTHDHYPSGRGRDPGWHDWRASGGGGSGGGGS |
| SEQ ID NO: 207 | MLSTKKTKTHDHYPSGRHRDPGWHDWRASGGGGSGGGGS |
| SEQ ID NO: 208 | MLSTKKTKTHDHYPSGRKRDPGWHDWRASGGGGSGGGGS |
| SEQ ID NO: 209 | MLSTKKTKTHDHYPSGRRRDPGWHDWRASGGGGSGGGGS |
| SEQ ID NO: 210 | MLSTKKTKTHDHYPSGRWRDPGWHDWRASGGGGSGGGGS |
| SEQ ID NO: 211 | MLSTKKTKTHDHYPSGRMRPPGWHDWRASGGGGSGGGGS |
| SEQ ID NO: 212 | MLSTKKTKTHDHYPSGRMRRPGWHDWRASGGGGSGGGGS |
| SEQ ID NO: 213 | MLSTKKTKTHDHYPSGRMRDPPWHDWRASGGGGSGGGGS |
| SEQ ID NO: 214 | MLSTKKTKTHDHYPSGRMRDPGWHPWRASGGGGSGGGGS |
| SEQ ID NO: 215 | MLSTKKTKTHDHYPSGRMRDPGWHRWRASGGGGSGGGGS |
| SEQ ID NO: 216 | MLSTKKTKTHDHYPSGRMRDPGWHDWWASGGGGSGGGGS |
| SEQ ID NO: 217 | MLSTKKTKTHDHYPSGRMRDPGWHDWRFSGGGGSGGGGS |
| SEQ ID NO: 218 | MLSTKKTKTHDHYPSGRMRDPGWHDWRGSGGGGSGGGGS |
| SEQ ID NO: 219 | MLSTKKTKTHDHYPSGRMRDPGWHDWRVSGGGGSGGGGS |
| SEQ ID NO: 220 | MLSTKKTKTHDHYPSGRMRDPGWHDWRYSGGGGSGGGGS |

TABLE 9
Activated T-cell Specific Binders

| Sequence | |
|---|---|
| Identifier | Sequence |
| SEQ ID NO: 221 | SLSTKKTKTHDHYPSGRMRDPGWHDWRASGGGGSG |
| | GGGS |
| SEQ ID NO: 222 | VLSTKKTKTHDHYPSGRMRDPGWHDWRASGGGGSG |
| | GGGS |
| SEQ ID NO: 223 | WLSTKKTKTHDHYPSGRMRDPGWHDWRASGGGGSG |
| | GGGS |
| SEQ ID NO: 224 | MFSTKKTKTHDHYPSGRMRDPGWHDWRASGGGGSG |
| | GGGS |
| SEQ ID NO: 225 | MNSTKKTKTHDHYPSGRMRDPGWHDWRASGGGGSG |
| | GGGS |
| SEQ ID NO: 226 | MVSTKKTKTHDHYPSGRMRDPGWHDWRASGGGGSG |
| | GGGS |
| SEQ ID NO: 227 | MLSAKKTKTHDHYPSGRMRDPGWHDWRASGGGGSG |
| | GGGS |
| SEQ ID NO: 228 | MLSGKKTKTHDHYPSGRMRDPGWHDWRASGGGGSG |
| | GGGS |
| SEQ ID NO: 229 | MLSTAKTKTHDHYPSGRMRDPGWHDWRASGGGGSG |
| | GGGS |
| SEQ ID NO: 230 | MLSTKKGKTHDHYPSGRMRDPGWHDWRASGGGGSG |
| | GGGS |
| SEQ ID NO: 231 | MLSTKKTKWHDHYPSGRMRDPGWHDWRASGGGGSG |
| | GGGS |
| SEQ ID NO: 232 | MLSTKKTKTHDHNPSGRMRDPGWHDWRASGGGGSG |
| | GGGS |
| SEQ ID NO: 233 | MLSTKKTKTHDHYGSGRMRDPGWHDWRASGGGGSG |
| | GGGS |
| SEQ ID NO: 234 | MLSTKKTKTHDHYWSGRMRDPGWHDWRASGGGGSG |
| | GGGS |
| SEQ ID NO: 235 | MLSTKKTKTHDHYPGGRMRDPGWHDWRASGGGGSG |
| | GGGS |
| SEQ ID NO: 236 | MLSTKKTKTHDHYPSGRYRDPGWHDWRASGGGGSG |
| | GGGS |
| SEQ ID NO: 237 | MLSTKKTKTHDHYPSGRMRDFGWHDWRASGGGGSG |
| | GGGS |
| SEQ ID NO: 238 | MLSTKKTKTHDHYPSGRMRDPKWHDWRASGGGGSG |
| | GGGS |
| SEQ ID NO: 239 | MLSTKKTKTHDHYPSGRMRDPWWHDWRASGGGGSG |
| | GGGS |
| SEQ ID NO: 240 | MLSTKKTKTHDHYPSGRMRDPGWHDWRWSGGGGSG |
| | GGGS |
| SEQ ID NO: 241 | MLSTKKTKTHDHYPSGRMRDPGWHDWRARGGGGSG |
| | GGGS |

### Example 5. Combinatorial Library Screening

Saturation mutagenesis analysis showed beneficial mutations as shown in Tables 8-10.

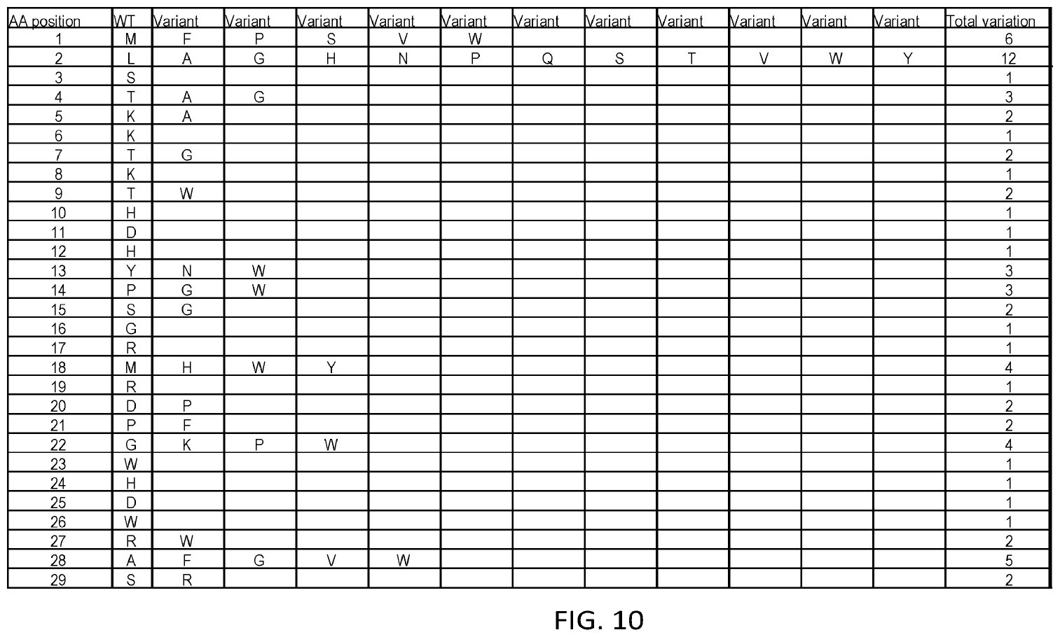

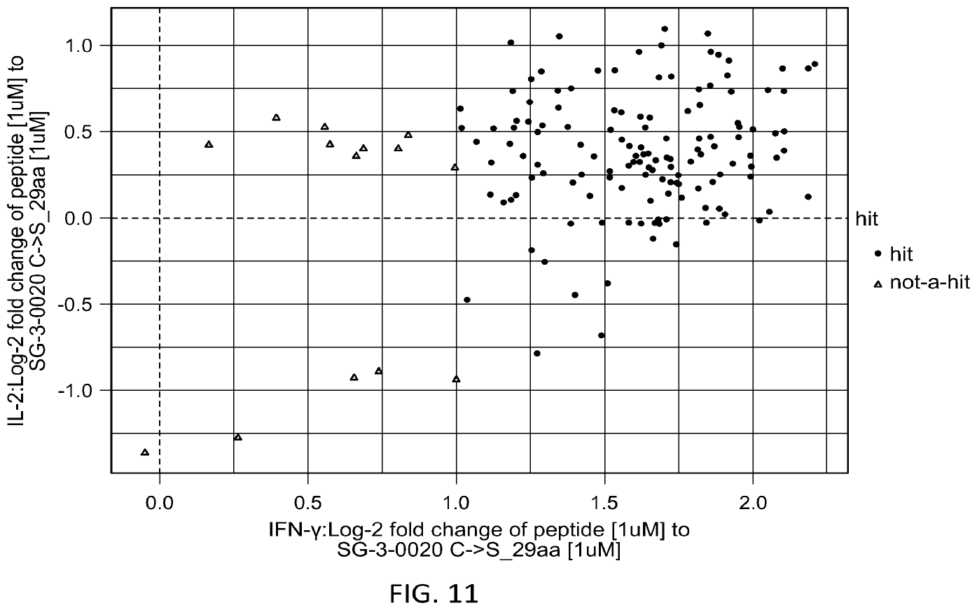

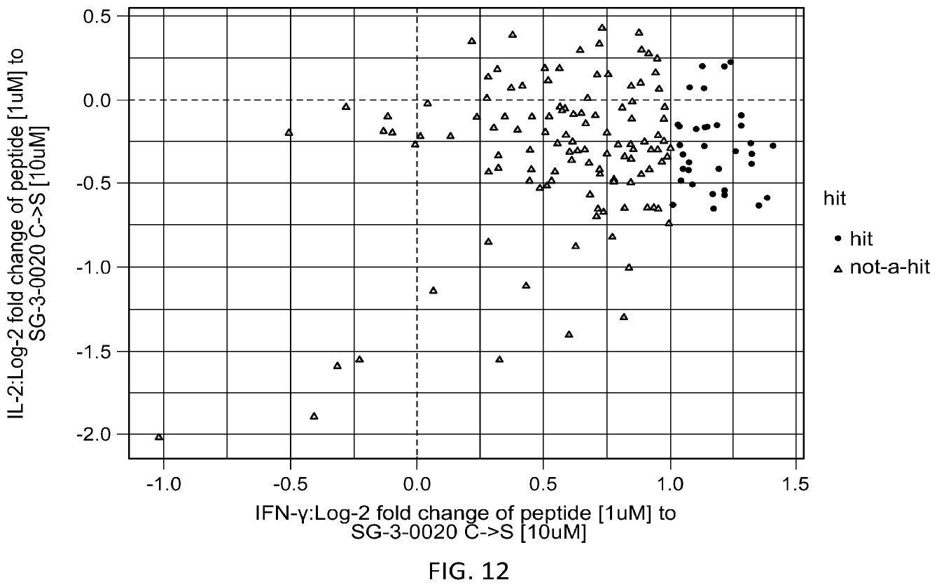

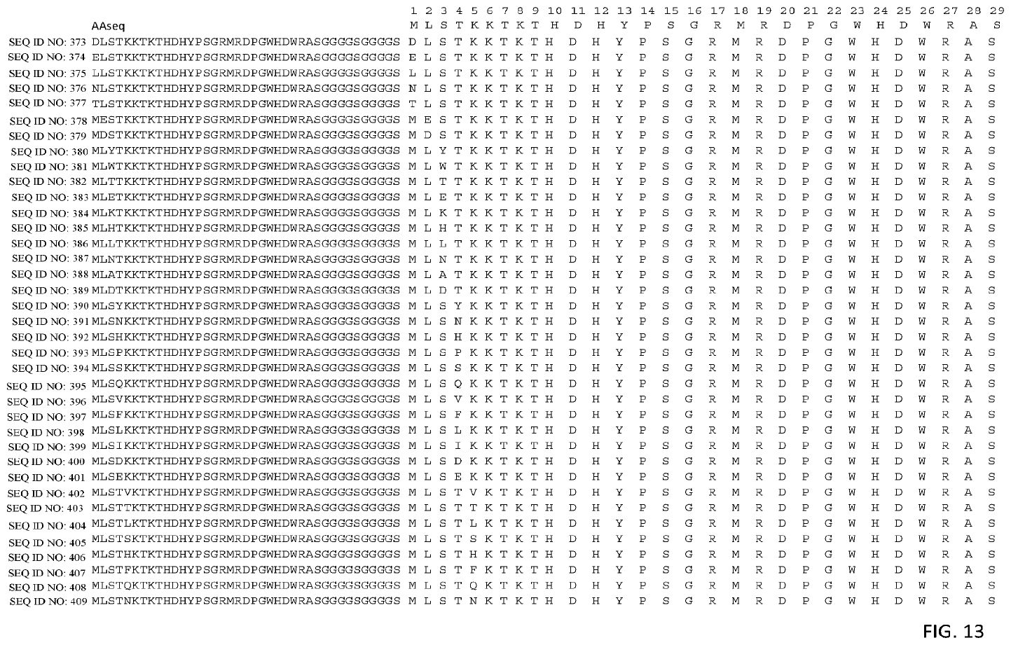

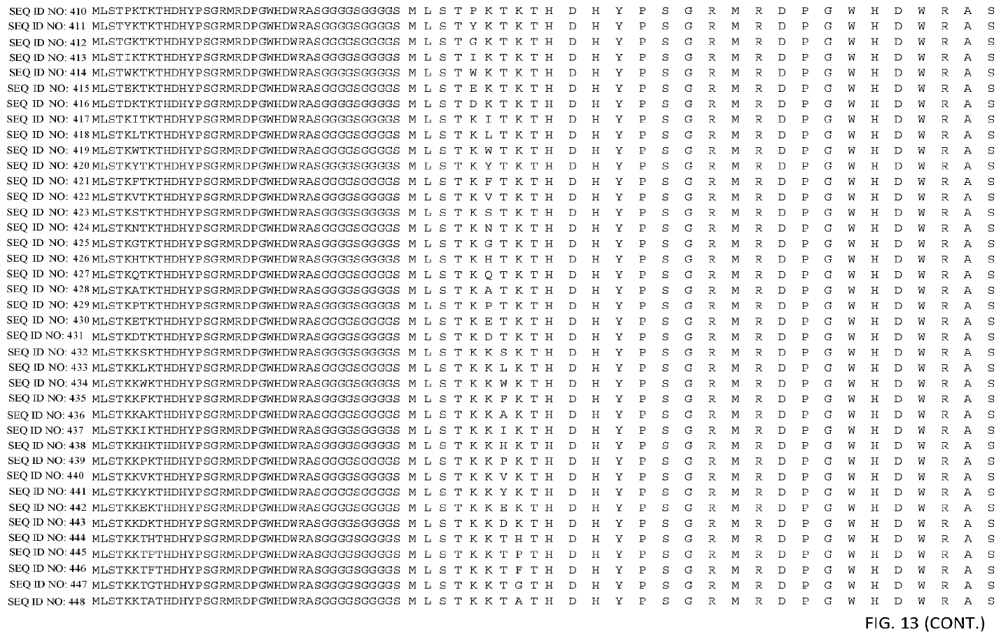

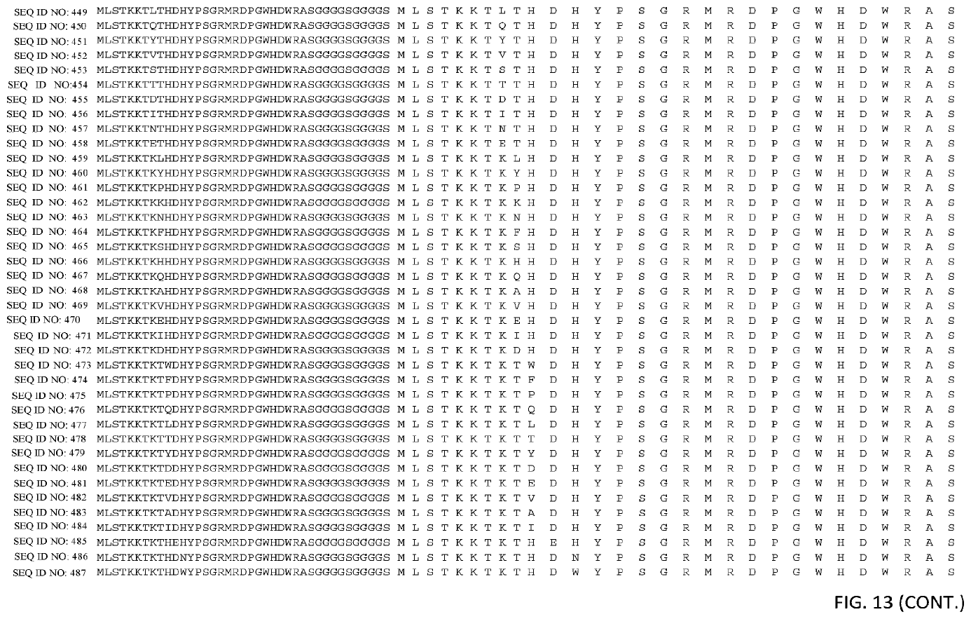

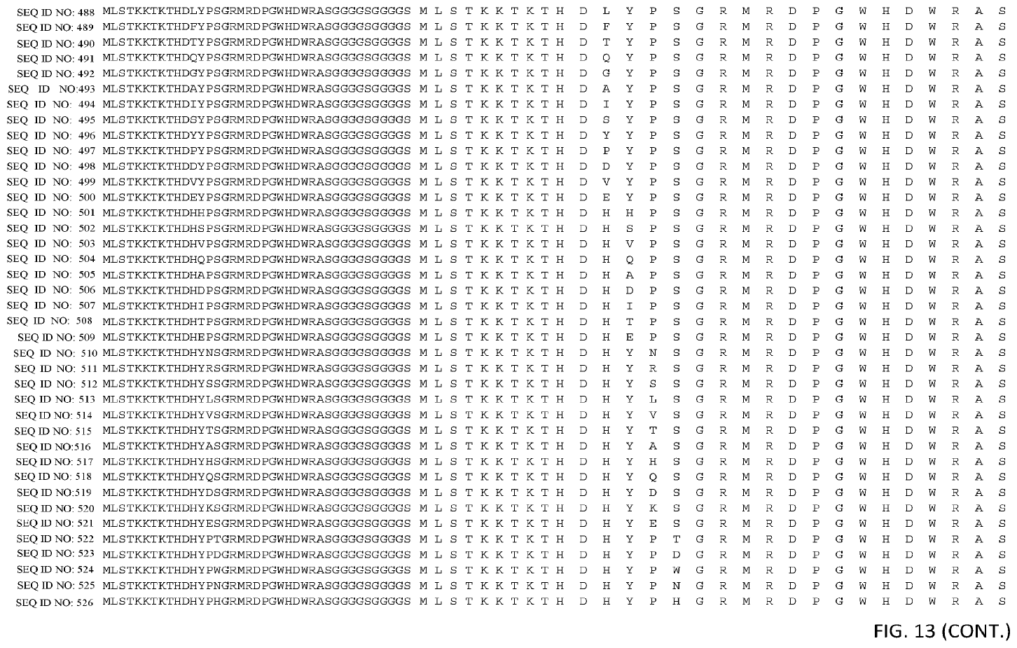

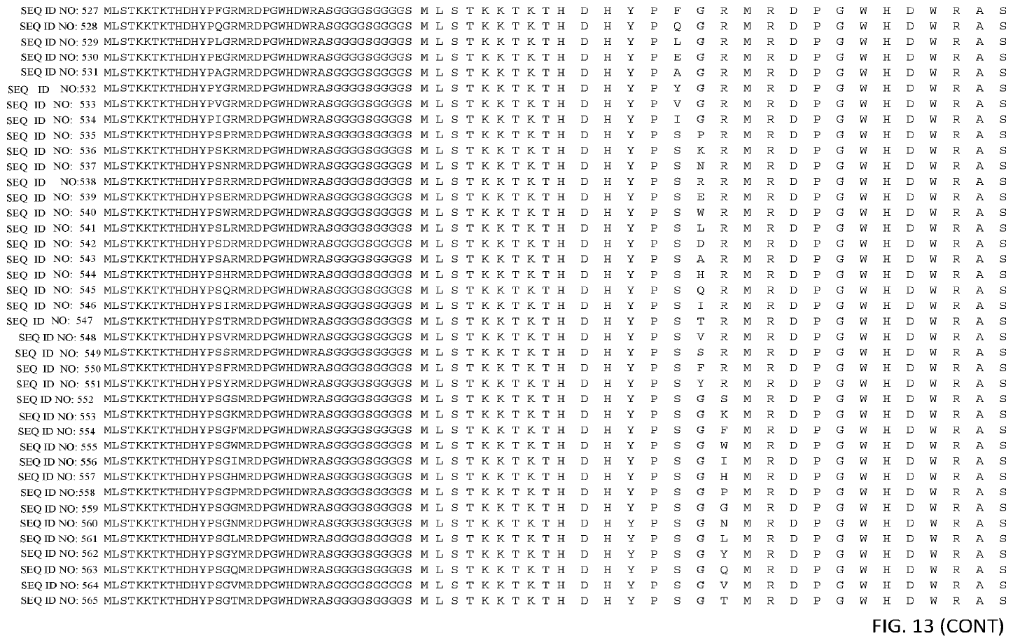

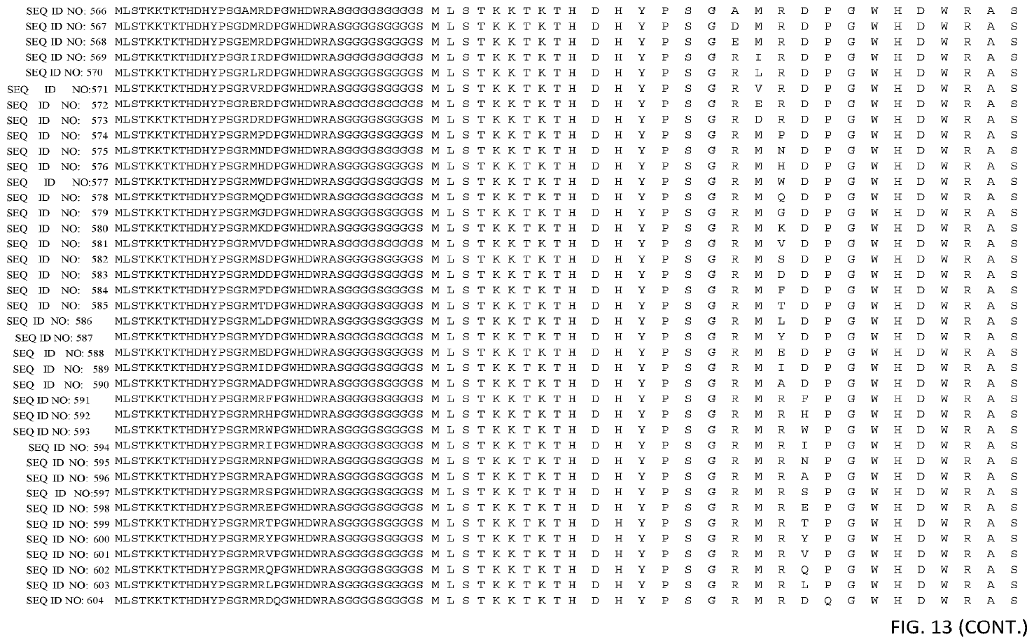

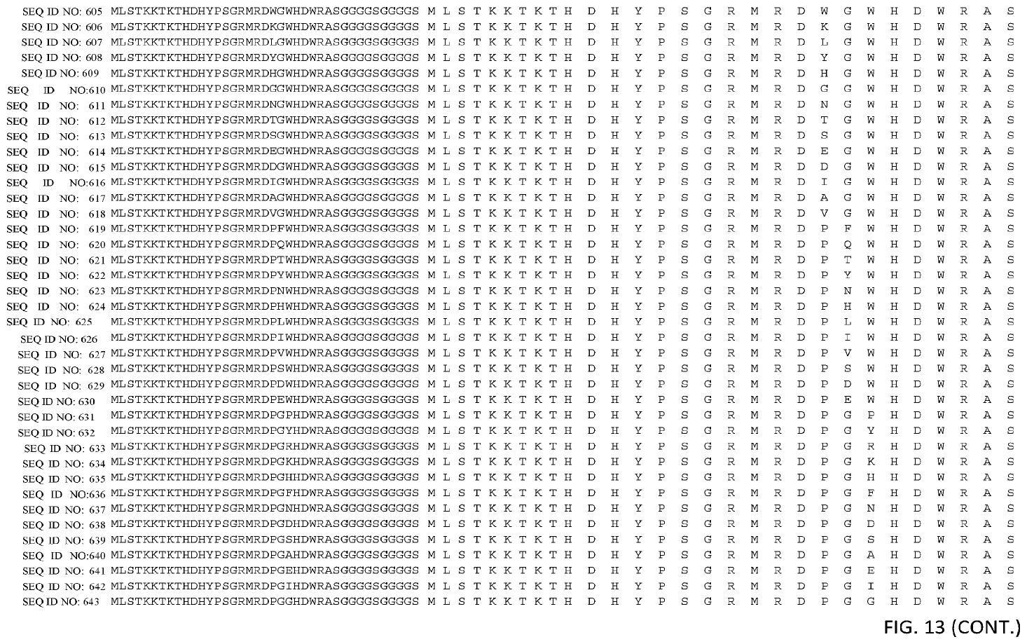

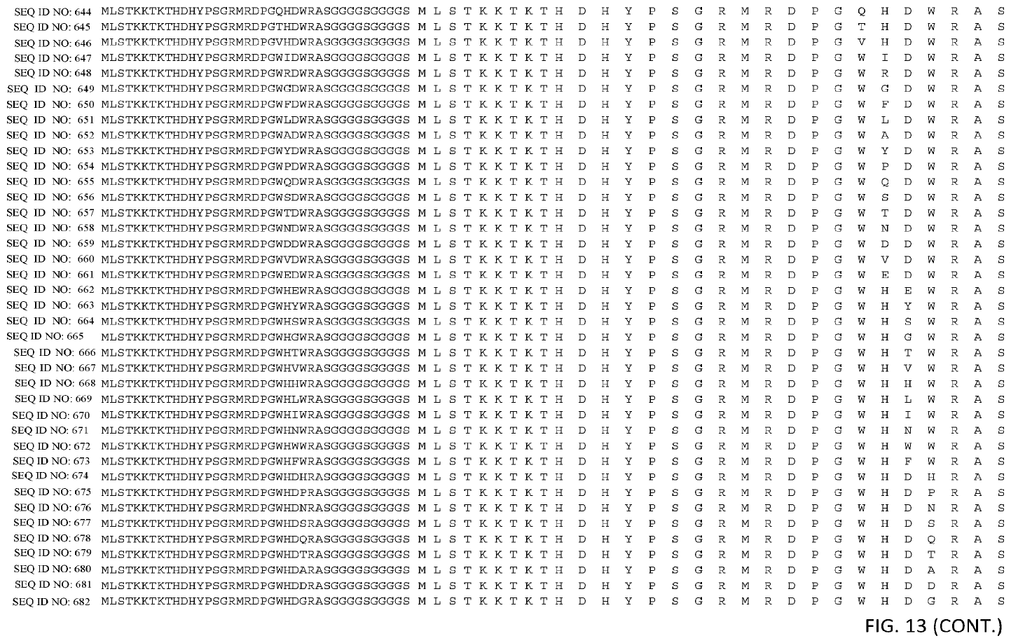

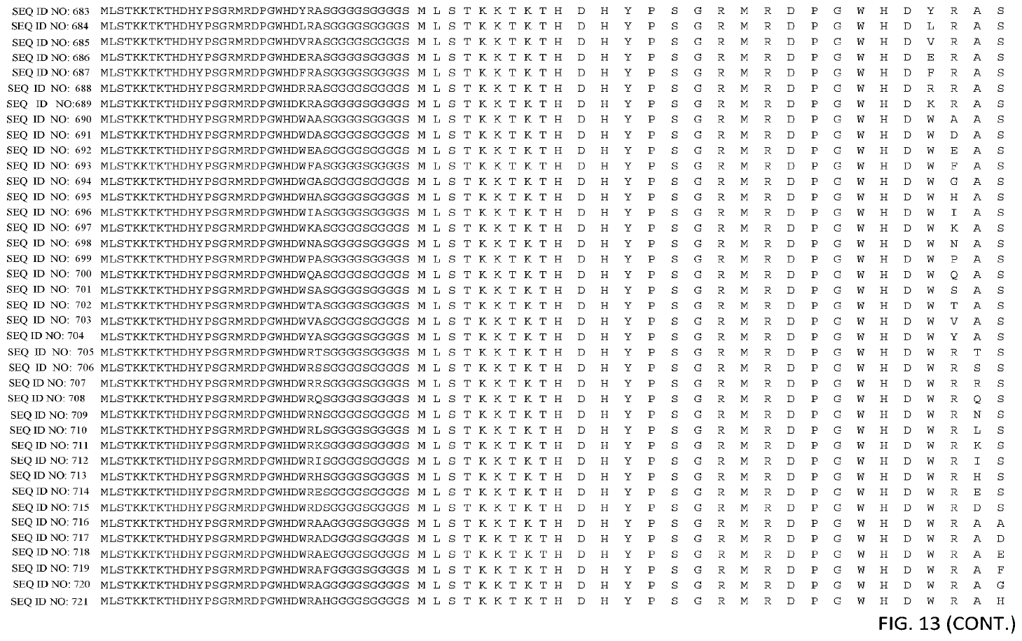

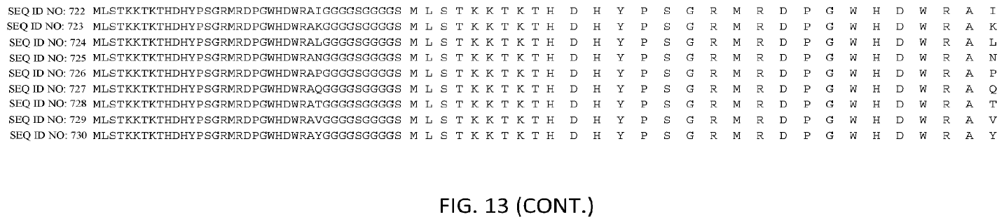

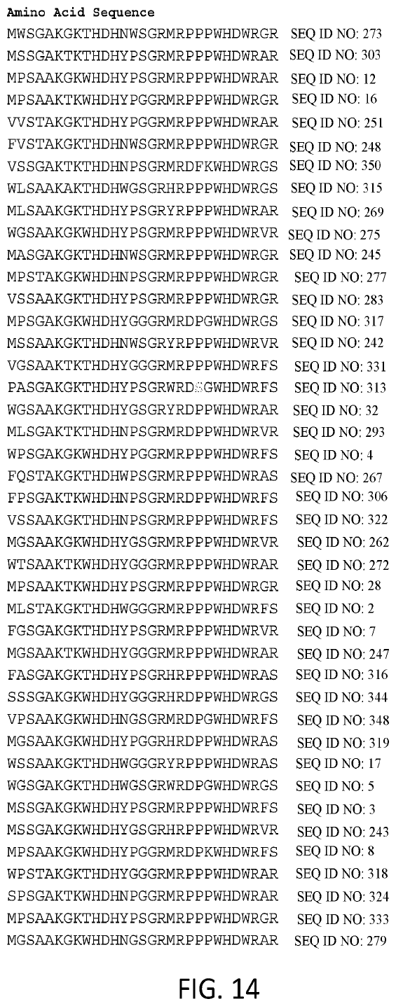

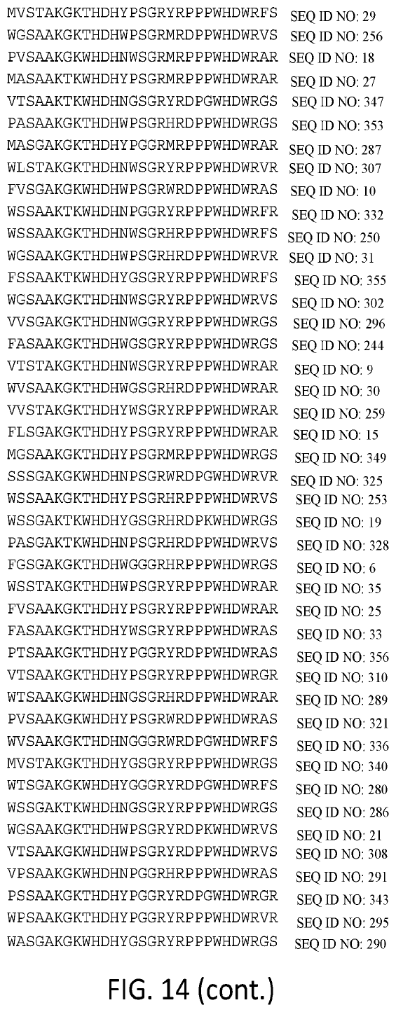

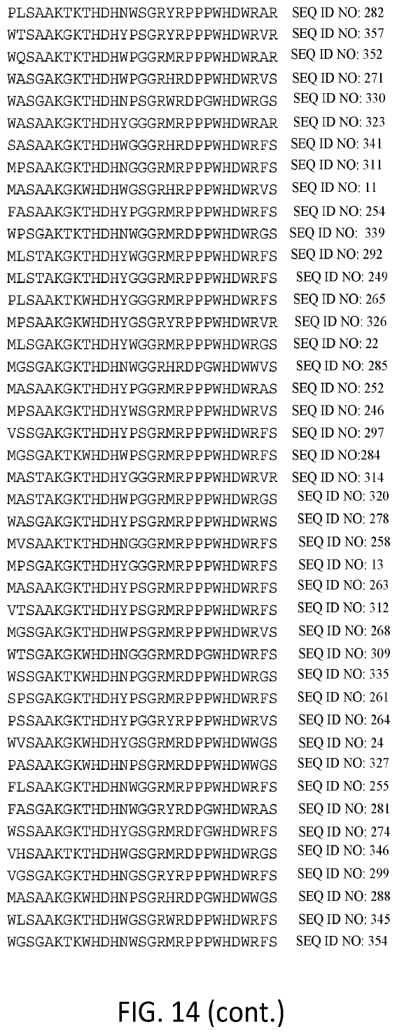

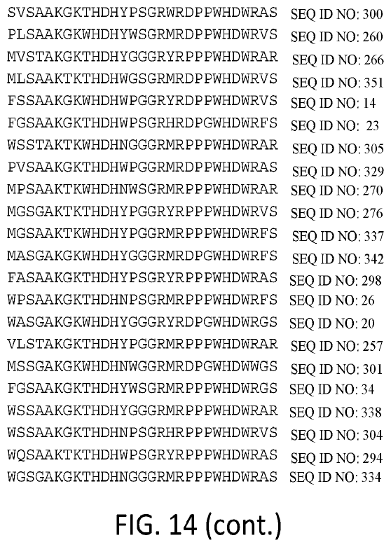

A combinatorial library was synthesized using SEQ ID NO:1 as a template. See FIG. 10. Polynucleotides with all possible combination (3.98×10⁷ variants) with 24-base length flanking region at the both 5′ and 3′ ends were chemically synthesized by Twist Bioscience and cloned phagemid vector. Phage library was prepared and phage panning to naïve and activated human T-cell was performed as described above. Eluted phage was amplified and analyzed by NGS as described above. Sequence counts of each mutant from active and naïve human T-cell were analyzed and 150 mutants were selected (see Table 10), top 50 mutants enriched in active T-cell, 39 mutants from top 200 mutants enriched in active T-cell without methionine residue containing and 61 mutants from enriched in active T-cell and not observed in top 500 mutants enriched in naïve T-cell. See also Tables 10-11 and FIGS. 11-14.

TABLE 10

| | | Sequence |
|---|---|---|
| mean_ES_IFN-γ | Sequence | Identifier |
| 1.408204774 | MLSTAKGKTHDHWGGGRMRPPPWHDWRFS | SEQ ID NO: 2 |
| 1.384115185 | MSSGAKGKWHDHYPSGRMRPPPWHDWRFS | SEQ ID NO: 3 |
| 1.350729236 | WPSGAKGKWHDHYPGGRMRPPPWHDWRFS | SEQ ID NO: 4 |
| 1.323750558 | WGSGAKGKTHDHWGSGRWRDPGWHDWRGS | SEQ ID NO: 5 |
| 1.320605759 | FGSGAKGKTHDHWGGGRHRPPPWHDWRGS | SEQ ID NO: 6 |
| 1.318629531 | FGSGAKGKTHDHYPSGRMRPPPWHDWRVR | SEQ ID NO: 7 |
| 1.282381254 | MPSAAKGKWHDHYPGGRMRDPKWHDWRFS | SEQ ID NO: 8 |
| 1.280103826 | VTSTAKGKTHDHNWSGRYRPPPWHDWRAR | SEQ ID NO: 9 |
| 1.26032041 | FVSGAKGKWHDHWPSGRWRDPPWHDWRAS | SEQ ID NO: 10 |
| 1.236924622 | MASAAKGKWHDHWGSGRHRPPPWHDWRVS | SEQ ID NO: 11 |
| 1.216102903 | MPSAAKGKWHDHYPSGRMRPPPWHDWRAR | SEQ ID NO: 12 |
| 1.215664885 | MPSGAKGKTHDHYGGGRMRPPPWHDWRFS | SEQ ID NO: 13 |
| 1.214913594 | FSSAAKGKWHDHWPGGRYRDPPWHDWRVS | SEQ ID NO: 14 |
| 1.193103249 | FLSGAKGKTHDHYPSGRYRPPPWHDWRAR | SEQ ID NO: 15 |
| 1.185693762 | MPSAAKTKWHDHYPGGRMRPPPWHDWRGR | SEQ ID NO: 16 |
| 1.172406636 | WSSAAKGKTHDHWGGGRYRPPPWHDWRAS | SEQ ID NO: 17 |
| 1.168651327 | PVSAAKGKWHDHNWSGRMRPPPWHDWRAR | SEQ ID NO: 18 |
| 1.14735731 | WSSGAKTKWHDHYGSGRHRDPKWHDWRGS | SEQ ID NO: 19 |
| 1.134635539 | WASGAKGKWHDHYGGGRYRDPGWHDWRGS | SEQ ID NO: 20 |
| 1.134437398 | WGSAAKGKTHDHWPSGRYRDPKWHDWRVS | SEQ ID NO: 21 |
| 1.133835902 | MLSGAKGKTHDHYWGGRMRPPPWHDWRGS | SEQ ID NO: 22 |
| 1.129375178 | FGSAAKGKTHDHWPSGRHRDPGWHDWRFS | SEQ ID NO: 23 |
| 1.104085676 | WVSAAKGKWHDHYGSGRMRDPPWHDWWGS | SEQ ID NO: 24 |
| 1.087993078 | FVSAAKGKTHDHYPSGRYRPPPWHDWRAR | SEQ ID NO: 25 |
| 1.078478691 | WPSAAKGKTHDHNPSGRMRPPPWHDWRFS | SEQ ID NO: 26 |
| 1.075356247 | MASAAKTKWHDHYPSGRMRPPPWHDWRAR | SEQ ID NO: 27 |
| 1.07145419 | MPSAAKTKWHDHYPSGRMRPPPWHDWRGR | SEQ ID NO: 28 |
| 1.051932717 | MVSTAKGKTHDHYPSGRYRPPPWHDWRFS | SEQ ID NO: 29 |
| 1.051581331 | WVSAAKGKTHDHWGSGRHRDPPWHDWRAR | SEQ ID NO: 30 |
| 1.043839446 | WGSAAKGKTHDHWPSGRHRDPPWHDWRVR | SEQ ID NO: 31 |
| 1.038239035 | WGSAAKGKTHDHYGSGRYRDPPWHDWRAR | SEQ ID NO: 32 |
| 1.035817099 | FASAAKGKTHDHYWSGRYRPPPWHDWRAS | SEQ ID NO: 33 |
| 1.0281755 | FGSAAKGKTHDHYWSGRMRPPPWHDWRGS | SEQ ID NO: 34 |
| 1.009032089 | WSSTAKGKTHDHWPSGRYRPPPWHDWRAR | SEQ ID NO: 35 |

TABLE 11

| | | Sequence |
|---|---|---|
| mean_ES_IFN-γ | Sequence | Identifier |
| 1.408204774 | MLSTAKGKTHDHWGGGRMRPPPWHDWRFS | SEQ ID NO: 2 |
| 1.384115185 | MSSGAKGKWHDHYPSGRMRPPPWHDWRFS | SEQ ID NO: 3 |
| 1.350729236 | WPSGAKGKWHDHYPGGRMRPPPWHDWRFS | SEQ ID NO: 4 |
| 1.323750558 | WGSGAKGKTHDHWGSGRWRDPGWHDWRGS | SEQ ID NO: 5 |
| 1.320605759 | FGSGAKGKTHDHWGGGRHRPPPWHDWRGS | SEQ ID NO: 6 |
| 1.318629531 | FGSGAKGKTHDHYPSGRMRPPPWHDWRVR | SEQ ID NO: 7 |
| 1.282381254 | MPSAAKGKWHDHYPGGRMRDPKWHDWRFS | SEQ ID NO: 8 |
| 1.280103826 | VTSTAKGKTHDHNWSGRYRPPPWHDWRAR | SEQ ID NO: 9 |
| 1.26032041 | FVSGAKGKWHDHWPSGRWRDPPWHDWRAS | SEQ ID NO: 10 |
| 1.236924622 | MASAAKGKWHDHWGSGRHRPPPWHDWRVS | SEQ ID NO: 11 |
| 1.216102903 | MPSAAKGKWHDHYPSGRMRPPPWHDWRAR | SEQ ID NO: 12 |
| 1.215664885 | MPSGAKGKTHDHYGGGRMRPPPWHDWRFS | SEQ ID NO: 13 |
| 1.214913594 | FSSAAKGKWHDHWPGGRYRDPPWHDWRVS | SEQ ID NO: 14 |
| 1.193103249 | FLSGAKGKTHDHYPSGRYRPPPWHDWRAR | SEQ ID NO: 15 |
| 1.185693762 | MPSAAKTKWHDHYPGGRMRPPPWHDWRGR | SEQ ID NO: 16 |
| 1.172406636 | WSSAAKGKTHDHWGGGRYRPPPWHDWRAS | SEQ ID NO: 17 |
| 1.168651327 | PVSAAKGKWHDHNWSGRMRPPPWHDWRAR | SEQ ID NO: 18 |
| 1.14735731 | WSSGAKTKWHDHYGSGRHRDPKWHDWRGS | SEQ ID NO: 19 |
| 1.134635539 | WASGAKGKWHDHYGGGRYRDPGWHDWRGS | SEQ ID NO: 20 |
| 1.134437398 | WGSAAKGKTHDHWPSGRYRDPKWHDWRVS | SEQ ID NO: 21 |
| 1.133835902 | MLSGAKGKTHDHYWGGRMRPPPWHDWRGS | SEQ ID NO: 22 |
| 1.129375178 | FGSAAKGKTHDHWPSGRHRDPGWHDWRFS | SEQ ID NO: 23 |
| 1.104085676 | WVSAAKGKWHDHYGSGRMRDPPWHDWWGS | SEQ ID NO: 24 |
| 1.087993078 | FVSAAKGKTHDHYPSGRYRPPPWHDWRAR | SEQ ID NO: 25 |
| 1.078478691 | WPSAAKGKTHDHNPSGRMRPPPWHDWRFS | SEQ ID NO: 26 |
| 1.075356247 | MASAAKTKWHDHYPSGRMRPPPWHDWRAR | SEQ ID NO: 27 |
| 1.07145419 | MPSAAKTKWHDHYPSGRMRPPPWHDWRGR | SEQ ID NO: 28 |
| 1.051932717 | MVSTAKGKTHDHYPSGRYRPPPWHDWRFS | SEQ ID NO: 29 |
| 1.051581331 | WVSAAKGKTHDHWGSGRHRDPPWHDWRAR | SEQ ID NO: 30 |
| 1.043839446 | WGSAAKGKTHDHWPSGRHRDPPWHDWRVR | SEQ ID NO: 31 |
| 1.038239035 | WGSAAKGKTHDHYGSGRYRDPPWHDWRAR | SEQ ID NO: 32 |
| 1.035817099 | FASAAKGKTHDHYWSGRYRPPPWHDWRAS | SEQ ID NO: 33 |
| 1.0281755 | FGSAAKGKTHDHYWSGRMRPPPWHDWRGS | SEQ ID NO: 34 |
| 1.009032089 | WSSTAKGKTHDHWPSGRYRPPPWHDWRAR | SEQ ID NO: 35 |
| 0.99932129 | MSSAAKGKTHDHNWSGRYRPPPWHDWRVR | SEQ ID NO: 242 |
| 0.992585072 | MSSGAKGKWHDHYGSGRHRPPPWHDWRVR | SEQ ID NO: 243 |
| 0.988225009 | FASAAKGKTHDHWGSGRYRPPPWHDWRGS | SEQ ID NO: 244 |
| 0.980050215 | MASGAKGKTHDHNWSGRMRPPPWHDWRGR | SEQ ID NO: 245 |
| 0.975277012 | MPSAAKGKTHDHYWSGRMRPPPWHDWRVS | SEQ ID NO: 246 |
| 0.974382335 | MGSAAKTKWHDHYGGGRMRPPPWHDWRAR | SEQ ID NO: 247 |
| 0.96678512 | FVSTAKGKTHDHNWSGRMRPPPWHDWRGR | SEQ ID NO: 248 |
| 0.956074317 | MLSTAKGKTHDHYGGGRMRPPPWHDWRFS | SEQ ID NO: 249 |
| 0.952720465 | WSSAAKGKTHDHNWSGRHRPPPWHDWRFS | SEQ ID NO: 250 |
| 0.952015546 | VVSTAKGKTHDHYPGGRMRPPPWHDWRAR | SEQ ID NO: 251 |
| 0.948285692 | MASAAKGKTHDHYPGGRMRPPPWHDWRAS | SEQ ID NO: 252 |
| 0.946907907 | WSSAAKGKTHDHYPSGRHRPPPWHDWRVS | SEQ ID NO: 253 |
| 0.942612516 | FASAAKGKTHDHYPGGRMRPPPWHDWRFS | SEQ ID NO: 254 |
| 0.934953609 | FLSAAKGKTHDHNWGGRMRPPPWHDWRFS | SEQ ID NO: 255 |
| 0.92538183 | WGSAAKGKTHDHWPSGRMRDPPWHDWRVS | SEQ ID NO: 256 |
| 0.917986909 | VLSTAKGKTHDHYPGGRMRPPPWHDWRAR | SEQ ID NO: 257 |
| 0.91347202 | MVSAAKTKTHDHNGGGRMRPPPWHDWRFS | SEQ ID NO: 258 |
| 0.910108543 | VVSTAKGKTHDHYWSGRYRPPPWHDWRAR | SEQ ID NO: 259 |
| 0.898631112 | PLSAAKGKWHDHYWSGRMRDPPWHDWRVS | SEQ ID NO: 260 |
| 0.887129832 | SPSGAKGKTHDHYPSGRMRPPPWHDWRFS | SEQ ID NO: 261 |
| 0.88652873 | MGSAAKGKWHDHYGSGRMRPPPWHDWRVR | SEQ ID NO: 262 |
| 0.884786726 | MASAAKGKTHDHYPSGRMRPPPWHDWRFS | SEQ ID NO: 263 |
| 0.877162961 | PSSAAKGKTHDHYPGGRYRPPPWHDWRVS | SEQ ID NO: 264 |
| 0.853441609 | PLSAAKTKWHDHYGGGRMRPPPWHDWRFS | SEQ ID NO: 265 |
| 0.849759193 | MVSTAKGKTHDHYGGGRYRPPPWHDWRAR | SEQ ID NO: 266 |
| 0.848656015 | FQSTAKGKTHDHWPSGRMRPPPWHDWRAS | SEQ ID NO: 267 |
| 0.846552374 | MGSGAKGKTHDHWPSGRMRPPPWHDWRVS | SEQ ID NO: 268 |
| 0.846411919 | MLSAAKGKTHDHYPSGRYRPPPWHDWRAR | SEQ ID NO: 269 |
| 0.844562849 | MPSAAKTKWHDHNWSGRMRPPPWHDWRAR | SEQ ID NO: 270 |
| 0.843149119 | WASGAKGKTHDHWPGGRHRDPPWHDWRVS | SEQ ID NO: 271 |
| 0.837907367 | WTSAAKTKWHDHYGGGRMRPPPWHDWRAR | SEQ ID NO: 272 |
| 0.820294321 | FPSGAKTKWHDHNPSGRMRDPPWHDWRFSa | SEQ ID NO: 273 |
| 0.819092378 | WSSAAKGKTHDHYGSGRMRDFGWHDWRFS | SEQ ID NO: 274 |
| 0.817845298 | WGSAAKGKWHDHYPSGRMRPPPWHDWRVR | SEQ ID NO: 275 |
| 0.810500369 | MGSGAKTKTHDHYPGGRYRPPPWHDWRVS | SEQ ID NO: 276 |
| 0.79340345 | MPSTAKGKWHDHNPSGRMRPPPWHDWRGR | SEQ ID NO: 277 |
| 0.778425405 | WASGAKGKTHDHYPSGRMRPPPWHDWRWS | SEQ ID NO: 278 |
| 0.778238717 | MGSAAKGKWHDHNGSGRMRPPPWHDWRAR | SEQ ID NO: 279 |
| 0.771565139 | WTSGAKGKWHDHYGGGRYRDPGWHDWRFS | SEQ ID NO: 280 |
| 0.754814866 | FASGAKGKTHDHNWGGRYRDPGWHDWRAS | SEQ ID NO: 281 |
| 0.751402827 | PLSAAKTKTHDHNWSGRYRPPPWHDWRAR | SEQ ID NO: 282 |
| 0.747523684 | VSSAAKGKTHDHYPSGRMRPPPWHDWRGR | SEQ ID NO: 283 |
| 0.737449367 | MGSGAKTKWHDHWPSGRMRPPPWHDWRFS | SEQ ID NO: 284 |
| 0.731921848 | MGSGAKGKTHDHNWGGRHRDPGWHDWWVS | SEQ ID NO: 285 |
| 0.724052728 | WSSGAKTKWHDHNGSGRYRPPPWHDWRGS | SEQ ID NO: 286 |
| 0.720776516 | MASGAKGKTHDHYPGGRMRPPPWHDWRAR | SEQ ID NO: 287 |
| 0.720658021 | MASAAKGKWHDHNPSGRHRDPGWHDWWGS | SEQ ID NO: 288 |
| 0.715057636 | WTSAAKGKWHDHNGSGRHRDPPWHDWRAR | SEQ ID NO: 289 |
| 0.711988995 | WASGAKGKWHDHYGSGRYRPPPWHDWRGS | SEQ ID NO: 290 |
| 0.711944726 | VPSAAKGKWHDHNPGGRHRPPPWHDWRAS | SEQ ID NO: 291 |
| 0.704875988 | MLSTAKGKTHDHYWGGRMRPPPWHDWRFS | SEQ ID NO: 292 |
| 0.704689362 | MLSGAKTKTHDHNPSGRMRDPPWHDWRVR | SEQ ID NO: 293 |
| 0.683729617 | WQSAAKTKTHDHWPSGRYRPPPWHDWRAS | SEQ ID NO: 294 |
| 0.678204562 | WPSAAKGKTHDHYPGGRYRPPPWHDWRVR | SEQ ID NO: 295 |
| 0.672935269 | VVSGAKGKTHDHNWGGRYRPPPWHDWRGS | SEQ ID NO: 296 |
| 0.666197617 | VSSGAKGKTHDHYPSGRMRPPPWHDWRFS | SEQ ID NO: 297 |
| 0.662541125 | FASAAKGKTHDHYPSGRYRPPPWHDWRAS | SEQ ID NO: 298 |
| 0.649679238 | VGSGAKGKTHDHNGSGRYRPPPWHDWRFS | SEQ ID NO: 299 |
| 0.644987482 | SVSAAKGKTHDHYPSGRWRDPPWHDWRAS | SEQ ID NO: 300 |
| 0.634757799 | MSSGAKGKWHDHNWGGRMRDPGWHDWWGS | SEQ ID NO: 301 |
| 0.626396149 | WGSAAKGKTHDHNWSGRYRPPPWHDWRVS | SEQ ID NO: 302 |
| 0.618397521 | MSSGAKTKTHDHYPSGRMRPPPWHDWRAR | SEQ ID NO: 303 |
| 0.613680213 | WSSAAKGKTHDHNPSGRHRPPPWHDWRVS | SEQ ID NO: 304 |
| 0.611962487 | WSSTAKTKWHDHNGGGRMRPPPWHDWRAR | SEQ ID NO: 305 |
| 0.60235346 | FPSGAKTKWHDHNPSGRMRDPPWHDWRFS | SEQ ID NO: 306 |
| 0.600492461 | WLSTAKGKTHDHNWSGRYRPPPWHDWRVR | SEQ ID NO: 307 |
| 0.588014464 | VTSAAKGKWHDHWPSGRYRDPPWHDWRVS | SEQ ID NO: 308 |
| 0.585109127 | WTSGAKGKWHDHNGGGRMRDPGWHDWRFS | SEQ ID NO: 309 |
| 0.571811524 | VTSAAKGKTHDHYPSGRYRPPPWHDWRGR | SEQ ID NO: 310 |
| 0.563074961 | MPSAAKGKTHDHNGGGRMRPPPWHDWRFS | SEQ ID NO: 311 |
| 0.561612038 | VTSAAKGKTHDHYPSGRMRPPPWHDWRFS | SEQ ID NO: 312 |
| 0.553631613 | PASGAKGKTHDHYPSGRWRDSGWHDWRFS | SEQ ID NO: 313 |
| 0.545831706 | MASTAKGKTHDHYGGGRMRPPPWHDWRVR | SEQ ID NO: 314 |
| 0.530183277 | WLSAAKAKTHDHWGSGRHRPPPWHDWRGS | SEQ ID NO: 315 |
| 0.523076714 | FASGAKGKTHDHYPSGRHRPPPWHDWRAS | SEQ ID NO: 316 |
| 0.519118407 | MPSGAKGKWHDHYGGGRMRDPGWHDWRGS | SEQ ID NO: 317 |
| 0.513689379 | WPSTAKGKTHDHYGGGRMRPPPWHDWRAR | SEQ ID NO: 318 |
| 0.508575373 | MGSAAKGKWHDHYPGGRHRDPPWHDWRAS | SEQ ID NO: 319 |
| 0.506290739 | MASTAKGKTHDHWPGGRMRPPPWHDWRGS | SEQ ID NO: 320 |
| 0.488167958 | PVSAAKGKWHDHYPSGRWRDPPWHDWRAS | SEQ ID NO: 321 |
| 0.453759987 | VSSAAKGKTHDHNPSGRMRPPPWHDWRFS | SEQ ID NO: 322 |
| 0.450508836 | WASAAKGKTHDHYGGGRMRPPPWHDWRAR | SEQ ID NO: 323 |
| 0.445912032 | SPSGAKTKWHDHNPGGRMRPPPWHDWRAR | SEQ ID NO: 324 |
| 0.443475625 | SSSGAKGKWHDHNPSGRWRDPGWHDWRVR | SEQ ID NO: 325 |
| 0.430019303 | MPSAAKGKWHDHYGSGRYRPPPWHDWRVR | SEQ ID NO: 326 |
| 0.416150437 | PASAAKGKWHDHNPSGRMRDPPWHDWWGS | SEQ ID NO: 327 |
| 0.396711188 | PASGAKTKWHDHNPSGRHRDPPWHDWRVS | SEQ ID NO: 328 |
| 0.376790518 | PVSAAKGKTHDHWPGGRMRDPPWHDWRAS | SEQ ID NO: 329 |
| 0.370470366 | WASGAKGKTHDHNPSGRWRDPGWHDWRGS | SEQ ID NO: 330 |
| 0.346829799 | VGSAAKTKTHDHYGGGRMRPPPWHDWRFS | SEQ ID NO: 331 |
| 0.32575327 | WSSAAKTKWHDHNPGGRYRPPPWHDWRFR | SEQ ID NO: 332 |
| 0.323399568 | MPSAAKGKTHDHYPSGRMRPPPWHDWRGR | SEQ ID NO: 333 |
| 0.321676328 | WGSGAKGKTHDHNGGGRMRPPPWHDWRAS | SEQ ID NO: 334 |
| 0.31562167 | WSSGAKTKWHDHNPGGRMRDPPWHDWRGS | SEQ ID NO: 335 |
| 0.303206195 | WVSAAKGKTHDHNGGGRWRDPGWHDWRFS | SEQ ID NO: 336 |
| 0.283561428 | MGSAAKTKWHDHYPGGRMRPPPWHDWRFS | SEQ ID NO: 337 |
| 0.282911531 | WSSAAKGKTHDHYGGGRMRPPPWHDWRAR | SEQ ID NO: 338 |
| 0.281424681 | WPSGAKTKTHDHNWGGRMRDPPWHDWRGS | SEQ ID NO: 339 |
| 0.276029472 | MVSTAKGKTHDHYGSGRYRPPPWHDWRGS | SEQ ID NO: 340 |
| 0.274617004 | SASAAKGKTHDHWGGGRHRDPPWHDWRFS | SEQ ID NO: 341 |
| 0.232475479 | MASGAKGKWHDHYGGGRMRDPGWHDWRFS | SEQ ID NO: 342 |
| 0.214105528 | PSSAAKGKTHDHYPGGRYRDPGWHDWRGR | SEQ ID NO: 343 |
| 0.132577291 | SSSGAKGKWHDHYGGGRHRDPPWHDWRGS | SEQ ID NO: 344 |
| 0.065638761 | WLSAAKGKTHDHWGSGRWRDPPWHDWRFS | SEQ ID NO: 345 |
| 0.042915557 | VHSAAKTKTHDHWGSGRMRDPPWHDWRGS | SEQ ID NO: 346 |
| 0.014990515 | VTSAAKTKWHDHNGSGRYRDPGWHDWRGS | SEQ ID NO: 347 |
| −0.009348878 | VPSAAKGKWHDHNGSGRMRDPGWHDWRFS | SEQ ID NO: 348 |
| −0.097552918 | MGSAAKGKTHDHYPSGRMRPPPWHDWRGS | SEQ ID NO: 349 |
| −0.115335808 | VSSGAKTKTHDHNPSGRMRDFKWHDWRGS | SEQ ID NO: 350 |
| −0.133077239 | MLSAAKTKTHDHWGSGRMRDPPWHDWRVS | SEQ ID NO: 351 |
| −0.230452053 | WQSAAKTKTHDHWPGGRMRPPPWHDWRAR | SEQ ID NO: 352 |
| −0.27883329 | PASAAKGKTHDHWPSGRHRDPPWHDWRGS | SEQ ID NO: 353 |
| −0.314977465 | WGSGAKTKWHDHNWSGRMRPPPWHDWRFS | SEQ ID NO: 354 |
| −0.408574643 | FSSAAKTKWHDHYGSGRYRPPPWHDWRFS | SEQ ID NO: 355 |
| −0.50552113 | PTSAAKGKTHDHYPGGRYRDPPWHDWRAS | SEQ ID NO: 356 |
| −1.020272208 | WTSAAKGKTHDHYPSGRYRPPPWHDWRVR | SEQ ID NO: 357 |

### Example 6: Identifying Human Targets of SG-3-0020

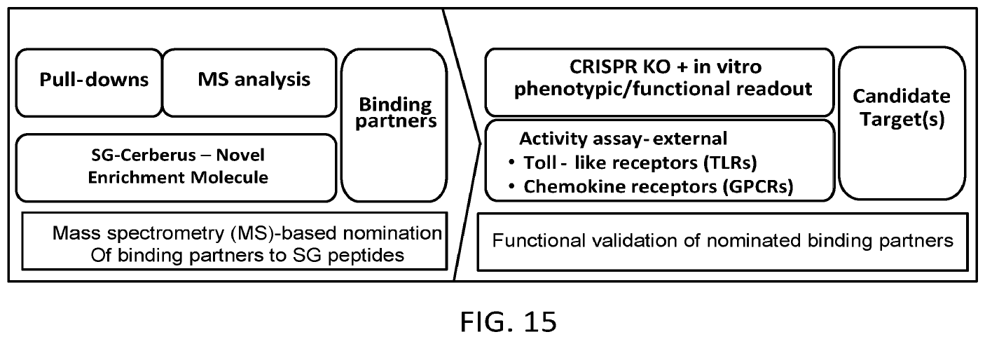

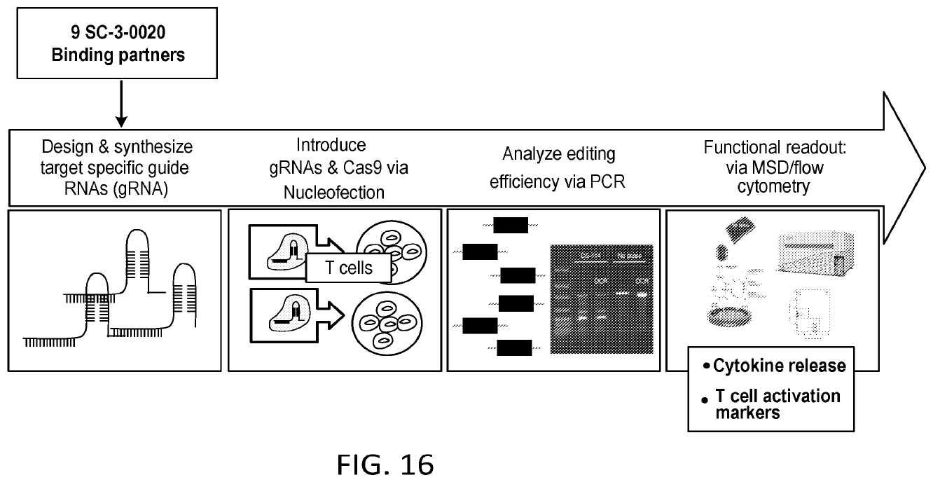

To identify binding partners of SG-3-0020 in human cells, pull-down and mass spectrometry analysis were used (FIG. 15). Multiple target capture experiments were performed with SG-3-0020 and controls. Binding conditions and cross-linkers were varied to achieve maximum enrichment of peptide over controls. Nine putative binding partners to SG-3-0020 were identified using MS and were nominated for confirmation using CRISPR Knock Out (KO) Experiments (Table 12, FIG. 16). The enrichment ratio was calculated as described below:

$$Enrichment ratio = \frac{quantity of target pulled down with SG - 3 - 0020}{quantity of target pulled down in control}$$

An enrichment ratio of 100.0 indicated that no target protein was observed in the control pulldown.

TABLE 12
Putative binding partners of SG-2-0549

| Protein name | Enrichment ratio |
|---|---|
| Protein AMBP | 69.1 |
| Prohibitin | 1.9 |
| CD2 (cluster of differentiation 2) | 1.9 |
| Low-density lipoprotein receptor | 100.0 |
| Bone marrow stromal antigen 2 | 34.5 |
| Tumor necrosis factor receptor superfamily | 100.0 |
| member 4 | |
| Cation-independent mannose-6-phosphate | 2.1 |
| receptor | |
| Tumor necrosis factor | 67.2 |
| Teneurin-3 | 3.4 |

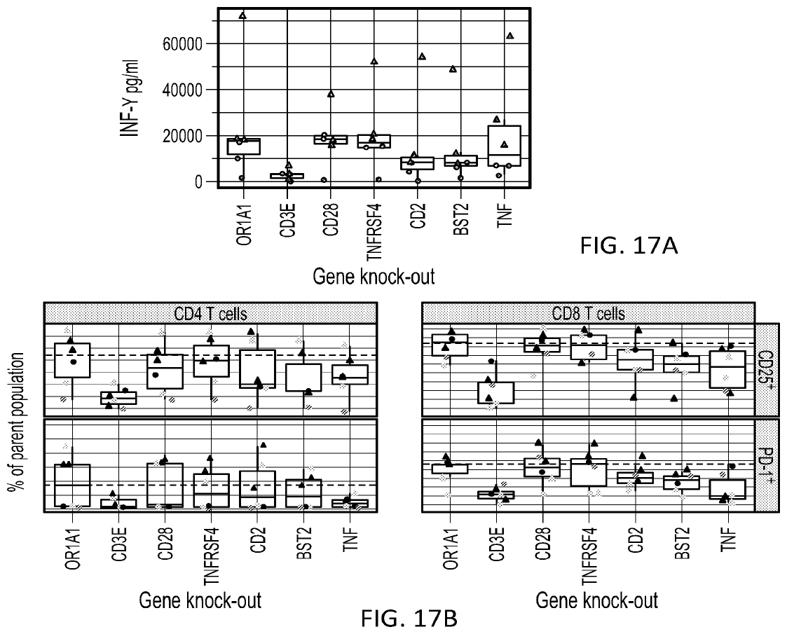

Functional validation was performed by CRISPR knock-out to confirm target effects on SG-3-0020-mediated activity. Ribonucleoprotein (RNP)-mediated CRISPR genome editing was performed to induce small indels, which resulted in protein knock-down. RNPs were introduced into primary human unstimulated T cells via Nucleofection and cells were cultured for 96 hours. OR1A1 served as negative control and CD3E was used as positive control. Post knock-down T cells were stimulated in vitro culture of Purified T cells with anti-CD3 (0.1 μg/mL) and SG-3-05429 (mutant SG-3-0020 with improved activity) (10 μM) and was incubated for 48 hours. A decrease in IFN-γ secretion (FIG. 17A) and a decrease in activated CD4 and CD8 T cell subsets was also observed (CD25+, PD-1+=Markers of T cell activation) (FIG. 17B) was observed after target knock-down compared to the negative control (OR1A1). Three human targets, CD2, BST2, and TNF that were identified from mass spectrometry got SG-3-0020 were confirmed via CRISPR KO experiments. Activation of CD2 has the potential to be a therapeutic opportunity.

### Example 7. Identifying Additional Peptides that Induce CXCL10 Production

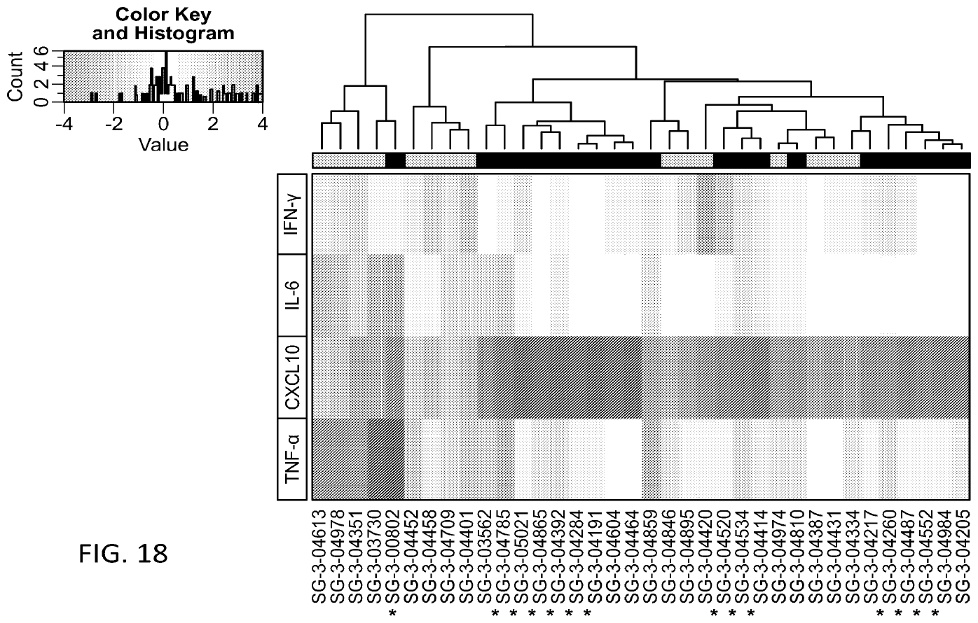

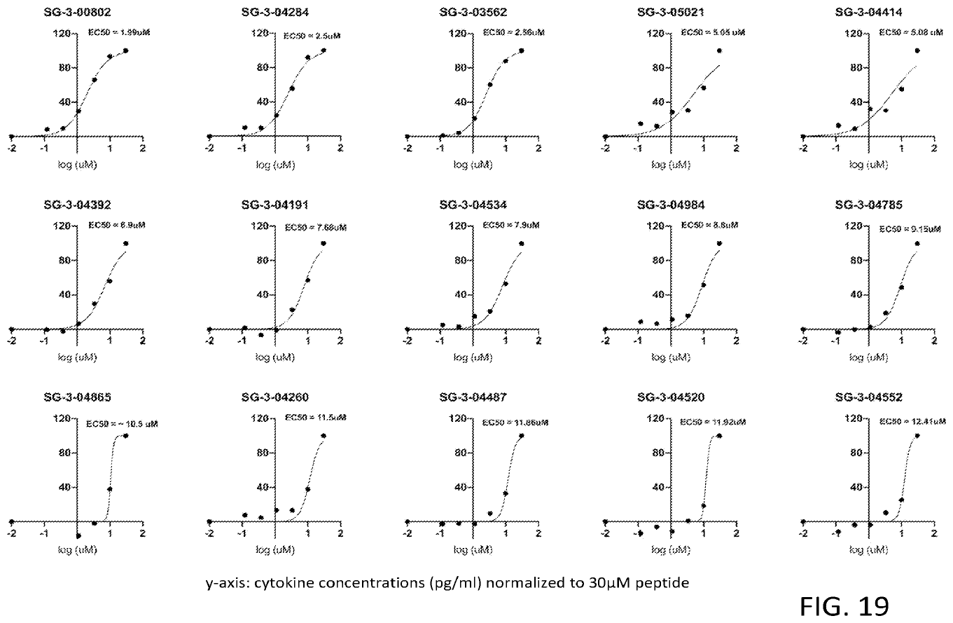

Studies were performed to identify target peptides that induced CXCL10 production. Target peptides identified from human microbiomes isolated from humans that responded to anti-PD-1 therapy were each tested in a three point dose response curve with TNF-α, CXCL10, IL6, and INF-γ. Fifteen peptides with high CXCL10 induction were identified for additional testing, indicated with asterisks in the figure (FIG. 18). CXCL10 is up-regulated for all peptides indicated with an asterisk compared to other peptides (FIG. 18). Peptides SG-3-00802 and SG-3-05021 were identified as high CXCL10 induction (FIG. 19). Peptide sequences and characteristics can be found in Tables 13-15.

TABLE 13
Peptide Sequence and Strains

| Peptide | Peptide | | |
|---|---|---|---|
| ID | name | Sequence | Strain or RDM |
| SEQ ID | SG-3- | MKVRPSVKPMCEK | oscillibacter.sp |
| NO: 117 | 00802 | CKIIRRKGRVMVI | t_155102 |
| | | CENPKHKQRQG | (GCF_000765235) |
| SEQ ID | SG-3- | LGKTDWIEKYFKV | bacteroides.clarus. |
| NO: 118 | 05021 | KKEKIDKMQRFLQ | dsm.22519 |
| | | G | t_58760 |
| | | | (GCF_000195615) |

TABLE 14
Peptide Characteristics

| | Full Length vs | | |
|---|---|---|---|
| Peptide ID | fragment? | Contained/overlap with a PFAM region? | PFAM notes |
| SEQ ID NO: | Full | | |
| 117 | | | |
| SEQ ID NO: | TMHMM-o | Contained within PFAM region. Source protein has 2 | DUF2238 relates to |
| 118 | | PFAM regions (DUF2238 and SNARE_assoc) from | an inner membrane |
| | | positions 51-136 and 35-147. The TMHMM-o | protein |
| | | fragment here is from 73-100 | |

TABLE 15
Peptide Characteristics.

| | Secreted: | Secreted: | Secreted: | | Length | |
|---|---|---|---|---|---|---|
| Peptide ID | SigP | SecP | PsortB | pI | (aa) | Binder? |
| SEQ ID | No | No | No | 10.69469949 | 37 | DC |
| NO: 117 | | prediction | | | | |
| SEQ ID | No | Yes | No | 9.777983413 | 27 | DC |
| NO: 118 | | | | | | |

### Example 8. Down-Regulation of DC-SIGN by SG-3-00802 and SG-3-05021 Peptides

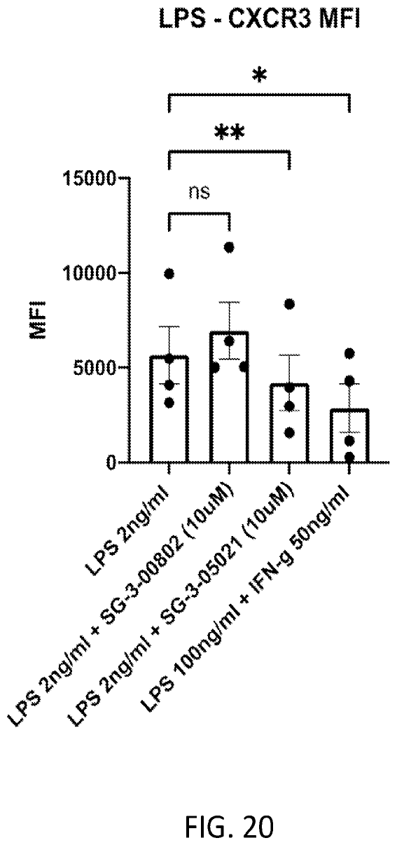

Human monocyte-derived dendritic cells (moDCs) were stimulated with low dose lipopolysaccharide (LPS) (2 ng/mL) alone, LPS with peptides SG-3-00802 (10 μM) or SG-3-05021 (10 μM), and LPS (2 ng/mL) with IFNg (50 ng/mL) as a positive control. After stimulation of moDCs, flow cytometry was used to determine induction of the CSCR3 receptor. Treatment with peptides SG-3-00802/and SG-3-05021 did not induce CXCR3 receptor internalization (FIG. 20) and down-regulate DC-SIGN.

### Example 9. Assaying Agonist and Antagonist Function of SG-3-00802 and SG-3-05021 Peptides

Peptides SG-3-00802 and SG-3-05021 were screened for agonist and antagonist activity of the chemokine receptors CCR7, CXCR3, and CXCR4.

In agonist assays, chemokine receptor expressing U2OS cells are thawed and resuspended in Assay Media (DMEM, 1% dialyzed FBS, 25 mM HEPES pH 7.3, 0.1 mM NEAA, 100 U/mL/100 μg/mL Pen/Strep) to a concentration of 312,500 cells/mL. 4 μL of a 10×serial dilution of I-TAC (control agonist starting concentration, 500 nM) or compounds are added to appropriate wells of a 384-well TC-Treated assay plate. 32 μL of cell suspension (10,000 cells) is added to each well. 4 μL of Assay Media is added to all wells to bring the final assay volume to 40 μL. The plate is incubated for 16-24 hours at 37° C./5% CO₂ in a humidified incubator. 8 μL of 1 μM Substrate+Solution D Loading Solution is added to each well and the plate is incubated for 2 hours at room temperature. The plate is read on a fluorescence plate reader.

In antagonist assays, CXCR3-bla U2OS cells were thawed and prepared as described above for the Agonist assay. 4 μL of 10×compounds or assay media was added to appropriate wells of a TC-Treated assay plate. 32 μL of cell suspension was added to the wells and pre-incubated at 37° C./5% CO₂ in a humidified incubator with compounds and control antagonist titration for 30 minutes. 4 μL of 10×control agonist I-TAC at the pre-determined EC80 concentration is added to wells containing the control antagonist or compounds. The plate is incubated for 16-24 hours at 37° C./5% CO₂ in a humidified incubator. 8 μL of 1 μM Substrate+Solution D Loading Solution is added to each well and the plate is incubated for 2 hours at room temperature. The plate is read on a fluorescence plate reader. At this time, the CXCR3-bla U2OS assay does not have an antagonist control.

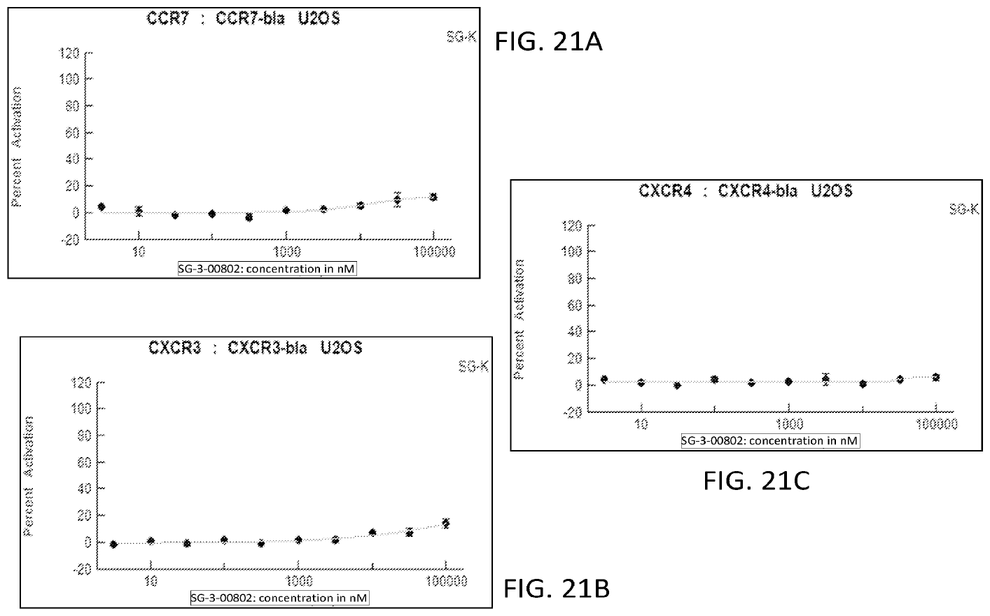

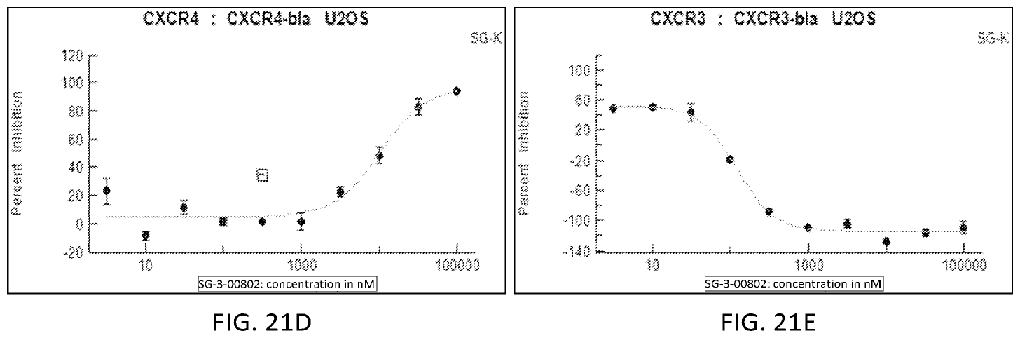

SG-3-00802 showed weak agonistic activity for CCR7 (FIG. 21A) and CXCR3 (FIG. 21B), no agonistic activity for CXCR4 (FIG. 21C), a moderate antagonistic activity for CXCR4 (FIG. 21D). SG-3-00802 shows potent reverse antagonistic activity (sensitization) for CXCR3 (FIG. 21E).

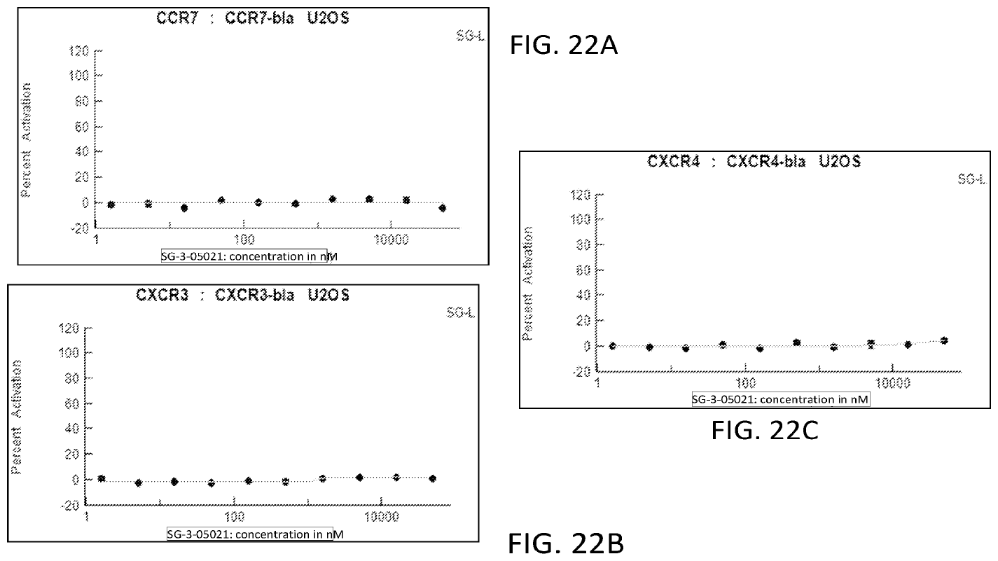

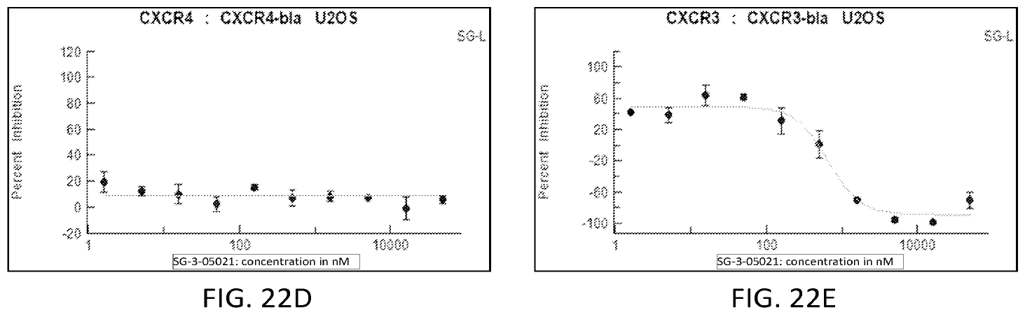

SG-3-05021 showed no agonistic activity for CCR7 (FIG. 22A), CXCR3 (FIG. 22B), or CXCR4 (FIG. 22C). SG-3-00802 showed no antagonistic activity for CXCR4 (FIG. 22D), and reverse antagonistic activity (sensitization) for CXCR3 (FIG. 22E).

### Example 10. Prediction of SG-3-00802 Binding to Dendritic Cells

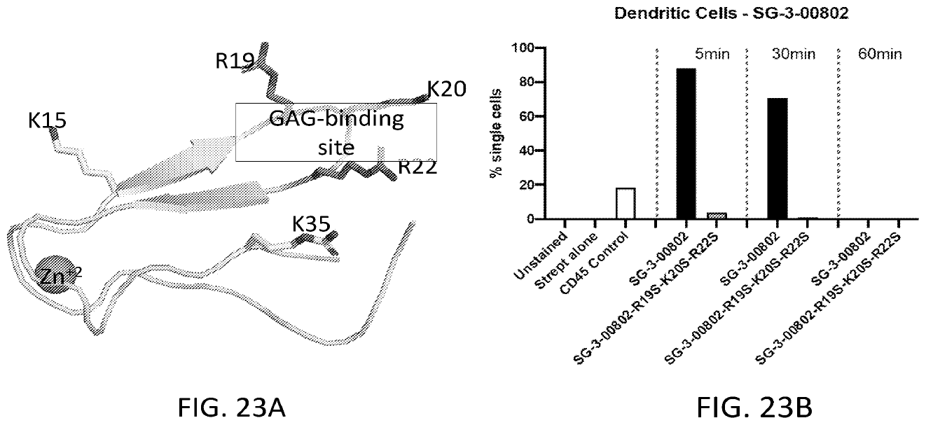

A predicted structure of SG-3-00802 was created by homology modeling using SWISS-MODEL (template PDB ID: 1DFE) and was superimposed with a homologous structure (PDB ID: 6FGP). The BBXB motif in glycosaminoglycans was present (FIG. 23A) (GAG-binding site, R19, K20, R22, are labeled). Positively charged residues (K15 and R35) could bind to the BBXB motif to form a cluster that could bind negatively charged GAG efficiently. The gray sphere indicates a zinc 2⁺ ion.

The binding of parental SG-3-00802 and SG-3-00802 where the GAG-binding site was mutated (SG-3-00802-R195-K205-R22S) to human dendritic cells was tested by flow cytometry. Mutation of the R19, K205, R22S amino-acids that comprise the GAG-binding motif abolished binding of mutant SG-3-00802 to dendritic cells (FIG. 23B).

### Example 11. Assaying Chemokine Release In Vitro by SG-3-00802 and SG-3-05021

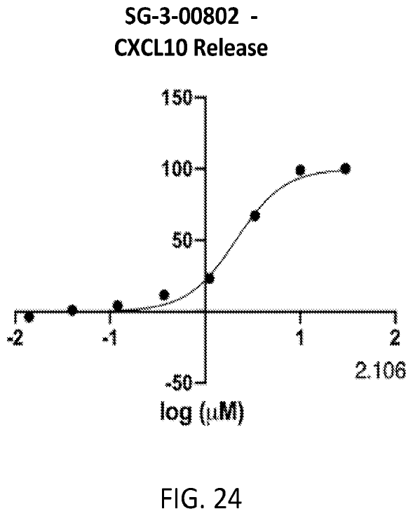

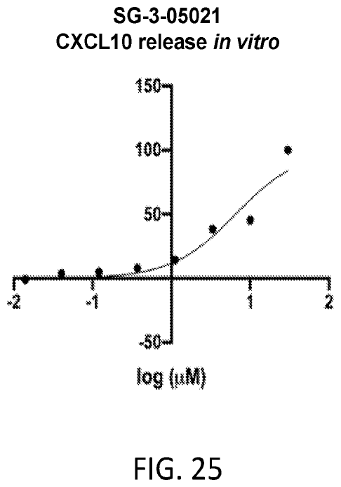

Using an in vitro culture of human purified monocyte-derived dendritic cells, chemokine release was assessed in a dose-dependent manner. LPS was used at low concentrations for a suboptimal activation of dendritic cells in the presence of SG-3-00802 peptide or SG-3-05021 peptide. There was a dose-dependent increase in CXCL10 chemokine when treated with SG-3-00802 in LPS stimulated dendritic cells (FIG. 24) or when treated with SG-3-05021 in LPS stimulated dendritic cells (FIG. 25).

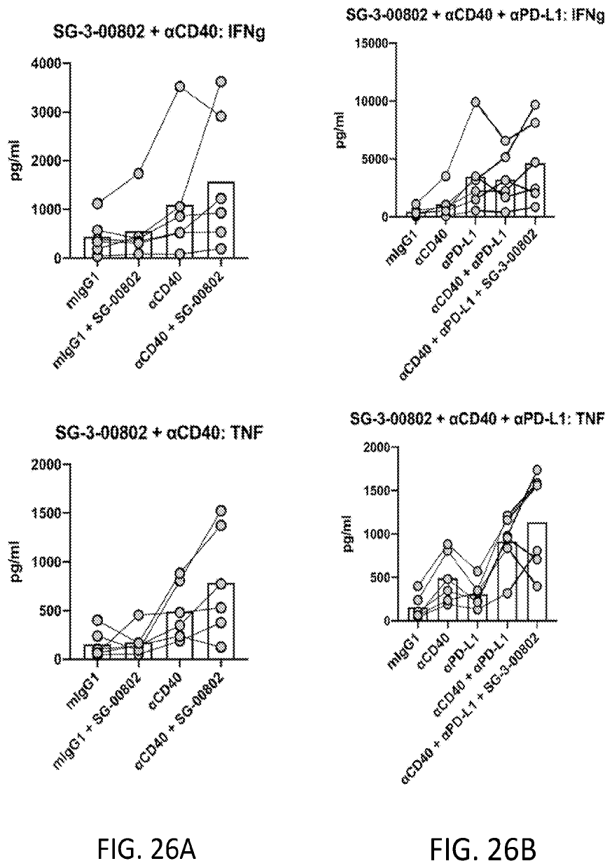

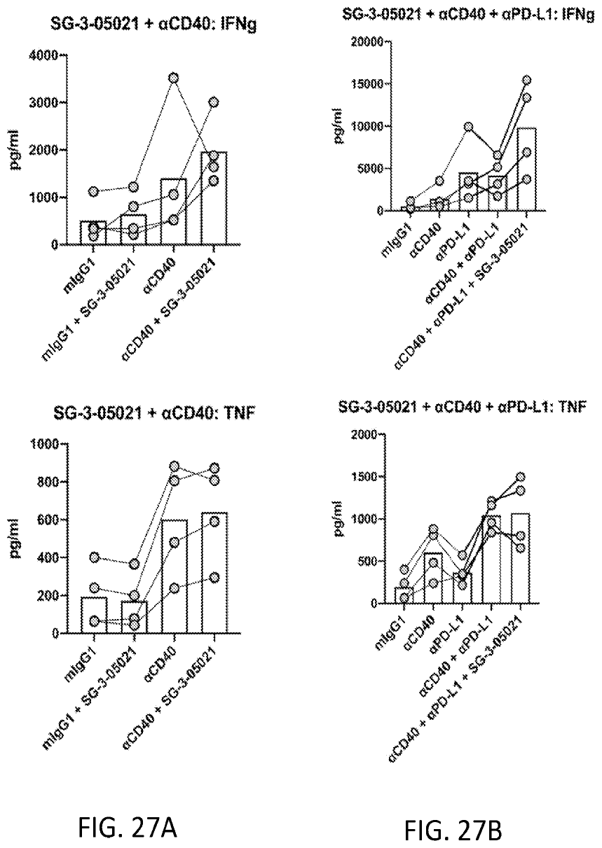

In another study, immature monocyte-derived dendritic cells (moDCs) were pre-stimulated with anti-CD40 agonist antibody or the mouse IgG1 isotype control antibody. Twenty-four hours later, T cells isolated from allogeneic donors were added to the moDCs with the peptide of interest, SG-3-00802 or SG-3-05021, alone or in combination with anti-PD-L1 monoclonal antibody. The co-culture of cells was incubated for 72 hours, after which cell supernatants were harvested and analyzed for secretion of cytokines, IFN-g and TNF-α. Both SG-3-00802 and SG-3-05021 induced an increase in IFN-γ and TNF-α after stimulation with anti-CD40 either alone or in combination with anti PD1 (FIGS. 26A-26B and 27A-27B). Data shown for experiment conducted in >4 Donor PBMCs.

### Example 12. Assaying Anti-Tumor Activity of SG-3-00802 in Combination with Anti-PD-1 Antibody In Vivo

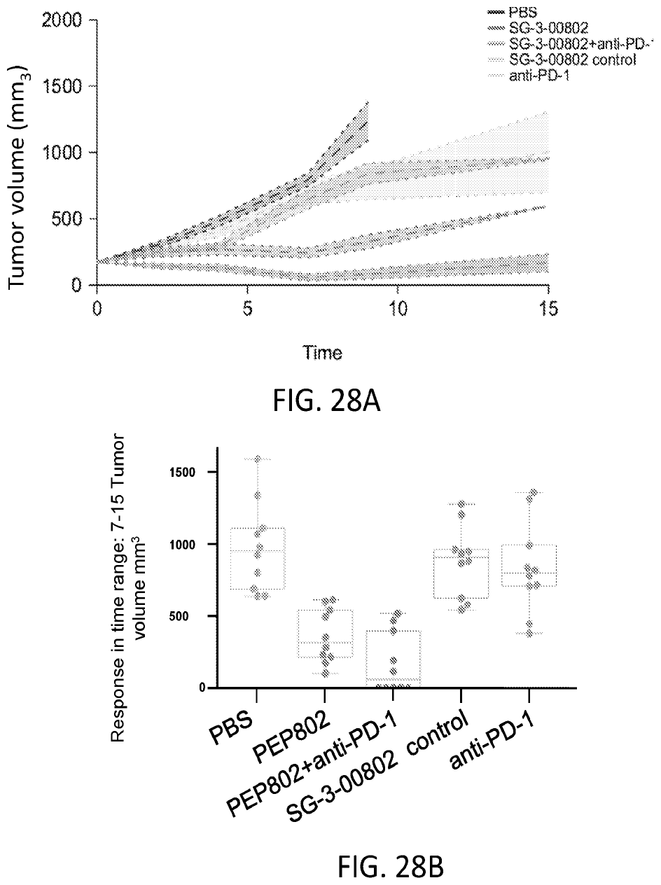

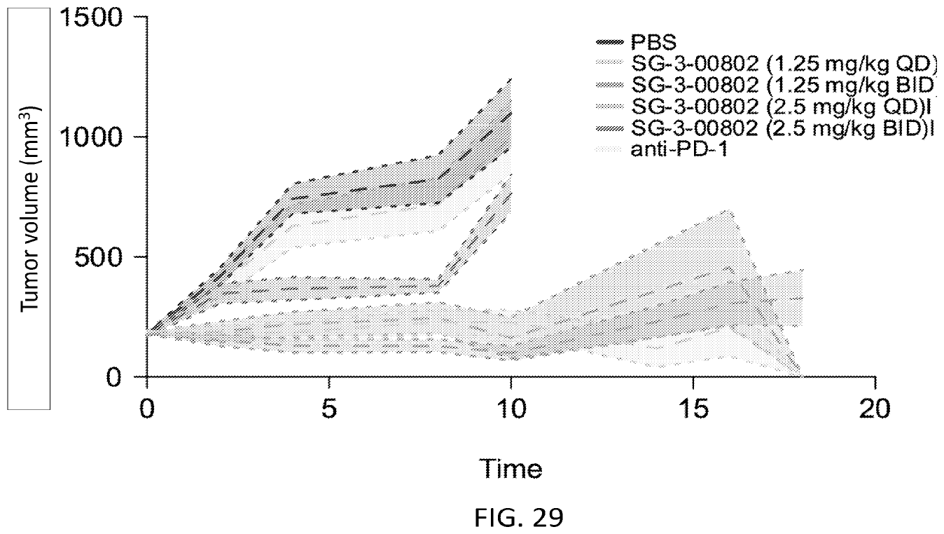

To determine anti-tumor activity of SG-3-00802 in vivo, the RENCA murine adenocarcinoma model was used. It is a syngeneic, standardized experimental model of metastatic RCC. Briefly, BALB/c female mice were inoculated subcutaneously with RENCA cells. Established tumors were treated daily with 2.5 mg/kg SG-3-00802 peritumorally, alone or in combination with intraperitoneal administration of 10 mg/kg anti-PD-1 antibody. A robust decrease in tumor volume either alone or in combination with anti-PD-1 compared to the phosphate buffered saline (PBS) treated control mice was observed over time, where time is in days after dosing initiation (FIGS. 28A-28B). In addition, SG-3-00802 reduced volume of syngeneic RENCA tumors in a dose-dependent manner (FIG. 29). QD indicates administration of the peptide once a day. BID indicates administration of the peptide twice a day.

Additional studies were completed to determine whether SG-3-00802 peptide treatment in combination with PD-1 increased survival of RENCA tumor bearing mice. Balb/c female mice were implanted with RENCA cells in the left flank. When tumors were established and reach ˜100 mm³ or 200 mm³ mice were allocated into dosing groups. Each dosing group had 10-12 animals. Peptides were dosed peritumoral at 2.5 mg/kg BID for 7 d and anti-PD-1 was dosed intraperitoneally at 10 mg/kg TIW for a total of 3 doses. Mice were monitored for tumor growth and survival. Tumor volumes were measured twice a week. A survival event (censoring) was reached when tumor volume reached 1000 mm³ and additional survival events were defined as animals that had to be euthanized due to ulceration. SG-3-00802, in combination with PD-1, increased survival of RENCA tumor bearing mice in a dose-dependent manner (FIGS. 30A-30B).

Additional studies were performed to test survival and tumor volume responses to a re-challenge event. In one study, mice were allocated into dosing groups when tumors reached ˜100 mm³. During the initial dosing study, time 0 was the first day of seven days of dosing. Initial dosing showed increased survival with either SG-3-00802 peptide or SG-3-00802 peptide in combination with anti-PD-1 antibodies (FIG. 31A). Re-implantation was completed on day 39 after dosing completion. Treated mice were tumor free for >20 days before being re-challenge. RENCA cells were implanted on the opposite flank (right flank) of the previously tumor bearing Balb/c mice and mice that did not receive any previous treatment were used as control. Tumor volume and survival was been monitored as previously described. In the re-challenge experiment, time 0 is day 10 after re-challenge dosing. Re-challenge showed an increased percent of mice surviving after dosing with SG-3-00802 alone or in combination with anti-PD-1 antibody treatment (FIG. 31B).

In the initial dosing experiment, tumor volumes were smaller in both the SG-3-00802 alone treatment and SG-3-00802 with anti-PD-1 antibody combinatorial treatment 40 days after dosing started (FIGS. 32A-32B). After a re-challenge, tumor volumes of SG-3-00802 alone or SG-3-00802 with anti-PD-1 antibody combinatorial treatment remained smaller than PBS-treated control mice throughout the study (FIGS. 33A-33B). Results are summarized in Table 16.

TABLE 16
Summary of tumor-free animals in re-challenge study.

| | # of animals with | # of animals without |
|---|---|---|
| | complete tumor | tumor progression out |
| | regression out of total | of re-challenged |
| Treatment | animals per group | animals per group |
| Group | (primary study) | (re-challenge study) |
| PBS | 0/12 | 0/10* |
| SG-3-00802 | 2/12 | 2/2 |
| Anti-PD-1 | 0/10 | n/a |
| SG-3-00802 + | 4/12 | 3/4 |
| Anti-PD-1 | | |

*newly implanted tumors in tumor naive animals

In another re-challenge study, mice were instead allocated into dosing groups when tumors reached ˜200 mm³. The same procedure as described above was used, except where indicated. Initial dosing showed increased survival with either SG-3-00802 peptide or SG-3-00802 peptide in combination with anti-PD-1 antibodies (FIG. 34A). Re-implantation was completed on day 37 after dosing completion, and time 0 is day 12 post re-challenge. Re-challenge showed an increased percent of mice surviving after dosing with SG-3-00802 in combination with anti-PD-1 antibody treatment (FIG. 34B). In the initial dosing experiment, tumor volumes were smaller in both the SG-3-00802 alone treatment and SG-3-00802 with anti-PD-1 antibody combinatorial treatment 15 days after dosing started (FIGS. 35A-35B). After a re-challenge, tumor volumes of SG-3-00802 with anti-PD-1 antibody combinatorial treatment remained smaller than PBS-treated control mice throughout the study (FIGS. 36A-36B). Results are summarized in Table 17.

TABLE 17
Summary of tumor-free animals in re-challenge study.

| | # of animals with | # of animals without |
|---|---|---|
| | complete tumor | tumor progression out |
| | regression out of total | of re-challenged |
| Treatment | animals per group | animals per group |
| Group | (primary study) | (re-challenge study) |
| PBS | 0/10 | 0/10* |
| SG-3-00802 | 0/10 | n/a |
| Anti-PD-1 | 0/10 | n/a |
| SG-3-00802 + | 5/10 | 5/5 |
| Anti-PD-1 | | |

*newly implanted tumors in tumor naive animals

### Example 13. Assaying Anti-Tumor Activity of SG-3-00802 in Combination with Anti-PD-1 Antibody In Vivo

To determine anti-tumor activity of SG-3-00802 in vivo, the RENCA murine adenocarcinoma model was used. Briefly, BALB/c female mice were inoculated subcutaneously with RENCA cells. Established tumors were treated daily with 2.5 mg/kg SG-3-00802 peritumorally, alone or in combination with intraperitoneal administration of 10 mg/kg anti-PD-1 antibody. Mice survived a longer time (days) following treatment with either the SG-3-00802 either alone or with anti-PD-1 antibody therapy compared to mice treated with phosphate buffered saline (PBS) or anti-PD-1 antibody therapy alone (FIG. 34A-34B).

### Example 14. Assaying Anti-Tumor Activity of SG-3-05021 in Combination with Anti-PD-1 Antibody In Vivo

To determine anti-tumor activity of SG-3-05021 in vivo, the RENCA murine adenocarcinoma model was used. Briefly, BALB/c female mice were inoculated subcutaneously with RENCA cells. Established tumors were treated daily with 2.5 mg/kg SG-3-05021 peritumorally, alone or in combination with intraperitoneal administration of 10 mg/kg anti-PD-1 antibody. There was a decrease in tumor volume either alone with SG-3-05021 or in combination with anti-PD1 (FIG. 37). Mice survived a longer time (days) following treatment with either the SG-3-05021 either alone or with anti-PD-1 antibody therapy compared to mice treated with phosphate buffered saline (PBS) or anti-PD-1 antibody therapy alone (FIG. 38).

### Example 15. Identifying Human Targets of SG-3-00802 and SG-3-05021

Activity assays for CXCR4, CXCR3, and CCR7 were performed in agonist and antagonist mode for both SG-3-00802 and SG-3-05021 peptides. The addition of SG-3-00802 and SG-3-05021 of CXCR3 in the presence of its innate ligand CXCL11 enhanced binding, or sensitized CXCR3 (FIG. 39A-39B). SG-3-00802 and SG-3-05021 may allow for a mechanism to enhance CXCR3 activation already shown to be a critical pathway in numerous publications as mediating anti-tumor treatment.

### Example 16. Identifying Additional Human Targets of SG-3-00802 and SG-3-05021

The PathHunter® β-Arrestin Assay monitored activation of a panel of 168 G-protein coupled receptors (GPCRs) with fluorescent activation of the GPCR in agonist and antagonist mode.

$$\% {Activity}_{( Agonist mode )} = 100 * ( \frac{Mean {RLU}_{( test sample )} - Mean {RLU}_{( vehicle control )}}{Mean {MAX}_{( c o n trol ligand )} - Mean {RLU}_{( v e h icle control )}} ) \\ \% {Inhibition}_{( A n t a g o nist mode )} = 100 * ( 1 - \frac{Mean {RLU}_{( test sample )} - Mean {RLU}_{( v e h icle control )}}{Mean {RLU}_{( EC 80 control ligand )} - Mean {RLU}_{( v e h icle control )}} )$$

where the test sample is the peptide, the vehicle control=DMSO (0% activity), and the control ligand=control compound (100% activity).

Peptides SG-3-00802 and SG-3-05021 were identified as an agonist, an antagonist, a PAM (Positive Allosteric Modulator), or an inverse-agonist. Agonist, antagonist, PAM, and inverse-agonist were identified according to the guidelines in Table 18. The basal activity/noise is a % activity of −20% to 20% for both agonist and antagonist modes. A PAM (Positive Allosteric Modulator) binds to a receptor, at a different site than the agonist, to change the receptor's response to the agonist. An inverse-Agonist is a binding partner with agonistic shutting down of basal activity in the cell. An antagonist antagonizes the activity of a particular binding partner.

TABLE 18
GPCR assay interpretation guideline

| % Agonist Activity | % Antagonist Activity | Interpretation |
|---|---|---|
| >25-30% | basal activity/noise | Agonist |
| basal activity/noise | >50% | Antagonist |
| >25-30% | −% (see below for exceptions) | Confirmation of |
| | | positive agonist |
| >25-30% | −% (larger negative | PAM |
| | number = more likely) | |
| basal activity/noise | ~−30% | PAM |
| basal activity/noise | −70.8 | strong PAM |
| −25 to −30%-−80% | >50% | Inverse-Agonist |

CXCR3 and CXCR4 (DualSystems and Thermo SelectScreen assays) confirmed as hits for SG-3-00802. Six additional putative targets for SG-3-00802 were identified as were eleven putative targets for SG-3-05021 (see Tables 19-22).

TABLE 19
GPCR target assay results.

| | | |
|---|---|---|
| GPCR | SG-3-00802 | SG-3-05021 |

| | | |
|---|---|---|
| ADRA2A | | Agonist |
| ADRB2 | | PAM^{A} |
| CCR6 | | PAM |
| CCR9 | PAM | |
| CHRM5 | Agonist | PAM |
| CXCR3 | PAM | |

| | |
|---|---|
| CXCR4 | Antagonist |

| | |
|---|---|
| EDG6 | PAM |

| | |
|---|---|
| HCRTR2 | Antagonist |

| | | |
|---|---|---|
| HRH4 | | PAM |
| MRGPRX2 | Agonist | |
| MTNR1A | | PAM |
| NPFFR1 | | PAM |

| | | |
|---|---|---|
| SSTR1 | Inverse-agonist | Inverse-agonist and PAM |

| | |
|---|---|
| SSTR3 | PAM |
| TRHR | PAM |

| | |
|---|---|
| TSHR(L) | Antagonist |

^{A}PAM = positive allosteric modulator

TABLE 20
SG-3-00802 target assay results.

| | SG-3-00802 | | | |
|---|---|---|---|---|
| | gpcrMAX | | SelectScreen | |
| | | Antagonist | | Antagonist |
| Gene | Agonist Mode | Mode | Agonist Mode | Mode |
| CCR7 | 0% | 0% | No | NA |
| CXCR3 | −4% | −32% | No | Sensitizer |
| CXCR4 | −14% | 99% | No | Yes |

TABLE 21
SG-3-00802 assay results.

| | | % Antagonist | |
|---|---|---|---|
| Gene | % Agonist Activity | Activity | Interpretation |
| CCR9 | 2% | −48% | PAM |
| CHRM5 | 28% | 11% | Agonist? |
| CXCR3 | −4% | −32% | PAM |
| CXCR4 | −14% | 99% | Antagonist |
| HCRTR2 | 0% | 67% | Antagonist |
| MRGPRX2 | 66% | −9% | Agonist |
| SSTR1 | −137% | 61% | Inverse-Agonist |
| TSHR(L) | 3% | 77% | Antagonist |

TABLE 22
SG-3-05021 target assay results

| | SG-3-05021 | | | |
|---|---|---|---|---|
| | gpcrMAX | | SelectScreen | |
| | Agonist | Antagonist | Agonist | Antagonist |
| Gene | Mode | Mode | Mode | Mode |
| CCR7 | 0% | −18% | No | NA |
| CXCR3 | −4% | −20% | No | Sensitizer |
| | | | | (<potent than 802) |
| CXCR4 | 1% | 10% | No | NO |

### Example 17. Fecal Microbiome Based Composite Biomarker can Predicts Response to Immune Checkpoint Inhibitors in Metastatic Melanoma

The gut microbiome is emerging as an important source of biomarkers predictive of response to immune checkpoint inhibitor (ICI) therapy. ICI therapy targets inhibitory receptors on T cells to re-invigorate anti-tumor immune response. Only small percentage of patients are long-term responders to the therapy. ICI therapy is thought to alter systemic immune function via local changes within the gut mucosa and gut-associated lymphoid tissue. The interaction of PAMPs with APCs and innate effectors via TLRs can help prime an adaptive immune response. Cytokines and microbial metabolites produced locally can act systemically. Diversity and composition of the gut microbiome is emerging as having an influences on response to ICI therapy.

Several publications have demonstrated differences in microbiome composition of patients who respond to ICI therapy compared to patients who do not respond. (See, for example, Gopalakrishnan et al., Science (2018); Matson et al., Science (2018); Peters et al., Genome Medicine (2019); and Frankel et al., Neoplasia, (2017)). There is a lack of concordance between studies for differentiating strains for a variety of factors. Possible reasons include: 1) differences in data processing and the annotation databases used, 2) the variation in microbiome composition because of patient region, diet, sex, and additional factors, and 3) large study cohorts are needed capture all of the variability that may be present in the population.

Also, previous studies often implicated increased alpha-diversity along with major bacterial phyla associated with clinical responses. In contrast, the gut microbiota of non-responding patients seemed to be less diverse, with certain genera being prominent in non-responding patients. To account for these difference, the raw data was obtained from different published studies by passing it through the proprietary pipeline, create taxonomic and functional features using BioCyC and KEGG.

A meta-cohort with public and proprietary data was created and state of the art machine learning methods were applied to the compiled dataset. Cohorts include melanoma or pan-cancer patients undergoing checkpoint inhibitor treatment. Stool samples in these studies were collected from patients prior to start of checkpoint inhibitor therapy. Patients were excluded if: a patient's tumors were surgically resected prior to start of therapy and the type of checkpoint inhibitor was switched during course of treatment. Patients preferably had response data at six months post-start of checkpoint inhibitor therapy. Response data at three months post-start of checkpoint inhibitor therapy was used when six month response was unavailable. Six studies were collated for a total of 164 patients (Table 23, FIG. 40A-40B).

TABLE 23
Characteristics of the geographically diverse melanoma
meta-cohort for microbial biomarker discovery (N = 164)

| Cohort | Endpoint | Recruitment Site | N |
|---|---|---|---|
| Ahn et al, Genome | PFS > 6 months | New York, USA | 16 |
| Medicine (2019) | classified as R; | | |
| | PFS < 6 months as NR. | | |
| Frankel et al, | RECIST 1.1 | Texas, USA | 38 |
| Neoplasia (2017) | assessed 6 months | | |
| Gajewski et al, | after start of ICI | Illinois, USA | 38 |
| Science (2018) | treatment. | | |
| Levesque et al, | Complete or partial | Zurich, Switzerland | 17 |
| assembled herein | response classified | | |
| Rios et al, | as R; Stable or | California, USA | 11 |
| assembled herein | progressive disease | | |
| Wargo et al, Science | classified as NR. | Texas, USA | 23 |
| (2018) | | | |

Machine learning-based exploration of microbiome features across patient subpopulations enabled identification of biomarkers predictive of ICI response. The MTG data was systematically re-processed from each study. Functional features generated by mapping sequences against KEGG/BioCyc, and taxonomic features against the StrainSelect database [publicly available]. A total 96 models were trained (FIG. 41).

The biomarker model predicted response to ICI in metastatic melanoma using a six-feature microbial signature. The microbial signature was reduced down to six features, with the model retaining a high classification accuracy (AUC=0.90) (FIG. 42). The model classified response to all ICI therapy groups in metastatic melanoma, with the inclusion of partial responders in the responder group.

A composite biomarker was identified based on total information gain over all splits and was stable across cohorts. The six-feature model was re-trained leaving one cohort out at a time and its accuracy measured for each left out cohort. Results showed that this composite biomarker is able to predict ICI response in each individual cohort (FIG. 43). Functional features indicated that certain reactions or pathways may be important for ICI therapy outcome rather than particular strains (FIG. 44, Table 24). A mix of functional and taxonomic features are selected for prediction of CPI response. Most functional features were detected in >75% of the samples (FIG. 45). Strain feature (Faecalibacterium prausnitzii, t_179363) showed higher prevalence in responders in many cohorts.

TABLE 24
Biomarker model features.

| | Feature | | |
|---|---|---|---|
| ID | Type | Database ID | Description |
| Acinetobacter | Functional - | TRANS- | Membrane sugar transporter |
| Tetrose transporter | BioCyC | RXN19WO-4 | (Acinetobacter sp.) |
| dgoD | Functional - | K01684 | Galactonate dehydratase |
| | KEGG | | |
| GraR | Functional - | K19078 | Response regulator protein, part of |
| | KEGG | | Cationic antimicrobial peptide |
| | | | (CAMP) resistance. Host defense |
| | | | against invasive bacterial infection. |
| B- | Functional - | BETA- | Uracil metabolism |
| ureidopropionase | BioCyC | UREIDOPROPIONASE- | |
| reaction | | RXN | |
| Faecalibacterium | Taxonomic - | t_179363 | faecalibacterium.prausnitzii.apc942.8.14.2 |
| prausnitzii | strain | | |
| trans-2-enoyl-CoA | Functional - | RXN-12558 | Pyruvate fermentation to butanol |
| reductase | BioCyC | | |

The composite biomarker model was validated in an independent cohort. Based on validation AUC of 0.68, this model outperforms reported AUCs for established biomarkers in melanoma (Tumor Mutational Burden (TMB), AUC=0.602; T-cell Inflamed Gene Expression Profile (GEP), AUC=0.638) (FIG. 46). (See, for example, Cristescu R, et al. Genomic biomarkers will help to elucidate which cancer patients will benefit from PD-1 blockade immunotherapy. Science. 2018; 362(6411)).

Meta-analysis is a powerful method in the context of microbial biomarker discovery, where there is substantial variability in microbiome composition. Careful curation and standardization of the clinical data from individual cohorts is crucial to this end. This stool-based biomarker provides a robust and non-invasive method to guide therapeutic strategies for patients being considered for immune checkpoint inhibitors.

What is claimed is:

1. A method for identifying a subject as having a decreased likelihood of positively responding to treatment with an immunomodulator, the method comprising:
identifying a subject having a sample that has one or more of:
(i) a decreased level of the expression of dgoD, graR, or both relative to the same in a reference sample;
(ii) a decreased level of activity of a trans-2-enoyl-CoA reductase, an Acinetobacter tetrose transporter, or both relative to the same in a reference sample;
(iii) a decreased flux through the B-ureidopropionase reaction relative to the same in a reference sample;
as having a decreased likelihood of having a positive response to treatment with an immunomodulator.

2. The method of claim 1, wherein the method further comprises identifying the subject having a sample that has an increased level of one or more bacterial species selected from the group consisting of: Clostridium clostridioforme, Prevotella sp., Streptococcus parasanguinis, Anaerostipes hadrus, Parasutterella excrementihominis, and Eisenbergiella massiliensis relative to the same in a reference sample.

3. The method of claim 1, wherein the method further comprises identifying the subject having a sample that has a decreased level of one or more bacterial species selected from the group consisting of: Bifidobacterium sp., Collinsella sp., Methanobrevibacter smithii, Oscillibacter sp., Faecalibacterium prausnitzii C, Faecalibacterium prausnitzii I, Intestinimonas timonensis, Faecalibacterium prausnitzii, Bacteroides caccae, Barnesiella intestinihominis, Clostridiaceae bacterium, Clostridium sp., and Bifidobacterium adolescentis relative to the same in a reference sample

4. A method for identifying a subject as having an increased likelihood of having a positive response to treatment with an immunomodulator, the method comprising:
identifying a subject having a sample that has one or more of:
(i) an increased level of the expression of dgoD, graR, or both relative to the same in a reference sample;
(ii) an increased level of activity of a trans-2-enoyl-CoA reductase, an Acinetobacter tetrose transporter, or both relative to the same in a reference sample;
(iii) an increased flux through the B-ureidopropionase reaction relative to the same in a reference sample;
as having an increased likelihood of having a positive response to treatment with an immunomodulator.

5. The method of claim 4, wherein the method further comprises identifying the subject having a sample that has a decreased level of one or more bacterial species selected from the group consisting of: Clostridium clostridioforme, Prevotella sp., Streptococcus parasanguinis, Anaerostipes hadrus, Parasutterella excrementihominis, and Eisenbergiella massiliensis relative to the same in a reference sample.

6. The method of claim 4, wherein the method further comprises identifying the subject having a sample that has an increased level of one or more bacterial species selected from the group consisting of: Bifidobacterium sp., Collinsella sp., Methanobrevibacter smithii, Oscillibacter sp., Faecalibacterium prausnitzii C, Faecalibacterium prausnitzii I, Intestinimonas timonensis, Faecalibacterium prausnitzii, Bacteroides caccae, Barnesiella intestinihominis, Clostridiaceae bacterium, Clostridium sp., and Bifidobacterium adolescentis relative to the same in a reference sample

7. The method of any one of claims 1-6, wherein the immunomodulator is an immune checkpoint inhibitor selected from the group consisting of: ipilimumab, nivolumab, pembrolizumab, atezolizumab, avelumab, durvalumab, cemiplimab, and a combination thereof.

8. The method of any one of claims 1-7, wherein the immunomodulator is a co-stimulatory immune checkpoint agent selected from the group consisting of: IBI101, utomilumab, MEDI1873, and a combination thereof.

9. The method of any one of claims 1-8, wherein the cell therapy is a CAR T cell therapy.

10. The method of any one of claims 1-9, wherein the immunomodulator targets one or more of: CTLA-4, PD-1, PD-L1, BTLA, LAG-3, A2AR, TIM-3, B7-H3, VISTA, IDO, OX40, 4-1BB, and GITR.

11. A peptide comprising an amino acid sequence having at least 60% sequence identity to SEQ ID NO: 1.

12. The peptide of claim 11, wherein the peptide comprises an amino acid sequence having at least 70%, at least 80%, at least 85%, at least 90%, at least 95%, or at least 99% sequence identity to SEQ ID NO: 1.

13. The peptide of claim 11, wherein the peptide comprises the amino acid sequence of SEQ ID NO: 1.

14. A peptide comprising the amino acid sequence of SEQ ID NO: 1, or a variant thereof comprising one to 15 amino acid substitutions.

15. The peptide of any one of claims 11-14, wherein the methionine at position at 1 of SEQ ID NO:1 is substituted with an amino acid selected from the group consisting of: W, F, V, P, K, R, and S.

16. The peptide of any one of claims 11-15, wherein the leucine at position at 2 of SEQ ID NO:1 is substituted with an amino acid selected from the group consisting of: S, P, G, T, V, A, K, Q, R, W, Y, F, and N.

17. The peptide of any one of claims 11-16, wherein the serine at position at 3 of SEQ ID NO:1 is substituted with an amino acid selected from the group consisting of: Q and R.

18. The peptide of any one of claims 11-17, wherein the threonine at position at 4 of SEQ ID NO:1 is substituted with an amino acid selected from the group consisting of: G, A, and R.

19. The peptide of any one of claims 11-18, wherein the lysine at position at 5 of SEQ ID NO:1 is substituted with an amino acid selected from the group consisting of: A and R.

20. The peptide of any one of claims 11-19, wherein the lysine at position at 6 of SEQ ID NO:1 is substituted with an amino acid selected from the group consisting of: R, T, and A.

21. The peptide of any one of claims 11-20, wherein the threonine at position at 7 of SEQ ID NO:1 is substituted with an amino acid selected from the group consisting of: G, K, and R.

22. The peptide of any one of claims 11-21, wherein the threonine at position at 9 of SEQ ID NO:1 is substituted with an amino acid selected from the group consisting of: W and R.

23. The peptide of any one of claims 11-22, wherein the histidine at position at 10 of SEQ ID NO:1 is substituted with an amino acid selected from the group consisting of: K and R.

24. The peptide of any one of claims 11-23, wherein the aspartic acid at position at 11 of SEQ ID NO:1 is substituted with an amino acid selected from the group consisting of: F, G, H, I, K, P, R, T, V, W, and Y.

25. The peptide of any one of claims 11-24, wherein the histidine at position at 12 of SEQ ID NO:1 is substituted with an amino acid selected from the group consisting of: K and R.

26. The peptide of any one of claims 11-25, wherein the tyrosine at position at 13 of SEQ ID NO:1 is substituted with an amino acid selected from the group consisting of: W, N, G, K, R, and W.

27. The peptide of any one of claims 11-26, wherein the proline at position at 14 of SEQ ID NO:1 is substituted with an amino acid selected from the group consisting of: G and W.

28. The peptide of any one of claims 11-27, wherein the serine at position at 15 of SEQ ID NO:1 is substituted with an amino acid selected from the group consisting of: G and R.

29. The peptide of any one of claims 11-28, wherein the methionine at position at 18 of SEQ ID NO:1 is substituted with an amino acid selected from the group consisting of: W, H, Y, G, and R.

30. The peptide of any one of claims 11-29, wherein the aspartic acid at position at 20 of SEQ ID NO:1 is substituted with an amino acid selected from the group consisting of: P and R.

31. The peptide of any one of claims 11-30, wherein the proline at position at 21 of SEQ ID NO:1 is F.

32. The peptide of any one of claims 11-31, wherein the glycine at position at 22 of SEQ ID NO:1 is substituted with an amino acid selected from the group consisting of: P, K, and W.

33. The peptide of any one of claims 11-32, wherein the aspartic acid at position at 25 of SEQ ID NO:1 is substituted with an amino acid selected from the group consisting of: P and R.

34. The peptide of any one of claims 11-33, wherein the arginine at position at 27 of SEQ ID NO:1 is W.

35. The peptide of any one of claims 11-34, wherein the alanine at position at 28 of SEQ ID NO:1 is substituted with an amino acid selected from the group consisting of: F, G, V, Y, and W.

36. The peptide of any one of claims 11-35, wherein the serine at position at 29 of SEQ ID NO:1 is substituted with R.

37. The peptide of any one of claims 11-36, wherein the peptide comprises an amino acid sequence selected from the group consisting of: SEQ ID NOs: 2-35.

38. The peptide of any one of claims 11-37, wherein the peptide comprises an amino acid sequence selected from the group consisting of: SEQ ID NOs: 36-93.

39. The peptide of any one of claims 11-38, wherein the peptide comprises an amino acid sequence selected from the group consisting of: SEQ ID NOs: 94-114.

40. A peptide comprising the amino acid sequence set forth in, X₁X₂SX₄AKX₇KX₈HDHX₁₂X₁₃X₁₄GRX₁₅RX₁₆PX₁₈WHDWX₂₀X₂₁X₂₂ (SEQ ID NO:115), wherein each of X₁-X₂₂ is independently selected from any naturally occurring amino acid.

41. The peptide of claim 180, wherein X₁ is an amino acid selected from the group consisting of: M, W, F, V, and P.

42. The peptide of any one of claims 40-41, wherein X₂ is an amino acid selected from the group consisting of: L, S, P, G, T, V, and A.

43. The peptide of any one of claims 40-42, wherein X₄ is an amino acid selected from the group consisting of: T, G, and A.

44. The peptide of any one of claims 40-43, wherein X₇ is an amino acid selected from the group consisting of: G and T.

45. The peptide of any one of claims 40-44, wherein X₈ is an amino acid selected from the group consisting of: T and W.

46. The peptide of any one of claims 40-45, wherein X₁₂ is an amino acid selected from the group consisting of: Y, W, and N.

47. The peptide of any one of claims 40-46, wherein X₁₃ is an amino acid selected from the group consisting of: P, G, and W.

48. The peptide of any one of claims 40-47, wherein X₁₄ is an amino acid selected from the group consisting of: S and G.

49. The peptide of any one of claims 40-48, wherein X₁₅ is an amino acid selected from the group consisting of: M, W, H, and Y.

50. The peptide of any one of claims 40-49, wherein X₁₆ is an amino acid selected from the group consisting of: D and P.

51. The peptide of any one of claims 40-50, wherein X₁₈ is an amino acid selected from the group consisting of: G, P, and K.

52. The peptide of any one of claims 40-51, wherein X₂₀ is an amino acid selected from the group consisting of: R and W.

53. The peptide of any one of claims 40-52, wherein X₂₁ is an amino acid selected from the group consisting of: A, F, G, and V.

54. The peptide of any one of claims 40-53, wherein X₂₂ is an amino acid selected from the group consisting of: R and W.

55. The peptide of any one of claims 11-54, wherein the peptide increases activity of a CD2 protein, a BST2 protein, or a TNF protein.

56. The peptide of any one of claims 11-54, wherein the peptide binds to a CD2 protein, a BST2 protein, or a TNF protein.

57. A peptide comprising an amino acid sequence having at least 60% sequence identity to SEQ ID NO: 117 or SEQ ID NO: 162.

58. The peptide of claim 57, wherein the peptide comprises an amino acid sequence having at least 70%, at least 80%, at least 85%, at least 90%, at least 95%, or at least 99% sequence identity to SEQ ID NO: 117 or SEQ ID NO: 162.

59. The peptide of claim 57, wherein the peptide comprises the amino acid sequence of SEQ ID NO: 117 or SEQ ID NO: 162.

60. The peptide of any one of claims 57-59, wherein the peptide comprises an amino acid sequence selected from the group consisting of: SEQ ID NOs: 117-160.

61. A peptide comprising the amino acid sequence set forth in, X₁KX₃X₄X₅SVKX₉X₁₀CX₁₂X₁₃CX₁₄X₁₅X₁₆IXBRX₂₀GX₂₂X₂₃X₂₄X₂₅IX₂₇X₂₈X₂₉PX₃₁HKQX₃₅QX₃₇ (SEQ ID NO:161), wherein X₁ is optional, each of X₂-X₂₅ and X₂₈-X₃₅ is independently a naturally occurring amino acid, X₂₇ is selected from the group consisting of C and CP; and X₃₇ is selected from the group consisting of: G, GN, and DRH.

62. The peptide of claim 61, wherein X₁ is the amino acid M.

63. The peptide of any one of claims 61-62, wherein X₃ is an amino acid selected from the group consisting of: V, I, and T.

64. The peptide of any one of claims 61-63, wherein X₄ is an amino acid selected from the group consisting of: R, K, and Q.

65. The peptide of any one of claims 61-64, wherein X₅ is an amino acid selected from the group consisting of: P, S, and A.

66. The peptide of any one of claims 61-65, wherein X₉ is an amino acid selected from the group consisting of: P, T, and K.

67. The peptide of any one of claims 61-66, wherein X₁₀ is an amino acid selected from the group consisting of: M and I.

68. The peptide of any one of claims 61-67, wherein X₁₂ is an amino acid selected from the group consisting of: E and D.

69. The peptide of any one of claims 61-68, wherein X₁₃ is an amino acid selected from the group consisting of: K and Y.

70. The peptide of any one of claims 61-69, wherein X₁₅ is an amino acid selected from the group consisting of: K and R.

71. The peptide of any one of claims 61-70, wherein X₁₆ is an amino acid selected from the group consisting of: V and I.

72. The peptide of any one of claims 61-71, wherein X₁₈ is an amino acid selected from the group consisting of: K and R.

73. The peptide of any one of claims 61-72, wherein X₂₀ is an amino acid selected from the group consisting of: K, N, and H.

74. The peptide of any one of claims 61-73, wherein X₂₂ is an amino acid selected from the group consisting of: R, K, H, S, and I.

75. The peptide of any one of claims 61-74, wherein X₂₃ is an amino acid selected from the group consisting of: V and I.

76. The peptide of any one of claims 61-75, wherein X₂₄ is an amino acid selected from the group consisting of: M, R, A, and L.

77. The peptide of any one of claims 61-76, wherein X₂₅ is an amino acid selected from the group consisting of: V and I.

78. The peptide of any one of claims 61-77, wherein X₂₈ is selected from the group consisting of: E, Q, A, and T.

79. The peptide of any one of claims 61-78, wherein X₂₉ is an amino acid selected from the group consisting of: N and E.

80. The peptide of any one of claims 61-79, wherein X₃₁ is an amino acid selected from the group consisting of: K and R.

81. The peptide of any one of claims 61-80, wherein X₃₅ is an amino acid selected from the group consisting of: K and R.

82. The peptide of any one of claims 57-81, wherein the peptide modulates activity of a CCR9 protein, a CHRM5 protein, a CXCR3 protein, a CXCR4 protein, a HCRTR2 protein, a MRGPRX2 protein, a SSTR1 protein, or a TSHR(L) protein.

83. The peptide of any one of claims 57-81, wherein the peptide binds to a CCR9 protein, a CHRM5 protein, a CXCR3 protein, a CXCR4 protein, a HCRTR2 protein, a MRGPRX2 protein, a SSTR1 protein, or a TSHR(L) protein.

84. A recombinant host cell comprising an exogenous polynucleotide, the polynucleotide encoding a peptide of any one of claims 11-83.

85. The recombinant host cell of claim 84, wherein the exogenous polynucleotide further encodes a host cell specific signal sequence.

86. The recombinant host cell of claim 84 or claim 85, wherein the exogenous polynucleotide further encodes a heterologous promoter.

87. The recombinant host cell of claim 86, wherein the heterologous promoter is a constitutive promoter.

88. The recombinant host cell of claim 86, wherein the heterologous promoter is an inducible promoter.

89. The recombinant host cell of any one of claims 84-88, wherein the host cell is a prokaryotic cell, a eukaryotic cell, or a fungal cell.

90. The recombinant host cell of claim 89, wherein the host cell is selected from the group consisting of: an Escherichia coli cell, a Lactococcus lactis cell, a Streptomyces coelicolor cell, a Streptomyces lividans cell, a Streptomyces albus cell, a Streptomyces venezuelae cell, or a Bacillus subtilis cell.

91. The recombinant host cell of claim 89, wherein the host cell is a Saccharomyces cerevisiae cell, a Pichia pastoris cell, a Yarrowia lipolytica cell, an Aspergillus niger cell, or a Hansenula polymorpha cell.

92. The recombinant host cell of claim 89, wherein the host cell is a Chinese Hamster Ovary cell.

93. A pharmaceutical composition comprising:
(a) a peptide of any one of claims 11-83; or a plurality of recombinant host cells of any one of claims 84-92; and
(b) a pharmaceutically acceptable carrier.

94. The pharmaceutical composition of claim 93, wherein the pharmaceutical composition further comprises a therapeutically effective amount of a bacterial species selected from the group consisting of: Bifidobacterium sp., Collinsella sp., Methanobrevibacter smithii, Oscillibacter sp., Barnesiella intestinihominis, Faecalibacterium prausnitzii C, Faecalibacterium prausnitzii I, Ruminococcaceae bacterium, Intestinimonas timonensis, Faecalibacterium prausnitzii, Bacteroides caccae, Clostridiaceae bacterium, Clostridium sp., Bifidobacterium adolescentis, and a combination thereof.

95. The pharmaceutical composition of claim 93 or claim 94, wherein the pharmaceutical composition is formulated for oral administration.

96. A nucleic acid construct comprising a polynucleotide, wherein the polynucleotide encodes a peptide of any one of claims 11-83.

97. A method of producing a peptide, the method comprising culturing the recombinant host cell of any one of claims 84-92, under conditions sufficient for expression of the encoded peptide.

98. A method for treating a disease in a subject in need thereof, the method comprising administering to the subject:
(a) a peptide of any one of claims 11-83;
(b) a recombinant host of any one of claims 84-92;
(c) a pharmaceutical composition of any one of claim 93-95; or
(d) a nucleic acid construct of claim 96.

99. The method of claim 98, wherein the peptide modulates the production of at least one cytokine in the subject.

100. The method of claim 98 or claim 99, wherein the cytokine is selected from the group consisting of TNF-α, IL-17, IL-1β, IL-2, IFN-γ, IL-6, IL-12, IL-25, IL-33, IL-8, MCP-1, MIP-3α, CXCL1, IL-23, IL-4, IL-10, IL-13, IFN-α, and TGF-β.

101. The method of any one of claims 98-100, wherein the peptide induces the production of at least one pro-inflammatory cytokine in the subject.

102. The method of claim 101, wherein the at least one pro-inflammatory cytokine is selected from the group consisting of TNF-α, IL-17, IL-1β, IL-2, IFN-γ, IL-6, IL-12, IL-25, IL-33, IL-8, MCP-1, MIP-3α, CXCL1, and IL-23.

103. The method of any one of claims 98-100, wherein the peptide suppresses the production of at least one anti-inflammatory cytokine in the subject.

104. The method of claim 103, wherein the at least one anti-inflammatory cytokine is selected from the group consisting of IL-4, IL-10, IL-13, IFN-α, and TGF-β.

105. The method of any one of claims 98-104, wherein the peptide increases Th1 activation in the subject.

106. The method of any one of claims 98-105, wherein the peptide increases dendritic cell maturation in the subject.

107. The method of any one of claims 98-106, wherein the peptide increases CD70 expression in the subject.

108. The method of any one of claims 98-107, wherein the peptide increases the clonal expansion of T_{eff} in the subject.

109. The method of any one of claims 98-108, wherein the peptide increases activity of a CD2 protein, a BST2 protein, or a TNF protein.

110. The method of any one of claims 98-108, wherein the peptide increases activity of a CXCL3 protein.

111. The method of any one of claims 98-108, wherein the peptide binds to a CD2 protein, a BST2 protein, or a TNF protein.

112. The method of any one of claims 98-108, wherein the peptide binds to a CXCL3 protein.

113. The method of any one of claims 98-112, wherein the disease is a neoplasm.

114. The method of any one of claims 98-113, wherein the disease is cancer.

115. The method of any one of claims 98-114, wherein the disease is at least one selected from the group consisting of: basal cell carcinoma, biliary tract cancer, bladder cancer, bone cancer, brain and central nervous system cancer, breast cancer, cervical cancer, choriocarcinoma, colon and rectum cancer, connective tissue cancer, cancer of the digestive system, endometrial cancer, esophageal cancer, eye cancer, cancer of the head and neck, gastric cancer, intra-epithelial neoplasm, kidney cancer, larynx cancer, leukemia, liver cancer, small-cell lung cancer, non-small-cell lung cancer, Hodgkin's lymphoma, non-Hodgkins lymphoma, melanoma, myeloma, neuroblastoma, oral cavity cancer, ovarian cancer, pancreatic cancer, prostate cancer, retinoblastoma, rhabdomyosarcoma, rectal cancer, renal cancer, cancer of the respiratory system, sarcoma, skin cancer, stomach cancer, testicular cancer, thyroid cancer, uterine cancer, and cancer of the urinary system.

116. The method of claim 115, further comprising administering a treatment for cancer.

117. A method of treating cancer in a subject, the method comprising:
(a) identifying a subject having a sample that has one or more of:
(i) a decreased level of the expression of dgoD, graR, or both relative to the same in a reference sample;
(ii) a decreased level of activity of a trans-2-enoyl-CoA reductase, an Acinetobacter tetrose transporter, or both relative to the same in a reference sample;
(iii) a decreased flux through the B-ureidopropionase reaction relative to the same in a reference sample;
(iv) an increased level of one or more bacterial species selected from the group consisting of: Clostridium clostridioforme, Prevotella sp., Streptococcus parasanguinis, Anaerostipes hadrus, Parasutterella excrementihominis, and Eisenbergiella massiliensis relative to the same in a reference sample; and
(v) a decreased level of one or more bacterial species selected from the group consisting of: Bifidobacterium sp., Collinsella sp., Methanobrevibacter smithii, Oscillibacter sp., Faecalibacterium prausnitzii C, Faecalibacterium prausnitzii I, Intestinimonas timonensis, Faecalibacterium prausnitzii, Bacteroides caccae, Barnesiella intestinihominis, Clostridiaceae bacterium, Clostridium sp., and Bifidobacterium adolescentis relative to the same in a reference sample; and
(b) administering a therapy to the identified subject, the therapy comprising a peptide of any one of claims 11-83 or a pharmaceutical composition thereof, or a recombinant host of any one of claims 84-91.

118. A method of treating cancer in a subject, the method comprising administering to a subject identified as having one or more of:
(i) a decreased level of the expression of dgoD, graR, or both relative to the same in a reference sample;
(ii) a decreased level of activity of a trans-2-enoyl-CoA reductase, an Acinetobacter tetrose transporter, or both relative to the same in a reference sample;
(iii) a decreased flux through the B-ureidopropionase reaction relative to the same in a reference sample;
(iv) an increased level of one or more bacterial species selected from the group consisting of: Clostridium clostridioforme, Prevotella sp., Streptococcus parasanguinis, Anaerostipes hadrus, Parasutterella excrementihominis, and Eisenbergiella massiliensis relative to the same in a reference sample; and
(v) a decreased level of one or more bacterial species selected from the group consisting of: Bifidobacterium sp., Collinsella sp., Methanobrevibacter smithii, Oscillibacter sp., Faecalibacterium prausnitzii C, Faecalibacterium prausnitzii I, Intestinimonas timonensis, Faecalibacterium prausnitzii, Bacteroides caccae, Barnesiella intestinihominis, Clostridiaceae bacterium, Clostridium sp., and Bifidobacterium adolescentis relative to the same in a reference sample
a therapy comprising a peptide of any one of claims 11-83 or a pharmaceutical composition thereof, or a recombinant host of any one of claims 84-91.

119. A method of treating a cancer in a subject that has previously received one or more doses of an immunomodulator, wherein the method comprises administering to a subject identified as having one or more of:
(i) a decreased level of the expression of dgoD, graR, or both relative to the same in a reference sample;
(ii) a decreased level of activity of a trans-2-enoyl-CoA reductase, an Acinetobacter tetrose transporter, or both relative to the same in a reference sample;
(iii) a decreased flux through the B-ureidopropionase reaction relative to the same in a reference sample;
(iv) an increased level of one or more bacterial species selected from the group consisting of: Clostridium clostridioforme, Prevotella sp., Streptococcus parasanguinis, Anaerostipes hadrus, Parasutterella excrementihominis, and Eisenbergiella massiliensis relative to the same in a reference sample; and
(v) a decreased level of one or more bacterial species selected from the group consisting of: Bifidobacterium sp., Collinsella sp., Methanobrevibacter smithii, Oscillibacter sp., Faecalibacterium prausnitzii C, Faecalibacterium prausnitzii I, Intestinimonas timonensis, Faecalibacterium prausnitzii, Bacteroides caccae, Barnesiella intestinihominis, Clostridiaceae bacterium, Clostridium sp., and Bifidobacterium adolescentis relative to the same in a reference sample
a therapy comprising a peptide of any one of claims 11-83 or a pharmaceutical composition thereof, or a recombinant host of any one of claims 84-91.

120. A method of treating cancer in a subject, the method comprising:
(a) administering to the subject one or more doses of an immunomodulator for a period of time;
(b) after (a), determining if a sample obtained from the subject has one or more of:
(i) a decreased level of the expression of dgoD, graR, or both relative to the same in a reference sample;
(ii) a decreased level of activity of a trans-2-enoyl-CoA reductase, an Acinetobacter tetrose transporter, or both relative to the same in a reference sample;
(iii) a decreased flux through the B-ureidopropionase reaction relative to the same in a reference sample;
(iv) an increased level of one or more bacterial species selected from the group consisting of: Clostridium clostridioforme, Prevotella sp., Streptococcus parasanguinis, Anaerostipes hadrus, Parasutterella excrementihominis, and Eisenbergiella massiliensis relative to the same in a reference sample; and
(v) a decreased level of one or more bacterial species selected from the group consisting of: Bifidobacterium sp., Collinsella sp., Methanobrevibacter smithii, Oscillibacter sp., Faecalibacterium prausnitzii C, Faecalibacterium prausnitzii I, Intestinimonas timonensis, Faecalibacterium prausnitzii, Bacteroides caccae, Barnesiella intestinihominis, Clostridiaceae bacterium, Clostridium sp., and Bifidobacterium adolescentis relative to the same in a reference sample; and
(c) administering a therapy to the identified subject, the therapy comprising a peptide of any one of claims 11-83 or a pharmaceutical composition thereof, or a recombinant host of any one of claims 84-91.

121. The method of claim 12, further comprising administering a treatment for cancer.

122. A method of treating cancer in a subject, the method comprising:
(a) administering to the subject one or more doses of an immunomodulator for a period of time;
(b) after (a), identifying a subject having a sample that has one or more of:
(i) an increased level of the expression of dgoD, graR, or both relative to the same in a reference sample;
(ii) an increased level of activity of a trans-2-enoyl-CoA reductase, an Acinetobacter tetrose transporter, or both relative to the same in a reference sample;
(iii) an increased flux through the B-ureidopropionase reaction relative to the same in a reference sample;
(iv) a decreased level of one or more bacterial species selected from the group consisting of: Clostridium clostridioforme, Prevotella sp., Streptococcus parasanguinis, Anaerostipes hadrus, Parasutterella excrementihominis, and Eisenbergiella massiliensis relative to the same in a reference sample; and
(v) an increased level of one or more bacterial species selected from the group consisting of: Bifidobacterium sp., Collinsella sp., Methanobrevibacter smithii, Oscillibacter sp., Faecalibacterium prausnitzii C, Faecalibacterium prausnitzii I, Intestinimonas timonensis, Faecalibacterium prausnitzii, Bacteroides caccae, Barnesiella intestinihominis, Clostridiaceae bacterium, Clostridium sp., and Bifidobacterium adolescentis relative to the same in a reference sample; and
(c) administering a therapy to the identified subject, the therapy comprising a peptide of any one of claims 11-83 or a pharmaceutical composition thereof, or a recombinant host of any one of claims 84-91.

123. A method of treating cancer in a subject, the method comprising administering to a subject identified as having one or more of:
(i) an increased level of the expression of dgoD, graR, or both relative to the same in a reference sample;
(ii) an increased level of activity of a trans-2-enoyl-CoA reductase, an Acinetobacter tetrose transporter, or both relative to the same in a reference sample;
(iii) an increased flux through the B-ureidopropionase reaction relative to the same in a reference sample;
(iv) a decreased level of one or more bacterial species selected from the group consisting of: Clostridium clostridioforme, Prevotella sp., Streptococcus parasanguinis, Anaerostipes hadrus, Parasutterella excrementihominis, and Eisenbergiella massiliensis relative to the same in a reference sample; and
(v) an increased level of one or more bacterial species selected from the group consisting of: Bifidobacterium sp., Collinsella sp., Methanobrevibacter smithii, Oscillibacter sp., Faecalibacterium prausnitzii C, Faecalibacterium prausnitzii I, Intestinimonas timonensis, Faecalibacterium prausnitzii, Bacteroides caccae, Barnesiella intestinihominis, Clostridiaceae bacterium, Clostridium sp., and Bifidobacterium adolescentis relative to the same in a reference sample
a therapy comprising a peptide of any one of claims 11-83 or a pharmaceutical composition thereof, or a recombinant host of any one of claims 84-91;
wherein the subject has received a therapeutically effective amount of an immunomodulator.

124. The method of any of claims 117-123, the therapy further comprising one or more of:
a) a therapeutically effective amount of an immunomodulator;
b) an effective amount of one or more bacterial species selected from the group consisting of: Bifidobacterium sp., Collinsella sp., Methanobrevibacter smithii, Oscillibacter sp., Faecalibacterium prausnitzii C, Faecalibacterium prausnitzii I, Ruminococcaceae bacterium, Intestinimonas timonensis, Faecalibacterium prausnitzii, Bacteroides caccae, Barnesiella intestinihominis, Clostridiaceae bacterium, Ruminococcaceae bacterium, Clostridium sp., and Bifidobacterium adolescentis; and
c) an additional treatment of cancer excluding an immunomodulator.

125. A method of modulating the activity of one or more target proteins in a subject, the method comprising administering to the subject a peptide of any one of claims 11-83; or a plurality of recombinant host cells of any one of claims 84-92; wherein the one or more target protein is selected from the group consisting of a CD2 protein, a BST2 protein, a TNF protein, a CXCL3 protein, a ADRA2A protein, a ADRB2 protein, a CCR6 protein, a CCR9 protein, a CHRM5 protein, a CXCR3 protein, a CXCR4 protein, a EDGE protein, a HCRTR2 protein, a HRH4 protein, a MRGPRX2 protein, a MTNR1A protein, a NPFFR1 protein, a SSTR1 protein, a SSTR3 protein, a TRHR protein, and a TSHR(L) protein.

126. The method of claim 125, wherein the one or more target proteins is selected form the group consisting of a CD2 protein, a BST2 protein, and a TNF protein.

127. The method of any one of claims 125-126, wherein the one or more target proteins is a CXCL3 protein.

128. The method of any one of claims 125-127, wherein the one or more target proteins is selected from the group consisting of a CCR9 protein, a CHRM5 protein, a CXCR3 protein, a CXCR4 protein, a HCRTR2 protein, a MRGPRX2 protein, a SSTR1 protein, and a TSHR(L) protein.

129. The peptide of any one of claims 125-129, wherein the one or more target proteins is selected from the group consisting of a CCR9 protein, a CHRM5 protein, a CXCR3 protein, a CXCR4 protein, a HCRTR2 protein, a MRGPRX2 protein, a SSTR1 protein, or a TSHR(L) protein.

130. The method of any one of claims 117-129, wherein the method further comprises detecting the level of one or more bacterial species, RNA transcripts, protein activity, or flux though a metabolic pathway in a sample from the subject.

131. A method for increasing the response to an immunomodulator in a subject in need thereof comprising administering to the subject a composition comprising a peptide of any one of claims 11-83 or a pharmaceutical composition thereof, or a recombinant host of any one of claims 84-91.

132. The method of claim 131, wherein the subject has cancer.

133. A method for treating cancer in a subject, the method comprising:
(a) detecting a dysbiosis associated with response to therapy with an immunomodulator in a sample from the subject; and
(b) administering to the subject a composition comprising a peptide of any one of claims 11-83 or a pharmaceutical composition thereof, or a recombinant host of any one of claims 84-91.

134. The method of any one of claim 1-10, 117, 120-122, or 130-133, wherein the sample is a fecal sample.

135. The method of claim 134, wherein the sample is a tumor biopsy sample.

136. The method of any one of claims 133-135, wherein detecting the dysbiosis associated with response to therapy with an immunomodulator comprises determining bacterial gene expression in the sample from the subject.

137. The method of any one of claims 133-136, wherein detecting the dysbiosis associated with response to therapy with an immunomodulator comprises determining bacterial composition in the sample from the subject.

138. The method of any one of claims 133-137, wherein detecting the dysbiosis associated with response to therapy with an immunomodulator comprises determining bacterial protein activity in the sample from the subject.

139. The method of any one of claim 1-10, 119-122, 124, 130-132, or 136-138, wherein the immunomodulator targets one or more of: CTLA-4, PD-1, PD-L1, BTLA, LAG-3, A2AR, TIM-3, B7-H3, VISTA, IDO, OX40, 4-1BB, and GITR.

140. The method of any one of claims 98-139, wherein the subject has a solid tumor.

141. The method of claim 140, wherein the subject has a solid tumor selected from the group consisting of: melanoma, lung cancer, kidney cancer, bladder cancer, a head and neck cancer, Merkel cell carcinoma, urothelial cancer, breast cancer, glioblastoma, gastric cancer, a nasopharyngeal neoplasm, colorectal cancer, hepatocellular carcinoma, ovarian cancer, and pancreatic cancer.

142. The method of any one of claims 98-141, wherein the subject has a hematological malignancy.

143. The method of claim 142, wherein the subject has a hematological malignancy selected from the group consisting of: multiple myeloma, non-Hodgkin lymphoma, Hodgkin lymphoma, diffuse large B-cell lymphoma, and chronic lymphocytic leukemia/small lymphocytic lymphoma (CLL/SLL).

144. The method of any one of claims 98-143, wherein the subject has a cancer selected from one or more of: melanoma, non-small cell lung cancer (NSCLC), small cell lung cancer, squamous cell lung carcinoma, kidney cancer, bladder cancer, a head and neck cancer, Hodgkin lymphoma, Merkel cell carcinoma, urothelial cancer, breast cancer, glioblastoma, gastric adenocarcinoma, transitional cell carcinoma, a biliary tract neoplasm, a nasopharyngeal neoplasm, colorectal cancer, hepatocellular carcinoma, renal cell carcinoma, ovarian cancer, and pancreatic cancer.

145. The method of any of claim 115, 116, or 141-144, wherein the melanoma is unresectable or metastatic melanoma.

146. The method of any one of claims 133-145, wherein the method comprises administering the composition to the subject once, twice, or three times per day.

147. The method of any one of claims 133-146, wherein the composition is formulated for oral administration.

148. The method of any one of claims 133-147, wherein the composition is formulated as a tablet, a capsule, a powder, or a liquid.

149. The method of any one of claims 133-148, wherein the composition is formulated as a tablet.

150. The method of claim 149, wherein the tablet is coated.

151. The method of claim 150, wherein the coating comprises an enteric coating.

152. The method of any one of claims 133-152, wherein the composition is formulated for rectal administration.

153. The method of any one of claims 133-152, wherein the composition is formulated for intravenous administration.

154. The method of any one of claims 133-152, wherein the composition is formulated for intratumoral administration.

155. The method of any one of claims 133-152, wherein the method further comprises administering a treatment for cancer, an additional treatment for cancer, and/or other adjunct therapy to the subject.

156. The method of claim 155, wherein the composition comprising the bacterial strain treatment and the treatment for cancer and/or adjunct therapy are administered simultaneously.

157. The method of claim 156, wherein the composition comprising the bacterial strain treatment and the treatment for cancer and/or adjunct therapy are administered sequentially.

158. The method of any one of claims 156-157, wherein the treatment for cancer and/or adjunct therapy comprises a probiotic.

159. The method of any one of claims 116, 121, 156-159, wherein the treatment for cancer and/or adjunct therapy comprises surgery, radiation therapy, or a combination thereof.

160. The method of any one of claims 116, 121, 155-149, wherein the treatment for cancer and/or adjunct therapy comprises a therapeutic agent.

161. The method of claim 160, wherein the therapeutic agent comprises a chemotherapeutic agent, a targeted therapy, an immunotherapy, or a combination thereof.

162. The method of claim 161, wherein the chemotherapeutic agent comprises carboplatin, cisplatin, gemcitabine, methotrexate, paclitaxel, pemetrexed, lomustine, temozolomide, dacarbazine, or a combination thereof.

163. The method of claim 161 or claim 162 wherein the targeted therapy comprises afatinib dimaleate, bevacizumab, cetuximab, crizotinib, erlotinib, gefitinib, sorafenib, sunitinib, pazopanib, everolimus, dabrafenib, aldesleukin, interferon alfa-2b, ipilimumab, peginterferon alfa-2b, trametinib, vemurafenib, or a combination thereof.

164. The method of claim 161-163, wherein the immunotherapy comprises a cell therapy, a therapy with an immunomodulator, or a combination thereof.

165. The method of claim 164, wherein the immunomodulator is an immune checkpoint inhibitor selected from the group consisting of: ipilimumab, nivolumab, pembrolizumab, atezolizumab, avelumab, durvalumab, cemiplimab, and a combination thereof.

166. The method of claim 165, wherein the immunomodulator is a co-stimulatory immune checkpoint agent selected from the group consisting of: IBI101, utomilumab, MEDI1873, and a combination thereof.

167. The method of claim 164, wherein the cell therapy is a CART cell therapy

168. The method of any one of claims 98-167, wherein the subject is a human.
